(19) United States
(12) Patent Application Publication
(10) Pub. No.: US 2026/0110002 A1
(43) Pub. Date: Apr. 23, 2026

(54) Differential Knockout of An Allele of A Heterozygous Apolipoprotein A1 (APO1A) Gene

(71) Applicant: EmendoBio Inc., Wilmington, DE (US)

(72) Inventors: David BARAM, Tel Aviv (IL); Lior IZHAR, Tel Aviv (IL); Asael HERMAN, Ness-Ziona (IL); Rafi EMMANUEL, Ramla (IL); Michal GOLAN-MASHIACH, Ness-Ziona (IL); Joseph GEORGESON, Rehovot (IL)

(21) Appl. No.: 19/368,562

(22) Filed: Oct. 24, 2025

Related U.S. Application Data
(63) Continuation of application No. 17/041,661, now Pat. No. 12,454,705.
(60) Provisional application No. 62/647,745, filed on Mar. 25, 2018.

(51) Int. Cl. C12N 15/90; A61K 35/13; C12N 15/11

(52) U.S. Cl. CPC C12N 15/907; A61K 35/13; C12N 15/11; C12N 2310/20; C12N 2800/80

(57) ABSTRACT

RNA molecules comprising a guide sequence portion having 17-20 nucleotides in the sequence of 17-20 contiguous nucleotides set forth in any one of SEQ ID Nos: 1-1313 and compositions, methods, and uses thereof.

This application claims the benefit of U.S. patent application Ser. No. 17/041,661, filed Sep. 25, 2020, which is a § 371 national stage of PCT/US2019/023715, filed Mar. 22, 2019, which in turn claims the benefit of U.S. Prov. App. No. 62/647,745, filed Mar. 25, 2018, the contents of each of which are hereby incorporated by reference.

Throughout this application, various publications are referenced; including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences in the filed named “5798_0018US02_KO_APOAL.xml”, which is 1,665,065 bytes in size, and which was created on Oct. 23, 2025, in the IBM-PC machine format, having an operating system compatibility with MS-Windows, which is contained in the xml file filed Oct. 24, 2025, as part of this application.

### BACKGROUND OF INVENTION

There are several classes of DNA variation in the human genome, including insertions and deletions, differences in the copy number of repeated sequences, and single nucleotide polymorphisms (SNPs). A SNP is a DNA sequence variation occurring when a single nucleotide (adenine (A), thymine (T), cytosine (C), or guanine (G)) in the genome differs between human subjects or paired chromosomes in an individual. Over the years, the different types of DNA variations have been the focus of the research community either as markers in studies to pinpoint traits or disease causation or as potential causes of genetic disorders.

A genetic disorder is caused by one or more abnormalities in the genome. Genetic disorders may be regarded as either “dominant” or “recessive.” Recessive genetic disorders are those which require two copies (i.e., two alleles) of the abnormal/defective gene to be present. In contrast, a dominant genetic disorder involves a gene or genes which exhibit(s) dominance over a normal (functional/healthy) gene or genes. As such, in dominant genetic disorders only a single copy (i.e., allele) of an abnormal gene is required to cause or contribute to the symptoms of a particular genetic disorder. Such mutations include, for example, gain-of-function mutations in which the altered gene product possesses a new molecular function or a new pattern of gene expression. Other examples include dominant negative mutations, which have a gene product that acts antagonistically to the wild-type allele.

### Amyloidosis

Amyloidosis is a protein mis-folding disorder, in which normally soluble proteins undergo conformational changes and are deposited in the extracellular space as abnormal insoluble fibrils that progressively disrupt tissue structure and function. apoA-1 is a plasma protein of 28 kDa synthesized by the liver and the small intestine. ApoA-1 is the main protein of high-density lipoprotein particles and important for the formation and metabolism of high-density lipoprotein cholesterol esters. Mature apoA-1 consist of 243 amino acids encoded by exons 3 and 4 of the APOA1 gene. Mutations in the APOA1 gene were shown to be associated with apoA-1 related amyloidosis (AApoA1) which is an autosomal dominant disease that may cause, inter alia, hereditary renal amyloidosis.

### SUMMARY OF THE INVENTION

Disclosed is an approach for knocking out the expression of a dominant-mutant allele by disrupting the dominant-mutant allele or degrading the resulting mRNA.

The present disclosure provides a method for utilizing at least one naturally occurring heterozygous nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutant allele”), and the other allele encoding for a functional protein (“functional allele”).

As used herein, the term “heterozygous single nucleotide polymorphism” or “SNP” refers to a single nucleotide position in a genome that differs between paired chromosomes within a population. As used herein the most common or most prevalent nucleotide base at the position is referred to as the reference (REF), wild-type (WT), common, or major form. Less prevalent nucleotide bases at the position are referred to as the alternative (ALT), minor, rare, or variant forms.

Embodiments of the present invention provide methods for utilizing at least one heterozygous SNP in a gene expressing a dominant mutant allele in a given cell or subject. In embodiments of the present invention, the SNP utilized may or may not be associated with a disease phenotype. In embodiments of the present invention, an RNA molecule comprising a guide sequence targets only the mutant allele of the gene by targeting the nucleotide base present at a heterozygous SNP in the mutant allele of the gene and therefore having a different nucleotide base in the functional allele of the gene.

In some embodiments, the method further comprises the step of knocking out expression of the mutated protein and allowing expression of the functional protein.

According to embodiments of the present invention, there is provided a first RNA molecule comprising a guide sequence portion having 17-20 nucleotides in the sequence of 17-20 contiguous nucleotides set forth in any one of SEQ ID NOs: 1-1313.

According to embodiments of the present invention, there is provided a first RNA molecule comprising a guide sequence portion having 17-20 contiguous nucleotides set forth in any one of SEQ ID NOs: 1-1313.

According to some embodiments of the present invention, there is provided a composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided a method for inactivating a mutant APOA1 allele in a cell, the method comprising delivering to the cell a composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided a method for treating apoA-1 related amyloidosis, the method comprising delivering to a subject having apoA-1 related amyloidosis a composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided use of a composition comprising an RNA molecule comprising a guide sequence portion having 17-20 nucleotides in the sequence of 17-20 contiguous nucleotides set forth in any one of SEQ ID NOS: 1-1313 and a CRISPR nuclease for inactivating a mutant APOA1 allele in a cell, comprising delivering to the cell the composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to embodiments of the present invention, there is provided a medicament comprising an RNA molecule comprising a guide sequence portion having 17-20 nucleotides in the sequence of 17-20 contiguous nucleotides set forth in any one of SEQ ID NOs: 1-1313 and a CRISPR nuclease for use in inactivating a mutant APOA1 allele in a cell, wherein the medicament is administered by delivering to the cell the composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided use of a composition comprising an RNA molecule comprising a guide sequence portion having 17-20 nucleotides in the sequence of 17-20 contiguous nucleotides set forth in any one of SEQ ID NOs: 1-1313 and a CRISPR nuclease for treating ameliorating or preventing apoA-1 related amyloidosis, comprising delivering to a subject having or at risk of having apoA-1 related amyloidosis the composition of comprising an RNA molecule comprising a guide sequence portion having 17-20 nucleotides in the sequence of 17-20 contiguous nucleotides set forth in any one of SEQ ID NOS: 1-1313 and a CRISPR nuclease.

According to some embodiments of the present invention, there is provided a medicament comprising the composition comprising an RNA molecule comprising a guide sequence portion having 17-20 nucleotides in the sequence of 17-20 contiguous nucleotides set forth in any one of SEQ ID NOs: 1-1313 and a CRISPR nuclease for use in treating ameliorating or preventing apoA-1 related amyloidosis, wherein the medicament is administered by delivering to a subject having or at risk of having apoA-1 related amyloidosis the composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided a kit for inactivating a mutant APOA1 allele in a cell, comprising an RNA molecule comprising a guide sequence portion having 17-20 nucleotides in the sequence of 17-20 contiguous nucleotides set forth in any one of SEQ ID NOs: 1-1313, a CRISPR nuclease, and/or a tracrRNA molecule; and instructions for delivering the RNA molecule; CRISPR nuclease, and/or the tracrRNA to the cell.

According to some embodiments of the present invention, there is provided a kit for treating apoA-1 related amyloidosis in a subject, comprising an RNA molecule comprising a guide sequence portion having 17-20 nucleotides in the sequence of 17-20 contiguous nucleotides set forth in any one of SEQ ID NOs: 1-1313, a CRISPR nuclease, and/or a tracrRNA molecule; and instructions for delivering the RNA molecule; CRISPR nuclease, and/or the tracrRNA to a subject having or at risk of having apoA-1 related amyloidosis.

According to some embodiments of the present invention, there are provided cells modified by the RNA molecules, compositions, or methods of the present invention.

According to some embodiments of the present invention, there are provided cells modified by the RNA molecules, compositions, or methods of the present invention for use in treating apoA-1 related amyloidosis in a subject having or at risk of having apoA-1 related amyloidosis.

According to some embodiments of the present invention, there is provided a medicament comprising the modified cells of the present invention for treating apoA-1 related amyloidosis in a subject having or at risk of having apoA-1 related amyloidosis.

According to some embodiments of the present invention, there is provided a method for inactivating a mutant allele of the APOA1 gene in a cell, the method comprising the steps of:
a) selecting a cell with an APOA1 gene mutation associated with in apoA-1 related amyloidosis and which cell is heterozygous at one or more polymorphic sites in the APOA1 gene selected from: rs11216158, rs1 1216157, rs2727784, rs613808, rs4018880, rs1374117, rs670, rs5069, rs5070, rs2070665, rs5072, rs7116797, rs5076;
b) introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
thereby inactivating only the mutant allele of the APOA1 gene in the cell.

According to embodiments of the present invention, there is provided a method for inactivating a mutant allele of the APOA1 gene in a cell, the method comprising the steps of:
a) selecting a cell with an APOA1 gene mutation associated with an apoA-1 related amyloidosis and which cell is heterozygous at one or more polymorphic sites in the APOA1 gene selected from: rs11216158, rs11216157, rs2727784, rs613808, rs4018880, rs1374117, rs670, rs5069, rs5070, rs2070665, rs5072, rs7116797, rs5076, rs28931574;
b) introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
and wherein the method further comprises introduction of a second RNA molecule comprising a guide sequence portion capable of complexing with a CRISPR nuclease, wherein the complex of second RNA molecule and the CRISPR nuclease affects a second double strand break in the APOA1gene;
thereby inactivating only the mutant allele of the APOA1 gene in the cell.

According to embodiments of the present invention, there is provided a method for inactivating a mutant allele of the APOA1 gene in a cell with an APOA1 gene mutation associated with apoA-1 related amyloidosis and which cell is heterozygous at one or more polymorphic sites in the APOA1 gene selected from: rs11216158, rs11216157, rs2727784, rs613808, rs4018880, rs1374117, rs670, rs5069, rs5070, rs2070665, rs5072, rs7116797, rs5076, the method comprising
introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
thereby inactivating only the mutant allele of the APOA1 gene in the cell.

According to embodiments of the present invention, there is provided a method for inactivating a mutant allele of the APOA1 gene in a cell with an APOA1 gene mutation associated with apoA-1 related amyloidosis and heterozygous at one or more polymorphic sites in the APOA1 gene selected from: rs11216158, rs1 1216157, rs2727784, rs613808, rs4018880, rs1374117, rs670, rs5069, rs5070, rs2070665, rs5072, rs7116797, rs5076, rs28931574, the method comprising:
introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
and wherein the method further comprises introduction of a second RNA molecule comprising a guide sequence portion capable of complexing with a CRISPR nuclease, wherein the complex of the second RNA molecule and CRISPR nuclease affects a second double strand break in the APOA1gene;
thereby inactivating only the mutant allele of the APOA1 gene in the cell.

### BRIEF DESCRIPTION OF THE DRAWINGS

FIG. 1: Removal of, inter alia, exon 2 of the APOA1 gene which encodes at least a portion of the signal peptide (residues 1-18) may result in a protein that will not be secreted or alternatively RNA decay may be triggered resulting in knockout of the expression of the mutant allele. For example, utilization of two guide sequences, one targeting a heterozygous SNP present in the APOA1 gene and located at exon 1 and the other targeting a heterozygous SNP present on the APOA1 gene located at intron 2, wherein each RNA molecule targets the nucleotide base, REF or ALT, of each SNP present in only the mutant allele of the APOA1 gene to remove at least a portion of exon 1 which encodes the 5′ UTR and exon 2.

FIG. 2: Utilization of two guide sequences, one targeting intron 2 of the APOA1 gene and the other targeting intron 3 of the APOA1 gene, to remove exon 3 of the APOA1 gene, which encodes a region prone to amyloidosis, to form a truncated apoA-1 which will not form aggregates/deposition as fibrils, or alternatively RNA decay may be triggered resulting in knockout of the expression of the mutant allele, wherein at least one of the guide sequences targets a heterozygous SNP present in the APOA1 gene wherein the RNA molecule targets the nucleotide base, REF or ALT, of the SNP present in only the mutant allele of the APOA1 gene.

FIG. 3: Utilization of two guide sequences to remove exon 2, intron 2, and exons 3 of the APOA1 gene may result in the formation of a truncated apoA-1 which optionally will not secrete from the cells and/or form aggregates/deposit as fibrils, or alternatively RNA decay may be triggered resulting in knockout of the expression of the mutant allele. For example, utilization of a first guide sequence targeting a sequence located at exon 1 of the APOA1 gene and a second guide sequence targeting a sequence of intron of the APOA1 gene, wherein at least one of the guide sequences targets a heterozygous SNP present in the APOA1 gene wherein the RNA molecule targets the nucleotide base, REF or ALT, of the SNP present in only the mutant allele of the APOA1 gene.

FIG. 4: Removal of, inter alia, exon 2 of the APOA1 gene which encodes at least a portion of the signal peptide (residues 1-18) may result in a protein that will not be secreted or alternatively RNA decay may be triggered resulting in knockout of the expression of the mutant allele. For example, utilization of two guide sequences, one targeting a sequence of intron 1 of the APOA1 gene and the other targeting a sequence of intron 2 of the APOA1 gene, to remove at least a portion of exon 1 which encodes the 5′ UTR and exon 2, wherein at least one of the guide sequences targets a heterozygous SNP present in the APOA1 gene wherein the RNA molecule targets the nucleotide base, REF or ALT, of the SNP present in only the mutant allele of the APOA1 gene.

FIG. 5: Utilization of two guide sequences to remove exon 2, intron 2, and exons 3 of the APOA1 may result in the formation of a truncated apoA-1 which optionally will not secrete from the cells and/or form aggregates/deposit as fibrils, or alternatively RNA decay may be triggered resulting in knockout of the expression of the mutant allele. For example, utilization of two guide sequences, one targeting a sequence of intron 1 of the APOA1 gene and the other targeting a sequence of intron 3 of the APOA1 gene, wherein at least one of the guide sequences targets a heterozygous SNP present in the APOA1 gene wherein the RNA molecule targets the nucleotide base, REF or ALT, of the SNP present in only the mutant allele of the APOA1 gene.

FIG. 6: Utilization of two guide sequences to remove exon 2 or exons 2 and 3 of the APOA1 gene by targeting rs670 in exon 1 or rs5069 in intron 1 of the mutant allele of APOA1 gene and a non-coding sequence in intron 2 of the mutant allele of APOA1 gene or common to both alleles of the APOA1 gene.

FIG. 7: Utilization of two guide sequences to remove exon 2, exon 3, or exons 2 and 3 of the APOA1 gene by targeting a non-coding sequence in exon 1 (5′ UTR), intron 1 or intron 3 of the mutant allele of APOA1 gene or common to both alleles of the APOA1 gene and rs5070 of intron 2 of the mutant allele of APOA1 gene.

FIG. 8: Utilization of two guide sequences to remove exons 1-3, exons 2 and 3, or exon 3 of the APOA1 gene by targeting a non-coding sequence in exon 1 (5′ UTR), intron 1, or intron 2 of the mutant allele of APOA1 gene or common to both alleles of the APOA1 gene and one of rs2070665, rs5072, rs5076, or rs7116797 in intron 9 of the mutant allele of APOA1 gene.

FIG. 9: Utilization of two guide sequences to remove exons 1 and 2, or exons 1-3 of the APOA1 gene by targeting a non-coding sequence in intron 2 or intron 3 of the mutant allele of APOA1 gene or common to both alleles of the APOA1 gene and one of rs11216158, rs11216157, rs2727784, rs613808, rs4018880, or rs1374117 upstream of intron 2 or intron 3 on the mutant allele of APOA1 gene.

FIG. 10: Utilization of two guide sequences to remove exons 1 and 2, or exons 2 or 3 of the APOA1 gene by targeting a non-coding sequence in exon 1 (5′ UTR), intron 1, intron 2 or intron 3 of the mutant allele of APOA1 gene or common to both alleles of the APOA1 gene and rs28931574 in exon 3 of the mutant allele of APOA1 gene.

FIG. 11: Seven SNPs located on the APOA1 gene and total heterozygosity in the population from the selected SNPs.

FIG. 12: Thirteen SNPs located on the APOA1 gene and total heterozygosity in the population from the selected SNPs.

FIG. 13: Two exemplary strategies are proposed to tackle AAopA1 with spCas9 at a genomic DNA level with two guide sequences. The first strategy involves targeting exon 1 or intron 1 with a first guide sequence and intron 3 with a second guide sequence in order to excise exons 1, exon2, and exon 3 of the mutant APOA1 gene. The second strategy involves targeting intron 2 with a first guide sequence and intron 3 with a second guide sequence in order to remove exon 3 of the mutant APOA1 gene.

FIG. 14: 20 different guide sequences were screened for high on target activity using spCas9 in HeLa cells. In brief, spCas9 coding plasmid (390 ng) was co-transfected with each of the guide sequence expression plasmids (120 ng) in 24 well plate format using Turbofect reagent (Thermo fisher scientific). Cells were harvested 72 h post DNA transfection. On target activity was determined by capillary electroporation analysis. The graph represents the average STDV of 2 independent experiments.

### DETAILED DESCRIPTION

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or”′ rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The “guide sequence portion” of an RNA molecule refers to a nucleotide sequence that is capable of hybridizing to a specific target DNA sequence, e.g., the guide sequence portion has a nucleotide sequence which is fully complementary to said target DNA sequence. In some embodiments, the guide sequence portion is 17, 18, 19, 20, 21, 22, 23, or 24 nucleotides in length, or approximately 17-24, 18-22, 19-22, 18-20, or 17-20 nucleotides in length. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence.

In embodiments of the present invention, an RNA molecule comprises a guide sequence portion having 17-20 nucleotides in the sequence of 17-20 contiguous nucleotides set forth in any one of SEQ ID NOs: 1-1313, or SEQ ID NOs: 1-269, or SEQ ID NOs: 270-1056, or SEQ ID NOs: 1057-1102, or SEQ ID NOs: 1103-1313, or SEQ ID NOs: 1-269 and SEQ ID NOs: 1057-1102, or SEQ ID NOs 270-1056 and SEQ ID NOs: 1103-1313. It is understood that in any of the embodiments of the present invention the guide sequence portion of an RNA molecule may comprise 17-20 contiguous nucleotides set forth in any single sequence of SEQ ID NOs: 1-1313, or in any single sequence from the following groups of sequences SEQ ID NOs: 1-269, or SEQ ID NOs: 270-1056, or, or SEQ ID NOs: 1057-1102, or SEQ ID NOs: 1103-1313, or SEQ ID NOs: 1-269 and SEQ ID NOs: 1057-1102, or SEQ ID NOs 270-1056 and SEQ ID NOs: 1103-1313, or SEQ ID NOs: 1-1313.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 20 nucleotides in length and consists of 20 nucleotides in the sequence of 20 contiguous nucleotides set forth in any one of SEQ ID NOs: 1-1313. In embodiments of the present invention, the guide sequence portion may be less than 20 nucleotides in length. For example, in embodiments of the present invention the guide sequence portion may be 17, 18, or 19 nucleotides in length. In such embodiments the guide sequence portion may consist of 17, 18, or 19 nucleotides, respectively, in the sequence of 17-20 contiguous nucleotides set forth in any one of SEQ ID NOs: 1-1313. For example, a guide sequence portion having 17 nucleotides in the sequence of 17 contiguous nucleotides set forth in SEQ ID NO: 1 may consist of any one of the following nucleotide sequences (nucleotides excluded from the contiguous sequence are marked in strike-through):

SEQ ID NO: 1
AAAGCUGCAGGCUCCGCAAG
17 nucleotide guide sequence 1: [symbol]
GCUGCAGGCUCCGCAAG
17 nucleotide guide sequence 2: [symbol]
AGCUGCAGGCUCCGCAA [symbol]
17 nucleotide guide sequence 3: [symbol]
AAGCUGCAGGCUCCGCA [symbol]
17 nucleotide guide sequence 4:
AAAGCUGCAGGCUCCGC

[symbol]

In embodiments of the present invention, the guide sequence portion may be greater than 20 nucleotides in length. For example, in embodiments of the present invention the guide sequence portion may be 21, 22, 23, or 24 nucleotides in length. In such embodiments the guide sequence portion comprises 20 nucleotides in the sequence of 20 contiguous nucleotides set forth in any one of SEQ ID NOs: 1-1313 and additional nucleotides fully complimentary to a nucleotide or sequence of nucleotides adjacent to the 3′ end of the target sequence, 5′ end of the target sequence, or both.

In embodiments of the present invention, a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule.

In embodiments of the present invention, the RNA molecule may further comprise the sequence of a tracrRNA molecule. Such embodiments may be designed as a synthetic fusion of the guide portion of the RNA molecule and the trans-activating crRNA (tracrRNA). (See Jinek (2012) Science). Embodiments of the present invention may also form CRISPR complexes utilizing a separate tracrRNA molecule and a separate RNA molecule comprising a guide sequence portion. In such embodiments the tracrRNA molecule may hybridize with the RNA molecule via base pairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

A skilled artisan will appreciate that in all of the embodiments of the present invention, each of the RNA molecules of the present invention are capable of complexing with a CRISPR nuclease such as to associate with a target genomic DNA sequence of interest next to a protospacer adjacent motif (PAM). The CRISPR nuclease then mediates cleavage of target DNA to create a double-stranded break within the protospacer. Accordingly, in embodiments of the present invention, the guide sequences and RNA molecules of the present invention may target a location 1, 2, 3, 4, 5, 6, 7, 8, 9, 10, 11, 12, 13, 14, 15, 16, 17, 18, 19, 20, 21, 22, 23, 24, 25, 26, 27, or 28 nucleotides upstream or downstream from a PAM site In embodiments of the present invention, the guide sequences and RNA molecules of the present invention may target a location that is within the PAM site.

Therefore, in embodiments of the present invention, the RNA molecules of the present invention may affect a double strand break in an allele of a gene 1, 2, 3, 4, 5, 6, 7, 8, 9, 10, 11, 12, 13, 14, 15, 16, 17, 18, 19, 20, 21, 22, 23, 24, 25, 26, 27, 28 upstream or downstream from a polymorphic site. In further embodiments of the invention, the polymorphic site is within the PAM site. A skilled artisan will appreciate that where a heterozygous polymorphic site is present, an RNA molecule may be designed to affect a double stranded break in only the REF or ALT nucleotide base of the heterozygous polymorphic site.

In embodiments of the present invention, an RNA molecule is designed to target a heterozygous polymorphic site present in the APOA1 gene, wherein the RNA molecule targets only the nucleotide base, REF or ALT, of the heterozygous polymorphic site present in only the mutant allele of the APOA1 gene

### EMBODIMENTS

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutant allele”), and the other allele encoding for a functional protein (“functional allele”). The method further comprises the step of knocking out expression of the mutated protein and allowing expression of the functional protein. In some embodiments, the method is for treating, ameliorating, or preventing a dominant negative genetic disorder.

Embodiments of the present invention provide methods for utilizing at least one heterozygous SNP in a gene expressing a dominant mutant allele in a given cell or subject. In embodiments of the present invention, the SNP utilized may or may not be associated with a disease phenotype. In embodiments of the present invention, an RNA molecule comprising a guide sequence targets only the mutant allele of the gene by targeting the nucleotide base present at a heterozygous SNP in the mutant allele of the gene and therefore having a different nucleotide base in the functional allele of the gene.

According to embodiments of the present invention, there is provided a first RNA molecule comprising a guide sequence portion having 17-20 nucleotides in the sequence of 17-20 contiguous nucleotides set forth in any one of SEQ ID NOs: 1-1313.

According to embodiments of the present invention, there is provided a first RNA molecule comprising a guide sequence portion having 17-20 contiguous nucleotides set forth in any one of SEQ ID NOs: 1-1313.

According to embodiments of the present invention, an RNA molecule may further comprise a portion having a sequence which binds to a CRISPR nuclease.

According to embodiments of the present invention, the sequence which binds to a CRISPR nuclease is a tracrRNA sequence.

According to embodiments of the present invention, an RNA molecule may further comprise a portion having a tracr mate sequence.

According to embodiments of the present invention, an RNA molecule may further comprise one or more linker portions.

According to embodiments of the present invention, an RNA molecule may be up to 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 200 up to 300 nucleotides in length, 100 to 200 nucleotides in length, or 150 up to 250 nucleotides in length. Each possibility represents a separate embodiment.

According to some embodiments of the present invention, there is provided a composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to embodiments of the present invention, the composition may comprise a second RNA molecule comprising a guide sequence portion.

According to embodiments of the present invention, the guide sequence portion of the second RNA molecule comprises 17-20 nucleotides in the sequence of 17-20 contiguous nucleotides set forth in any one of SEQ ID NOs: 1-1313.

According to embodiments of the present invention, the 17-20 nucleotides of the guide sequence portion of the second RNA molecule are in a different sequence from the sequence of the guide sequence portion of the first RNA molecule.

According to embodiments of the present invention, the 17-20 nucleotides of the guide sequence portion of the second RNA molecule, when complexed with a CRISPR nuclease, target a non-coding region of the APOA1 gene. In some embodiments, the non-coding regions selected from, exon 1, intron 1, intron 2, and intron 3.

Embodiments of the present invention may comprise a tracrRNA molecule.

According to some embodiments of the present invention, there is provided a method for inactivating a mutant APOA1 allele in a cell, the method comprising delivering to the cell a composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided a method for treating apoA-1 related amyloidosis, the method comprising delivering to a subject having apoA-1 related amyloidosis a composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to embodiments of the present invention, the composition comprises a second RNA molecule comprising a guide sequence portion having 17-20 nucleotides in the sequence of 17-20 contiguous nucleotides set forth in any one of SEQ ID NOs: 1-1313.

According to embodiments of the present invention, the 17-20 nucleotides of the guide sequence portion of the second RNA molecule are in a different sequence from the sequence of the guide sequence portion of the first RNA molecule

According to embodiments of the present invention, the CRISPR nuclease and the RNA molecule or RNA molecules are delivered to the subject and/or cells substantially at the same time or at different times.

According to embodiments of the present invention, the tracrRNA is delivered to the subject and/or cells substantially at the same time or at different times as the CRISPR nuclease and RNA molecule or RNA molecules.

According to embodiments of the present invention, the first RNA molecule targets a first heterozygous SNP present in an exon or promoter of the APOA1 gene wherein the first RNA molecule targets the nucleotide base, REF or ALT, of the first SNP present in only the mutant allele of the APOA1 gene, and wherein the second RNA molecule targets a second heterozygous SNP present in the same or a different exon or an intron of the APOA1 gene wherein the second RNA molecule targets the nucleotide base, REF or ALT, of the second SNP present in only the mutant allele of the APOA1 gene, or a the second RNA molecule targets a sequence in an intron present in both the mutant or functional allele.

According to embodiments of the present invention, the first RNA molecule or the first and the second RNA molecules target a heterozygous SNP present in the promoter region, the start codon, or the untranslated region (UTR) of the APOA1 gene wherein the RNA molecule targets the nucleotide base, REF or ALT, of the SNP present in only the mutant allele of the APOA1 gene.

According to embodiments of the present invention, the first RNA molecule or the first and the second RNA molecules targets at least a portion of the promoter and/or the start codon and/or a portion of the UTR of the mutant allele of the APOA1 gene.

According to embodiments of the present invention, the first RNA molecule targets a portion of the promoter, a first heterozygous SNP present in the promoter of the APOA1 gene, or a heterozygous SNP present upstream to the promoter of the APOA1 gene and the second RNA molecule targets a second heterozygous SNP, which is present in the APOA1 gene downstream of the first heterozygous SNP, and is in the promoter, in the UTR, or in an intron or in an exon of the APOA1 gene, wherein the first RNA molecule targets the nucleotide base, REF or ALT, of the first SNP present in only the mutant allele of the of the APOA1 gene, wherein the second RNA molecule targets the nucleotide base, REF or ALT, of the second SNP present in only the mutant allele of the APOA1 gene.

According to embodiments of the present invention, the first RNA molecule targets a heterozygous SNP present in the promoter, upstream of the promoter, or the UTR of a the APO1 gene wherein the RNA molecule targets the nucleotide base, REF or ALT, of the SNP present in only the mutant allele of the APOA1 gene and the second RNA molecule is designed to target a sequence which is present in an intron of both the mutant allele and the functional allele of the APOA1 gene.

According to embodiments of the present invention, the first RNA molecule targets a sequence upstream of the promotor which is present in both a mutant and functional allele of the APOA1 gene and the second RNA molecule targets a heterozygous SNP present in any location of the of the APOA1 gene wherein the second RNA molecule targets the nucleotide base, REF or ALT, of the SNP present in only the mutant allele of the APOA1 gene.

According to embodiments of the present invention, there is provided a method comprising removing an exon containing a disease-causing mutation from a mutant allele, wherein the first RNA molecule or the first and the second RNA molecules target regions flanking an entire exon or a portion of the exon.

According to embodiments of the present invention, there is provided a method comprising removing multiple exons, the entire open reading frame of a gene, or removing the entire gene.

According to embodiments of the present invention, the first RNA molecule targets a first heterozygous SNP present in an exon or promoter of the APOA1 gene, and wherein the second RNA molecule targets a second heterozygous SNP present in the same or a different exon or in an intron of the APOA1 gene wherein the second RNA molecule targets the nucleotide base, REF or ALT, of the second SNP present in only the mutant allele of the APOA1 gene, or the second RNA molecule targets a sequence in an intron present in both the mutant and functional allele of the APOA1 gene.

According to embodiments of the present invention, the first RNA molecule or the first and the second RNA molecules target an alternative splicing signal sequence between an exon and an intron of a mutant allele.

According to embodiments of the present invention, the second RNA molecule targets a sequence present in both a mutant allele and a functional allele of the APOA1 gene.

According to embodiments of the present invention, the second RNA molecule targets an intron.

According to embodiments of the present invention, there is provided a method comprising subjecting the mutant allele to insertion or deletion by an error prone non-homologous end joining (NHEJ) mechanism, generating a frameshift in the mutant allele's sequence.

According to embodiments of the present invention, the frameshift results in inactivation or knockout of the mutant allele.

According to embodiments of the present invention, the frameshift creates an early stop codon in the mutant allele.

According to embodiments of the present invention, the frameshift results in nonsense-mediated mRNA decay of the transcript of the mutant allele.

According to embodiments of the present invention, the inactivating or treating results in a truncated protein encoded by the mutant allele and a functional protein encoded by the functional allele.

According to some embodiments of the present invention, there is provided use of a composition comprising an RNA molecule comprising a guide sequence portion having 17-20 nucleotides in the sequence of 17-20 contiguous nucleotides set forth in any one of SEQ ID NOs: 1-1313 and a CRISPR nuclease inactivating a mutant APOA1 allele in a cell, comprising delivering to the cell the RNA molecule comprising a guide sequence portion having 17-20 nucleotides in the sequence of 17-20 contiguous nucleotides set forth in any one of SEQ ID NOs: 1-1313 and the CRISPR nuclease.

According to embodiments of the present invention, there is provided a medicament comprising an RNA molecule comprising a guide sequence portion having 17-20 nucleotides in the sequence of 17-20 contiguous nucleotides set forth in any one of SEQ ID NOs: 1-1313 and a CRISPR nuclease for use in inactivating a mutant APOA1 allele in a cell, wherein the medicament is administered by delivering to the cell the composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided use of a composition comprising an RNA molecule comprising a guide sequence portion having 17-20 nucleotides in the sequence of 17-20 contiguous nucleotides set forth in any one of SEQ ID NOs: 1-1313 and a CRISPR nuclease for treating ameliorating or preventing apoA-1 related amyloidosis, comprising delivering to a subject having or at risk of having apoA-1 related amyloidosis the composition of comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided a medicament comprising the composition comprising an RNA molecule comprising a guide sequence portion having 17-20 nucleotides in the sequence of 17-20 contiguous nucleotides set forth in any one of SEQ ID NOs: 1-1313 and a CRISPR nuclease for use in treating ameliorating or preventing apoA-1 related amyloidosis, wherein the medicament is administered by delivering to a subject having or at risk of having apoA-1 related amyloidosis: the composition comprising an RNA molecule comprising a guide sequence portion having 17-20 nucleotides in the sequence of 17-20 contiguous nucleotides set forth in any one of SEQ ID NOs: 1-1313 and a CRISPR nuclease.

According to some embodiments of the present invention, there is provided a kit for inactivating a mutant APOA1 allele in a cell, comprising an RNA molecule comprising a guide sequence portion having 17-20 nucleotides in the sequence of 17-20 contiguous nucleotides set forth in any one of SEQ ID NOs: 1-1313, a CRISPR nuclease, and/or a tracrRNA molecule; and instructions for delivering the RNA molecule; CRISPR nuclease, and/or the tracrRNA to the cell.

According to some embodiments of the present invention, there is provided a kit for treating apoA-1 related amyloidosis in a subject, comprising an RNA molecule comprising a guide sequence portion having 17-20 nucleotides in the sequence of 17-20 contiguous nucleotides set forth in any one of SEQ ID NOs: 1-1313, a CRISPR nuclease, and/or a tracrRNA molecule; and instructions for delivering the RNA molecule; CRISPR nuclease, and/or the tracrRNA to a subject having or at risk of having apoA-1 related amyloidosis.

In embodiments of the present invention, the RNA molecule comprises a guide sequence portion having 17-20 nucleotides in the sequence of 17-20 contiguous nucleotides set forth in any one of SEQ ID NOs: 1-269, or SEQ ID NOs: 270-1056, or SEQ ID NOs: 1057-1102, or SEQ ID NOs: 1103-1313, or SEQ ID NOs: 1-269 and SEQ ID NOs: 1057-1102, or SEQ ID NOs 270-1056 and SEQ ID NOs: 1103-1313, or SEQ ID NOs 1-1313. It is understood that in any of the embodiments of the present invention the guide sequence portion of an RNA molecule may comprise 17-20 contiguous nucleotides set forth in any single sequence of SEQ ID NOs: 1-1313, or in any single sequence from the following groups of sequences SEQ ID NOs: 1-269, SEQ ID NOs: 270-1056, or SEQ ID NOs: 1057-1102, or SEQ ID NOs: 1103-1313, or SEQ ID NOs: 1-269 and SEQ ID NOs: 1057-1102, or SEQ ID NOs 270-1056 and SEQ ID NOs: 1103-1313, or SEQ ID NOs: 1-1313.

The compositions and methods of the present disclosure may be utilized for treating, preventing, ameliorating, or slowing progression of amyloidosis, such as renal amyloidosis.

In some embodiments, a mutant allele is deactivated by delivering to a cell an RNA molecule which targets a heterozygous SNP present in the promoter region, the start codon, or the untranslated region (UTR) of the APOA1 gene wherein the RNA molecule targets the nucleotide base, REF or ALT, of the SNP present in only the mutant allele of the APOA1 gene.

In some embodiments, a mutant allele is inactivated by removing at least a portion of the promoter and/or removing the start codon and/or a portion of the UTR. In some embodiments, the method of deactivating a mutant allele comprises removing at least a portion of the promoter. In such embodiments one RNA molecule may be designed for targeting a first heterozygous SNP present in the promoter or upstream to the promoter of the APOA1 gene and another RNA molecule is designed to target a second heterozygous SNP, which is downstream of the first SNP, and is present in the promoter, in the UTR, or in an intron or in an exon of the APO1 gene. Alternatively, one RNA molecule may be designed for targeting a heterozygous SNP present in the promoter, or upstream of the promoter, or the UTR of the APOA1 gene and another RNA molecule is designed to target a sequence which is present in an intron of both the mutant allele and the functional allele of the APOA1 gene. Alternatively, one RNA molecule may be designed for targeting a sequence upstream of the promotor which is present in both the mutant and functional allele and the other guide is designed to target a heterozygous SNP present in any location of the APOA1 gene e.g., in an exon, intron, UTR, or downstream of the promoter of the APOA1 gene wherein the RNA molecule targets the nucleotide base, REF or ALT, of the SNP present in only the mutant allele of the APOA1 gene.

In some embodiments, the method of deactivating a mutant allele comprises an exon skipping step comprising removing an exon containing a disease-causing mutation from the mutant allele. Removing an exon containing a disease-causing mutation in the mutant allele requires two RNA molecules which target regions flanking the entire exon or a portion of the exon. Removal of an exon containing the disease-causing mutation may be designed to eliminate the disease-causing action of the protein while allowing for expression of the remaining protein product which retains some or all of the wild-type activity. As an alternative to single exon skipping, multiple exons, the entire open reading frame or the entire gene can be excised using two RNA molecules flanking the region desired to be excised.

In some embodiments, the method of deactivating a mutant allele comprises delivering two RNA molecules to a cell, wherein one RNA molecule targets a first heterozygous SNP present in an exon or promoter of the APOA1 gene wherein the RNA molecule targets the nucleotide base, REF or ALT, of the first SNP present in only the mutant allele of the APOA1 gene, and wherein the other RNA molecule targets a second heterozygous SNP present in the same or a different exon or in an intron of the APOA1 gene wherein the RNA molecule targets the nucleotide base, REF or ALT, of the second SNP present in only the mutant allele of the APOA1 gene, or the second RNA molecule targets a sequence in an intron present in both the mutant or functional allele.

In some embodiments, an RNA molecule is used to target a CRISPR nuclease to an alternative splicing signal sequence between an exon and an intron of a mutant allele, thereby destroying the alternative splicing signal sequence in the mutant allele.

Any one of, or combination of, the above-mentioned strategies for deactivating a mutant allele may be used in the context of the invention.

Additional strategies may be used to deactivate a mutant allele. For example, in embodiments of the present invention, an RNA molecule is used to direct a CRISPR nuclease to an exon or a splice site of a mutant allele in order to create a double-stranded break (DSB), leading to insertion or deletion of nucleotides by an error-prone non-homologous end-joining (NHEJ) mechanism and formation of a frameshift mutation in the mutant allele. The frameshift mutation may result in: (1) inactivation or knockout of the mutant allele by generation of an early stop codon in the mutant allele, resulting in generation of a truncated protein; or (2) nonsense mediated mRNA decay of the transcript of the mutant allele. In further embodiments, one RNA molecule is used to direct a CRISPR nuclease to a promotor of a mutant allele.

In some embodiments, the method of deactivating a mutant allele further comprises enhancing activity of the functional protein such as by providing a protein/peptide, a nucleic acid encoding a protein/peptide, or a small molecule such as a chemical compound, capable of activating/enhancing activity of the functional protein.

According to some embodiments, the present disclosure provides an RNA molecule which binds to/associates with and/or directs the RNA guided DNA nuclease e.g., CRISPR nuclease to a sequence comprising at least one nucleotide which differs between a mutant allele and a functional allele (e.g., heterozygous SNP) of a gene of interest (i.e., a sequence of the mutant allele which is not present in the functional allele).

In some embodiments, the method comprises the steps of: contacting a mutant allele of a gene of interest with an allele-specific RNA molecule and a CRISPR nuclease e.g., a Cas9 protein, wherein the allele-specific RNA molecule and the CRISPR nuclease e.g., Cas9 associate with a nucleotide sequence of the mutant allele of the gene of interest which differs by at least one nucleotide from a nucleotide sequence of a functional allele of the gene of interest, thereby modifying or knocking-out the mutant allele.

In some embodiments, the allele-specific RNA molecule and a CRISPR nuclease is introduced to a cell encoding the gene of interest. In some embodiments, the cell encoding the gene of interest is in a mammalian subject. In some embodiments, the cell encoding the gene of interest is in a plant.

In some embodiments, the cleaved mutant allele is further subjected to insertion or deletion (indel) by an error prone non-homologous end joining (NHEJ) mechanism, generating a frameshift in the mutant allele's sequence. In some embodiments, the generated frameshift results in inactivation or knockout of the mutant allele. In some embodiments, the generated frameshift creates an early stop codon in the mutant allele and results in generation of a truncated protein. In such embodiments, the method results in the generation of a truncated protein encoded by the mutant allele and a functional protein encoded by the functional allele. In some embodiments, a frameshift generated in a mutant allele using the methods of the invention results in nonsense-mediated mRNA decay of the transcript of the mutant allele.

In some embodiments, the mutant allele is an allele of apolipoprotein A1 (APOA1) gene. In some embodiments, the RNA molecule targets a heterozygous SNP of the APOA1 gene which co-exists with/is genetically linked to the mutated sequence associated with apoA-1 related amyloidosis genetic disorder. In some embodiments, the RNA molecule targets a heterozygous SNP of the APOA1 gene, wherein the heterozygosity of said SNP is highly prevalent in the population. In embodiments of the present invention, the REF nucleotide is prevalent in the mutant allele and not in the functional allele of an individual subject to be treated. In embodiments of the present invention, the ALT nucleotide is prevalent in the mutant allele and not in the functional allele of an individual subject to be treated. In some embodiments, a disease-causing mutation within a mutant APOA1 allele is targeted.

In embodiments of the present invention, the heterozygous SNP may or may not be associated with an APOA1 related disease phenotype. In embodiments of the present invention, the heterozygous SNP is associated with an APOA1 related disease phenotype. In embodiments of the present invention, the SNP is not associated with an APOA1 related disease phenotype

In some embodiments, the heterozygous SNP is within an exon of the gene of interest. In such embodiments, a guide sequence portion of an RNA molecule may be designed to associate with a sequence of the exon of the gene of interest.

In some embodiments, a heterozygous SNP is within an intron or an exon of the gene of interest. In some embodiments, a heterozygous SNP is in a splice site between the intron and the exon:

A skilled artisan will appreciate that in all of the embodiments of the present invention, each of the RNA molecules of the present invention are capable of complexing with a CRISPR nuclease such as to associate with a target genomic DNA sequence of interest next to a protospacer adjacent motif (PAM). The CRISPR nuclease then mediates cleavage of target DNA to create a double-stranded break within the protospacer. Accordingly, in embodiments of the present invention, the guide sequences and RNA molecules of the present invention may target a location 1, 2, 3, 4, 5, 6, 7, 8, 9, 10, 11, 12, 13, 14, 15, 16, 17, 18, 19, 20, 21, 22, 23, 24, 25, 26, 27, or 28 nucleotides upstream or downstream from a PAM site. In embodiments of the present invention, the guide sequences and RNA molecules of the present invention may target a location that is within the PAM site.

Therefore, in embodiments of the present invention, the RNA molecules of the present invention may affect a double strand break in an allele of a gene 1, 2, 3, 4, 5, 6, 7, 8, 9, 10, 11, 12, 13, 14, 15, 16, 17, 18, 19, 20, 21, 22, 23, 24, 25, 26, 27, 28 upstream or downstream from a polymorphic site. In further embodiments of the invention, the polymorphic site is within the PAM site. A skilled artisan will appreciate that where a heterozygous polymorphic site is present, an RNA molecule may be designed to affect a double stranded break in only the REF or ALT nucleotide base of the heterozygous polymorphic site.

In embodiments of the present invention, an RNA molecule is designed to target a heterozygous polymorphic site present in the APOA1 gene, wherein the RNA molecule targets only the nucleotide base, REF or ALT, of the heterozygous polymorphic site present in only the mutant allele of the APOA1 gene

Each possibility represents a separate embodiment of the present invention. In some embodiments, a guide sequence portion of an RNA molecule may be designed to associate with a sequence of the gene of interest which comprises the splice site.

In some embodiments, the method is utilized for treating a subject having a disease phenotype resulting from the heterozygote APOA1 gene. In such embodiments, the method results in improvement, amelioration or prevention of the disease phenotype.

Embodiments referred to above refer to a CRISPR nuclease, RNA molecule(s), and tracrRNA being effective in a subject or cells at the same time. The CRISPR, RNA molecule(s), and tracrRNA can be delivered substantially at the same time or can be delivered at different times but have effect at the same time. For example, this includes delivering the CRISPR nuclease to the subject or cells before the RNA molecule and/or tracr RNA is substantially extant in the subject or cells.

In some embodiments, the cell is a liver cell. In some embodiments, the cell is a hepatocyte cell.

According to embodiments of the present invention, there is provided a method for inactivating a mutant allele of the APOA1 gene in a cell, the method comprising the stops of:
a) selecting a cell with an APOA1 gene mutation associate with apoA-1 related amyloidosis and who is heterozygous at one or more polymorphic sites in the APOA1 gene selected from: rs11216158, rs11216157, rs2727784, rs613808, rs4018880, rs1374117, rs670, rs5069, rs5070, rs2070665, rs5072, rs7116797, rs5076;
b) introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
thereby inactivating only the mutant allele of the APOA1 gene in the cell.

According to embodiments of the present invention, there is provided a method for inactivating a mutant allele of the APOA1 gene in a cell, the method comprising the steps of:
a) selecting a cell with an APOA1 gene mutation associated with apoA-1 related amyloidosis and who is heterozygous at one or more polymorphic sites in the APOA1 gene selected from: rs11216158, rs11216157, rs2727784, rs613808, rs4018880, rs1374117, rs670, rs5069, rs5070, rs2070665, rs5072, rs7116797, rs5076, rs28931574;
b) introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
and wherein the method further comprises introduction of a second RNA molecule comprising a guide sequence portion capable of complexing with a CRISPR nuclease, wherein the complex of the second RNA molecule and the CRISPR nuclease affects a second double strand break in the APOA1gene;
   thereby inactivating only the mutant allele of the APOA1 gene in the cell.

According to embodiments of the present invention, there is provided a method for inactivating a mutant allele of the APOA1 gene in a cell with an APOA1 gene mutation associated with apoA-1 related amyloidosis and which cell is heterozygous at one or more polymorphic sites in the APOA1 gene selected from: rs11216158, rs11216157, rs2727784, rs613808, rs4018880, rs1374117, rs670, rs5069, rs5070, rs2070665, rs5072, rs7116797, rs5076, the method comprising
introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
thereby inactivating only the mutant allele of the APOA1 gene in the cell.

According to embodiments of the present invention, there is provided a method for inactivating a mutant allele of the APOA1 gene in a cell with an APOA1 gene mutation associated with apoA-1 related amyloidosis and heterozygous at one or more polymorphic sites in the APOA1 gene selected from: rs11216158, rs11216157, rs2727784, rs613808, rs4018880, rs1374117, rs670, rs5069, rs5070, rs2070665, rs5072, rs7116797, rs5076, rs28931574, the method comprising:
introducing to the cell a composition comprising:
   a CRISPR nuclease, and
   a first RNA molecule comprising a guide sequence portion having 17-20 nucleotides,
wherein a complex of the CRISPR nuclease and the first RNA molecule affects a double strand break in only the mutant allele of the APO1 gene and not in the functional allele of the APOA1 gene in the cell;
and wherein the method further comprises introduction of a second RNA molecule comprising a guide sequence portion capable of complexing with a CRISPR nuclease, wherein the complex of the second RNA molecule and CRISPR nuclease affects a second double strand break in the APOA1gene;
thereby inactivating only the mutant allele of the APOA1 gene in the cell.

In embodiments of the present invention, the guide sequence portion of the first RNA molecule comprises 17-20 contiguous nucleotides as set forth in any one of SEQ ID NOs: 1-1313.

In embodiments of the present invention, the second double strand break is within a non-coding region of the APOA1 gene.

In embodiments of the present invention, the non-coding region of the APOA1 gene is exon 1, intron 1, intron 2, or intron 3.

In embodiments of the present invention, the cell is also heterozygous at least one additional polymorphic site in the APOA1 gene selected from: rs11216158, rs11216157, rs2727784, rs613808, rs4018880, rs1374117, rs670, rs5069, rs5070, rs2070665, rs5072, rs7116797, rs5076, rs28931574.

In embodiments of the present invention, the complex of the second RNA molecule and CRISPR nuclease affects a double-stranded break in only the mutant allele of the APOA1 gene of the cell.

In embodiments of the present invention, the composition may comprise 1, 2, 3 or more CRISPR nucleases. In embodiments of the present invention, introducing a composition to the cell may comprise introducing 1, 2, 3, or more compositions to the cell. In embodiments of the present invention, each composition may comprise a different CRISPR nuclease or the same CRISPR nuclease. In embodiments of the present invention involving two RNA molecules, the second RNA molecule may form a complex with the same CRISPR nuclease as the first RNA molecule, or may form a complex with another CRISPR nuclease.

In embodiments of the present invention, the guide sequence portion of the second RNA molecule comprises 17-20 nucleotides of a sequence of 17-20 contiguous nucleotides as set forth in any one of SEQ ID NOs: 1-1313.

In embodiments of the present invention, a cell with an APOA1 gene mutation associated with apoA-1 related amyloidosis may be from a subject with the APOA1 gene mutation and/or afflicted with apoA-1 related amyloidosis. Accordingly, selecting a cell with an APOA1 gene mutation may comprise selecting a subject with the APOA1 gene mutation. In further embodiments of the present invention, selecting a cell may comprise selecting a cell from a subject with the APOA1 gene mutation. In embodiments of the present invention, introducing the compositions of the subject invention to the cell may comprise introducing the compositions of the invention to the cell of a subject afflicted with the APOA1 gene mutation.

Accordingly, in embodiments of the present invention, there is provided a method for inactivating a mutant allele of the APOA1 gene in the cell of a subject, the method comprising the step of selecting a subject with an APOA1 gene mutation resulting in apoA-1 related amyloidosis and who is heterozygous at one or more polymorphic sites in the APOA1 gene selected from: rs11216158, rs11216157, rs2727784, rs613808, rs4018880, rs1374117, rs670, rs5069, rs5070, rs2070665, rs5072, rs7116797, rs5076, rs28931574; and introducing into the cell of the subject the compositions of the instant invention.

Accordingly, embodiments of the present invention encompass the screening of subjects or cells for the APOA1 gene. A person having ordinary skill in the art would readily understand methods of screening for mutations within the APOA1 gene in the art, by way of non-limiting examples, e.g., karyotyping, Fluorescence In situ Hybridization, and/or microarray testing.

In embodiments of the present invention, the cell is heterozygous at one or more polymorphic sites in the APOA1 gene selected from: rs670 and rs5069, and wherein the complex of the second RNA molecule and the CRISPR nuclease affects a double strand break in a non-coding region of the APOA1 gene selected from: intron 2 and intron 3.

In some embodiments, the cell is heterozygous at one or more polymorphic sites in the APOA1 gene selected from: rs670 and rs5069, and a complex of a first RNA molecule comprising a guide sequence portion having 17-20 nucleotides and a CRISPR nuclease affects a double strand break in only the mutant allele of the APO1 gene and not in the functional allele of the APOA1 gene, and wherein a complex of the second RNA molecule and the CRISPR nuclease affects a double strand break in a non-coding region of the APOA1 gene selected from: intron 2 and intron 3. In such embodiments the guide sequence portion of the first RNA molecule comprises having 17-20 nucleotides may comprise a sequence of 17-20 contiguous nucleotides as set forth in any one of SEQ ID NOs: 10, 11, 17, 24, 28, 35, 39, 43, 48, 49, 70, 71, 75, 82, 85, 89, 91, 100, 101, 113, 120, 134, 141, 142, 146, 148, 150, 161, 176, 178, 180, 186, 194, 195, 201, 203, 204, 214, 260, 261, 267, 268, 279, 280, 295, 305, 329, 330, 331, 367, 372, 377, 378, 380, 381, 398, 400, 403, 404, 405, 410, 411, 415, 429, 430, 457, 458, 480, 481, 484, 485, 488, 496, 498, 499, 500, 503, 505, 512, 513, 542, 543, 546, 547, 550, 552, 553, 555, 564, 566, 567, 572, 573, 583, 597, 598, 599, 603, 621, 643, 644, 645, 649, 667, 683, 705, 731, 732, 744, 745, 752, 753, 757, 758, 769, 770, 781, 784, 785, 787, 788, 791, 792, 794, 805, 806, 817, 818, 837, 851, 854, 859, 861, 864, 865, 867, 883, 884, 913, 914, 925, 926, 927, 928, 951, 952, 955, 956, 989, 990, 994, 995, 1002, 1003, 1029, 1031, 1057, 1064, 1078, 1110, 1130, 1136, 1163, 1174, 1200, 1254, 1300, 1068, 1070, 1077, 1128, 1134, 1176, 1181, 1197, and 1255.

In some embodiments, the cell is heterozygous at one or more polymorphic sites in the APOA1 gene selected from: rs11216158, rs11216157, rs2727784, rs613808, rs4018880, and rs1374117, and a complex of a first RNA molecule comprising a guide sequence portion having 17-20 nucleotides and a CRISPR nuclease affects a double strand break in only the mutant allele of the APO1 gene and not in the functional allele of the APOA1 gene, and wherein a complex of the second RNA molecule and the CRISPR nuclease affects a double strand break in a non-coding region of the APOA1 gene selected from: intron 2 and intron 3. In such embodiments the guide sequence portion of the first RNA molecule comprises having 17-20 nucleotides may comprise a sequence of 17-20 contiguous nucleotides as set forth in any one of SEQ ID NOs: 2, 4, 7, 8, 9, 19, 21, 22, 23, 25, 26, 27, 31, 32, 33, 34, 36, 44, 45, 46, 47, 51, 52, 53, 54, 55, 56, 57, 58, 62, 63, 64, 65, 66, 67, 68, 69, 72, 73, 74, 76, 77, 78, 86, 87, 88, 94, 95, 97, 98, 99, 104, 105, 106, 107, 116, 117, 118, 121, 124, 125, 126, 127, 128, 129, 130, 131, 133, 135, 136, 137, 143, 147, 153, 154, 159, 160, 163, 164, 165, 166, 172, 174, 175, 177, 185, 187, 197, 202, 206, 207, 208, 209, 215, 216, 217, 218, 219, 220, 221, 223, 226, 227, 228, 229, 232, 233, 234, 235, 236, 237, 239, 241, 242, 243, 244, 250, 256, 257, 262, 263, 264, 265, 266, 276, 277, 278, 284, 285, 291, 292, 297, 298, 299, 300, 301, 302, 303, 306, 307, 308, 309, 310, 311, 325, 334, 335, 336, 337, 338, 339, 345, 346, 351, 352, 357, 358, 363, 364, 368, 373, 374, 383, 384, 387, 388, 389, 392, 393, 394, 396, 397, 406, 407, 413, 416, 417, 418, 419, 422, 424, 425, 426, 427, 431, 433, 434, 435, 437, 438, 442, 445, 446, 447, 462, 463, 471, 474, 475, 476, 482, 483, 490, 491, 494, 495, 501, 502, 514, 517, 518, 519, 520, 522, 525, 529, 530, 531, 538, 539, 540, 541, 544, 545, 551, 559, 562, 563, 570, 576, 577, 578, 581, 582, 585, 586, 587, 590, 591, 592, 593, 594, 601, 602, 604, 605, 606, 607, 608, 611, 612, 613, 614, 615, 617, 618, 622, 623, 625, 626, 627, 629, 630, 631, 633, 635, 636, 641, 646, 655, 661, 662, 663, 668, 669, 670, 671, 672, 674, 675, 676, 679, 680, 681, 682, 684, 685, 686, 687, 688, 694, 695, 697, 698, 699, 700, 707, 708, 709, 710, 711, 712, 714, 716, 717, 718, 720, 734, 741, 749, 756, 759, 761, 762, 765, 768, 772, 774, 775, 776, 777, 778, 782, 783, 795, 796, 799, 803, 804, 807, 809, 814, 815, 816, 821, 822, 823, 828, 833, 838, 840, 852, 853, 855, 856, 862, 863, 866, 873, 885, 899, 904, 907, 908, 917, 918, 933, 938, 939, 940, 943, 946, 947, 948, 949, 950, 957, 958, 959, 960, 961, 962, 963, 964, 966, 967, 969, 970, 971, 973, 974, 977, 978, 979, 986, 987, 991, 992, 993, 996, 997, 998, 999, 1000, 1001, 1009, 1012, 1013, 1014, 1015, 1019, 1020, 1022, 1023, 1024, 1025, 1026, 1032, 1036, 1037, 1040, 1041, 1042, 1044, 1049, 1050, 1051, 1053, 1054, 1055, 1056, 1061, 1062, 1063, 1083, 1095, 1104, 1105, 1108, 1109, 1111, 1124, 1127, 1144, 1145, 1150, 1151, 1186, 1193, 1198, 1214, 1216, 1217, 1222, 1225, 1246, 1268, 1272, 1273, 1307, 1312, 1313, 1079, 1081, 1187, 1284, 1310, 1059, 1066, 1067, 1073, 1074, 1075, 1082, 1087, 1088, 1090, 1119, 1125, 1146, 1182, 1184, 1188, 1189, 1191, 1192, 1201, 1202, 1203, 1215, 1223, 1227, 1232, 1242, 1243, 1245, 1277, 1285, 1289, 1290, 1291, 1294, 1308, 1311, 1099, 1102, 1114, 1143, 1149, 1170, 1194, 1211, 1212, 1264, 1266, 1270, 1271, 1304, 1084, 1086, 1116, 1120, 1126, 1129, 1133, 1140, 1142, 1147, 1155, 1156, 1158, 1161, 1164, 1165, 1166, 1168, 1169, 1171, 1179, 1180, 1183, 1190, 1210, 1213, 1224, 1226, 1229, 1236, 1237, 1238, 1239, 1240, 1248, 1249, 1252, 1260, 1267, 1269, 1275, 1276, 1278, 1280, 1281, 1282, 1298, 1299, 1303, 1306, 1092, 1097, 1098, 1123, 1173, 1196, 1204, 1209, 1219, and 1279.

In some embodiments, the cell is heterozygous at one or more polymorphic sites in the APOA1 gene selected from: rs670, and rs5069, and a complex of a first RNA molecule comprising a guide sequence portion having 17-20 nucleotides and a CRISPR nuclease affects a double strand break in only the mutant allele of the APO1 gene and not in the functional allele of the APOA1 gene, and wherein a complex of the second RNA molecule and the CRISPR nuclease affects a double strand break in a non-coding region of the APOA1 gene selected from: intron 2 and intron 3. In such embodiments the guide sequence portion of the first RNA molecule having 17-20 nucleotides may comprise a sequence of 17-20 contiguous nucleotides as set forth in any one of SEQ ID NOs: 10, 11, 17, 24, 28, 35, 39, 43, 48, 49, 70, 71, 75, 82, 85, 89, 91, 100, 101, 113, 120, 134, 141, 142, 146, 148, 150, 161, 176, 178, 180, 186, 194, 195, 201, 203, 204, 214, 260, 261, 267, 268, 279, 280, 295, 305, 329, 330, 331, 367, 372, 377, 378, 380, 381, 398, 400, 403, 404, 405, 410, 411, 415, 429, 430, 457, 458, 480, 481, 484, 485, 488, 496, 498, 499, 500, 503, 505, 512, 513, 542, 543, 546, 547, 550, 552, 553, 555, 564, 566, 567, 572, 573, 583, 597, 598, 599, 603, 621, 643, 644, 645, 649, 667, 683, 705, 731, 732, 744, 745, 752, 753, 757, 758, 769, 770, 781, 784, 785, 787, 788, 791, 792, 794, 805, 806, 817, 818, 837, 851, 854, 859, 861, 864, 865, 867, 883, 884, 913, 914, 925, 926, 927, 928, 951, 952, 955, 956, 989, 990, 994, 995, 1002, 1003, 1029, 1031, 1057, 1064, 1078, 1110, 1130, 1136, 1163, 1174, 1200, 1254, 1300, 1068, 1070, 1077, 1128, 1134, 1176, 1181, 1197, and 1255.

In some embodiments, cell is heterozygous at the polymorphic site in the APOA1 gene, rs5070, and a complex of a first RNA molecule comprising a guide sequence portion having 17-20 nucleotides and a CRISPR nuclease affects a double strand break in only the mutant allele of the APO1 gene and not in the functional allele of the APOA1 gene, and wherein a complex of the second RNA molecule and the CRISPR nuclease affects a double strand break in a non-coding region of the APOA1 gene selected from: exon 1, intron 1, and intron 3. In such embodiments, the guide sequence portion having 17-20 nucleotides of the first RNA molecule may comprise a sequence of 17-20 contiguous nucleotides as set forth in any one of SEQ ID NOs: 12, 13, 119, 139, 140, 145, 155, 162, 169, 170, 182, 183, 258, 259, 269, 275, 281, 282, 296, 304, 340, 341, 355, 356, 375, 376, 379, 399, 408, 409, 443, 444, 453, 454, 459, 472, 527, 528, 532, 533, 537, 560, 628, 647, 648, 650, 651, 713, 719, 736, 737, 746, 747, 779, 780, 836, 843, 844, 850, 860, 868, 874, 875, 876, 877, 878, 886, 888, 889, 890, 891, 892, 930, 931, 932, 975, 976, 1052, 1091, 1137, 1153, 1157, 1172, 1218, 1253, 1257, 1258, and 1309.

In some embodiments, the cell is heterozygous at one or more polymorphic sites in the APOA1 gene selected from: rs2070665, rs5072, rs7116797, and rs5076, and a complex of a first RNA molecule comprising a guide sequence portion having 17-20 nucleotides and a CRISPR nuclease affects a double strand break in only the mutant allele of the APO1 gene and not in the functional allele of the APOA1 gene, and wherein a complex of the second RNA molecule and the CRISPR nuclease affects a double strand break in a non-coding region of the APOA1 gene selected from: exon 1, intron 1 and intron 2. In such embodiments, the guide sequence portion having 17-20 nucleotides of the first RNA molecule may comprise a sequence of 17-20 contiguous nucleotides as set forth in any one of SEQ ID NOs: 3, 5, 6, 14, 15, 16, 20, 29, 30, 37, 38, 40, 41, 42, 59, 60, 80, 81, 83, 84, 90, 92, 93, 96, 102, 103, 108, 109, 110, 111, 112, 114, 115, 122, 123, 132, 138, 144, 151, 152, 156, 157, 158, 167, 168, 171, 173, 179, 181, 184, 188, 189, 190, 191, 192, 193, 196, 198, 199, 200, 205, 213, 224, 225, 230, 231, 238, 240, 245, 246, 247, 248, 249, 251, 252, 253, 254, 255, 270, 271, 272, 283, 286, 287, 289, 290, 314, 315, 316, 319, 320, 321, 322, 326, 327, 328, 332, 333, 342, 343, 344, 348, 349, 350, 353, 354, 359, 362, 365, 366, 369, 370, 371, 382, 390, 391, 395, 401, 402, 412, 414, 423, 428, 436, 439, 440, 441, 448, 449, 451, 452, 460, 461, 464, 465, 468, 469, 470, 473, 477, 478, 479, 486, 487, 489, 497, 504, 506, 507, 508, 509, 510, 511, 515, 516, 521, 523, 526, 534, 535, 536, 548, 549, 554, 556, 557, 558, 561, 565, 568, 569, 571, 574, 575, 579, 580, 584, 588, 589, 595, 600, 609, 610, 616, 619, 620, 624, 637, 638, 640, 652, 653, 654, 657, 658, 659, 660, 666, 689, 690, 692, 696, 702, 703, 704, 706, 715, 723, 724, 727, 728, 729, 730, 733, 735, 738, 739, 740, 742, 743, 748, 750, 754, 755, 760, 763, 764, 771, 773, 786, 789, 790, 793, 797, 798, 800, 801, 802, 819, 820, 826, 827, 829, 830, 831, 832, 834, 835, 839, 845, 846, 848, 849, 857, 858, 871, 872, 879, 880, 881, 882, 887, 893, 894, 895, 896, 897, 898, 900, 901, 902, 903, 905, 906, 910, 911, 912, 915, 919, 920, 921, 922, 923, 924, 929, 934, 935, 937, 944, 945, 953, 954, 965, 968, 982, 983, 988, 1004, 1005, 1007, 1016, 1017, 1018, 1030, 1033, 1034, 1035, 1043, 1045, 1046, 1047, and 1048, 1058, 1080, 1100, 1162, 1167, 1185, 1235, 1241, 1259, 1262, 1292, 1106, 1118, 1121, 1135, 1208, 1228, 1233, 1261, 1265, 1302, 1085, 1103, 1178, 1207, 1230, 1250, 1263, 1283, 1293, 1065, 1069, 1101, 1117, 1138, 1139, 1148, 1175, 1177, and 1288.

In embodiments of the present invention, the cell is heterozygous at the polymorphic sites in the APOA1 gene rs28931574. In such embodiments the guide sequence portion having 17-20 nucleotides of the first or second RNA molecule may comprise a sequence of 17-20 contiguous nucleotides as set forth in any one of SEQ ID NOs: 1, 18, 50, 61, 79, 149, 210, 211, 212, 222, 273, 274, 288, 293, 294, 312, 313, 317, 318, 323, 324, 347, 360, 361, 385, 386, 420, 421, 432, 450, 455, 456, 466, 467, 492, 493, 524, 596, 632, 634, 639, 642, 656, 664, 665, 673, 677, 678, 691, 693, 701, 721, 722, 725, 726, 751, 766, 767, 808, 810, 811, 812, 813, 824, 825, 841, 842, 847, 869, 870, 909, 916, 936, 941, 942, 972, 980, 981, 984, 985, 1006, 1008, 1010, 1011, 1021, 1027, 1028, 1038, 1039, 1060, 1071, 1072, 1076, 1089, 1093, 1094, 1096, 1107, 1112, 1113, 1115, 1122, 1131, 1132, 1141, 1152, 1154, 1159, 1160, 1195, 1199, 1205, 1206, 1220, 1221, 1231, 1234, 1244, 1247, 1251, 1256, 1274, 1286, 1287, 1295, 1296, 1297, 1301, and 1305.

In embodiments of the present invention, the double strand break is 1, 2, 3, 4, 5, 6, 7, 8, 9, 10, 11, 12, 13, 14, 15, 16, 17, 18, 19, 20, 21, 22, 23, 24, 25, 26, 27, 28 nucleotides upstream or downstream from the heterozygous polymorphic site.

### Dominant Genetic Disorders

One of skill in the art will appreciate that all subjects with any type of heterozygote genetic disorder (e.g., dominant genetic disorder) may be subjected to the methods described herein. In one embodiment, the present invention may be used to target a gene involved in, associated with, or causative of dominant genetic disorders such as, for example, apoA-1 related amyloidosis. In some embodiments, the dominant genetic disorder is apoA-1 related amyloidosis. In some embodiments, the target gene is the APOA1 gene (Entrez Gene, gene ID No: 335).

### CRISPR Nucleases and PAM Recognition

In some embodiments, the sequence specific nuclease is selected from CRISPR nucleases, or a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to the sequence comprising at least one nucleotide which differs between a mutant allele and its counterpart functional allele (e.g., SNP). In some embodiments, the CRISPR complex does not further comprise a tracrRNA. In a non-limiting example, in which the RNA guided DNA nuclease is a CRISPR protein, the at least one nucleotide which differs between the dominant mutant allele and the functional allele may be within the PAM site and/or proximal to the PAM site within the region that the RNA molecule is designed to hybridize to. A skilled artisan will appreciate that RNA: molecules can be engineered to bind to a target of choice in a genome by commonly known methods in the art.

In embodiments of the present invention, a type II CRISPR system utilizes a mature crRNA: tracrRNA complex directs a CRISPR nuclease, e.g. Cas9, to the target DNA via Watson-Crick base-pairing between the crRNA and the protospacer on the target DNA next to the protospacer adjacent motif (PAM), an additional requirement for target recognition. The CRISPR nuclease then mediates cleavage of target DNA to create a double-stranded break within the protospacer. A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NNNNGATT for Neisseria meningitidis (NmCas9); or TTTV for Cpf1. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break at a desired location in the genome of a cell. The most commonly used RNA-guided DNA nucleases are derived from CRISPR systems, however, other RNA-guided DNA nucleases arc also contemplated for use in the genome editing compositions and methods described herein. For instance, see U.S. Patent Publication No. 2015-0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Caso, Cas6e, Cas6f, Cas7, Cas8al, Cas8a2, Cas8b, Cas8c, Cas9, Cas10, Casl Od, CasF, CasG, CasH, Csy1, Csy2, Csy3, Csel (or CasA), Csc2 (or CasB), Csc3 (or CasE), Cse4 (or CasC), Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, Csx10, Csx16, CsaX, Csx3, Cszl, Csx15, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei. Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acctohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chihonoplastes, Oscillatoria sp., Petrotoga mobilis, Thermosipho africanus, Acaryochloris marina, or any species which encodes a CRISPR nuclease with a known PAM. sequence. CRISPR nucleases encoded by uncultured bacteria may also be used in the context of the invention. (See Burstein et al. Nature, 2017). Variants of CRIPSR proteins having known PAM sequences e.g., spCas9 D1135E variant, spCas9 VQR variant, spCas9 EQR variant, or spCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al. (2015) Cell.).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl) uridine, 2′-O-methylcytidine, 5-carboxymethylaminomethyl-2-thiouridine, 5-carboxymethylaminomethyluridine, dihydrouridinc, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, I-methylinosine, “2,2-dimethylguanosinc”, 2-methyladenosine, 2-methylguanosinc, 3-methylcytidine, 5-methylcytidine, N6-methyladenosine, 7-methylguanosine, 5-methylaminomethyluridine, 5-methoxyaminomethyl-2-thiouridine, “beta. D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl) carbamoyl) thrconine, N-((9-beta-D-ribofuranosylpurinc-6-yl)N-methylcarbamoyl) threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl) threonine, 2′-O-methyl-5-methyluridine, 2′-O-methyluridine, wybutosine, “3-(3-amino-3-carboxy-propyl) uridine, (acp3) u”, 2-O-methyl (M), 3′-phosphorothioate (MS), 3′-thioPACE (MSP), pseudouridine, or 1-methyl pseudo-uridine. Each possibility represents a separate embodiment of the present invention.

Guide Sequences which Specifically Target a Mutant Allele

A given gene may contain thousands of SNPs. Utilizing a 24 base pair target window for targeting each SNP in a gene would require hundreds of thousands of guide sequences. Any given guide sequence when utilized to target a SNP may result in degradation of the guide sequence, limited activity, no activity, or off-target effects. Accordingly, suitable guide sequences are necessary for targeting a given gene. By the present invention, a novel set of guide sequences have been identified for knocking out expression of a mutated apoA-1 protein, inactivating a mutant APOA1 gene allele, and treating apoA-1 related amyloidosis.

The present disclosure provides guide sequences capable of specifically targeting a mutant allele for inactivation while leaving the functional allele unmodified. The guide sequences of the present invention arc designed to, and are most likely to, specifically differentiate between a mutant allele and a functional allele. Of all possible guide sequences which target a mutant allele desired to be inactivated, the specific guide sequences disclosed herein are specifically effective to function with the disclosed embodiments.

Briefly, the guide sequences may have properties as follows: (1) target a heterozygous SNP/insertion/deletion/indel with a high prevalence in the general population, in a specific ethnic population or in a patient population is above 1% and the SNP/insertion/deletion/indel heterozygosity rate in the same population is above 1%; (2) target a location of a SNP/insertion/deletion/indel proximal to a portion of the gene e.g., within 5k bases of any portion of the gene, for example, a promoter, a UTR, an exon or an intron; and (3) target a mutant allele using an RNA molecule which targets a founder or common pathogenic mutations for the disease/gene. In some embodiments, the prevalence of the SNP/insertion/deletion/indel in the general population, in a specific ethnic population or in a patient population is above 1%, 2%, 3%, 4%, 5%, 6%, 7%, 8%, 9%, 10%, 11%, 12%, 13%, 14%, or 15% and the SNP/insertion/deletion/indel heterozygosity rate in the same population is above 1%, 2%, 3%, 4%, 5%, 6%, 7%, 8%, 9%, 10%, 11%, 12%, 13%, 14%, or 15%. Each possibility represents a separate embodiment and may be combined at will.

For each gene, according to SNP/insertion/deletion/indel any one of the following strategies may be used to deactivate the mutant allele: (1) Knockout strategy using one RNA molecule-one RNA molecule is utilized to direct a CRISPR nuclease to a mutant allele and create a double-strand break (DSB) leading to formation of a frameshift mutation in an exon or in a splice site region of the mutant allele; (2) Knockout strategy using two RNA molecules-two RNA molecules are utilized. A first RNA molecule targets a region in the promoter or an upstream region of a mutant allele and another RNA molecule targets downstream of the first RNA molecule in a promoter, exon, or intron of the mutant allele; (3) Exon(s) skipping strategy-one RNA molecule may be used to target a CRISPR nuclease to a splice site region, either at the 5′end of an intron (donor sequence) or the 3′ end of an intron (acceptor sequence), in order to destroy the splice site. Alternatively, two RNA molecules may be utilized such that a first RNA molecule targets an upstream region of an exon and a second RNA molecule targets a region downstream of the first RNA molecule, thereby excising the exon(s). Based on the locations of identified SNPs/insertions/deletions/indels for each mutant allele, any one of, or a combination of, the above-mentioned methods to deactivate the mutant allele may be utilized.

When only one RNA molecule is used is that the location of the SNP is in an exon or in close proximity (e.g., within 20 basepairs) to a splice site between the intron and the exon. When two RNA molecules are used, guide sequences may target two SNPs such that the first SNP is upstream of exon 1 e.g., within the 5′ untranslated region, or within the promoter or within the first 2 kilobases 5′ of the transcription start site, and the second SNP is downstream of the first SNP e.g., within the first 2 kilobases 5′ of the transcription start site, or within intron 1, 2 or 3, or within exon 1, exon 2, or exon 3.

Guide sequences of the present invention may target a SNP in the upstream portion of the targeted gene, preferably upstream of the last exon of the targeted gene. Guide sequences may target a SNP upstream to exon 1, for example within the 5′ untranslated region, or within the promoter or within the first 4-5 kilobases 5′ of the transcription start site.

Guide sequences of the present invention may also target a SNP within close proximity (e.g., within 50 basepairs, more preferably with 20 basepairs) to a known protospacer adjacent motif (PAM) site.

Guide sequences of the present invention also may target: (1) a heterozygous SNP for the targeted gene; (2) a heterozygous SNPs upstream and downstream of the gene; (3) a SNPs with a prevalence of the SNP/insertion/deletion/indel in the general population, in a specific ethnic population, or in a patient population above 1%; (4) have a guanine-cytosine content of greater than 30% and less than 85%; (5) have no repeat of 4 or more thymine/uracil or 8 or more guanine, cytosine, or adenine; (6) having no off-target identified by off-target analysis; and (7) preferably target Exons over Introns or be upstream of a SNP rather than downstream of a SNP.

In embodiments of the present invention, the SNP may be upstream or downstream of the gene. In embodiments of the present invention, the SNP is within 4,000 base pairs upstream or downstream of the gene.

The at least one nucleotide which differs between the mutant allele and the functional allele, may be upstream, downstream or within the sequence of the disease-causing mutation of the gene of interest. The at least one nucleotide which differs between the mutant allele and the functional allele, may be within an exon or within an intron of the gene of interest. In some embodiments, the at least one nucleotide which differs between the mutant allele and the functional allele is within an exon of the gene of interest. In some embodiments, the at least one nucleotide which differs between the mutant allele and the functional allele is within an intron or an exon of the gene of interest, in close proximity to a splice site between the intron and the exon e.g., 1, 2, 3, 4, 5, 6, 7, 8, 9, 10, 11, 12, 13, 14, 15, 16, 17, 18, 19, or 20 nucleotides upstream or downstream to the splice site.

In some embodiments, the at least one nucleotide is a single nucleotide polymorphisms (SNPs). In some embodiments, each of the nucleotide variants of the SNP may be expressed in the mutant allele. In some embodiments, the SNP may be a founder or common pathogenic mutation.

Guide sequences may target a SNP which has both (1) a high prevalence in the general population e.g., above 1% in the population; and (2) a high heterozygosity rate in the population, e.g., above 1%. Guide sequences may target a SNP that is globally distributed. A SNP may be a founder or common pathogenic mutation. In some embodiments, the prevalence in the general population is above 1%, 2%, 3%, 4%, 5%, 6%, 7%, 8%, 9%, 10%, 11%, 12%, 13%, 14%, or 15%. Each possibility represents a separate embodiment. In some embodiments, the heterozygosity rate in the population is above 1%, 2%, 3%, 4%, 5%, 6%, 7%, 8%, 9%, 10%, 11%, 12%, 13%, 14%, or 15%. Each possibility represents a separate embodiment.

In some embodiments, the at least one nucleotide which differs between the mutant allele and the functional allele is linked to/co-exists with the disease-causing mutation in high prevalence in a population. In such embodiments, “high prevalence” refers to at least 92%, 93%, 94%, 95%, 96%, 97%, 98%, 99%, or 100%. Each possibility represents a separate embodiment of the present invention. In one embodiment, the at least one nucleotide which differs between the mutant allele and the functional allele, is a disease-associated mutation. In some embodiments, the SNP is highly prevalent in the population. In such embodiments, “highly prevalent” refers to at least 10%, 11%, 12%, 13%, 14%, 15%, 20%, 30%, 40%, 50%, 60%, or 70% of a population. Each possibility represents a separate embodiment of the present invention.

Guide sequences of the present invention may satisfy any one of the above criteria and are most likely to differentiate between a mutant allele from its corresponding functional allele.

In some embodiments the RNA molecule targets a heterozygous SNP present in the APOA1 gene from the SNPs as shown in Table 1 below. The SNP details are indicated in the 1^{st} column and include: SNP ID No. (based on NCBI's 2018 database of Single Nucleotide Polymorphisms (dbSNP)). For variants with no available rs number variants characteristic are indicated based on gnomAD 2018 browser database. The 2^{nd} column indicates an assigned identifier for each SNP. The 3^{rd} column indicates the location of each SNP on the APOA1 gene.

TABLE 1
APOA1 gene SNPs

| RSID | SNP No. | SNP location in the gene |
|---|---|---|
| rs7116797 | s1 | Intron_3 of 3 |
| rs5072 | s2 | Intron_3 of 3 |
| rs28931574 | s3 | Exon_3_of_4 |
| rs5070 | s4 | Intron_2 of 3 |
| rs2727784 | s5 | upstream-2475 bp |
| rs11216158 | s6 | upstream-2684 bp |
| rs613808 | s7 | upstream-2302 bp |
| rs670 | s8 | Exon_1 of 4 |
| rs5076 | s9 | Intron_3 of 3 |
| rs1374117 | s10 | upstream-772 bp |
| rs5069 | s11 | Intron_1 of 3 |
| rs2070665 | s12 | Intron_3 of 3 |
| rs4018880 | s13 | upstream-1501 bp |
| rs11216157 | s14 | upstream-2514 bp |

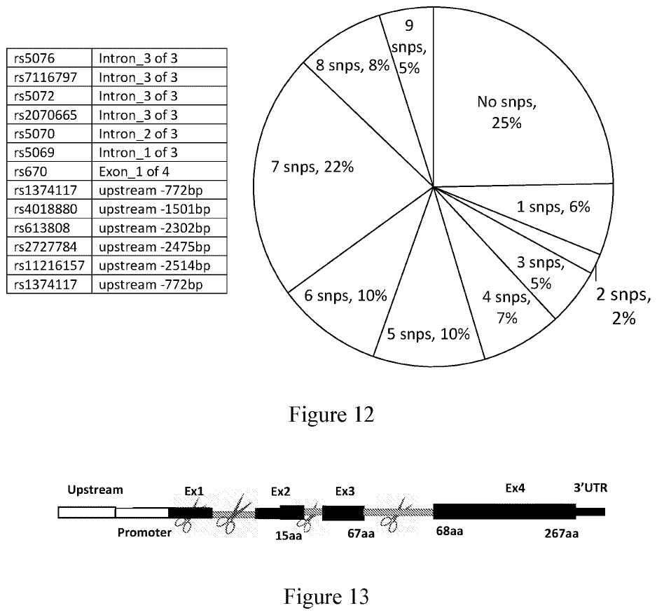

FIG. 11 and FIG. 12 disclose the heterogenicity of given selections of SNPs from Table 1 in the human population.

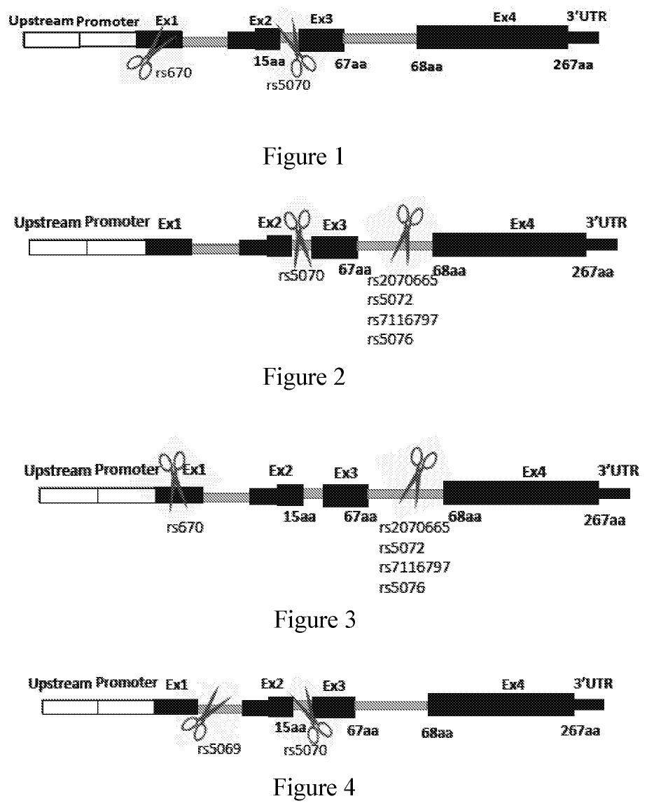

In some embodiments removal of, inter alia, exon 2 of the APOA1 gene which encodes at least a portion of the signal peptide (residues 1-18) may result in a protein that will not be secreted or alternatively RNA decay may be triggered resulting in knockout of the expression of the mutant allele. In embodiments of the present invention, two guide sequences are utilized, one targeting a heterozygous SNP present in the APOA1 gene and located at exon 1, e.g. at rs670, and the other targeting a heterozygous SNP present in the APOA1 gene located at intron 2 e.g. at rs5070, wherein each RNA molecule targets the nucleotide base, REF or ALT, of each SNP present in only the mutant allele of the APOA1 gene to remove at least a portion of exon 1 which encodes the 5′ UTR and exon 2, e.g. at rs5070. (FIG. 1)

In some embodiments, two guide sequences are utilized, one targeting intron 2 of the APOA1 gene and the other targeting intron 3 of the APOA1 gene to remove exon 3 of the APOA1 gene, which encodes a region prone to amyloidosis, to form a truncated apoA-1 which will not form aggregates/deposition as fibrils, or alternatively RNA decay may be triggered resulting in knockout of the expression of the mutant allele, wherein at least one of the guide sequences targets a heterozygous SNP present in the APOA1 gene wherein the RNA molecule targets the nucleotide base, REF or ALT, of the SNP present in only the mutant allele of the APOA1 gene. (FIG. 2)

In some embodiments, removal of exon 2, intron 2, and exons 3 of the APOA1 gene may result in the formation of a truncated apoA-1 which optionally will not secrete from the cells and/or form aggregates/deposit as fibrils, or alternatively RNA decay may be triggered resulting in knockout of the expression of the mutant allele. In embodiments of the present invention, two guide sequence are utilized, a first guide sequence targeting a sequence located at exon 1 of the APOA1 gene and a second guide sequence targeting a sequence of intron of the APOA1 gene, wherein at least one of the guide sequences targets a heterozygous SNP present in the APOA1 gene wherein the RNA molecule targets the nucleotide base, REF or ALT, of the SNP present in only the mutant allele of the APOA1 gene (FIG. 3).

In some embodiments, removal of, inter alia, exon 2 of the APOA1 gene which encodes at least a portion of the signal peptide (residues 1-18) may result in a protein that will not be secreted or alternatively RNA decay may be triggered resulting in knockout of the expression of the mutant allele. In embodiments of the present invention, a two guide sequences are utilized, one targeting a sequence of intron 1 of the APOA1 gene and the other targeting a sequence of intron 2 of the APOA1 gene, to remove at least a portion of exon 1 which encodes the 5′ UTR and exon 2, wherein at least one of the guide sequences targets a heterozygous SNP present in the APOA1 gene wherein the RNA molecule targets the nucleotide base, REF or ALT, of the SNP present in only the mutant allele of the APOA1 gene. (FIG. 4).

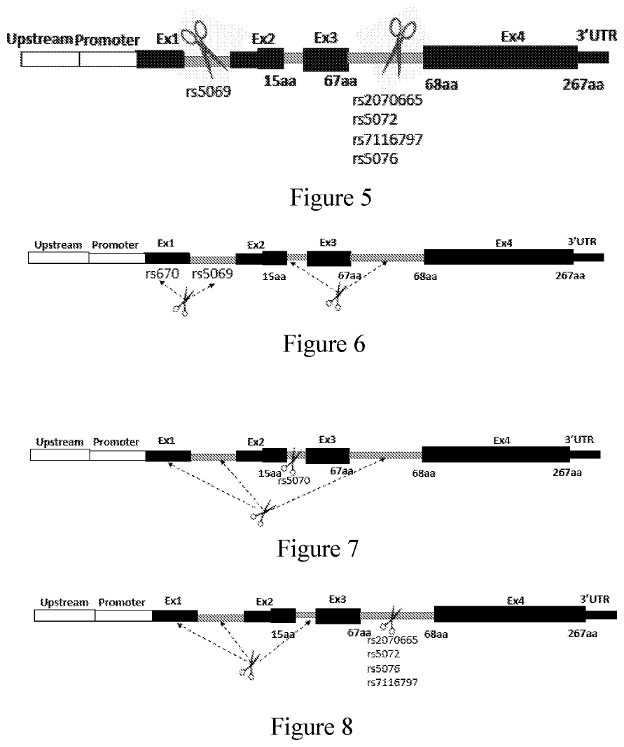

In some embodiments, removal of exon 2, intron 2, and exons 3 of the APOA1 may result in the formation of a truncated apoA-1 which optionally will not secrete from the cells and/or form aggregates/deposit as fibrils, or alternatively RNA decay may be triggered resulting in knockout of the expression of the mutant allele. In embodiments of the present invention, two guide sequences are utilized, one targeting a sequence of intron 1 of the APOA1 gene and the other targeting a sequence of intron 3 of the APOA1 gene, wherein at least one of the guide sequences targets a heterozygous SNP present in the APOA1 gene wherein the RNA molecule targets the nucleotide base, REF or ALT, of the SNP present in only the mutant allele of the APOA1 gene . . . (FIG. 5).

In some embodiments, two guide sequences are utilized to remove exon 2 or exons 2 and 3 of the APOA1 gene by targeting rs670 in exon 1 or rs5069 in intron 1 of the mutant allele of APOA1 gene and a non-coding sequence in intron 2 of the mutant allele of APOA1 gene or common to both alleles of the APOA1 gene. (FIG. 6).

In some embodiments, two guide sequences are utilized to remove exon 2, exon 3, or exons 2 and 3 of the APOA1 gene by targeting a non-coding sequence in exon 1 (5′ UTR), intron 1 or intron 3 of the mutant allele of APOA1 gene or common to both alleles of the APOA1 gene and rs5070 of intron 2 of the mutant allele of APOA1 gene. (FIG. 7).

In some embodiments, two guide sequences arc utilized to remove exons 1-3, exons 2 and 3, or exon 3 of the APOA1 gene by targeting a non-coding sequence in exon 1 (5′ UTR), intron 1, or intron 2 of the mutant allele of APOA1 gene or common to both alleles of the APOA1 gene and one of rs2070665, rs5072, rs5076, or rs7116797 in intron 9 of the mutant allele of APOA1 gene. (FIG. 8).

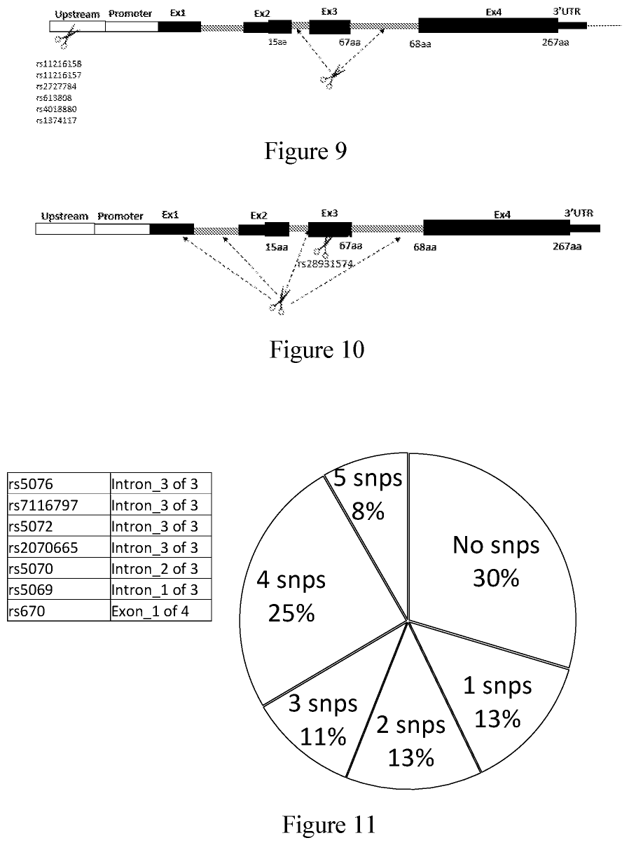

In some embodiments, two guide sequences are utilized to remove exons 1 and 2, or exons 1-3 of the APOA1 gene by targeting a non-coding sequence in intron 2 or intron 3 of the mutant allele of APOA1 gene or common to both alleles of the APOA1 gene and one of rs11216158, rs11216157, rs2727784, rs613808, rs4018880, or rs1374117 upstream of intron 2 or intron 3 on the mutant allele of APOA1 gene. (FIG. 9).

In some embodiments, two guide sequences are utilized to remove exons 1 and 2, or exons 2 or 3 of the APOA1 gene by targeting a non-coding sequence in exon 1 (5′ UTR), intron 1, intron: 2 or intron 3 of the mutant allele of APOA1 gene or common to both alleles of the APOA1 gene and rs2893 1574 in exon 3 of the mutant allele of APOA1 gene. (FIG. 10).

### Delivery to Cells

It is understood that in the methods embodied, the RNA molecules and compositions described herein may be delivered to a target cell or subject by any suitable means. The following embodiments provide non-limiting examples of methods of delivery of the RNA molecules and composition of the present invention.

In some embodiments, RNA molecule compositions of the present invention may be targeted to any cell which contains and/or expresses a dominant negative allele, including any mammalian or plant cell. For example, in one embodiment the RNA molecule specifically targets a mutant APOA1 allele and the target cell is a hepatocyte cell.

In some embodiments, the RNA molecule comprises a chemical modification. Non-limiting examples of suitable chemical modifications include 2′-O-methyl (M), 2′-O-methyl, 3′phosphorothioate (MS) or 2′-O-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6 (10): 1149-1154; Vigne (1995) Restorative Neurology and Neuroscience 8:35-36; Kremer & Perricaudet (1995) British Medical Bulletin 51 (1): 31-44; Haddada et al. (1995) in Current Topics in Microbiology and Immunology Doerfler and Bohm (eds.); and Yu et al. (1994) Gene Therapy 1:13-26.

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid: nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al. (2006) Trends Plant Sci. 11 (1): 1-4). Sonoporation using, e.g., the Sonitron 2000 system (Rich-Mar), can also be used for delivery of nucleic acids. Cationic-lipid mediated delivery of proteins and/or nucleic acids is also contemplated as an in vivo or in vitro delivery method. (See Zuris et al. (2015) Nat. Biotechnol. 33 (1): 73-80; see also Coelho et al. (2013) N. Engl. J. Med. 369, 819-829; Judge et al. (2006) Mol. Ther. 13, 494-505; and Basha et al. (2011) Mol. Ther. 19, 2186-2200).

Additional exemplary nucleic acid delivery systems include those provided by Amaxa.RTM. Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam.TM., Lipofectin. TM. and Lipofectamine.TM. RNAiMAX). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid: nucleic acid complexes, including targeted liposomes such as immunolipid complexes, is well known to one of skill in the art (See, e.g., Crystal (1995) Science 270:404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al (2009) Nature Biotechnology 27 (7): 643).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex. vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992) J. Virol. 66:2731-2739; Johann et al. (1992) J. Virol. 66:1635-1640; Sommerfelt et al. (1990) Virol. 176:58-59; Wilson et al. (1989) J. Virol. 63:2374-2378; Miller et al. (1991) J. Virol. 65:2220-2224; PCT/US94/05700).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al. (1995) Blood 85:3048-305; Kohn et al. (1995) Nat. Med. 1:1017-102; Malech et al. (1997) PNAS 94:22 12133-12138). PA317/pLASN was the first therapeutic vector used in a gene therapy trial. (Blaese et al. (1995). Transduction efficiencies of 50% or greater have been observed for MFG-S packaged vectors. (Ellem et al. (1997) Immunol Immunother. 44 (1): 10-20; Dranoff et al. (1997) Hum. Gene Ther. 1:111-2).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751, reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney et al. (1994) Culture of Animal Cells, A Manual of Basic Technique, 3rd ed, and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO—S, CHO-K1, CHO-DG44, CHO-DUXB11, CHO-DUKX, CHOKISV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (as a non-limiting example see Inaba et al. (1992) J. Exp. Med. 176:1693-1702). Stem cells that have been modified may also be used in some embodiments.

Any one of the RNA molecule compositions described herein is suitable for genome editing in post-mitotic cells or any cell which is not actively dividing, e.g., arrested cells. Examples of post-mitotic cells which may be edited using an RNA molecule composition of the present invention include, but are not limited to, a hepatocyte cell.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009-0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

In accordance with some embodiments, there is provided an RNA molecule which binds to/associates with and/or directs the RNA guided DNA nuclease to a sequence comprising at least one nucleotide which differs between a mutant allele and a functional allele (e.g., SNP) of a gene of interest (i.e., a sequence of the mutant allele which is not present in the functional allele). The sequence may be within the disease associated mutation. The sequence may be upstream or downstream to the disease associated mutation. Any sequence difference between the mutant allele and the functional allele may be targeted by an RNA molecule of the present invention to inactivate the mutant allele, or otherwise disable its dominant disease-causing effects, while preserving the activity of the functional allele.

The disclosed compositions and methods may also be used in the manufacture of a medicament for treating dominant genetic disorders in a patient.

### Mechanisms of Action for Several Embodiments Disclosed Herein

APOA1 encodes a 267 amino acid prepropeptide, which is sequentially cleaved to yield the mature 243 amino acid protein (exons 3 and 4). Typically, about 95% of plasma apoA-1 circulates in a stable-helical conformation bound to HDL and remaining portion forms a lipid free monomer (“free”). Free apoA-1 is a transient species that rapidly binds to lipoproteins or is recruited to the plasma membrane for HDL biogenesis.

Alternatively, free apoA-I may be either or alternatively misfolds and deposits as fibrils in two human diseases. It was previously demonstrated that fragments 1-83 to 1-93 of mutant apoA-I deposit as fibers in vital organs (kidney, liver, heart, nerves, 5 skin, spleen, testes, etc.) causing organ damage. Studies showed patients with gene mutations affecting residues 1 to 75 may suffer from hepatic and renal amyloidosis, while mutations in codons 173 to 178 mainly cause AApoA1 amyloidosis of the heart, larynx, and skin. Typically, AApoA1 patients have lower than normal plasma levels of apoA-I and HDL resulting from reduced secretion or enhanced degradation of the protein. Hence, unlike many other amyloid diseases, AApoA1 is not due to protein overproduction.

Without being bound by any theory or mechanism, the instant invention may be utilized to apply a CRISPR nuclease to process the mutant pathogenic APOA1 allele and not the functional APOA1 allele, such as to prevent expression of the mutant pathogenic allele or to produce a truncated non-pathogenic peptide from the mutant pathogenic allele, in order to prevent apoA-I related amyloidosis.

In some embodiments, particularly those targeting exon 2 of the APOA1 gene, the resultant peptide will lack at least a portion of the signal peptide essential for secretion. In some embodiments, particularly those targeting exon 3 of the APOA1 gene, the resultant peptide will lack a region demonstrated as prone to amyloidosis.

Outcomes of the embodiments disclosed herein may be examined to identify whether the mutant allele is expressed. In case the mutant allele is expressed, its effect on cells, such as induced stress/toxicity, may be examined by the creation of amyloids. Further its ability to secrete from cells, may be assessed, inter alia, by the presence of aggregates outside the cells. In addition, residual activity of a resultant truncated apoA-1 may be assessed.

### Examples of RNA Guide Sequences which Specifically Target Mutant Alleles of ApoA1 Gene

Although a large number of guide sequences can be designed to target a mutant allele, the nucleotide sequences described in Tables 2 identified by SEQ ID NOs: 1-1313 below were specifically selected to effectively implement the methods set forth herein and to effectively discriminate between alleles.

Referring to columns 1-4, each of SEQ ID NOs: 1-1313 indicated in column 1 corresponds to an engineered guide sequence. The corresponding SNP details are indicated in column 2. The SNP details indicated in the 2nd column include the assigned identifier for each SNP corresponding to a SNP ID indicated in Table 1. Column 3 indicates whether the target of each guide sequence is the APOA1 gene polymorph or wild type sequence where indicated. Column 4 indicates the guanine-cytosine content of each guide sequence where indicated.

Table 2 shows guide sequences designed for use as described in the embodiments above to associate with different SNPs within a sequence of a mutant APOA1 allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 2
Guide sequences designed associate with specific
SNPs of the APOA1 gene

| SEQ ID | SNP ID | Target | |
|---|---|---|---|
| NO: | (Table 1) | (ALT/REF) | % GC |
| 1 | s3 | REF | 50% |
| 2 | s5 | ALT | 60% |
| 3 | s1 | BOTH | 55% |
| 4 | s5 | REF | 60% |
| 5 | s9 | ALT | 60% |
| 6 | s1 | BOTH | 55% |
| 7 | s5 | REF | 65% |
| 8 | s7 | ALT | 55% |
| 9 | s7 | REF | 50% |
| 10 | s8 | ALT | 70% |
| 11 | s8 | REF | 75% |
| 12 | s4 | ALT | 70% |
| 13 | s4 | REF | 65% |
| 14 | s9 | REF | 65% |
| 15 | s9 | ALT | 60% |
| 16 | s2 | ALT | 60% |
| 17 | s11 | BOTH | 65% |
| 18 | s3 | REF | 50% |
| 19 | s13 | ALT | 50% |
| 20 | s2 | REF | 55% |
| 21 | s14 | BOTH | 50% |
| 22 | s6 | REF | 45% |
| 23 | s6 | ALT | 40% |
| 24 | s11 | BOTH | 60% |
| 25 | s13 | ALT | 50% |
| 26 | s13 | REF | 55% |
| 27 | s13 | REF | 50% |
| 28 | s8 | REF | 75% |
| 29 | s9 | REF | 65% |
| 30 | s9 | REF | 65% |
| 31 | s5 | REF | 60% |
| 32 | s5 | REF | 50% |
| 33 | s10 | REF | 75% |
| 34 | s10 | ALT | 70% |
| 35 | s11 | ALT | 70% |
| 36 | s10 | ALT | 65% |
| 37 | s12 | ALT | 80% |
| 38 | s12 | REF | 75% |
| 39 | s8 | ALT | 60% |
| 40 | s12 | ALT | 80% |
| 41 | s12 | REF | 75% |
| 42 | s9 | ALT | 60% |
| 43 | s11 | REF | 75% |
| 44 | s10 | REF | 50% |
| 45 | s10 | ALT | 55% |
| 46 | s10 | REF | 60% |
| 47 | s5 | REF | 50% |
| 48 | s8 | ALT | 60% |
| 49 | s8 | REF | 65% |
| 50 | s3 | REF | 50% |
| 51 | s7 | REF | 45% |
| 52 | s7 | ALT | 50% |
| 53 | s13 | ALT | 45% |
| 54 | s14 | BOTH | 60% |
| 55 | s7 | ALT | 55% |
| 56 | s7 | REF | 50% |
| 57 | s13 | ALT | 50% |
| 58 | s13 | REF | 65% |
| 59 | s2 | ALT | 50% |
| 60 | s2 | REF | 45% |
| 61 | s3 | REF | 55% |
| 62 | s5 | REF | 65% |
| 63 | s7 | REF | 30% |
| 64 | s7 | ALT | 35% |
| 65 | s13 | ALT | 50% |
| 66 | s10 | REF | 65% |
| 67 | s10 | ALT | 60% |
| 68 | s13 | REF | 55% |
| 69 | s5 | REF | 60% |
| 70 | s11 | ALT | 75% |
| 71 | s11 | REF | 80% |
| 72 | s5 | REF | 50% |
| 73 | s14 | ALT | 65% |
| 74 | s14 | REF | 60% |
| 75 | s8 | ALT | 65% |
| 76 | s13 | ALT | 50% |
| 77 | s14 | REF | 60% |
| 78 | s14 | ALT | 65% |
| 79 | s3 | REF | 55% |
| 80 | s2 | REF | 45% |
| 81 | s2 | ALT | 50% |
| 82 | s8 | BOTH | 65% |
| 83 | s2 | REF | 50% |
| 84 | s2 | ALT | 55% |
| 85 | s11 | ALT | 80% |
| 86 | s10 | REF | 55% |
| 87 | s13 | REF | 65% |
| 88 | s10 | ALT | 50% |
| 89 | s8 | ALT | 70% |
| 90 | s2 | ALT | 65% |
| 91 | s8 | REF | 70% |
| 92 | s9 | ALT | 60% |
| 93 | s9 | REF | 65% |
| 94 | s10 | ALT | 50% |
| 95 | s10 | REF | 55% |
| 96 | s2 | BOTH | 60% |
| 97 | s10 | BOTH | 70% |
| 98 | s13 | ALT | 55% |
| 99 | s13 | REF | 60% |
| 100 | s11 | REF | 80% |
| 101 | s11 | ALT | 75% |
| 102 | s2 | ALT | 60% |
| 103 | s12 | REF | 70% |
| 104 | s14 | REF | 65% |
| 105 | s13 | ALT | 55% |
| 106 | s10 | REF | 60% |
| 107 | s10 | ALT | 55% |
| 108 | s12 | ALT | 80% |
| 109 | s12 | ALT | 75% |
| 110 | s2 | REF | 60% |
| 111 | s2 | ALT | 50% |
| 112 | s2 | REF | 45% |
| 113 | s8 | ALT | 65% |
| 114 | s12 | ALT | 75% |
| 115 | s12 | REF | 70% |
| 116 | s7 | ALT | 35% |
| 117 | s7 | REF | 30% |
| 118 | s14 | REF | 50% |
| 119 | s4 | ALT | 55% |
| 120 | s11 | ALT | 75% |
| 121 | s5 | REF | 65% |
| 122 | s2 | ALT | 55% |
| 123 | s2 | REF | 50% |
| 124 | s6 | REF | 45% |
| 125 | s6 | ALT | 50% |
| 126 | s7 | REF | 45% |
| 127 | s7 | ALT | 50% |
| 128 | s10 | REF | 65% |
| 129 | s10 | ALT | 60% |
| 130 | s13 | ALT | 50% |
| 131 | s14 | BOTH | 50% |
| 132 | s2 | REF | 45% |
| 133 | s13 | REF | 55% |
| 134 | s8 | BOTH | 65% |
| 135 | s10 | ALT | 70% |
| 136 | s6 | REF | 55% |
| 137 | s5 | REF | 55% |
| 138 | s2 | ALT | 50% |
| 139 | s4 | REF | 55% |
| 140 | s4 | ALT | 60% |
| 141 | s8 | ALT | 70% |
| 142 | s8 | REF | 75% |
| 143 | s5 | REF | 55% |
| 144 | s12 | ALT | 75% |
| 145 | s4 | BOTH | 60% |
| 146 | s11 | ALT | 80% |
| 147 | s13 | ALT | 65% |
| 148 | s11 | REF | 85% |
| 149 | s3 | REF | 55% |
| 150 | s11 | REF | 75% |
| 151 | s9 | ALT | 60% |
| 152 | s9 | REF | 65% |
| 153 | s14 | ALT | 75% |
| 154 | s14 | REF | 70% |
| 155 | s4 | ALT | 60% |
| 156 | s9 | BOTH | 75% |
| 157 | s9 | BOTH | 70% |
| 158 | s9 | REF | 65% |
| 159 | s14 | REF | 65% |
| 160 | s14 | ALT | 70% |
| 161 | s11 | ALT | 70% |
| 162 | s4 | REF | 55% |
| 163 | s6 | ALT | 45% |
| 164 | s6 | REF | 50% |
| 165 | s6 | ALT | 50% |
| 166 | s6 | REF | 55% |
| 167 | s1 | REF | 55% |
| 168 | s1 | ALT | 60% |
| 169 | s4 | REF | 65% |
| 170 | s4 | ALT | 70% |
| 171 | s1 | BOTH | 60% |
| 172 | s5 | REF | 65% |
| 173 | s12 | ALT | 75% |
| 174 | s13 | ALT | 55% |
| 175 | s13 | REF | 60% |
| 176 | s11 | ALT | 80% |
| 177 | s13 | REF | 70% |
| 178 | s11 | REF | 85% |
| 179 | s9 | REF | 65% |
| 180 | s8 | BOTH | 65% |
| 181 | s12 | ALT | 80% |
| 182 | s4 | BOTH | 65% |
| 183 | s4 | ALT | 60% |
| 184 | s9 | REF | 65% |
| 185 | s10 | REF | 70% |
| 186 | s8 | REF | 65% |
| 187 | s10 | REF | 75% |
| 188 | s12 | ALT | 75% |
| 189 | s12 | REF | 70% |
| 190 | s12 | REF | 75% |
| 191 | s9 | ALT | 60% |
| 192 | s12 | ALT | 75% |
| 193 | s12 | REF | 70% |
| 194 | s11 | ALT | 70% |
| 195 | s11 | REF | 75% |
| 196 | s12 | REF | 70% |
| 197 | s10 | BOTH | 50% |
| 198 | s9 | REF | 70% |
| 199 | s9 | ALT | 65% |
| 200 | s9 | ALT | 60% |
| 201 | s11 | BOTH | 70% |
| 202 | s10 | ALT | 60% |
| 203 | s11 | ALT | 70% |
| 204 | s11 | REF | 75% |
| 205 | s12 | REF | 70% |
| 206 | s13 | REF | 55% |
| 207 | s14 | ALT | 65% |
| 208 | s14 | REF | 60% |
| 209 | s10 | BOTH | 50% |
| 210 | s3 | REF | 55% |
| 211 | s3 | REF | 60% |
| 212 | s3 | BOTH | 50% |
| 213 | s9 | ALT | 60% |
| 214 | s11 | ALT | 70% |
| 215 | s14 | REF | 60% |
| 216 | s14 | ALT | 65% |
| 217 | s10 | ALT | 55% |
| 218 | s10 | REF | 60% |
| 219 | s7 | ALT | 35% |
| 220 | s7 | REF | 35% |
| 221 | s7 | ALT | 40% |
| 222 | s3 | REF | 50% |
| 223 | s7 | REF | 30% |
| 224 | s9 | ALT | 60% |
| 225 | s9 | REF | 65% |
| 226 | s10 | ALT | 50% |
| 227 | s10 | REF | 55% |
| 228 | s13 | REF | 55% |
| 229 | s14 | REF | 60% |
| 230 | s2 | REF | 45% |
| 231 | s2 | ALT | 50% |
| 232 | s7 | REF | 60% |
| 233 | s10 | ALT | 50% |
| 234 | s7 | ALT | 65% |
| 235 | s10 | ALT | 45% |
| 236 | s14 | REF | 65% |
| 237 | s14 | ALT | 65% |
| 238 | s2 | REF | 45% |
| 239 | s7 | REF | 55% |
| 240 | s12 | ALT | 80% |
| 241 | s14 | ALT | 75% |
| 242 | s14 | REF | 70% |
| 243 | s6 | ALT | 40% |
| 244 | s6 | REF | 45% |
| 245 | s2 | REF | 55% |
| 246 | s2 | REF | 45% |
| 247 | s2 | ALT | 50% |
| 248 | s2 | ALT | 50% |
| 249 | s2 | REF | 45% |
| 250 | s13 | BOTH | 65% |
| 251 | s1 | REF | 55% |
| 252 | s1 | ALT | 60% |
| 253 | s12 | ALT | 70% |
| 254 | s12 | REF | 65% |
| 255 | s9 | ALT | 65% |
| 256 | s7 | REF | 35% |
| 257 | s7 | ALT | 40% |
| 258 | s4 | REF | 60% |
| 259 | s4 | ALT | 65% |
| 260 | s11 | REF | 70% |
| 261 | s11 | ALT | 65% |
| 262 | s13 | ALT | 50% |
| 263 | s14 | REF | 65% |
| 264 | s14 | ALT | 70% |
| 265 | s14 | REF | 50% |
| 266 | s14 | ALT | 55% |
| 267 | s11 | REF | 75% |
| 268 | s11 | ALT | 70% |
| 269 | s4 | REF | 50% |
| 270 | s1 | BOTH | 50% |
| 271 | s1 | BOTH | 50% |
| 272 | s2 | ALT | 50% |
| 273 | s3 | BOTH | 45% |
| 274 | s3 | ALT | 50% |
| 275 | s4 | REF | 65% |
| 276 | s6 | ALT | 40% |
| 277 | s6 | REF | 45% |
| 278 | s7 | BOTH | 50% |
| 279 | s8 | ALT | 70% |
| 280 | s8 | REF | 75% |
| 281 | s4 | ALT | 65% |
| 282 | s4 | REF | 60% |
| 283 | s2 | ALT | 55% |
| 284 | s10 | ALT | 60% |
| 285 | s10 | REF | 65% |
| 286 | s1 | REF | 50% |
| 287 | s2 | REF | 50% |
| 288 | s3 | BOTH | 45% |
| 289 | s1 | ALT | 65% |
| 290 | s1 | REF | 60% |
| 291 | s7 | REF | 25% |
| 292 | s7 | ALT | 30% |
| 293 | s3 | REF | 50% |
| 294 | s3 | ALT | 50% |
| 295 | s8 | ALT | 70% |
| 296 | s4 | REF | 65% |
| 297 | s5 | ALT | 65% |
| 298 | s6 | ALT | 45% |
| 299 | s6 | REF | 50% |
| 300 | s6 | ALT | 45% |
| 301 | s6 | REF | 50% |
| 302 | s10 | REF | 70% |
| 303 | s10 | ALT | 65% |
| 304 | s4 | ALT | 70% |
| 305 | s11 | REF | 60% |
| 306 | s5 | REF | 50% |
| 307 | s6 | ALT | 45% |
| 308 | s7 | REF | 40% |
| 309 | s7 | ALT | 45% |
| 310 | s7 | REF | 30% |
| 311 | s7 | ALT | 35% |
| 312 | s3 | REF | 55% |
| 313 | s3 | ALT | 50% |
| 314 | s1 | REF | 60% |
| 315 | s1 | ALT | 65% |
| 316 | s2 | REF | 50% |
| 317 | s3 | REF | 55% |
| 318 | s3 | ALT | 50% |
| 319 | s12 | REF | 70% |
| 320 | s12 | ALT | 75% |
| 321 | s9 | REF | 65% |
| 322 | s9 | ALT | 60% |
| 323 | s3 | REF | 55% |
| 324 | s3 | ALT | 50% |
| 325 | s13 | ALT | 45% |
| 326 | s1 | ALT | 55% |
| 327 | s1 | REF | 50% |
| 328 | s1 | ALT | 60% |
| 329 | s8 | BOTH | 60% |
| 330 | s11 | ALT | 75% |
| 331 | s11 | REF | 80% |
| 332 | s12 | REF | 70% |
| 333 | s2 | BOTH | 60% |
| 334 | s5 | REF | 70% |
| 335 | s10 | REF | 60% |
| 336 | s10 | ALT | 55% |
| 337 | s10 | ALT | 65% |
| 338 | s10 | REF | 70% |
| 339 | s13 | REF | 55% |
| 340 | s4 | REF | 50% |
| 341 | s4 | ALT | 55% |
| 342 | s1 | ALT | 60% |
| 343 | s1 | REF | 55% |
| 344 | s1 | REF | 55% |
| 345 | s13 | REF | 60% |
| 346 | s5 | REF | 60% |
| 347 | s3 | BOTH | 50% |
| 348 | s1 | ALT | 65% |
| 349 | s1 | REF | 60% |
| 350 | s1 | ALT | 55% |
| 351 | s7 | REF | 30% |
| 352 | s7 | ALT | 35% |
| 353 | s2 | ALT | 50% |
| 354 | s2 | REF | 45% |
| 355 | s4 | REF | 60% |
| 356 | s4 | ALT | 65% |
| 357 | s7 | REF | 40% |
| 358 | s7 | ALT | 45% |
| 359 | s1 | REF | 55% |
| 360 | s3 | REF | 55% |
| 361 | s3 | ALT | 50% |
| 362 | s1 | ALT | 55% |
| 363 | s10 | REF | 60% |
| 364 | s5 | REF | 60% |
| 365 | s1 | REF | 60% |
| 366 | s1 | ALT | 65% |
| 367 | s11 | REF | 75% |
| 368 | s13 | ALT | 45% |
| 369 | s2 | REF | 45% |
| 370 | s2 | ALT | 50% |
| 371 | s2 | BOTH | 45% |
| 372 | s8 | ALT | 70% |
| 373 | s13 | ALT | 45% |
| 374 | s13 | REF | 50% |
| 375 | s4 | ALT | 70% |
| 376 | s4 | REF | 65% |
| 377 | s8 | REF | 75% |
| 378 | s8 | ALT | 70% |
| 379 | s4 | REF | 70% |
| 380 | s8 | REF | 75% |
| 381 | s8 | ALT | 70% |
| 382 | s12 | BOTH | 70% |
| 383 | s7 | ALT | 40% |
| 384 | s7 | REF | 35% |
| 385 | s3 | REF | 55% |
| 386 | s3 | ALT | 50% |
| 387 | s6 | REF | 50% |
| 388 | s6 | ALT | 45% |
| 389 | s5 | ALT | 70% |
| 390 | s1 | REF | 60% |
| 391 | s1 | ALT | 65% |
| 392 | s6 | ALT | 45% |
| 393 | s6 | REF | 50% |
| 394 | s6 | ALT | 45% |
| 395 | s1 | ALT | 60% |
| 396 | s6 | REF | 50% |
| 397 | s13 | BOTH | 55% |
| 398 | s11 | ALT | 75% |
| 399 | s4 | ALT | 70% |
| 400 | s11 | REF | 80% |
| 401 | s12 | REF | 75% |
| 402 | s12 | ALT | 80% |
| 403 | s11 | REF | 80% |
| 404 | s11 | ALT | 75% |
| 405 | s8 | ALT | 60% |
| 406 | s5 | REF | 70% |
| 407 | s14 | REF | 65% |
| 408 | s4 | REF | 65% |
| 409 | s4 | BOTH | 70% |
| 410 | s8 | ALT | 65% |
| 411 | s8 | REF | 70% |
| 412 | s9 | REF | 70% |
| 413 | s6 | REF | 50% |
| 414 | s9 | REF | 65% |
| 415 | s11 | BOTH | 65% |
| 416 | s13 | ALT | 45% |
| 417 | s13 | REF | 50% |
| 418 | s14 | ALT | 60% |
| 419 | s14 | REF | 55% |
| 420 | s3 | REF | 55% |
| 421 | s3 | ALT | 50% |
| 422 | s6 | ALT | 50% |
| 423 | s1 | REF | 55% |
| 424 | s13 | ALT | 45% |
| 425 | s13 | REF | 50% |
| 426 | s14 | REF | 55% |
| 427 | s14 | ALT | 60% |
| 428 | s2 | REF | 45% |
| 429 | s8 | REF | 70% |
| 430 | s8 | ALT | 65% |
| 431 | s7 | REF | 30% |
| 432 | s3 | ALT | 50% |
| 433 | s13 | REF | 55% |
| 434 | s5 | REF | 50% |
| 435 | s6 | BOTH | 45% |
| 436 | s9 | ALT | 60% |
| 437 | s10 | ALT | 50% |
| 438 | s10 | REF | 55% |
| 439 | s12 | BOTH | 70% |
| 440 | s2 | REF | 45% |
| 441 | s2 | ALT | 50% |
| 442 | s7 | REF | 55% |
| 443 | s4 | ALT | 65% |
| 444 | s4 | REF | 60% |
| 445 | s13 | ALT | 50% |
| 446 | s14 | ALT | 70% |
| 447 | s14 | REF | 65% |
| 448 | s2 | ALT | 50% |
| 449 | s2 | REF | 45% |
| 450 | s3 | ALT | 50% |
| 451 | s2 | REF | 45% |
| 452 | s2 | ALT | 50% |
| 453 | s4 | REF | 55% |
| 454 | s4 | ALT | 60% |
| 455 | s3 | REF | 55% |
| 456 | s3 | ALT | 55% |
| 457 | s8 | ALT | 75% |
| 458 | s8 | REF | 80% |
| 459 | s4 | REF | 60% |
| 460 | s1 | ALT | 55% |
| 461 | s1 | REF | 50% |
| 462 | s7 | REF | 35% |
| 463 | s7 | ALT | 40% |
| 464 | s12 | REF | 70% |
| 465 | s12 | ALT | 75% |
| 466 | s3 | REF | 55% |
| 467 | s3 | ALT | 55% |
| 468 | s1 | ALT | 60% |
| 469 | s9 | REF | 70% |
| 470 | s9 | ALT | 65% |
| 471 | s5 | BOTH | 70% |
| 472 | s4 | ALT | 55% |
| 473 | s1 | REF | 55% |
| 474 | s6 | REF | 45% |
| 475 | s6 | ALT | 40% |
| 476 | s13 | ALT | 55% |
| 477 | s1 | REF | 65% |
| 478 | s1 | ALT | 70% |
| 479 | s2 | BOTH | 50% |
| 480 | s8 | ALT | 70% |
| 481 | s8 | REF | 75% |
| 482 | s13 | ALT | 50% |
| 483 | s13 | REF | 55% |
| 484 | s8 | REF | 75% |
| 485 | s8 | ALT | 70% |
| 486 | s9 | ALT | 60% |
| 487 | s9 | REF | 65% |
| 488 | s11 | ALT | 70% |
| 489 | s12 | REF | 70% |
| 490 | s7 | ALT | 45% |
| 491 | s7 | REF | 40% |
| 492 | s3 | REF | 55% |
| 493 | s3 | ALT | 55% |
| 494 | s6 | REF | 55% |
| 495 | s6 | ALT | 50% |
| 496 | s11 | REF | 75% |
| 497 | s12 | REF | 75% |
| 498 | s11 | REF | 80% |
| 499 | s11 | ALT | 75% |
| 500 | s11 | ALT | 70% |
| 501 | s14 | REF | 65% |
| 502 | s7 | ALT | 40% |
| 503 | s8 | REF | 75% |
| 504 | s9 | REF | 65% |
| 505 | s8 | ALT | 65% |
| 506 | s12 | ALT | 80% |
| 507 | s12 | ALT | 75% |
| 508 | s12 | REF | 75% |
| 509 | s9 | ALT | 65% |
| 510 | s9 | REF | 70% |
| 511 | s9 | ALT | 60% |
| 512 | s11 | ALT | 70% |
| 513 | s11 | REF | 75% |
| 514 | s10 | BOTH | 50% |
| 515 | s9 | REF | 65% |
| 516 | s9 | ALT | 60% |
| 517 | s10 | ALT | 60% |
| 518 | s10 | REF | 65% |
| 519 | s13 | ALT | 45% |
| 520 | s13 | REF | 55% |
| 521 | s1 | ALT | 60% |
| 522 | s13 | REF | 55% |
| 523 | s2 | REF | 50% |
| 524 | s3 | ALT | 55% |
| 525 | s6 | BOTH | 50% |
| 526 | s12 | BOTH | 70% |
| 527 | s4 | ALT | 70% |
| 528 | s4 | REF | 65% |
| 529 | s13 | ALT | 50% |
| 530 | s13 | REF | 65% |
| 531 | s13 | ALT | 60% |
| 532 | s4 | ALT | 70% |
| 533 | s4 | REF | 65% |
| 534 | s1 | ALT | 60% |
| 535 | s1 | REF | 55% |
| 536 | s12 | ALT | 75% |
| 537 | s4 | BOTH | 55% |
| 538 | s6 | REF | 50% |
| 539 | s6 | ALT | 45% |
| 540 | s10 | REF | 70% |
| 541 | s10 | ALT | 65% |
| 542 | s11 | ALT | 80% |
| 543 | s11 | REF | 85% |
| 544 | s10 | REF | 50% |
| 545 | s13 | ALT | 55% |
| 546 | s8 | REF | 75% |
| 547 | s8 | ALT | 70% |
| 548 | s9 | ALT | 60% |
| 549 | s9 | REF | 65% |
| 550 | s11 | ALT | 75% |
| 551 | s7 | REF | 45% |
| 552 | s11 | REF | 75% |
| 553 | s11 | ALT | 70% |
| 554 | s9 | REF | 65% |
| 555 | s8 | ALT | 65% |
| 556 | s9 | ALT | 60% |
| 557 | s1 | ALT | 65% |
| 558 | s2 | REF | 55% |
| 559 | s6 | BOTH | 50% |
| 560 | s4 | ALT | 75% |
| 561 | s12 | BOTH | 75% |
| 562 | s10 | ALT | 70% |
| 563 | s10 | REF | 55% |
| 564 | s8 | REF | 80% |
| 565 | s9 | ALT | 60% |
| 566 | s11 | ALT | 75% |
| 567 | s11 | REF | 80% |
| 568 | s9 | REF | 65% |
| 569 | s2 | REF | 50% |
| 570 | s10 | REF | 55% |
| 571 | s9 | BOTH | 65% |
| 572 | s11 | REF | 85% |
| 573 | s11 | ALT | 80% |
| 574 | s12 | REF | 70% |
| 575 | s12 | ALT | 75% |
| 576 | s13 | ALT | 60% |
| 577 | s13 | REF | 65% |
| 578 | s14 | ALT | 65% |
| 579 | s12 | REF | 70% |
| 580 | s12 | ALT | 75% |
| 581 | s13 | ALT | 60% |
| 582 | s14 | ALT | 60% |
| 583 | s8 | REF | 70% |
| 584 | s12 | REF | 70% |
| 585 | s7 | REF | 60% |
| 586 | s14 | REF | 60% |
| 587 | s5 | REF | 55% |
| 588 | s12 | ALT | 75% |
| 589 | s2 | BOTH | 60% |
| 590 | s7 | ALT | 65% |
| 591 | s13 | ALT | 55% |
| 592 | s13 | REF | 60% |
| 593 | s5 | REF | 75% |
| 594 | s14 | ALT | 55% |
| 595 | s9 | REF | 70% |
| 596 | s3 | REF | 55% |
| 597 | s11 | REF | 80% |
| 598 | s11 | REF | 70% |
| 599 | s8 | REF | 70% |
| 600 | s2 | ALT | 60% |
| 601 | s7 | REF | 60% |
| 602 | s10 | ALT | 50% |
| 603 | s11 | BOTH | 70% |
| 604 | s7 | ALT | 55% |
| 605 | s7 | REF | 50% |
| 606 | s10 | REF | 65% |
| 607 | s10 | ALT | 60% |
| 608 | s14 | REF | 70% |
| 609 | s12 | REF | 75% |
| 610 | s12 | ALT | 80% |
| 611 | s14 | REF | 55% |
| 612 | s5 | REF | 55% |
| 613 | s14 | ALT | 70% |
| 614 | s14 | REF | '65% |
| 615 | s5 | REF | 60% |
| 616 | s12 | ALT | 75% |
| 617 | s10 | ALT | 70% |
| 618 | s10 | REF | 75% |
| 619 | s2 | REF | 55% |
| 620 | s2 | ALT | 60% |
| 621 | s8 | ALT | 65% |
| 622 | s14 | REF | 70% |
| 623 | s14 | ALT | 75% |
| 624 | s12 | ALT | 75% |
| 625 | s7 | ALT | 65% |
| 626 | s7 | ALT | 40% |
| 627 | s7 | REF | 35% |
| 628 | s4 | ALT | 55% |
| 629 | s5 | REF | 70% |
| 630 | s5 | REF | 55% |
| 631 | s14 | ALT | 55% |
| 632 | s3 | ALT | 50% |
| 633 | s13 | REF | 60% |
| 634 | s3 | ALT | 55% |
| 635 | s14 | ALT | 70% |
| 636 | s14 | REF | 65% |
| 637 | s9 | BOTH | 70% |
| 638 | s9 | BOTH | 70% |
| 639 | s3 | REF | 55% |
| 640 | s9 | ALT | 70% |
| 641 | s5 | REF | 65% |
| 642 | s3 | REF | 60% |
| 643 | s8 | REF | 65% |
| 644 | s11 | REF | 75% |
| 645 | s11 | REF | 70% |
| 646 | s14 | ALT | 70% |
| 647 | s4 | REF | 55% |
| 648 | s4 | ALT | 60% |
| 649 | s8 | REF | 70% |
| 650 | s4 | ALT | 65% |
| 651 | s4 | REF | 60% |
| 652 | s2 | ALT | 55% |
| 653 | s1 | ALT | 65% |
| 654 | s1 | REF | 60% |
| 655 | s13 | REF | 70% |
| 656 | s3 | BOTH | 50% |
| 657 | s1 | REF | 60% |
| 658 | s1 | REF | 55% |
| 659 | s9 | ALT | 60% |
| 660 | s9 | REF | 65% |
| 661 | s10 | ALT | 50% |
| 662 | s10 | REF | 55% |
| 663 | s14 | BOTH | 55% |
| 664 | s3 | REF | 55% |
| 665 | s3 | ALT | 55% |
| 666 | s9 | REF | 65% |
| 667 | s11 | ALT | 65% |
| 668 | s14 | REF | 60% |
| 669 | s14 | ALT | 65% |
| 670 | s7 | ALT | 50% |
| 671 | s7 | REF | 45% |
| 672 | s10 | BOTH | 65% |
| 673 | s3 | ALT | 55% |
| 674 | s14 | ALT | 75% |
| 675 | s14 | REF | 70% |
| 676 | s13 | REF | 65% |
| 677 | s3 | REF | 60% |
| 678 | s3 | ALT | 55% |
| 679 | s6 | REF | 55% |
| 680 | s6 | ALT | 50% |
| 681 | s14 | REF | 50% |
| 682 | s10 | ALT | 65% |
| 683 | s8 | REF | 70% |
| 684 | s5 | REF | 50% |
| 685 | s14 | ALT | 70% |
| 686 | s14 | REF | 65% |
| 687 | s5 | REF | 65% |
| 688 | s5 | REF | 55% |
| 689 | s12 | REF | 75% |
| 690 | s12 | ALT | 80% |
| 691 | s3 | REF | 60% |
| 692 | s2 | BOTH | 60% |
| 693 | s3 | ALT | 55% |
| 694 | s10 | REF | 75% |
| 695 | s10 | ALT | 70% |
| 696 | s2 | REF | 50% |
| 697 | s7 | ALT | 65% |
| 698 | s7 | REF | 60% |
| 699 | s14 | REF | 70% |
| 700 | s14 | ALT | 75% |
| 701 | s3 | BOTH | 55% |
| 702 | s1 | REF | 55% |
| 703 | s1 | ALT | 70% |
| 704 | s1 | REF | 65% |
| 705 | s11 | ALT | 65% |
| 706 | s1 | ALT | 60% |
| 707 | s6 | REF | 50% |
| 708 | s6 | ALT | 45% |
| 709 | s7 | REF | 35% |
| 710 | s13 | ALT | 50% |
| 711 | s13 | REF | 55% |
| 712 | s7 | BOTH | 30% |
| 713 | s4 | REF | 50% |
| 714 | s5 | REF | 50% |
| 715 | s9 | ALT | 60% |
| 716 | s6 | ALT | 45% |
| 717 | s6 | REF | 50% |
| 718 | s10 | REF | 70% |
| 719 | s4 | ALT | 70% |
| 720 | s5 | REF | 55% |
| 721 | s3 | REF | 55% |
| 722 | s3 | ALT | 55% |
| 723 | s1 | REF | 60% |
| 724 | s1 | ALT | 65% |
| 725 | s3 | REF | 55% |
| 726 | s3 | ALT | 55% |
| 727 | s9 | REF | 65% |
| 728 | s9 | ALT | 60% |
| 729 | s1 | ALT | 60% |
| 730 | s1 | REF | 55% |
| 731 | s8 | REF | 60% |
| 732 | s11 | BOTH | 65% |
| 733 | s1 | REF | 50% |
| 734 | s5 | REF | 60% |
| 735 | s1 | ALT | 55% |
| 736 | s4 | REF | 60% |
| 737 | s4 | ALT | 65% |
| 738 | s1 | REF | 55% |
| 739 | s1 | ALT | 60% |
| 740 | s1 | BOTH | 55% |
| 741 | s13 | ALT | 45% |
| 742 | s2 | REF | 45% |
| 743 | s2 | ALT | 50% |
| 744 | s8 | ALT | 70% |
| 745 | s8 | REF | 75% |
| 746 | s4 | ALT | 75% |
| 747 | s4 | REF | 70% |
| 748 | s9 | REF | 65% |
| 749 | s5 | REF | 60% |
| 750 | s1 | ALT | 70% |
| 751 | s3 | ALT | 50% |
| 752 | s11 | ALT | 75% |
| 753 | s11 | REF | 80% |
| 754 | s12 | ALT | 80% |
| 755 | s12 | REF | 75% |
| 756 | s13 | BOTH | 55% |
| 757 | s8 | REF | 75% |
| 758 | s8 | ALT | 70% |
| 759 | s13 | REF | 55% |
| 760 | s2 | ALT | 55% |
| 761 | s14 | ALT | 70% |
| 762 | s14 | REF | 65% |
| 763 | s2 | ALT | 55% |
| 764 | s2 | REF | 50% |
| 765 | s13 | BOTH | 55% |
| 766 | s3 | REF | 55% |
| 767 | s3 | ALT | 50% |
| 768 | s5 | REF | 70% |
| 769 | s8 | ALT | 75% |
| 770 | s8 | REF | 80% |
| 771 | s1 | ALT | 65% |
| 772 | s5 | BOTH | 70% |
| 773 | s12 | REF | 75% |
| 774 | s13 | ALT | 50% |
| 775 | s13 | REF | 55% |
| 776 | s14 | ALT | 65% |
| 777 | s14 | REF | 60% |
| 778 | s13 | REF | 55% |
| 779 | s4 | ALT | 70% |
| 780 | s4 | REF | 65% |
| 781 | s8 | ALT | 65% |
| 782 | s10 | REF | 75% |
| 783 | s10 | ALT | 70% |
| 784 | s8 | REF | 80% |
| 785 | s8 | ALT | 75% |
| 786 | s9 | BOTH | 70% |
| 787 | s11 | REF | 85% |
| 788 | s11 | ALT | 80% |
| 789 | s12 | REF | 75% |
| 790 | s12 | ALT | 80% |
| 791 | s8 | REF | 75% |
| 792 | s8 | ALT | 70% |
| 793 | s9 | REF | 70% |
| 794 | s8 | REF | 70% |
| 795 | s7 | ALT | 60% |
| 796 | s7 | REF | 55% |
| 797 | s12 | REF | 75% |
| 798 | s12 | ALT | 80% |
| 799 | s5 | REF | 60% |
| 800 | s2 | REF | 60% |
| 801 | s2 | ALT | 65% |
| 802 | s12 | BOTH | 75% |
| 803 | s7 | ALT | 40% |
| 804 | s7 | REF | 35% |
| 805 | s11 | REF | 85% |
| 806 | s11 | ALT | 80% |
| 807 | s5 | REF | 55% |
| 808 | s3 | ALT | 55% |
| 809 | s5 | REF | 70% |
| 810 | s3 | REF | 60% |
| 811 | s3 | REF | 60% |
| 812 | s3 | ALT | 55% |
| 813 | s3 | ALT | 55% |
| 814 | s6 | REF | 55% |
| 815 | s6 | ALT | 50% |
| 816 | s10 | ALT | 70% |
| 817 | s8 | REF | 70% |
| 818 | s8 | ALT | 65% |
| 819 | s1 | REF | 60% |
| 820 | s1 | ALT | 65% |
| 821 | s10 | REF | 75% |
| 822 | s10 | REF | 75% |
| 823 | s10 | ALT | 70% |
| 824 | s3 | REF | 60% |
| 825 | s3 | ALT | 55% |
| 826 | s9 | REF | 70% |
| 827 | s9 | ALT | 65% |
| 828 | s14 | BOTH | 55% |
| 829 | s2 | ALT | 55% |
| 830 | s2 | REF | 50% |
| 831 | s1 | REF | 60% |
| 832 | s1 | ALT | 65% |
| 833 | s13 | ALT | 50% |
| 834 | s2 | REF | 50% |
| 835 | s2 | ALT | 55% |
| 836 | s4 | ALT | 75% |
| 837 | s8 | BOTH | 65% |
| 838 | s6 | REF | 55% |
| 839 | s2 | ALT | 55% |
| 840 | s13 | BOTH | 55% |
| 841 | s3 | REF | 55% |
| 842 | s3 | ALT | 50% |
| 843 | s4 | REF | 55% |
| 844 | s4 | ALT | 60% |
| 845 | s1 | ALT | 65% |
| 846 | s1 | REF | 60% |
| 847 | s3 | REF | 55% |
| 848 | s12 | ALT | 80% |
| 849 | s12 | REF | 75% |
| 850 | s4 | BOTH | 65% |
| 851 | s8 | ALT | 65% |
| 852 | s10 | REF | 75% |
| 853 | s10 | ALT | 70% |
| 854 | s11 | REF | 75% |
| 855 | s7 | ALT | 65% |
| 856 | s7 | REF | 60% |
| 857 | s12 | REF | 75% |
| 858 | s12 | ALT | 80% |
| 859 | s11 | REF | 70% |
| 860 | s4 | ALT | 60% |
| 861 | s11 | REF | 85% |
| 862 | s14 | REF | 70% |
| 863 | s14 | ALT | 75% |
| 864 | s8 | REF | 70% |
| 865 | s8 | ALT | 65% |
| 866 | s5 | REF | 75% |
| 867 | s11 | ALT | 70% |
| 868 | s4 | REF | 55% |
| 869 | s3 | REF | 60% |
| 870 | s3 | ALT | 55% |
| 871 | s1 | REF | 60% |
| 872 | s1 | ALT | 65% |
| 873 | s14 | BOTH | 55% |
| 874 | s4 | REF | 70% |
| 875 | s4 | ALT | 75% |
| 876 | s4 | BOTH | 75% |
| 877 | s4 | REF | 55% |
| 878 | s4 | ALT | 60% |
| 879 | s1 | ALT | 70% |
| 880 | s1 | REF | 65% |
| 881 | s12 | ALT | 80% |
| 882 | s12 | REF | 75% |
| 883 | s8 | ALT | 70% |
| 884 | s8 | REF | 75% |
| 885 | s14 | ALT | 70% |
| 886 | s4 | REF | 55% |
| 887 | s12 | ALT | 80% |
| 888 | s4 | ALT | 60% |
| 889 | s4 | REF | 55% |
| 890 | s4 | ALT | 60% |
| 891 | s4 | REF | 55% |
| 892 | s4 | REF | 55% |
| 893 | s9 | ALT | 65% |
| 894 | s9 | REF | 70% |
| 895 | s9 | ALT | 65% |
| 896 | s9 | REF | 70% |
| 897 | s2 | ALT | 60% |
| 898 | s2 | REF | 55% |
| 899 | s6 | REF | 55% |
| 900 | s9 | ALT | 65% |
| 901 | s9 | REF | 70% |
| 902 | s12 | REF | 70% |
| 903 | s1 | ALT | 60% |
| 904 | s13 | ALT | 50% |
| 905 | s9 | REF | 70% |
| 906 | s9 | ALT | 65% |
| 907 | s6 | REF | 55% |
| 908 | s5 | REF | 50% |
| 909 | s3 | ALT | 55% |
| 910 | s2 | ALT | 55% |
| 911 | s2 | REF | 50% |
| 912 | s9 | ALT | 60% |
| 913 | s8 | ALT | 65% |
| 914 | s8 | REF | 70% |
| 915 | s9 | REF | 65% |
| 916 | s3 | ALT | 55% |
| 917 | s13 | ALT | 50% |
| 918 | s13 | REF | 55% |
| 919 | s9 | REF | 70% |
| 920 | s9 | ALT | 65% |
| 921 | s12 | ALT | 75% |
| 922 | s12 | REF | 70% |
| 923 | s1 | ALT | 60% |
| 924 | s1 | REF | 55% |
| 925 | s8 | REF | 75% |
| 926 | s8 | ALT | 70% |
| 927 | s11 | ALT | 70% |
| 928 | s11 | REF | 75% |
| 929 | s12 | REF | 65% |
| 930 | s4 | REF | 60% |
| 931 | s4 | ALT | 65% |
| 932 | s4 | REF | 65% |
| 933 | s7 | REF | 35% |
| 934 | s12 | REF | 70% |
| 935 | s1 | REF | 55% |
| 936 | s3 | ALT | 50% |
| 937 | s1 | REF | 50% |
| 938 | s7 | REF | 50% |
| 939 | s7 | ALT | 55% |
| 940 | s14 | BOTH | 55% |
| 941 | s3 | REF | 55% |
| 942 | s3 | ALT | 50% |
| 943 | s7 | BOTH | 35% |
| 944 | s9 | REF | 70% |
| 945 | s9 | ALT | 65% |
| 946 | s13 | REF | 50% |
| 947 | s13 | ALT | 50% |
| 948 | s5 | REF | 55% |
| 949 | s10 | ALT | 45% |
| 950 | s6 | REF | 50% |
| 951 | s11 | REF | 75% |
| 952 | s11 | ALT | 70% |
| 953 | s9 | ALT | 65% |
| 954 | s9 | REF | 70% |
| 955 | s11 | ALT | 65% |
| 956 | s11 | REF | 70% |
| 957 | s10 | REF | 60% |
| 958 | s14 | REF | 55% |
| 959 | s14 | ALT | 60% |
| 960 | s6 | REF | 50% |
| 961 | s6 | ALT | 45% |
| 962 | s13 | REF | 55% |
| 963 | s7 | ALT | 45% |
| 964 | s7 | REF | 40% |
| 965 | s12 | BOTH | 75% |
| 966 | s14 | REF | 55% |
| 967 | s5 | REF | 50% |
| 968 | s2 | ALT | 55% |
| 969 | s10 | BOTH | 65% |
| 970 | s14 | REF | 65% |
| 971 | s14 | ALT | 70% |
| 972 | s3 | ALT | 55% |
| 973 | s14 | ALT | 70% |
| 974 | s14 | REF | 65% |
| 975 | s4 | ALT | 65% |
| 976 | s4 | REF | 60% |
| 977 | s13 | REF | 65% |
| 978 | s14 | REF | 55% |
| 979 | s14 | ALT | 60% |
| 980 | s3 | REF | 55% |
| 981 | s3 | ALT | 50% |
| 982 | s12 | REF | 75% |
| 983 | s12 | ALT | 80% |
| 984 | s3 | REF | 55% |
| 985 | s3 | ALT | 55% |
| 986 | s6 | REF | 50% |
| 987 | s6 | ALT | 45% |
| 988 | s9 | ALT | 60% |
| 989 | s8 | ALT | 65% |
| 990 | s8 | REF | 70% |
| 991 | s14 | REF | 50% |
| 992 | s10 | ALT | 65% |
| 993 | s13 | ALT | 55% |
| 994 | s8 | REF | 70% |
| 995 | s8 | ALT | 65% |
| 996 | s5 | REF | 50% |
| 997 | s14 | ALT | 65% |
| 998 | s14 | REF | 60% |
| 999 | s5 | REF | 65% |
| 1000 | s5 | BOTH | 70% |
| 1001 | s5 | REF | 50% |
| 1002 | s11 | REF | 80% |
| 1003 | s11 | ALT | 75% |
| 1004 | s12 | REF | 75% |
| 1005 | s12 | ALT | 70% |
| 1006 | s3 | REF | 55% |
| 1007 | s2 | ALT | 60% |
| 1008 | s3 | ALT | 55% |
| 1009 | s5 | REF | 65% |
| 1010 | s3 | REF | 55% |
| 1011 | s3 | ALT | 55% |
| 1012 | s10 | ALT | 70% |
| 1013 | s10 | REF | 75% |
| 1014 | s10 | REF | 70% |
| 1015 | s10 | ALT | 65% |
| 1016 | s2 | ALT | 50% |
| 1017 | s2 | REF | 45% |
| 1018 | s1 | REF | 55% |
| 1019 | s13 | ALT | 45% |
| 1020 | s13 | REF | 50% |
| 1021 | s3 | BOTH | 50% |
| 1022 | s13 | BOTH | 55% |
| 1023 | s7 | ALT | 65% |
| 1024 | s7 | REF | 60% |
| 1025 | s14 | REF | 70% |
| 1026 | s14 | ALT | 75% |
| 1027 | s3 | REF | 55% |
| 1028 | s3 | ALT | 55% |
| 1029 | s8 | BOTH | 60% |
| 1030 | s1 | REF | 60% |
| 1031 | s11 | ALT | 65% |
| 1032 | s10 | REF | 70% |
| 1033 | s2 | ALT | 55% |
| 1034 | s2 | REF | 50% |
| 1035 | s1 | ALT | 55% |
| 1036 | s6 | REF | 50% |
| 1037 | s6 | ALT | 45% |
| 1038 | s3 | REF | 55% |
| 1039 | s3 | ALT | 55% |
| 1040 | s7 | ALT | 30% |
| 1041 | s7 | REF | 25% |
| 1042 | s7 | BOTH | 35% |
| 1043 | s9 | ALT | 60% |
| 1044 | s13 | REF | 55% |
| 1045 | s2 | REF | 45% |
| 1046 | s2 | ALT | 50% |
| 1047 | s12 | ALT | 75% |
| 1048 | s12 | REF | 70% |
| 1049 | s5 | REF | 60% |
| 1050 | s6 | ALT | 40% |
| 1051 | s6 | REF | 45% |
| 1052 | s4 | ALT | 65% |
| 1053 | s7 | REF | 30% |
| 1054 | s14 | REF | 60% |
| 1055 | s6 | ALT | 40% |
| 1056 | s6 | REF | 45% |
| 1057 | s11 | REF | 80% |
| 1058 | s2 | ALT | 50% |
| 1059 | s5 | ALT | 55% |
| 1060 | s3 | ALT | 45% |
| 1061 | s6 | REF | 40% |
| 1062 | s6 | BOTH | 25% |
| 1063 | s6 | ALT | 35% |
| 1064 | s8 | ALT | 70% |
| 1065 | s9 | ALT | 60% |
| 1066 | s5 | ALT | 55% |
| 1067 | s5 | ALT | 45% |
| 1068 | s11 | REF | 75% |
| 1069 | s9 | REF | 65% |
| 1070 | s11 | ALT | 70% |
| 1071 | s3 | ALT | 45% |
| 1072 | s3 | ALT | 50% |
| 1073 | s5 | ALT | 60% |
| 1074 | s5 | ALT | 55% |
| 1075 | s5 | ALT | 45% |
| 1076 | s3 | ALT | 50% |
| 1077 | s11 | REF | 85% |
| 1078 | s8 | REF | 75% |
| 1079 | S14 | ALT | 70% |
| 1080 | s12 | REF | 75% |
| 1081 | S14 | ALT | 55% |
| 1082 | s5 | ALT | 60% |
| 1083 | s6 | ALT | 40% |
| 1084 | s13 | REF | 60% |
| 1085 | s2 | ALT | 50% |
| 1086 | s13 | ALT | 50% |
| 1087 | s5 | ALT | 50% |
| 1088 | s5 | ALT | 50% |
| 1089 | s3 | ALT | 50% |
| 1090 | s5 | ALT | 60% |
| 1091 | s4 | REF | 55% |
| 1092 | s10 | REF | 65% |
| 1093 | s3 | ALT | 50% |
| 1094 | s3 | ALT | 55% |
| 1095 | s6 | BOTH | 30% |
| 1096 | s3 | ALT | 45% |
| 1097 | s10 | REF | 55% |
| 1098 | s10 | REF | 50% |
| 1099 | s7 | ALT | 60% |
| 1100 | s12 | REF | 75% |
| 1101 | s9 | REF | 70% |
| 1102 | s7 | BOTH | 50% |
| 1103 | s2 | REF | 45% |
| 1104 | s6 | REF | 45% |
| 1105 | s6 | ALT | 40% |
| 1106 | s1 | ALT | 55% |
| 1107 | s3 | ALT | 45% |
| 1108 | s6 | REF | 45% |
| 1109 | s6 | ALT | 40% |
| 1110 | s8 | REF | 75% |
| 1111 | s6 | BOTH | 30% |
| 1112 | s3 | ALT | 50% |
| 1113 | s3 | ALT | 50% |
| 1114 | s7 | ALT | 35% |
| 1115 | s3 | ALT | 50% |
| 1116 | s13 | ALT | 55% |
| 1117 | s9 | ALT | 65% |
| 1118 | s1 | REF | 50% |
| 1119 | s5 | ALT | 55% |
| 1120 | s13 | ALT | 60% |
| 1121 | s1 | ALT | 60% |
| 1122 | s3 | ALT | 50% |
| 1123 | s10 | ALT | 55% |
| 1124 | s6 | REF | 45% |
| 1125 | s5 | ALT | 55% |
| 1126 | s13 | ALT | 55% |
| 1127 | s6 | ALT | 40% |
| 1128 | s11 | ALT | 70% |
| 1129 | s13 | ALT | 50% |
| 1130 | s8 | REF | 75% |
| 1131 | s3 | ALT | 50% |
| 1132 | s3 | ALT | 50% |
| 1133 | s13 | ALT | 50% |
| 1134 | s11 | ALT | 65% |
| 1135 | s1 | REF | 55% |
| 1136 | s8 | REF | 65% |
| 1137 | s4 | ALT | 70% |
| 1138 | s9 | ALT | 65% |
| 1139 | s9 | ALT | 60% |
| 1140 | s13 | ALT | 50% |
| 1141 | s3 | ALT | 50% |
| 1142 | s13 | ALT | 50% |
| 1143 | s7 | REF | 50% |
| 1144 | s6 | ALT | 30% |
| 1145 | s6 | REF | 35% |
| 1146 | s5 | ALT | 45% |
| 1147 | s13 | ALT | 55% |
| 1148 | s9 | REF | 65% |
| 1149 | s7 | ALT | 60% |
| 1150 | s6 | ALT | 35% |
| 1151 | s6 | REF | 40% |
| 1152 | s3 | ALT | 50% |
| 1153 | s4 | ALT | 65% |
| 1154 | s3 | ALT | 50% |
| 1155 | s13 | ALT | 55% |
| 1156 | s13 | REF | 55% |
| 1157 | s4 | REF | 50% |
| 1158 | s13 | ALT | 55% |
| 1159 | s3 | ALT | 55% |
| 1160 | s3 | ALT | 50% |
| 1161 | s13 | ALT | 55% |
| 1162 | s12 | ALT | 80% |
| 1163 | s8 | ALT | 70% |
| 1164 | s13 | ALT | 55% |
| 1165 | s13 | ALT | 55% |
| 1166 | s13 | ALT | 55% |
| 1167 | s12 | REF | 70% |
| 1168 | s13 | ALT | 55% |
| 1169 | s13 | REF | 55% |
| 1170 | s7 | ALT | 50% |
| 1171 | s13 | ALT | 60% |
| 1172 | s4 | REF | 70% |
| 1173 | s10 | REF | 75% |
| 1174 | s8 | ALT | 75% |
| 1175 | s9 | REF | 65% |
| 1176 | s11 | ALT | 75% |
| 1177 | s9 | ALT | 60% |
| 1178 | s2 | ALT | 55% |
| 1179 | s13 | ALT | 65% |
| 1180 | s13 | ALT | 65% |
| 1181 | s11 | REF | 80% |
| 1182 | s5 | ALT | 50% |
| 1183 | s13 | ALT | 60% |
| 1184 | s5 | ALT | 70% |
| 1185 | s12 | REF | 70% |
| 1186 | s6 | REF | 40% |
| 1187 | S14 | ALT | 75% |
| 1188 | s5 | ALT | 50% |
| 1189 | s5 | ALT | 55% |
| 1190 | s13 | ALT | 60% |
| 1191 | s5 | ALT | 65% |
| 1192 | s5 | ALT | 60% |
| 1193 | s6 | BOTH | 30% |
| 1194 | s7 | REF | 55% |
| 1195 | s3 | ALT | 50% |
| 1196 | s10 | ALT | 50% |
| 1197 | s11 | REF | 70% |
| 1198 | s6 | REF | 35% |
| 1199 | s3 | ALT | 55% |
| 1200 | s8 | ALT | 65% |
| 1201 | s5 | ALT | 45% |
| 1202 | s5 | ALT | 60% |
| 1203 | s5 | ALT | 50% |
| 1204 | s10 | ALT | 70% |
| 1205 | s3 | ALT | 55% |
| 1206 | s3 | ALT | 50% |
| 1207 | s2 | ALT | 55% |
| 1208 | s1 | ALT | 60% |
| 1209 | s10 | REF | 70% |
| 1210 | s13 | ALT | 65% |
| 1211 | s7 | ALT | 60% |
| 1212 | s7 | ALT | 40% |
| 1213 | s13 | ALT | 55% |
| 1214 | s6 | ALT | 35% |
| 1215 | s5 | ALT | 50% |
| 1216 | s6 | REF | 50% |
| 1217 | s6 | ALT | 45% |
| 1218 | s4 | REF | 65% |
| 1219 | s10 | ALT | 65% |
| 1220 | s3 | ALT | 50% |
| 1221 | s3 | ALT | 50% |
| 1222 | s6 | REF | 45% |
| 1223 | s5 | ALT | 55% |
| 1224 | s13 | ALT | 60% |
| 1225 | s6 | ALT | 40% |
| 1226 | s13 | ALT | 55% |
| 1227 | s5 | ALT | 55% |
| 1228 | s1 | REF | 65% |
| 1229 | s13 | ALT | 55% |
| 1230 | s2 | REF | 50% |
| 1231 | s3 | ALT | 50% |
| 1232 | s5 | ALT | 65% |
| 1233 | s1 | REF | 60% |
| 1234 | s3 | ALT | 55% |
| 1235 | s12 | ALT | 80% |
| 1236 | s13 | ALT | 55% |
| 1237 | s13 | ALT | 50% |
| 1238 | s13 | ALT | 55% |
| 1239 | s13 | ALT | 70% |
| 1240 | s13 | REF | 70% |
| 1241 | s12 | ALT | 75% |
| 1242 | s5 | ALT | 55% |
| 1243 | s5 | ALT | 65% |
| 1244 | s3 | ALT | 55% |
| 1245 | s5 | ALT | 50% |
| 1246 | s6 | ALT | 50% |
| 1247 | s3 | ALT | 55% |
| 1248 | s13 | REF | 55% |
| 1249 | s13 | ALT | 55% |
| 1250 | s2 | REF | 50% |
| 1251 | s3 | ALT | 50% |
| 1252 | s13 | ALT | 60% |
| 1253 | s4 | ALT | 75% |
| 1254 | s8 | REF | 70% |
| 1255 | s11 | ALT | 80% |
| 1256 | s3 | ALT | 55% |
| 1257 | s4 | ALT | 60% |
| 1258 | s4 | ALT | 60% |
| 1259 | s12 | REF | 75% |
| 1260 | s13 | ALT | 70% |
| 1261 | s1 | ALT | 60% |
| 1262 | s12 | ALT | 75% |
| 1263 | s2 | ALT | 50% |
| 1264 | s7 | BOTH | 55% |
| 1265 | s1 | REF | 55% |
| 1266 | s7 | ALT | 55% |
| 1267 | s13 | ALT | 55% |
| 1268 | s6 | ALT | 50% |
| 1269 | s13 | ALT | 55% |
| 1270 | s7 | BOTH | 50% |
| 1271 | s7 | REF | 50% |
| 1272 | s6 | ALT | 35% |
| 1273 | s6 | REF | 40% |
| 1274 | s3 | ALT | 50% |
| 1275 | s13 | ALT | 45% |
| 1276 | s13 | ALT | 50% |
| 1277 | s5 | ALT | 50% |
| 1278 | s13 | ALT | 55% |
| 1279 | s10 | ALT | 55% |
| 1280 | s13 | ALT | 55% |
| 1281 | s13 | ALT | 50% |
| 1282 | s13 | ALT | 55% |
| 1283 | s2 | REF | 50% |
| 1284 | S14 | ALT | 60% |
| 1285 | s5 | ALT | 45% |
| 1286 | s3 | ALT | 50% |
| 1287 | s3 | ALT | 50% |
| 1288 | s9 | REF | 65% |
| 1289 | s5 | ALT | 45% |
| 1290 | s5 | ALT | 60% |
| 1291 | s5 | ALT | 45% |
| 1292 | s12 | ALT | 80% |
| 1293 | s2 | REF | 55% |
| 1294 | s5 | ALT | 60% |
| 1295 | s3 | ALT | 50% |
| 1296 | s3 | ALT | 50% |
| 1297 | s3 | ALT | 50% |
| 1298 | s13 | ALT | 50% |
| 1299 | s13 | ALT | 55% |
| 1300 | s8 | ALT | 60% |
| 1301 | s3 | ALT | 50% |
| 1302 | s1 | ALT | 65% |
| 1303 | s13 | ALT | 65% |
| 1304 | s7 | ALT | 55% |
| 1305 | s3 | ALT | 50% |
| 1306 | s13 | ALT | 55% |
| 1307 | s6 | ALT | 30% |
| 1308 | s5 | ALT | 55% |
| 1309 | s4 | REF | 60% |
| 1310 | S14 | ALT | 65% |
| 1311 | s5 | ALT | 45% |
| 1312 | s6 | ALT | 40% |
| 1313 | s6 | REF | 45% |

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: AApoA1 Correction Strategies

Two exemplary strategies are proposed to tackle AAopA1 with spCas9 at a genomic DNA level with two guide sequences. The first strategy involves targeting exon 1 or intron 1 with a first guide sequence and intron 3 with a second guide sequence in order to excise exons 1, exon2, and exon 3. The second strategy involves targeting intron 2 with a first guide sequence and intron 3 with a second guide sequence in order to remove exon 3. (FIG. 13).

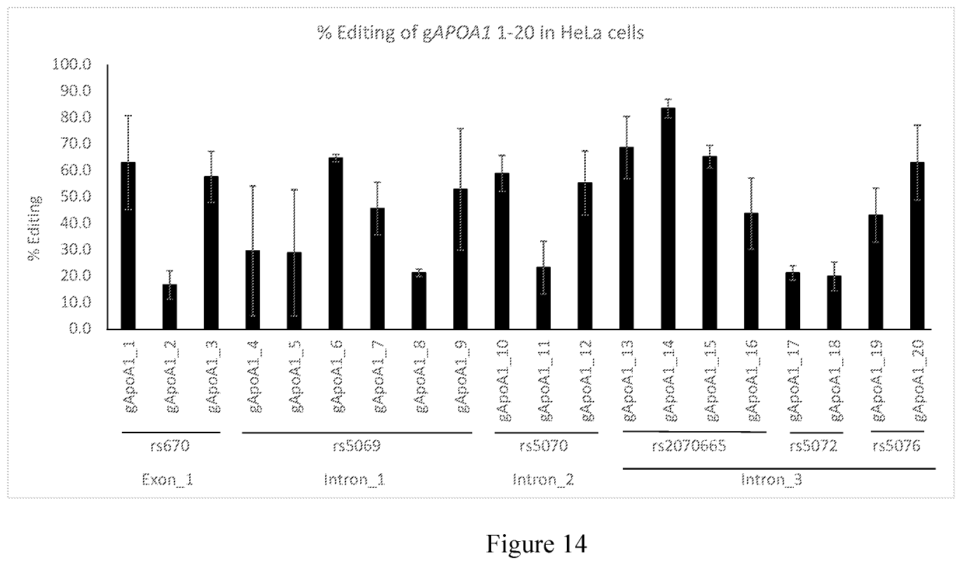

When using SpCas9, 20 different guide sequences, identified as gApoA1_1 through gApoA1_20, identified by SEQ ID NO. in Table 3, are screened in two experiment, experiment 474 and experiment 478, for high on target activity using spCas9 in HeLa cells. spCas9 coding plasmid (390 ng) was co-transfected with each of the guide sequence expression plasmids (120 ng) in 24 well plate format using Turbofect reagent (Thermo fisher scientific). Cells were harvested 72 h post DNA transfection. On target activity was determined by capillary electroporation analysis, as shown in FIG. 14. Percent editing in Hela cells for guide sequences in experiment 474 is shown in column 4 of Table 3 below, percent editing in HeLa cells for guide sequences in experiment 478 is shown in column 5 of Table 3, the average percent editing between the experiments is shown in column 5 of Table 3, and the standard deviation of editing is shown in column 6 in Table 3.

TABLE 3
gApoA1_1 through gApoA1_20 of Example 1 as identified by
SEQ ID NO.

| | | | % | % | | |
|---|---|---|---|---|---|---|
| | | SEQ | Editing- | Editing- | | |
| | Example 1 | ID | HeLa, | HeLa, | | |
| Guide sequence | gAPOA1 ID | NO: | Exp474 | Exp478 | Ave | STDV |
| AGUGAGCAGCAACAGGGCCG | gApoA1_1 | 49 | 50.3 | 75.4 | 62.9 | 17.75 |
| AGCAGCAACAGGGCCGGGGC | gApoA1_2 | 28 | 12.8 | 20.3 | 16.5 | 5.34 |
| GCAGCAACAGGGCCGGGGCU | gApoA1_3 | 142 | 50.7 | 64.3 | 57.5 | 9.61 |
| AGGUACCCAGAGGCCCGGCC | gApoA1_4 | 43 | 46.8 | 12.0 | 29.4 | 24.62 |
| GGUACCCAGAGGCCCGGCCU | gApoA1_5 | 195 | 45.6 | 11.8 | 28.7 | 23.89 |
| GUACCCAGAGGCCCGGCCUG | gApoA1_6 | 204 | 63.6 | 65.5 | 64.6 | 1.34 |
| UUCAGGCCUUGCCCCAGGCC | gApoA1_7 | 260 | 52.5 | 38.4 | 45.4 | 9.97 |
| CUUGCCCCAGGCCGGGCCUC | gApoA1_8 | 1057 | 22.1 | 20.2 | 21.2 | 1.35 |
| UUGCCCCAGGCCGGGCCUCU | gApoA1_9 | 267 | 36.4 | 69.0 | 52.7 | 23.05 |
| UUAGGGAGAAAGCCCCCCGA | gApoA1_10 | 258 | 63.6 | 54.0 | 58.8 | 6.82 |
| GGAGAAAGCCCCCCGAUGGU | gApoA1_11 | 169 | 30.1 | 16.0 | 23.1 | 9.98 |
| GCUUUCUCCCUAAAUCCCCG | gApoA1_12 | 162 | 46.6 | 63.7 | 55.2 | 12.10 |
| CUGGGGUUGAGGGCAGGGGC | gApoA1_13 | 114 | 60.3 | 76.9 | 68.6 | 11.76 |
| UGGGGUUGAGGGCAGGGGCA | gApoA1_14 | 253 | 85.9 | 80.9 | 83.4 | 3.51 |
| GGGGUUGAGGGCAGGGGCAG | gApoA1_15 | 188 | 68.1 | 62.1 | 65.1 | 4.23 |
| GGGUUGAGGGCAGGGGCAGG | gApoA1_16 | 192 | 34.0 | 53.1 | 43.5 | 13.46 |
| UCUGGAUGGAGAAACCGGAA | gApoA1_17 | 1058 | 19.1 | 23.1 | 21.1 | 2.78 |
| CUGGAUGGAGAAACCGGAAU | gApoA1_18 | 111 | 16.0 | 23.6 | 19.8 | 5.36 |
| AGCCUAUCAGGGGUGAGCCC | gApoA1_19 | 30 | 35.7 | 50.2 | 43.0 | 10.27 |
| GCCUAUCAGGGGUGAGCCCU | gApoA1_20 | 152 | 52.8 | 72.8 | 62.8 | 14.18 |

### Example 2: a Correction Analysis

Guide sequences comprising 17-20 nucleotides in the sequences of 17-20 contiguous nucleotides set forth in SEQ ID NOs: 1-1313 are screened for high on target activity. On target activity is determined by DNA capillary electrophoresis analysis.

According to DNA capillary electrophoresis analysis, guide sequences comprising 17-20 nucleotides in the sequences of 17-20 contiguous nucleotides set forth in SEQ ID NOs: 1-1313 are found to be suitable for correction of the APOA1 gene.

### DISCUSSION

The guide sequences of the present invention are determined to be suitable for targeting the APOA1 gene.

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20
2. Anders (1992) “Human gene therapy”, Science 256:808-13
3. Basha et al. (2011) “Influence of Cationic Lipid Composition on Gene Silencing Properties of Lipid Nanoparticle Formulations of siRNA in Antigen-Presenting Cells”, Mol. Ther. 19 (12): 2186-200
4. Behr (1994) Gene transfer with synthetic cationic amphiphiles: Prospects for gene therapy”, Bioconjuage Chem 5:382-89
5. Blaese (1995) “Vectors in cancer therapy: how will they deliver”, Cancer Gene Ther. 2:291-97
6. Blaese et al. (1995) “T lympocyte-directed gene therapy for ADA-SCID: initial trial results after 4 years”, Science 270 (5235): 475-80
7. Buchschacher and Panganiban (1992) “Human immunodeficiency virus vectors for inducible expression of foreign genes”, J. Virol. 66:2731-39
8. Burstein et al. (2017) “New CRISPR-Cas systems from uncultivated microbes”, Nature 542:237-41
9. Chung et al. (2006) “Agrobacterium is not alone: gene transfer to plants by viruses and other bacteria”, Trends Plant Sci. 11 (1): 1-4
10. Coelho et al. (2013) “Safety and Efficacy of RNAi Therapy for Transthyretin Amyloidosis”, N Engl J. Med 369:819-29
11. Crystal (1995) “Transfer of genes to humans: early lessons and obstacles to success”, Science 270 (5235): 404-10
12. Dillon (1993) “Regulation gene expression in gene therapy” Trends in Biotechnology 11 (5): 167-173
13. Dranoff et al. (1997) “A phase I study of vaccination with autologous, irradiated melanoma cells engineered to secrete human granulocyte macrophage colony stimulating factor”, Hum. Gene Ther. 8 (1): 111-23
14. Dunbar et al. (1995) “Retrovirally marked CD34-enriched peripheral blood and bone marrow cells contribute to long-term engraftment after autologous transplantation”, Blood 85:3048-57
15. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20
16. Gao and Huang (1995) “Cationic liposome-mediated gene transfer” Gene Ther. 2 (10): 710-22
17. Haddada et al. (1995) “Gene Therapy Using Adenovirus Vectors”, in: The Molecular Repertoire of Adenoviruses III: Biology and Pathogenesis, ed. Doerfler and Böhm, pp. 297-306
18. Han et al. (1995) “Ligand-directed retro-viral targeting of human breast cancer cells”, Proc Natl Acad Sci U.S.A. 92 (21): 9747-51
19. Inaba et al. (1992) “Generation of large numbers of dendritic cells from mouse bone marrow cultures supplemented with granulocyte/macrophage colony-stimulating factor”, J Exp Med. 176 (6): 1693-702
20. Jinek et al. (2012) “A programmable dual-RNA-guided DNA endonuclease in adaptive bacterial immunity,” Science 337 (6096): 816-21
21. Johan et al. (1992) “GLVR 1, a receptor for gibbon ape leukemia virus, is homologous to a phosphate permease of Neurospora crassa and is expressed at high levels in the brain and thymus”, J Virol 66 (3): 1635-40
22. Judge et al. (2006) “Design of noninflammatory synthetic siRNA mediating potent gene silencing in vivo”, Mol Ther. 13 (3): 494-505
23. Kohn et al. (1995) “Engraftment of gene-modified umbilical cord blood cells in neonates with adnosine deaminase deficiency”, Nature Medicine 1:1017-23
24. Kremer and Perricaudet (1995) “Adenovirus and adeno-associated virus mediated gene transfer”, Br. Med. Bull. 51 (1): 31-44
25. Macdiarmid et al. (2009) “Sequential treatment of drug-resistant tumors with targeted minicells containing siRNA or a cytotoxic drug”, Nat Biotehenol. 27 (7): 643-51
26. Malech et al. (1997) “Prolonged production of NADPH oxidase-corrected granulocyes after gene therapy of chronic granulomatous disease”, PNAS 94 (22): 12133-38
27. Miller et al. (1991) “Construction and properties of retrovirus packaging cells based on gibbon ape leukemia virus”, J Virol. 65 (5): 2220-24
28. Miller (1992) “Human gene therapy comes of age”, Nature 357:455-60
29. Mitani and Caskey (1993) “Delivering therapeutic genes-matching approach and application”, Trends in Biotechnology 11 (5): 162-66
30. Nabel and Felgner (1993) “Direct gene transfer for immunotherapy and immunization”, Trends in Biotechnology 11 (5): 211-15
31. Remy et al. (1994) “Gene Transfer with a Series of Lipphilic DNA-Binding Molecules”, Bioconjugate Chem. 5 (6): 647-54
32. Sentmanat et al. (2018) “A Survey of Validation Strategies for CRISPR-Cas9 Editing”, Scientific Reports 8:888, doi: 10.1038/s41598-018-19441-8
33. Sommerfelt et al. (1990) “Localization of the receptor gene for type D simian retroviruses on human chromosome 19”, J. Virol. 64 (12): 6214-20
34. Van Brunt (1988) “Molecular framing: transgenic animals as bioactors” Biotechnology 6:1149-54
35. Vigne et al. (1995) “Third-generation adenovectors for gene therapy”, Restorative Neurology and Neuroscience 8 (1,2): 35-36
36. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78
37. Yu et al. (1994) “Progress towards gene therapy for HIV infection”, Gene Ther. 1 (1): 13-26
38. Zetsche et al. (2015) “Cpf1 is a single RNA-guided endonuclease of a class 2 CRIPSR-Cas system” Coll 163 (3): 759-71
39. Zuris et al. (2015) “Cationic lipid-mediated delivery of proteins enables efficient protein based genome editing in vitro and in vivo” Nat Biotechnol. 33 (1): 73-80.

1. An isolated guide RNA (gRNA) that targets a mutant Apolipoprotein A1 (APOA1) allele, wherein the gRNA comprises a CRISPR RNA (crRNA) comprising a nucleic acid sequence of 17-20 nucleotides which comprise 17-20 contiguous nucleotides set forth in SEQ ID NOs: 43, 195, 204, 260, 267, or 1057.

2. A method for inactivating a mutant allele of the Apolipoprotein A1 (APOA1), the method comprising:
(a) delivering to an isolated human cell that comprises a mutant APOA1 allele and a functional APOA1 allele a composition comprising:
(i) a CRISPR nuclease, and
(ii) an isolated guide RNA molecule (gRNA) that targets the mutant human APOA1 allele, wherein the gRNA comprises a CRISPR RNA (crRNA) comprising a nucleic acid sequence of 17-20 nucleotides which comprise 17-20 contiguous nucleotides set forth in SEQ ID NOs: 43, 195, 204, 260, 267, or 1057; and
(b) culturing the cell obtained in step a) such that the mutant APOA1 allele is inactivated and the functional APOA1 allele remains intact.

3. The gRNA of claim 1, wherein the crRNA comprises 17-20 contiguous nucleotides as set forth in SEQ ID NO: 43.

4. The gRNA of claim 1, wherein the crRNA comprises 17-20 contiguous nucleotides as set forth in SEQ ID NO: 195.

5. The gRNA of claim 1, wherein the crRNA comprises 17-20 contiguous nucleotides as set forth in SEQ ID NO: 204.

6. The gRNA of claim 1, wherein the crRNA comprises 17-20 contiguous nucleotides as set forth in SEQ ID NO: 260.

7. The gRNA of claim 1, wherein the crRNA comprises 17-20 contiguous nucleotides as set forth in SEQ ID NO: 267.

8. The gRNA of claim 1, wherein the crRNA comprises 17-20 contiguous nucleotides as set forth in SEQ ID NO: 1057.

9. The method of claim 2, wherein the mutant APOA1 allele is inactivated by a frameshift mutation.

10. The method of claim 9, wherein the frameshift mutation creates an early stop codon in the mutant APOA1 allele.

11. The method of claim 9, wherein the frameshift mutation results in nonsense-mediated mRNA decay of a transcript of the mutant APOA1 allele.

12. The method of claim 2, wherein the inactivated mutant APOA1 allele expresses a truncated protein and the intact functional APOA1 allele expresses a functional protein.

13. A method for inactivating a mutant allele of the APOA1, the method comprising:
(a) delivering to an isolated human cell that comprises a mutant APOA1 allele and a functional APOA1 allele a composition comprising:
(i) a CRISPR nuclease, and
(ii) an isolated gRNA that targets a mutant human APOA1 allele, wherein the gRNA comprises a crRNA comprising a nucleic acid sequence which comprises (i) the 18 consecutive nucleotides shared by SEQ ID NOs: 43, 195, and 204 or (ii) the 19 consecutive nucleotides shared by SEQ ID NOs: 1057 and 267; and
(b) culturing the cell obtained in step a) such that the mutant APOA1 allele is inactivated and the functional APOA1 allele remains intact.

14. The method of claim 13, wherein the cRNA comprises a nucleic acid sequence comprising the 18 consecutive nucleotides shared by SEQ ID NOs: 43, 195, and 204.

15. The method of claim 13, wherein the cRNA comprises a nucleic acid sequence comprising the 19 consecutive nucleotides shared by SEQ ID NOs: 1057 and 267.

16. The method of claim 13, wherein the mutant APOA1 allele is inactivated by a frameshift mutation.

17. The method of claim 16, wherein the frameshift mutation creates an early stop codon in the mutant APOA1 allele.

18. The method of claim 16, wherein the frameshift mutation results in nonsense-mediated mRNA decay of a transcript of the mutant APOA1 allele.

19. The method of claim 13, wherein the inactivated mutant APOA1 allele expresses a truncated protein and the intact functional APOA1 allele expresses a functional protein.


SEQUENCE LISTING (1313 sequences)

SEQ ID NO: 1 - RNA, 20 nt, synthetic construct
  misc_feature 1..20: note = Synthetic Construct
  source 1..20: mol_type = other RNA
aaagacagcggcagagacta

SEQ ID NO: 2 - RNA, 20 nt, synthetic construct
  misc_feature 1..20: note = Synthetic Construct
  source 1..20: mol_type = other RNA
aaagctgcaggctccgcaag

SEQ ID NO: 3 - RNA, 20 nt, synthetic construct
  misc_feature 1..20: note = Synthetic Construct
  source 1..20: mol_type = other RNA
aaaggagacagagctgggac

SEQ ID NO: 4 - RNA, 20 nt, synthetic construct
  misc_feature 1..20: note = Synthetic Construct
  source 1..20: mol_type = other RNA
aaaggctgcaggctccgcaa

SEQ ID NO: 5 - RNA, 20 nt, synthetic construct
  misc_feature 1..20: note = Synthetic Construct
  source 1..20: mol_type = other RNA
aaccctgggtgacacctgtc

SEQ ID NO: 6 - RNA, 20 nt, synthetic construct
  misc_feature 1..20: note = Synthetic Construct
  source 1..20: mol_type = other RNA
aaggagacagagctgggact

SEQ ID NO: 7 - RNA, 20 nt, synthetic construct
  misc_feature 1..20: note = Synthetic Construct
  source 1..20: mol_type = other RNA
aaggctgcaggctccgcaag

SEQ ID NO: 8 - RNA, 20 nt, synthetic construct
  misc_feature 1..20: note = Synthetic Construct
  source 1..20: mol_type = other RNA
aatctggcctccagccttac

SEQ ID NO: 9 - RNA, 20 nt, synthetic construct
  misc_feature 1..20: note = Synthetic Construct
  source 1..20: mol_type = other RNA
aatctggcctccagccttat

SEQ ID NO: 10 - RNA, 20 nt, synthetic construct
  misc_feature 1..20: note = Synthetic Construct
  source 1..20: mol_type = other RNA
acagggccagggctgggctt

SEQ ID NO: 11 - RNA, 20 nt, synthetic construct
  misc_feature 1..20: note = Synthetic Construct
  source 1..20: mol_type = other RNA
acagggccggggctgggctt

SEQ ID NO: 12 - RNA, 20 nt, synthetic construct
  misc_feature 1..20: note = Synthetic Construct
  source 1..20: mol_type = other RNA
accatcggggggccttctcc

SEQ ID NO: 13 - RNA, 20 nt, synthetic construct
  misc_feature 1..20: note = Synthetic Construct
  source 1..20: mol_type = other RNA
accatcggggggctttctcc

SEQ ID NO: 14 - RNA, 20 nt, synthetic construct
  misc_feature 1..20: note = Synthetic Construct
  source 1..20: mol_type = other RNA
acccagggctcacccctgat

SEQ ID NO: 15 - RNA, 20 nt, synthetic construct
  misc_feature 1..20: note = Synthetic Construct
  source 1..20: mol_type = other RNA
acccagggttcacccctgat

SEQ ID NO: 16 - RNA, 20 nt, synthetic construct
  misc_feature 1..20: note = Synthetic Construct
  source 1..20: mol_type = other RNA
accggaatgggatctccagg

SEQ ID NO: 17 - RNA, 20 nt, synthetic construct
  misc_feature 1..20: note = Synthetic Construct
  source 1..20: mol_type = other RNA
acctcaggtacccagaggcc

SEQ ID NO: 18 - RNA, 20 nt, synthetic construct
  misc_feature 1..20: note = Synthetic Construct
  source 1..20: mol_type = other RNA
acgtggatgtgctcaaagac

SEQ ID NO: 19 - RNA, 20 nt, synthetic construct
  misc_feature 1..20: note = Synthetic Construct
  source 1..20: mol_type = other RNA
actcatgtgactgctggaag

SEQ ID NO: 20 - RNA, 20 nt, synthetic construct
  misc_feature 1..20: note = Synthetic Construct
  source 1..20: mol_type = other RNA
actggaatgggatctccagg

SEQ ID NO: 21 - RNA, 20 nt, synthetic construct
  misc_feature 1..20: note = Synthetic Construct
  source 1..20: mol_type = other RNA
acttctcagtttcctggctc

SEQ ID NO: 22 - RNA, 20 nt, synthetic construct
  misc_feature 1..20: note = Synthetic Construct
  source 1..20: mol_type = other RNA
agaaagacctccttcctttc

SEQ ID NO: 23 - RNA, 20 nt, synthetic construct
  misc_feature 1..20: note = Synthetic Construct
  source 1..20: mol_type = other RNA
agaaagacttccttcctttc

SEQ ID NO: 24 - RNA, 20 nt, synthetic construct
  misc_feature 1..20: note = Synthetic Construct
  source 1..20: mol_type = other RNA
agacctcaggtacccagagg

SEQ ID NO: 25 - RNA, 20 nt, synthetic construct
  misc_feature 1..20: note = Synthetic Construct
  source 1..20: mol_type = other RNA
agactcatgtgactgctgga

SEQ ID NO: 26 - RNA, 20 nt, synthetic construct
  misc_feature 1..20: note = Synthetic Construct
  source 1..20: mol_type = other RNA
agactcgtgactgctggaag

SEQ ID NO: 27 - RNA, 20 nt, synthetic construct
  misc_feature 1..20: note = Synthetic Construct
  source 1..20: mol_type = other RNA
agatcagactcgtgactgct

SEQ ID NO: 28 - RNA, 20 nt, synthetic construct
  misc_feature 1..20: note = Synthetic Construct
  source 1..20: mol_type = other RNA
agcagcaacagggccggggc

SEQ ID NO: 29 - RNA, 20 nt, synthetic construct
  misc_feature 1..20: note = Synthetic Construct
  source 1..20: mol_type = other RNA
agccctgggtgacacctgtc

SEQ ID NO: 30 - RNA, 20 nt, synthetic construct
  misc_feature 1..20: note = Synthetic Construct
  source 1..20: mol_type = other RNA
agcctatcaggggtgagccc

SEQ ID NO: 31 - RNA, 20 nt, synthetic construct
  misc_feature 1..20: note = Synthetic Construct
  source 1..20: mol_type = other RNA
agcctgcagcctttgcagtc

SEQ ID NO: 32 - RNA, 20 nt, synthetic construct
  misc_feature 1..20: note = Synthetic Construct
  source 1..20: mol_type = other RNA
agcctttgcagtctgatcag

SEQ ID NO: 33 - RNA, 20 nt, synthetic construct
  misc_feature 1..20: note = Synthetic Construct
  source 1..20: mol_type = other RNA
aggcccagacctcccccagc

SEQ ID NO: 34 - RNA, 20 nt, synthetic construct
  misc_feature 1..20: note = Synthetic Construct
  source 1..20: mol_type = other RNA
aggcccagacctccctcagc

SEQ ID NO: 35 - RNA, 20 nt, synthetic construct
  misc_feature 1..20: note = Synthetic Construct
  source 1..20: mol_type = other RNA
aggctgggcctctgggtacc

SEQ ID NO: 36 - RNA, 20 nt, synthetic construct
  misc_feature 1..20: note = Synthetic Construct
  source 1..20: mol_type = other RNA
agggaggtctgggccttcca

SEQ ID NO: 37 - RNA, 20 nt, synthetic construct
  misc_feature 1..20: note = Synthetic Construct
  source 1..20: mol_type = other RNA
agggcaggggcagggggcag

SEQ ID NO: 38 - RNA, 20 nt, synthetic construct
  misc_feature 1..20: note = Synthetic Construct
  source 1..20: mol_type = other RNA
agggcaggggtagggggcag

SEQ ID NO: 39 - RNA, 20 nt, synthetic construct
  misc_feature 1..20: note = Synthetic Construct
  source 1..20: mol_type = other RNA
agggctgggcttatcagcct

SEQ ID NO: 40 - RNA, 20 nt, synthetic construct
  misc_feature 1..20: note = Synthetic Construct
  source 1..20: mol_type = other RNA
aggggcagggggcagaggcc

SEQ ID NO: 41 - RNA, 20 nt, synthetic construct
  misc_feature 1..20: note = Synthetic Construct
  source 1..20: mol_type = other RNA
aggggtagggggcagaggcc

SEQ ID NO: 42 - RNA, 20 nt, synthetic construct
  misc_feature 1..20: note = Synthetic Construct
  source 1..20: mol_type = other RNA
agggttcacccctgataggc

SEQ ID NO: 43 - RNA, 20 nt, synthetic construct
  misc_feature 1..20: note = Synthetic Construct
  source 1..20: mol_type = other RNA
aggtacccagaggcccggcc

SEQ ID NO: 44 - RNA, 20 nt, synthetic construct
  misc_feature 1..20: note = Synthetic Construct
  source 1..20: mol_type = other RNA
aggtcctatcagtaacctgc

SEQ ID NO: 45 - RNA, 20 nt, synthetic construct
  misc_feature 1..20: note = Synthetic Construct
  source 1..20: mol_type = other RNA
agtaacctgctgagggaggt

SEQ ID NO: 46 - RNA, 20 nt, synthetic construct
  misc_feature 1..20: note = Synthetic Construct
  source 1..20: mol_type = other RNA
agtaacctgctgggggaggt

SEQ ID NO: 47 - RNA, 20 nt, synthetic construct
  misc_feature 1..20: note = Synthetic Construct
  source 1..20: mol_type = other RNA
agtccctgatcagactgcaa

SEQ ID NO: 48 - RNA, 20 nt, synthetic construct
  misc_feature 1..20: note = Synthetic Construct
  source 1..20: mol_type = other RNA
agtgagcagcaacagggcca

SEQ ID NO: 49 - RNA, 20 nt, synthetic construct
  misc_feature 1..20: note = Synthetic Construct
  source 1..20: mol_type = other RNA
agtgagcagcaacagggccg

SEQ ID NO: 50 - RNA, 20 nt, synthetic construct
  misc_feature 1..20: note = Synthetic Construct
  source 1..20: mol_type = other RNA
atagtctctgccgctgtctt

SEQ ID NO: 51 - RNA, 20 nt, synthetic construct
  misc_feature 1..20: note = Synthetic Construct
  source 1..20: mol_type = other RNA
atatccctataaggctggag

SEQ ID NO: 52 - RNA, 20 nt, synthetic construct
  misc_feature 1..20: note = Synthetic Construct
  source 1..20: mol_type = other RNA
atatccctgtaaggctggag

SEQ ID NO: 53 - RNA, 20 nt, synthetic construct
  misc_feature 1..20: note = Synthetic Construct
  source 1..20: mol_type = other RNA
atcagactcatgtgactgct

SEQ ID NO: 54 - RNA, 20 nt, synthetic construct
  misc_feature 1..20: note = Synthetic Construct
  source 1..20: mol_type = other RNA
atcctgatgcctcgcagtcc

SEQ ID NO: 55 - RNA, 20 nt, synthetic construct
  misc_feature 1..20: note = Synthetic Construct
  source 1..20: mol_type = other RNA
atctggcctccagccttaca

SEQ ID NO: 56 - RNA, 20 nt, synthetic construct
  misc_feature 1..20: note = Synthetic Construct
  source 1..20: mol_type = other RNA
atctggcctccagccttata

SEQ ID NO: 57 - RNA, 20 nt, synthetic construct
  misc_feature 1..20: note = Synthetic Construct
  source 1..20: mol_type = other RNA
atggcagtggagatcagact

SEQ ID NO: 58 - RNA, 20 nt, synthetic construct
  misc_feature 1..20: note = Synthetic Construct
  source 1..20: mol_type = other RNA
atggcccccttccagcagtc

SEQ ID NO: 59 - RNA, 20 nt, synthetic construct
  misc_feature 1..20: note = Synthetic Construct
  source 1..20: mol_type = other RNA
atggtctggatggagaaacc

SEQ ID NO: 60 - RNA, 20 nt, synthetic construct
  misc_feature 1..20: note = Synthetic Construct
  source 1..20: mol_type = other RNA
atggtctggatggagaaact

SEQ ID NO: 61 - RNA, 20 nt, synthetic construct
  misc_feature 1..20: note = Synthetic Construct
  source 1..20: mol_type = other RNA
atgtgctcaaagacagcggc

SEQ ID NO: 62 - RNA, 20 nt, synthetic construct
  misc_feature 1..20: note = Synthetic Construct
  source 1..20: mol_type = other RNA
caaaggctgcaggctccgca

SEQ ID NO: 63 - RNA, 20 nt, synthetic construct
  misc_feature 1..20: note = Synthetic Construct
  source 1..20: mol_type = other RNA
caacttagaatatccctata

SEQ ID NO: 64 - RNA, 20 nt, synthetic construct
  misc_feature 1..20: note = Synthetic Construct
  source 1..20: mol_type = other RNA
caacttagaatatccctgta

SEQ ID NO: 65 - RNA, 20 nt, synthetic construct
  misc_feature 1..20: note = Synthetic Construct
  source 1..20: mol_type = other RNA
cacatgagtctgatctccac

SEQ ID NO: 66 - RNA, 20 nt, synthetic construct
  misc_feature 1..20: note = Synthetic Construct
  source 1..20: mol_type = other RNA
cagacctcccccagcaggtt

SEQ ID NO: 67 - RNA, 20 nt, synthetic construct
  misc_feature 1..20: note = Synthetic Construct
  source 1..20: mol_type = other RNA
cagacctccctcagcaggtt

SEQ ID NO: 68 - RNA, 20 nt, synthetic construct
  misc_feature 1..20: note = Synthetic Construct
  source 1..20: mol_type = other RNA
cagactcgtgactgctggaa

SEQ ID NO: 69 - RNA, 20 nt, synthetic construct
  misc_feature 1..20: note = Synthetic Construct
  source 1..20: mol_type = other RNA
cagactgcaaaggctgcagg

SEQ ID NO: 70 - RNA, 20 nt, synthetic construct
  misc_feature 1..20: note = Synthetic Construct
  source 1..20: mol_type = other RNA
cagaggcccagcctggggca

SEQ ID NO: 71 - RNA, 20 nt, synthetic construct
  misc_feature 1..20: note = Synthetic Construct
  source 1..20: mol_type = other RNA
cagaggcccggcctggggca

SEQ ID NO: 72 - RNA, 20 nt, synthetic construct
  misc_feature 1..20: note = Synthetic Construct
  source 1..20: mol_type = other RNA
cagcctttgcagtctgatca

SEQ ID NO: 73 - RNA, 20 nt, synthetic construct
  misc_feature 1..20: note = Synthetic Construct
  source 1..20: mol_type = other RNA
cagtccccgagccaggaaac

SEQ ID NO: 74 - RNA, 20 nt, synthetic construct
  misc_feature 1..20: note = Synthetic Construct
  source 1..20: mol_type = other RNA
cagtccctgagccaggaaac

SEQ ID NO: 75 - RNA, 20 nt, synthetic construct
  misc_feature 1..20: note = Synthetic Construct
  source 1..20: mol_type = other RNA
cagtgagcagcaacagggcc

SEQ ID NO: 76 - RNA, 20 nt, synthetic construct
  misc_feature 1..20: note = Synthetic Construct
  source 1..20: mol_type = other RNA
cagtggagatcagactcatg

SEQ ID NO: 77 - RNA, 20 nt, synthetic construct
  misc_feature 1..20: note = Synthetic Construct
  source 1..20: mol_type = other RNA
cagtttcctggctcagggac

SEQ ID NO: 78 - RNA, 20 nt, synthetic construct
  misc_feature 1..20: note = Synthetic Construct
  source 1..20: mol_type = other RNA
cagtttcctggctcggggac

SEQ ID NO: 79 - RNA, 20 nt, synthetic construct
  misc_feature 1..20: note = Synthetic Construct
  source 1..20: mol_type = other RNA
catagtctctgccgctgtct

SEQ ID NO: 80 - RNA, 20 nt, synthetic construct
  misc_feature 1..20: note = Synthetic Construct
  source 1..20: mol_type = other RNA
cattccagtttctccatcca

SEQ ID NO: 81 - RNA, 20 nt, synthetic construct
  misc_feature 1..20: note = Synthetic Construct
  source 1..20: mol_type = other RNA
cattccggtttctccatcca

SEQ ID NO: 82 - RNA, 20 nt, synthetic construct
  misc_feature 1..20: note = Synthetic Construct
  source 1..20: mol_type = other RNA
ccagtgagcagcaacagggc

SEQ ID NO: 83 - RNA, 20 nt, synthetic construct
  misc_feature 1..20: note = Synthetic Construct
  source 1..20: mol_type = other RNA
ccattccagtttctccatcc

SEQ ID NO: 84 - RNA, 20 nt, synthetic construct
  misc_feature 1..20: note = Synthetic Construct
  source 1..20: mol_type = other RNA
ccattccggtttctccatcc

SEQ ID NO: 85 - RNA, 20 nt, synthetic construct
  misc_feature 1..20: note = Synthetic Construct
  source 1..20: mol_type = other RNA
cccagaggcccagcctgggg

SEQ ID NO: 86 - RNA, 20 nt, synthetic construct
  misc_feature 1..20: note = Synthetic Construct
  source 1..20: mol_type = other RNA
cccccagcaggttactgata

SEQ ID NO: 87 - RNA, 20 nt, synthetic construct
  misc_feature 1..20: note = Synthetic Construct
  source 1..20: mol_type = other RNA
ccccttccagcagtcacgag

SEQ ID NO: 88 - RNA, 20 nt, synthetic construct
  misc_feature 1..20: note = Synthetic Construct
  source 1..20: mol_type = other RNA
ccctcagcaggttactgata

SEQ ID NO: 89 - RNA, 20 nt, synthetic construct
  misc_feature 1..20: note = Synthetic Construct
  source 1..20: mol_type = other RNA
ccctggccctgttgctgctc

SEQ ID NO: 90 - RNA, 20 nt, synthetic construct
  misc_feature 1..20: note = Synthetic Construct
  source 1..20: mol_type = other RNA
ccggaatgggatctccaggc

SEQ ID NO: 91 - RNA, 20 nt, synthetic construct
  misc_feature 1..20: note = Synthetic Construct
  source 1..20: mol_type = other RNA
ccggccctgttgctgctcac

SEQ ID NO: 92 - RNA, 20 nt, synthetic construct
  misc_feature 1..20: note = Synthetic Construct
  source 1..20: mol_type = other RNA
cctatcaggggtgaaccctg

SEQ ID NO: 93 - RNA, 20 nt, synthetic construct
  misc_feature 1..20: note = Synthetic Construct
  source 1..20: mol_type = other RNA
cctatcaggggtgagccctg

SEQ ID NO: 94 - RNA, 20 nt, synthetic construct
  misc_feature 1..20: note = Synthetic Construct
  source 1..20: mol_type = other RNA
cctatcagtaacctgctgag

SEQ ID NO: 95 - RNA, 20 nt, synthetic construct
  misc_feature 1..20: note = Synthetic Construct
  source 1..20: mol_type = other RNA
cctatcagtaacctgctggg

SEQ ID NO: 96 - RNA, 20 nt, synthetic construct
  misc_feature 1..20: note = Synthetic Construct
  source 1..20: mol_type = other RNA
cctgcctggagatcccattc

SEQ ID NO: 97 - RNA, 20 nt, synthetic construct
  misc_feature 1..20: note = Synthetic Construct
  source 1..20: mol_type = other RNA
cctggaaggcccagacctcc

SEQ ID NO: 98 - RNA, 20 nt, synthetic construct
  misc_feature 1..20: note = Synthetic Construct
  source 1..20: mol_type = other RNA
ccttccagcagtcacatgag

SEQ ID NO: 99 - RNA, 20 nt, synthetic construct
  misc_feature 1..20: note = Synthetic Construct
  source 1..20: mol_type = other RNA
ccttccagcagtcacgagtc

SEQ ID NO: 100 - RNA, 20 nt, synthetic construct
  misc_feature 1..20: note = Synthetic Construct
  source 1..20: mol_type = other RNA
ccttgccccaggccgggcct

SEQ ID NO: 101 - RNA, 20 nt, synthetic construct
  misc_feature 1..20: note = Synthetic Construct
  source 1..20: mol_type = other RNA
ccttgccccaggctgggcct

SEQ ID NO: 102 - RNA, 20 nt, synthetic construct
  misc_feature 1..20: note = Synthetic Construct
  source 1..20: mol_type = other RNA
cggaatgggatctccaggca

SEQ ID NO: 103 - RNA, 20 nt, synthetic construct
  misc_feature 1..20: note = Synthetic Construct
  source 1..20: mol_type = other RNA
ctacccctgccctcaacccc

SEQ ID NO: 104 - RNA, 20 nt, synthetic construct
  misc_feature 1..20: note = Synthetic Construct
  source 1..20: mol_type = other RNA
ctcagggactgcgaggcatc

SEQ ID NO: 105 - RNA, 20 nt, synthetic construct
  misc_feature 1..20: note = Synthetic Construct
  source 1..20: mol_type = other RNA
ctcatgtgactgctggaagg

SEQ ID NO: 106 - RNA, 20 nt, synthetic construct
  misc_feature 1..20: note = Synthetic Construct
  source 1..20: mol_type = other RNA
ctcccccagcaggttactga

SEQ ID NO: 107 - RNA, 20 nt, synthetic construct
  misc_feature 1..20: note = Synthetic Construct
  source 1..20: mol_type = other RNA
ctccctcagcaggttactga

SEQ ID NO: 108 - RNA, 20 nt, synthetic construct
  misc_feature 1..20: note = Synthetic Construct
  source 1..20: mol_type = other RNA
ctctgccccctgcccctgcc

SEQ ID NO: 109 - RNA, 20 nt, synthetic construct
  misc_feature 1..20: note = Synthetic Construct
  source 1..20: mol_type = other RNA
ctgcccctgccctcaacccc

SEQ ID NO: 110 - RNA, 20 nt, synthetic construct
  misc_feature 1..20: note = Synthetic Construct
  source 1..20: mol_type = other RNA
ctggaatgggatctccaggc

SEQ ID NO: 111 - RNA, 20 nt, synthetic construct
  misc_feature 1..20: note = Synthetic Construct
  source 1..20: mol_type = other RNA
ctggatggagaaaccggaat

SEQ ID NO: 112 - RNA, 20 nt, synthetic construct
  misc_feature 1..20: note = Synthetic Construct
  source 1..20: mol_type = other RNA
ctggatggagaaactggaat

SEQ ID NO: 113 - RNA, 20 nt, synthetic construct
  misc_feature 1..20: note = Synthetic Construct
  source 1..20: mol_type = other RNA
ctggccctgttgctgctcac

SEQ ID NO: 114 - RNA, 20 nt, synthetic construct
  misc_feature 1..20: note = Synthetic Construct
  source 1..20: mol_type = other RNA
ctggggttgagggcaggggc

SEQ ID NO: 115 - RNA, 20 nt, synthetic construct
  misc_feature 1..20: note = Synthetic Construct
  source 1..20: mol_type = other RNA
ctggggttgagggcaggggt

SEQ ID NO: 116 - RNA, 20 nt, synthetic construct
  misc_feature 1..20: note = Synthetic Construct
  source 1..20: mol_type = other RNA
cttacagggatattctaagt

SEQ ID NO: 117 - RNA, 20 nt, synthetic construct
  misc_feature 1..20: note = Synthetic Construct
  source 1..20: mol_type = other RNA
cttatagggatattctaagt

SEQ ID NO: 118 - RNA, 20 nt, synthetic construct
  misc_feature 1..20: note = Synthetic Construct
  source 1..20: mol_type = other RNA
cttctcagtttcctggctca

SEQ ID NO: 119 - RNA, 20 nt, synthetic construct
  misc_feature 1..20: note = Synthetic Construct
  source 1..20: mol_type = other RNA
cttctccctaaatccccgtg

SEQ ID NO: 120 - RNA, 20 nt, synthetic construct
  misc_feature 1..20: note = Synthetic Construct
  source 1..20: mol_type = other RNA
cttgccccaggctgggcctc

SEQ ID NO: 121 - RNA, 20 nt, synthetic construct
  misc_feature 1..20: note = Synthetic Construct
  source 1..20: mol_type = other RNA
cttgcggagcctgcagcctt

SEQ ID NO: 122 - RNA, 20 nt, synthetic construct
  misc_feature 1..20: note = Synthetic Construct
  source 1..20: mol_type = other RNA
gaaaccggaatgggatctcc

SEQ ID NO: 123 - RNA, 20 nt, synthetic construct
  misc_feature 1..20: note = Synthetic Construct
  source 1..20: mol_type = other RNA
gaaactggaatgggatctcc

SEQ ID NO: 124 - RNA, 20 nt, synthetic construct
  misc_feature 1..20: note = Synthetic Construct
  source 1..20: mol_type = other RNA
gaaaggaaggaggtctttct

SEQ ID NO: 125 - RNA, 20 nt, synthetic construct
  misc_feature 1..20: note = Synthetic Construct
  source 1..20: mol_type = other RNA
gaagtctttctggacagagc

SEQ ID NO: 126 - RNA, 20 nt, synthetic construct
  misc_feature 1..20: note = Synthetic Construct
  source 1..20: mol_type = other RNA
gaatatccctataaggctgg

SEQ ID NO: 127 - RNA, 20 nt, synthetic construct
  misc_feature 1..20: note = Synthetic Construct
  source 1..20: mol_type = other RNA
gaatatccctgtaaggctgg

SEQ ID NO: 128 - RNA, 20 nt, synthetic construct
  misc_feature 1..20: note = Synthetic Construct
  source 1..20: mol_type = other RNA
gacctcccccagcaggttac

SEQ ID NO: 129 - RNA, 20 nt, synthetic construct
  misc_feature 1..20: note = Synthetic Construct
  source 1..20: mol_type = other RNA
gacctccctcagcaggttac

SEQ ID NO: 130 - RNA, 20 nt, synthetic construct
  misc_feature 1..20: note = Synthetic Construct
  source 1..20: mol_type = other RNA
gactcatgtgactgctggaa

SEQ ID NO: 131 - RNA, 20 nt, synthetic construct
  misc_feature 1..20: note = Synthetic Construct
  source 1..20: mol_type = other RNA
gacttctcagtttcctggct

SEQ ID NO: 132 - RNA, 20 nt, synthetic construct
  misc_feature 1..20: note = Synthetic Construct
  source 1..20: mol_type = other RNA
gagaaactggaatgggatct

SEQ ID NO: 133 - RNA, 20 nt, synthetic construct
  misc_feature 1..20: note = Synthetic Construct
  source 1..20: mol_type = other RNA
gagatcagactcgtgactgc

SEQ ID NO: 134 - RNA, 20 nt, synthetic construct
  misc_feature 1..20: note = Synthetic Construct
  source 1..20: mol_type = other RNA
gaggctgataagcccagccc

SEQ ID NO: 135 - RNA, 20 nt, synthetic construct
  misc_feature 1..20: note = Synthetic Construct
  source 1..20: mol_type = other RNA
gagggaggtctgggccttcc

SEQ ID NO: 136 - RNA, 20 nt, synthetic construct
  misc_feature 1..20: note = Synthetic Construct
  source 1..20: mol_type = other RNA
gaggtctttctggacagagc

SEQ ID NO: 137 - RNA, 20 nt, synthetic construct
  misc_feature 1..20: note = Synthetic Construct
  source 1..20: mol_type = other RNA
gatcagactgcaaaggctgc

SEQ ID NO: 138 - RNA, 20 nt, synthetic construct
  misc_feature 1..20: note = Synthetic Construct
  source 1..20: mol_type = other RNA
gatggtctggatggagaaac

SEQ ID NO: 139 - RNA, 20 nt, synthetic construct
  misc_feature 1..20: note = Synthetic Construct
  source 1..20: mol_type = other RNA
gatttagggagaaagccccc

SEQ ID NO: 140 - RNA, 20 nt, synthetic construct
  misc_feature 1..20: note = Synthetic Construct
  source 1..20: mol_type = other RNA
gatttagggagaaggccccc

SEQ ID NO: 141 - RNA, 20 nt, synthetic construct
  misc_feature 1..20: note = Synthetic Construct
  source 1..20: mol_type = other RNA
gcagcaacagggccagggct

SEQ ID NO: 142 - RNA, 20 nt, synthetic construct
  misc_feature 1..20: note = Synthetic Construct
  source 1..20: mol_type = other RNA
gcagcaacagggccggggct

SEQ ID NO: 143 - RNA, 20 nt, synthetic construct
  misc_feature 1..20: note = Synthetic Construct
  source 1..20: mol_type = other RNA
gcagcctttgcagtctgatc

SEQ ID NO: 144 - RNA, 20 nt, synthetic construct
  misc_feature 1..20: note = Synthetic Construct
  source 1..20: mol_type = other RNA
gcagggggcagaggcctgtg

SEQ ID NO: 145 - RNA, 20 nt, synthetic construct
  misc_feature 1..20: note = Synthetic Construct
  source 1..20: mol_type = other RNA
gccacggggatttagggaga

SEQ ID NO: 146 - RNA, 20 nt, synthetic construct
  misc_feature 1..20: note = Synthetic Construct
  source 1..20: mol_type = other RNA
gcccagcctggggcaaggcc

SEQ ID NO: 147 - RNA, 20 nt, synthetic construct
  misc_feature 1..20: note = Synthetic Construct
  source 1..20: mol_type = other RNA
gcccccttccagcagtcaca

SEQ ID NO: 148 - RNA, 20 nt, synthetic construct
  misc_feature 1..20: note = Synthetic Construct
  source 1..20: mol_type = other RNA
gcccggcctggggcaaggcc

SEQ ID NO: 149 - RNA, 20 nt, synthetic construct
  misc_feature 1..20: note = Synthetic Construct
  source 1..20: mol_type = other RNA
gccgctgtctttgagcacat

SEQ ID NO: 150 - RNA, 20 nt, synthetic construct
  misc_feature 1..20: note = Synthetic Construct
  source 1..20: mol_type = other RNA
gccgggcctctgggtacctg

SEQ ID NO: 151 - RNA, 20 nt, synthetic construct
  misc_feature 1..20: note = Synthetic Construct
  source 1..20: mol_type = other RNA
gcctatcaggggtgaaccct

SEQ ID NO: 152 - RNA, 20 nt, synthetic construct
  misc_feature 1..20: note = Synthetic Construct
  source 1..20: mol_type = other RNA
gcctatcaggggtgagccct

SEQ ID NO: 153 - RNA, 20 nt, synthetic construct
  misc_feature 1..20: note = Synthetic Construct
  source 1..20: mol_type = other RNA
gcctcgcagtccccgagcca

SEQ ID NO: 154 - RNA, 20 nt, synthetic construct
  misc_feature 1..20: note = Synthetic Construct
  source 1..20: mol_type = other RNA
gcctcgcagtccctgagcca

SEQ ID NO: 155 - RNA, 20 nt, synthetic construct
  misc_feature 1..20: note = Synthetic Construct
  source 1..20: mol_type = other RNA
gccttctccctaaatccccg

SEQ ID NO: 156 - RNA, 20 nt, synthetic construct
  misc_feature 1..20: note = Synthetic Construct
  source 1..20: mol_type = other RNA
gcgccccagcctatcagggg

SEQ ID NO: 157 - RNA, 20 nt, synthetic construct
  misc_feature 1..20: note = Synthetic Construct
  source 1..20: mol_type = other RNA
gcggacaggtgtcacccagg

SEQ ID NO: 158 - RNA, 20 nt, synthetic construct
  misc_feature 1..20: note = Synthetic Construct
  source 1..20: mol_type = other RNA
gctcacccctgataggctgg

SEQ ID NO: 159 - RNA, 20 nt, synthetic construct
  misc_feature 1..20: note = Synthetic Construct
  source 1..20: mol_type = other RNA
gctcagggactgcgaggcat

SEQ ID NO: 160 - RNA, 20 nt, synthetic construct
  misc_feature 1..20: note = Synthetic Construct
  source 1..20: mol_type = other RNA
gctcggggactgcgaggcat

SEQ ID NO: 161 - RNA, 20 nt, synthetic construct
  misc_feature 1..20: note = Synthetic Construct
  source 1..20: mol_type = other RNA
gctgggcctctgggtacctg

SEQ ID NO: 162 - RNA, 20 nt, synthetic construct
  misc_feature 1..20: note = Synthetic Construct
  source 1..20: mol_type = other RNA
gctttctccctaaatccccg

SEQ ID NO: 163 - RNA, 20 nt, synthetic construct
  misc_feature 1..20: note = Synthetic Construct
  source 1..20: mol_type = other RNA
ggaaaggaaggaagtctttc

SEQ ID NO: 164 - RNA, 20 nt, synthetic construct
  misc_feature 1..20: note = Synthetic Construct
  source 1..20: mol_type = other RNA
ggaaaggaaggaggtctttc

SEQ ID NO: 165 - RNA, 20 nt, synthetic construct
  misc_feature 1..20: note = Synthetic Construct
  source 1..20: mol_type = other RNA
ggaaggaagtctttctggac

SEQ ID NO: 166 - RNA, 20 nt, synthetic construct
  misc_feature 1..20: note = Synthetic Construct
  source 1..20: mol_type = other RNA
ggaaggaggtctttctggac

SEQ ID NO: 167 - RNA, 20 nt, synthetic construct
  misc_feature 1..20: note = Synthetic Construct
  source 1..20: mol_type = other RNA
ggactagtgcccagttacgt

SEQ ID NO: 168 - RNA, 20 nt, synthetic construct
  misc_feature 1..20: note = Synthetic Construct
  source 1..20: mol_type = other RNA
ggactggtgcccagttacgt

SEQ ID NO: 169 - RNA, 20 nt, synthetic construct
  misc_feature 1..20: note = Synthetic Construct
  source 1..20: mol_type = other RNA
ggagaaagccccccgatggt

SEQ ID NO: 170 - RNA, 20 nt, synthetic construct
  misc_feature 1..20: note = Synthetic Construct
  source 1..20: mol_type = other RNA
ggagaaggccccccgatggt

SEQ ID NO: 171 - RNA, 20 nt, synthetic construct
  misc_feature 1..20: note = Synthetic Construct
  source 1..20: mol_type = other RNA
ggagaccaacgtaactgggc

SEQ ID NO: 172 - RNA, 20 nt, synthetic construct
  misc_feature 1..20: note = Synthetic Construct
  source 1..20: mol_type = other RNA
ggagcctgcagcctttgcag

SEQ ID NO: 173 - RNA, 20 nt, synthetic construct
  misc_feature 1..20: note = Synthetic Construct
  source 1..20: mol_type = other RNA
ggcagggggcagaggcctgt

SEQ ID NO: 174 - RNA, 20 nt, synthetic construct
  misc_feature 1..20: note = Synthetic Construct
  source 1..20: mol_type = other RNA
ggcagtggagatcagactca

SEQ ID NO: 175 - RNA, 20 nt, synthetic construct
  misc_feature 1..20: note = Synthetic Construct
  source 1..20: mol_type = other RNA
ggcagtggagatcagactcg

SEQ ID NO: 176 - RNA, 20 nt, synthetic construct
  misc_feature 1..20: note = Synthetic Construct
  source 1..20: mol_type = other RNA
ggcccagcctggggcaaggc

SEQ ID NO: 177 - RNA, 20 nt, synthetic construct
  misc_feature 1..20: note = Synthetic Construct
  source 1..20: mol_type = other RNA
ggcccccttccagcagtcac

SEQ ID NO: 178 - RNA, 20 nt, synthetic construct
  misc_feature 1..20: note = Synthetic Construct
  source 1..20: mol_type = other RNA
ggcccggcctggggcaaggc

SEQ ID NO: 179 - RNA, 20 nt, synthetic construct
  misc_feature 1..20: note = Synthetic Construct
  source 1..20: mol_type = other RNA
ggctcacccctgataggctg

SEQ ID NO: 180 - RNA, 20 nt, synthetic construct
  misc_feature 1..20: note = Synthetic Construct
  source 1..20: mol_type = other RNA
gggaggctgataagcccagc

SEQ ID NO: 181 - RNA, 20 nt, synthetic construct
  misc_feature 1..20: note = Synthetic Construct
  source 1..20: mol_type = other RNA
gggcagggggcagaggcctg

SEQ ID NO: 182 - RNA, 20 nt, synthetic construct
  misc_feature 1..20: note = Synthetic Construct
  source 1..20: mol_type = other RNA
gggccacggggatttaggga

SEQ ID NO: 183 - RNA, 20 nt, synthetic construct
  misc_feature 1..20: note = Synthetic Construct
  source 1..20: mol_type = other RNA
gggccttctccctaaatccc

SEQ ID NO: 184 - RNA, 20 nt, synthetic construct
  misc_feature 1..20: note = Synthetic Construct
  source 1..20: mol_type = other RNA
gggctcacccctgataggct

SEQ ID NO: 185 - RNA, 20 nt, synthetic construct
  misc_feature 1..20: note = Synthetic Construct
  source 1..20: mol_type = other RNA
ggggaggtctgggccttcca

SEQ ID NO: 186 - RNA, 20 nt, synthetic construct
  misc_feature 1..20: note = Synthetic Construct
  source 1..20: mol_type = other RNA
ggggctgggcttatcagcct

SEQ ID NO: 187 - RNA, 20 nt, synthetic construct
  misc_feature 1..20: note = Synthetic Construct
  source 1..20: mol_type = other RNA
gggggaggtctgggccttcc

SEQ ID NO: 188 - RNA, 20 nt, synthetic construct
  misc_feature 1..20: note = Synthetic Construct
  source 1..20: mol_type = other RNA
ggggttgagggcaggggcag

SEQ ID NO: 189 - RNA, 20 nt, synthetic construct
  misc_feature 1..20: note = Synthetic Construct
  source 1..20: mol_type = other RNA
ggggttgagggcaggggtag

SEQ ID NO: 190 - RNA, 20 nt, synthetic construct
  misc_feature 1..20: note = Synthetic Construct
  source 1..20: mol_type = other RNA
gggtagggggcagaggcctg

SEQ ID NO: 191 - RNA, 20 nt, synthetic construct
  misc_feature 1..20: note = Synthetic Construct
  source 1..20: mol_type = other RNA
gggttcacccctgataggct

SEQ ID NO: 192 - RNA, 20 nt, synthetic construct
  misc_feature 1..20: note = Synthetic Construct
  source 1..20: mol_type = other RNA
gggttgagggcaggggcagg

SEQ ID NO: 193 - RNA, 20 nt, synthetic construct
  misc_feature 1..20: note = Synthetic Construct
  source 1..20: mol_type = other RNA
gggttgagggcaggggtagg

SEQ ID NO: 194 - RNA, 20 nt, synthetic construct
  misc_feature 1..20: note = Synthetic Construct
  source 1..20: mol_type = other RNA
ggtacccagaggcccagcct

SEQ ID NO: 195 - RNA, 20 nt, synthetic construct
  misc_feature 1..20: note = Synthetic Construct
  source 1..20: mol_type = other RNA
ggtacccagaggcccggcct

SEQ ID NO: 196 - RNA, 20 nt, synthetic construct
  misc_feature 1..20: note = Synthetic Construct
  source 1..20: mol_type = other RNA
ggtagggggcagaggcctgt

SEQ ID NO: 197 - RNA, 20 nt, synthetic construct
  misc_feature 1..20: note = Synthetic Construct
  source 1..20: mol_type = other RNA
ggtcctatcagtaacctgct

SEQ ID NO: 198 - RNA, 20 nt, synthetic construct
  misc_feature 1..20: note = Synthetic Construct
  source 1..20: mol_type = other RNA
ggtgtcacccagggctcacc

SEQ ID NO: 199 - RNA, 20 nt, synthetic construct
  misc_feature 1..20: note = Synthetic Construct
  source 1..20: mol_type = other RNA
ggtgtcacccagggttcacc

SEQ ID NO: 200 - RNA, 20 nt, synthetic construct
  misc_feature 1..20: note = Synthetic Construct
  source 1..20: mol_type = other RNA
ggttcacccctgataggctg

SEQ ID NO: 201 - RNA, 20 nt, synthetic construct
  misc_feature 1..20: note = Synthetic Construct
  source 1..20: mol_type = other RNA
ggttcaggccttgccccagg

SEQ ID NO: 202 - RNA, 20 nt, synthetic construct
  misc_feature 1..20: note = Synthetic Construct
  source 1..20: mol_type = other RNA
gtaacctgctgagggaggtc

SEQ ID NO: 203 - RNA, 20 nt, synthetic construct
  misc_feature 1..20: note = Synthetic Construct
  source 1..20: mol_type = other RNA
gtacccagaggcccagcctg

SEQ ID NO: 204 - RNA, 20 nt, synthetic construct
  misc_feature 1..20: note = Synthetic Construct
  source 1..20: mol_type = other RNA
gtacccagaggcccggcctg

SEQ ID NO: 205 - RNA, 20 nt, synthetic construct
  misc_feature 1..20: note = Synthetic Construct
  source 1..20: mol_type = other RNA
gtagggggcagaggcctgtg

SEQ ID NO: 206 - RNA, 20 nt, synthetic construct
  misc_feature 1..20: note = Synthetic Construct
  source 1..20: mol_type = other RNA
gtcacgagtctgatctccac

SEQ ID NO: 207 - RNA, 20 nt, synthetic construct
  misc_feature 1..20: note = Synthetic Construct
  source 1..20: mol_type = other RNA
gtccccgagccaggaaactg

SEQ ID NO: 208 - RNA, 20 nt, synthetic construct
  misc_feature 1..20: note = Synthetic Construct
  source 1..20: mol_type = other RNA
gtccctgagccaggaaactg

SEQ ID NO: 209 - RNA, 20 nt, synthetic construct
  misc_feature 1..20: note = Synthetic Construct
  source 1..20: mol_type = other RNA
gtcctatcagtaacctgctg

SEQ ID NO: 210 - RNA, 20 nt, synthetic construct
  misc_feature 1..20: note = Synthetic Construct
  source 1..20: mol_type = other RNA
gtctctgccgctgtctttga

SEQ ID NO: 211 - RNA, 20 nt, synthetic construct
  misc_feature 1..20: note = Synthetic Construct
  source 1..20: mol_type = other RNA
gtgctcaaagacagcggcag

SEQ ID NO: 212 - RNA, 20 nt, synthetic construct
  misc_feature 1..20: note = Synthetic Construct
  source 1..20: mol_type = other RNA
gtggatgtgctcaaagacag

SEQ ID NO: 213 - RNA, 20 nt, synthetic construct
  misc_feature 1..20: note = Synthetic Construct
  source 1..20: mol_type = other RNA
gttcacccctgataggctgg

SEQ ID NO: 214 - RNA, 20 nt, synthetic construct
  misc_feature 1..20: note = Synthetic Construct
  source 1..20: mol_type = other RNA
gttcaggccttgccccaggc

SEQ ID NO: 215 - RNA, 20 nt, synthetic construct
  misc_feature 1..20: note = Synthetic Construct
  source 1..20: mol_type = other RNA
gtttcctggctcagggactg

SEQ ID NO: 216 - RNA, 20 nt, synthetic construct
  misc_feature 1..20: note = Synthetic Construct
  source 1..20: mol_type = other RNA
gtttcctggctcggggactg

SEQ ID NO: 217 - RNA, 20 nt, synthetic construct
  misc_feature 1..20: note = Synthetic Construct
  source 1..20: mol_type = other RNA
taacctgctgagggaggtct

SEQ ID NO: 218 - RNA, 20 nt, synthetic construct
  misc_feature 1..20: note = Synthetic Construct
  source 1..20: mol_type = other RNA
taacctgctgggggaggtct

SEQ ID NO: 219 - RNA, 20 nt, synthetic construct
  misc_feature 1..20: note = Synthetic Construct
  source 1..20: mol_type = other RNA
tacagggatattctaagttg

SEQ ID NO: 220 - RNA, 20 nt, synthetic construct
  misc_feature 1..20: note = Synthetic Construct
  source 1..20: mol_type = other RNA
tagaatatccctataaggct

SEQ ID NO: 221 - RNA, 20 nt, synthetic construct
  misc_feature 1..20: note = Synthetic Construct
  source 1..20: mol_type = other RNA
tagaatatccctgtaaggct

SEQ ID NO: 222 - RNA, 20 nt, synthetic construct
  misc_feature 1..20: note = Synthetic Construct
  source 1..20: mol_type = other RNA
tagtctctgccgctgtcttt

SEQ ID NO: 223 - RNA, 20 nt, synthetic construct
  misc_feature 1..20: note = Synthetic Construct
  source 1..20: mol_type = other RNA
tatagggatattctaagttg

SEQ ID NO: 224 - RNA, 20 nt, synthetic construct
  misc_feature 1..20: note = Synthetic Construct
  source 1..20: mol_type = other RNA
tatcaggggtgaaccctggg

SEQ ID NO: 225 - RNA, 20 nt, synthetic construct
  misc_feature 1..20: note = Synthetic Construct
  source 1..20: mol_type = other RNA
tatcaggggtgagccctggg

SEQ ID NO: 226 - RNA, 20 nt, synthetic construct
  misc_feature 1..20: note = Synthetic Construct
  source 1..20: mol_type = other RNA
tatcagtaacctgctgaggg

SEQ ID NO: 227 - RNA, 20 nt, synthetic construct
  misc_feature 1..20: note = Synthetic Construct
  source 1..20: mol_type = other RNA
tatcagtaacctgctggggg

SEQ ID NO: 228 - RNA, 20 nt, synthetic construct
  misc_feature 1..20: note = Synthetic Construct
  source 1..20: mol_type = other RNA
tcagactcgtgactgctgga

SEQ ID NO: 229 - RNA, 20 nt, synthetic construct
  misc_feature 1..20: note = Synthetic Construct
  source 1..20: mol_type = other RNA
tcagggactgcgaggcatca

SEQ ID NO: 230 - RNA, 20 nt, synthetic construct
  misc_feature 1..20: note = Synthetic Construct
  source 1..20: mol_type = other RNA
tcccattccagtttctccat

SEQ ID NO: 231 - RNA, 20 nt, synthetic construct
  misc_feature 1..20: note = Synthetic Construct
  source 1..20: mol_type = other RNA
tcccattccggtttctccat

SEQ ID NO: 232 - RNA, 20 nt, synthetic construct
  misc_feature 1..20: note = Synthetic Construct
  source 1..20: mol_type = other RNA
tccctataaggctggaggcc

SEQ ID NO: 233 - RNA, 20 nt, synthetic construct
  misc_feature 1..20: note = Synthetic Construct
  source 1..20: mol_type = other RNA
tccctcagcaggttactgat

SEQ ID NO: 234 - RNA, 20 nt, synthetic construct
  misc_feature 1..20: note = Synthetic Construct
  source 1..20: mol_type = other RNA
tccctgtaaggctggaggcc

SEQ ID NO: 235 - RNA, 20 nt, synthetic construct
  misc_feature 1..20: note = Synthetic Construct
  source 1..20: mol_type = other RNA
tcctatcagtaacctgctga

SEQ ID NO: 236 - RNA, 20 nt, synthetic construct
  misc_feature 1..20: note = Synthetic Construct
  source 1..20: mol_type = other RNA
tcctgatgcctcgcagtccc

SEQ ID NO: 237 - RNA, 20 nt, synthetic construct
  misc_feature 1..20: note = Synthetic Construct
  source 1..20: mol_type = other RNA
tcggggactgcgaggcatca

SEQ ID NO: 238 - RNA, 20 nt, synthetic construct
  misc_feature 1..20: note = Synthetic Construct
  source 1..20: mol_type = other RNA
tctggatggagaaactggaa

SEQ ID NO: 239 - RNA, 20 nt, synthetic construct
  misc_feature 1..20: note = Synthetic Construct
  source 1..20: mol_type = other RNA
tctggcctccagccttatag

SEQ ID NO: 240 - RNA, 20 nt, synthetic construct
  misc_feature 1..20: note = Synthetic Construct
  source 1..20: mol_type = other RNA
tgagggcaggggcagggggc

SEQ ID NO: 241 - RNA, 20 nt, synthetic construct
  misc_feature 1..20: note = Synthetic Construct
  source 1..20: mol_type = other RNA
tgcctcgcagtccccgagcc

SEQ ID NO: 242 - RNA, 20 nt, synthetic construct
  misc_feature 1..20: note = Synthetic Construct
  source 1..20: mol_type = other RNA
tgcctcgcagtccctgagcc

SEQ ID NO: 243 - RNA, 20 nt, synthetic construct
  misc_feature 1..20: note = Synthetic Construct
  source 1..20: mol_type = other RNA
tggaaaggaaggaagtcttt

SEQ ID NO: 244 - RNA, 20 nt, synthetic construct
  misc_feature 1..20: note = Synthetic Construct
  source 1..20: mol_type = other RNA
tggaaaggaaggaggtcttt

SEQ ID NO: 245 - RNA, 20 nt, synthetic construct
  misc_feature 1..20: note = Synthetic Construct
  source 1..20: mol_type = other RNA
tggaatgggatctccaggca

SEQ ID NO: 246 - RNA, 20 nt, synthetic construct
  misc_feature 1..20: note = Synthetic Construct
  source 1..20: mol_type = other RNA
tggagatcccattccagttt

SEQ ID NO: 247 - RNA, 20 nt, synthetic construct
  misc_feature 1..20: note = Synthetic Construct
  source 1..20: mol_type = other RNA
tggagatcccattccggttt

SEQ ID NO: 248 - RNA, 20 nt, synthetic construct
  misc_feature 1..20: note = Synthetic Construct
  source 1..20: mol_type = other RNA
tggatggagaaaccggaatg

SEQ ID NO: 249 - RNA, 20 nt, synthetic construct
  misc_feature 1..20: note = Synthetic Construct
  source 1..20: mol_type = other RNA
tggatggagaaactggaatg

SEQ ID NO: 250 - RNA, 20 nt, synthetic construct
  misc_feature 1..20: note = Synthetic Construct
  source 1..20: mol_type = other RNA
tggcccccttccagcagtca

SEQ ID NO: 251 - RNA, 20 nt, synthetic construct
  misc_feature 1..20: note = Synthetic Construct
  source 1..20: mol_type = other RNA
tgggactagtgcccagttac

SEQ ID NO: 252 - RNA, 20 nt, synthetic construct
  misc_feature 1..20: note = Synthetic Construct
  source 1..20: mol_type = other RNA
tgggactggtgcccagttac

SEQ ID NO: 253 - RNA, 20 nt, synthetic construct
  misc_feature 1..20: note = Synthetic Construct
  source 1..20: mol_type = other RNA
tggggttgagggcaggggca

SEQ ID NO: 254 - RNA, 20 nt, synthetic construct
  misc_feature 1..20: note = Synthetic Construct
  source 1..20: mol_type = other RNA
tggggttgagggcaggggta

SEQ ID NO: 255 - RNA, 20 nt, synthetic construct
  misc_feature 1..20: note = Synthetic Construct
  source 1..20: mol_type = other RNA
tgtcacccagggttcacccc

SEQ ID NO: 256 - RNA, 20 nt, synthetic construct
  misc_feature 1..20: note = Synthetic Construct
  source 1..20: mol_type = other RNA
ttagaatatccctataaggc

SEQ ID NO: 257 - RNA, 20 nt, synthetic construct
  misc_feature 1..20: note = Synthetic Construct
  source 1..20: mol_type = other RNA
ttagaatatccctgtaaggc

SEQ ID NO: 258 - RNA, 20 nt, synthetic construct
  misc_feature 1..20: note = Synthetic Construct
  source 1..20: mol_type = other RNA
ttagggagaaagccccccga

SEQ ID NO: 259 - RNA, 20 nt, synthetic construct
  misc_feature 1..20: note = Synthetic Construct
  source 1..20: mol_type = other RNA
ttagggagaaggccccccga

SEQ ID NO: 260 - RNA, 20 nt, synthetic construct
  misc_feature 1..20: note = Synthetic Construct
  source 1..20: mol_type = other RNA
ttcaggccttgccccaggcc

SEQ ID NO: 261 - RNA, 20 nt, synthetic construct
  misc_feature 1..20: note = Synthetic Construct
  source 1..20: mol_type = other RNA
ttcaggccttgccccaggct

SEQ ID NO: 262 - RNA, 20 nt, synthetic construct
  misc_feature 1..20: note = Synthetic Construct
  source 1..20: mol_type = other RNA
ttccagcagtcacatgagtc

SEQ ID NO: 263 - RNA, 20 nt, synthetic construct
  misc_feature 1..20: note = Synthetic Construct
  source 1..20: mol_type = other RNA
ttcctggctcagggactgcg

SEQ ID NO: 264 - RNA, 20 nt, synthetic construct
  misc_feature 1..20: note = Synthetic Construct
  source 1..20: mol_type = other RNA
ttcctggctcggggactgcg

SEQ ID NO: 265 - RNA, 20 nt, synthetic construct
  misc_feature 1..20: note = Synthetic Construct
  source 1..20: mol_type = other RNA
ttctcagtttcctggctcag

SEQ ID NO: 266 - RNA, 20 nt, synthetic construct
  misc_feature 1..20: note = Synthetic Construct
  source 1..20: mol_type = other RNA
ttctcagtttcctggctcgg

SEQ ID NO: 267 - RNA, 20 nt, synthetic construct
  misc_feature 1..20: note = Synthetic Construct
  source 1..20: mol_type = other RNA
ttgccccaggccgggcctct

SEQ ID NO: 268 - RNA, 20 nt, synthetic construct
  misc_feature 1..20: note = Synthetic Construct
  source 1..20: mol_type = other RNA
ttgccccaggctgggcctct

SEQ ID NO: 269 - RNA, 20 nt, synthetic construct
  misc_feature 1..20: note = Synthetic Construct
  source 1..20: mol_type = other RNA
tttctccctaaatccccgtg

SEQ ID NO: 270 - RNA, 20 nt, synthetic construct
  misc_feature 1..20: note = Synthetic Construct
  source 1..20: mol_type = other RNA
aaaaaggagacagagctggg

SEQ ID NO: 271 - RNA, 20 nt, synthetic construct
  misc_feature 1..20: note = Synthetic Construct
  source 1..20: mol_type = other RNA
aaaaggagacagagctggga

SEQ ID NO: 272 - RNA, 20 nt, synthetic construct
  misc_feature 1..20: note = Synthetic Construct
  source 1..20: mol_type = other RNA
aaaccggaatgggatctcca

SEQ ID NO: 273 - RNA, 20 nt, synthetic construct
  misc_feature 1..20: note = Synthetic Construct
  source 1..20: mol_type = other RNA
aaactgggacacatagtctc

SEQ ID NO: 274 - RNA, 20 nt, synthetic construct
  misc_feature 1..20: note = Synthetic Construct
  source 1..20: mol_type = other RNA
aaagacagctcgcagagact

SEQ ID NO: 275 - RNA, 20 nt, synthetic construct
  misc_feature 1..20: note = Synthetic Construct
  source 1..20: mol_type = other RNA
aaagccccccgatggttggc

SEQ ID NO: 276 - RNA, 20 nt, synthetic construct
  misc_feature 1..20: note = Synthetic Construct
  source 1..20: mol_type = other RNA
aaaggaaggaagtctttctg

SEQ ID NO: 277 - RNA, 20 nt, synthetic construct
  misc_feature 1..20: note = Synthetic Construct
  source 1..20: mol_type = other RNA
aaaggaaggaggtctttctg

SEQ ID NO: 278 - RNA, 20 nt, synthetic construct
  misc_feature 1..20: note = Synthetic Construct
  source 1..20: mol_type = other RNA
aaatctggcctccagcctta

SEQ ID NO: 279 - RNA, 20 nt, synthetic construct
  misc_feature 1..20: note = Synthetic Construct
  source 1..20: mol_type = other RNA
aacagggccagggctgggct

SEQ ID NO: 280 - RNA, 20 nt, synthetic construct
  misc_feature 1..20: note = Synthetic Construct
  source 1..20: mol_type = other RNA
aacagggccggggctgggct

SEQ ID NO: 281 - RNA, 20 nt, synthetic construct
  misc_feature 1..20: note = Synthetic Construct
  source 1..20: mol_type = other RNA
aaccatcggggggccttctc

SEQ ID NO: 282 - RNA, 20 nt, synthetic construct
  misc_feature 1..20: note = Synthetic Construct
  source 1..20: mol_type = other RNA
aaccatcggggggctttctc

SEQ ID NO: 283 - RNA, 20 nt, synthetic construct
  misc_feature 1..20: note = Synthetic Construct
  source 1..20: mol_type = other RNA
aaccggaatgggatctccag

SEQ ID NO: 284 - RNA, 20 nt, synthetic construct
  misc_feature 1..20: note = Synthetic Construct
  source 1..20: mol_type = other RNA
aacctgctgagggaggtctg

SEQ ID NO: 285 - RNA, 20 nt, synthetic construct
  misc_feature 1..20: note = Synthetic Construct
  source 1..20: mol_type = other RNA
aacctgctgggggaggtctg

SEQ ID NO: 286 - RNA, 20 nt, synthetic construct
  misc_feature 1..20: note = Synthetic Construct
  source 1..20: mol_type = other RNA
aacgtaactgggcactagtc

SEQ ID NO: 287 - RNA, 20 nt, synthetic construct
  misc_feature 1..20: note = Synthetic Construct
  source 1..20: mol_type = other RNA
aactggaatgggatctccag

SEQ ID NO: 288 - RNA, 20 nt, synthetic construct
  misc_feature 1..20: note = Synthetic Construct
  source 1..20: mol_type = other RNA
aactgggacacatagtctct

SEQ ID NO: 289 - RNA, 20 nt, synthetic construct
  misc_feature 1..20: note = Synthetic Construct
  source 1..20: mol_type = other RNA
aactgggcaccagtcccagc

SEQ ID NO: 290 - RNA, 20 nt, synthetic construct
  misc_feature 1..20: note = Synthetic Construct
  source 1..20: mol_type = other RNA
aactgggcactagtcccagc

SEQ ID NO: 291 - RNA, 20 nt, synthetic construct
  misc_feature 1..20: note = Synthetic Construct
  source 1..20: mol_type = other RNA
aacttagaatatccctataa

SEQ ID NO: 292 - RNA, 20 nt, synthetic construct
  misc_feature 1..20: note = Synthetic Construct
  source 1..20: mol_type = other RNA
aacttagaatatccctgtaa

SEQ ID NO: 293 - RNA, 20 nt, synthetic construct
  misc_feature 1..20: note = Synthetic Construct
  source 1..20: mol_type = other RNA
aagacagcggcagagactat

SEQ ID NO: 294 - RNA, 20 nt, synthetic construct
  misc_feature 1..20: note = Synthetic Construct
  source 1..20: mol_type = other RNA
aagacagctcgcagagacta

SEQ ID NO: 295 - RNA, 20 nt, synthetic construct
  misc_feature 1..20: note = Synthetic Construct
  source 1..20: mol_type = other RNA
aagcccagccctggccctgt

SEQ ID NO: 296 - RNA, 20 nt, synthetic construct
  misc_feature 1..20: note = Synthetic Construct
  source 1..20: mol_type = other RNA
aagccccccgatggttggct

SEQ ID NO: 297 - RNA, 20 nt, synthetic construct
  misc_feature 1..20: note = Synthetic Construct
  source 1..20: mol_type = other RNA
aagctgcaggctccgcaagg

SEQ ID NO: 298 - RNA, 20 nt, synthetic construct
  misc_feature 1..20: note = Synthetic Construct
  source 1..20: mol_type = other RNA
aaggaaggaagtctttctgg

SEQ ID NO: 299 - RNA, 20 nt, synthetic construct
  misc_feature 1..20: note = Synthetic Construct
  source 1..20: mol_type = other RNA
aaggaaggaggtctttctgg

SEQ ID NO: 300 - RNA, 20 nt, synthetic construct
  misc_feature 1..20: note = Synthetic Construct
  source 1..20: mol_type = other RNA
aaggaagtctttctggacag

SEQ ID NO: 301 - RNA, 20 nt, synthetic construct
  misc_feature 1..20: note = Synthetic Construct
  source 1..20: mol_type = other RNA
aaggaggtctttctggacag

SEQ ID NO: 302 - RNA, 20 nt, synthetic construct
  misc_feature 1..20: note = Synthetic Construct
  source 1..20: mol_type = other RNA
aaggcccagacctcccccag

SEQ ID NO: 303 - RNA, 20 nt, synthetic construct
  misc_feature 1..20: note = Synthetic Construct
  source 1..20: mol_type = other RNA
aaggcccagacctccctcag

SEQ ID NO: 304 - RNA, 20 nt, synthetic construct
  misc_feature 1..20: note = Synthetic Construct
  source 1..20: mol_type = other RNA
aaggccccccgatggttggc

SEQ ID NO: 305 - RNA, 20 nt, synthetic construct
  misc_feature 1..20: note = Synthetic Construct
  source 1..20: mol_type = other RNA
aaggttcaggccttgcccca

SEQ ID NO: 306 - RNA, 20 nt, synthetic construct
  misc_feature 1..20: note = Synthetic Construct
  source 1..20: mol_type = other RNA
aagtccctgatcagactgca

SEQ ID NO: 307 - RNA, 20 nt, synthetic construct
  misc_feature 1..20: note = Synthetic Construct
  source 1..20: mol_type = other RNA
aagtctttctggacagagct

SEQ ID NO: 308 - RNA, 20 nt, synthetic construct
  misc_feature 1..20: note = Synthetic Construct
  source 1..20: mol_type = other RNA
aatatccctataaggctgga

SEQ ID NO: 309 - RNA, 20 nt, synthetic construct
  misc_feature 1..20: note = Synthetic Construct
  source 1..20: mol_type = other RNA
aatatccctgtaaggctgga

SEQ ID NO: 310 - RNA, 20 nt, synthetic construct
  misc_feature 1..20: note = Synthetic Construct
  source 1..20: mol_type = other RNA
acaacttagaatatccctat

SEQ ID NO: 311 - RNA, 20 nt, synthetic construct
  misc_feature 1..20: note = Synthetic Construct
  source 1..20: mol_type = other RNA
acaacttagaatatccctgt

SEQ ID NO: 312 - RNA, 20 nt, synthetic construct
  misc_feature 1..20: note = Synthetic Construct
  source 1..20: mol_type = other RNA
acacatagtctctgccgctg

SEQ ID NO: 313 - RNA, 20 nt, synthetic construct
  misc_feature 1..20: note = Synthetic Construct
  source 1..20: mol_type = other RNA
acacatagtctctgcgagct

SEQ ID NO: 314 - RNA, 20 nt, synthetic construct
  misc_feature 1..20: note = Synthetic Construct
  source 1..20: mol_type = other RNA
acagagctgggactagtgcc

SEQ ID NO: 315 - RNA, 20 nt, synthetic construct
  misc_feature 1..20: note = Synthetic Construct
  source 1..20: mol_type = other RNA
acagagctgggactggtgcc

SEQ ID NO: 316 - RNA, 20 nt, synthetic construct
  misc_feature 1..20: note = Synthetic Construct
  source 1..20: mol_type = other RNA
acagatggtctggatggaga

SEQ ID NO: 317 - RNA, 20 nt, synthetic construct
  misc_feature 1..20: note = Synthetic Construct
  source 1..20: mol_type = other RNA
acagcggcagagactatgtg

SEQ ID NO: 318 - RNA, 20 nt, synthetic construct
  misc_feature 1..20: note = Synthetic Construct
  source 1..20: mol_type = other RNA
acagctcgcagagactatgt

SEQ ID NO: 319 - RNA, 20 nt, synthetic construct
  misc_feature 1..20: note = Synthetic Construct
  source 1..20: mol_type = other RNA
acaggcctctgccccctacc

SEQ ID NO: 320 - RNA, 20 nt, synthetic construct
  misc_feature 1..20: note = Synthetic Construct
  source 1..20: mol_type = other RNA
acaggcctctgccccctgcc

SEQ ID NO: 321 - RNA, 20 nt, synthetic construct
  misc_feature 1..20: note = Synthetic Construct
  source 1..20: mol_type = other RNA
acaggtgtcacccagggctc

SEQ ID NO: 322 - RNA, 20 nt, synthetic construct
  misc_feature 1..20: note = Synthetic Construct
  source 1..20: mol_type = other RNA
acaggtgtcacccagggttc

SEQ ID NO: 323 - RNA, 20 nt, synthetic construct
  misc_feature 1..20: note = Synthetic Construct
  source 1..20: mol_type = other RNA
acatagtctctgccgctgtc

SEQ ID NO: 324 - RNA, 20 nt, synthetic construct
  misc_feature 1..20: note = Synthetic Construct
  source 1..20: mol_type = other RNA
acatagtctctgcgagctgt

SEQ ID NO: 325 - RNA, 20 nt, synthetic construct
  misc_feature 1..20: note = Synthetic Construct
  source 1..20: mol_type = other RNA
acatgagtctgatctccact

SEQ ID NO: 326 - RNA, 20 nt, synthetic construct
  misc_feature 1..20: note = Synthetic Construct
  source 1..20: mol_type = other RNA
accaacgtaactgggcacca

SEQ ID NO: 327 - RNA, 20 nt, synthetic construct
  misc_feature 1..20: note = Synthetic Construct
  source 1..20: mol_type = other RNA
accaacgtaactgggcacta

SEQ ID NO: 328 - RNA, 20 nt, synthetic construct
  misc_feature 1..20: note = Synthetic Construct
  source 1..20: mol_type = other RNA
accagtcccagctctgtctc

SEQ ID NO: 329 - RNA, 20 nt, synthetic construct
  misc_feature 1..20: note = Synthetic Construct
  source 1..20: mol_type = other RNA
accagtgagcagcaacaggg

SEQ ID NO: 330 - RNA, 20 nt, synthetic construct
  misc_feature 1..20: note = Synthetic Construct
  source 1..20: mol_type = other RNA
acccagaggcccagcctggg

SEQ ID NO: 331 - RNA, 20 nt, synthetic construct
  misc_feature 1..20: note = Synthetic Construct
  source 1..20: mol_type = other RNA
acccagaggcccggcctggg

SEQ ID NO: 332 - RNA, 20 nt, synthetic construct
  misc_feature 1..20: note = Synthetic Construct
  source 1..20: mol_type = other RNA
acccctgccctcaaccccag

SEQ ID NO: 333 - RNA, 20 nt, synthetic construct
  misc_feature 1..20: note = Synthetic Construct
  source 1..20: mol_type = other RNA
accctgcctggagatcccat

SEQ ID NO: 334 - RNA, 20 nt, synthetic construct
  misc_feature 1..20: note = Synthetic Construct
  source 1..20: mol_type = other RNA
acccttgcggagcctgcagc

SEQ ID NO: 335 - RNA, 20 nt, synthetic construct
  misc_feature 1..20: note = Synthetic Construct
  source 1..20: mol_type = other RNA
acctcccccagcaggttact

SEQ ID NO: 336 - RNA, 20 nt, synthetic construct
  misc_feature 1..20: note = Synthetic Construct
  source 1..20: mol_type = other RNA
acctccctcagcaggttact

SEQ ID NO: 337 - RNA, 20 nt, synthetic construct
  misc_feature 1..20: note = Synthetic Construct
  source 1..20: mol_type = other RNA
acctgctgagggaggtctgg

SEQ ID NO: 338 - RNA, 20 nt, synthetic construct
  misc_feature 1..20: note = Synthetic Construct
  source 1..20: mol_type = other RNA
acctgctgggggaggtctgg

SEQ ID NO: 339 - RNA, 20 nt, synthetic construct
  misc_feature 1..20: note = Synthetic Construct
  source 1..20: mol_type = other RNA
acgagtctgatctccactgc

SEQ ID NO: 340 - RNA, 20 nt, synthetic construct
  misc_feature 1..20: note = Synthetic Construct
  source 1..20: mol_type = other RNA
acggggatttagggagaaag

SEQ ID NO: 341 - RNA, 20 nt, synthetic construct
  misc_feature 1..20: note = Synthetic Construct
  source 1..20: mol_type = other RNA
acggggatttagggagaagg

SEQ ID NO: 342 - RNA, 20 nt, synthetic construct
  misc_feature 1..20: note = Synthetic Construct
  source 1..20: mol_type = other RNA
acgtaactgggcaccagtcc

SEQ ID NO: 343 - RNA, 20 nt, synthetic construct
  misc_feature 1..20: note = Synthetic Construct
  source 1..20: mol_type = other RNA
acgtaactgggcactagtcc

SEQ ID NO: 344 - RNA, 20 nt, synthetic construct
  misc_feature 1..20: note = Synthetic Construct
  source 1..20: mol_type = other RNA
actagtcccagctctgtctc

SEQ ID NO: 345 - RNA, 20 nt, synthetic construct
  misc_feature 1..20: note = Synthetic Construct
  source 1..20: mol_type = other RNA
actcgtgactgctggaaggg

SEQ ID NO: 346 - RNA, 20 nt, synthetic construct
  misc_feature 1..20: note = Synthetic Construct
  source 1..20: mol_type = other RNA
actgcaaaggctgcaggctc

SEQ ID NO: 347 - RNA, 20 nt, synthetic construct
  misc_feature 1..20: note = Synthetic Construct
  source 1..20: mol_type = other RNA
actgggacacatagtctctg

SEQ ID NO: 348 - RNA, 20 nt, synthetic construct
  misc_feature 1..20: note = Synthetic Construct
  source 1..20: mol_type = other RNA
actgggcaccagtcccagct

SEQ ID NO: 349 - RNA, 20 nt, synthetic construct
  misc_feature 1..20: note = Synthetic Construct
  source 1..20: mol_type = other RNA
actgggcactagtcccagct

SEQ ID NO: 350 - RNA, 20 nt, synthetic construct
  misc_feature 1..20: note = Synthetic Construct
  source 1..20: mol_type = other RNA
actggtgcccagttacgttg

SEQ ID NO: 351 - RNA, 20 nt, synthetic construct
  misc_feature 1..20: note = Synthetic Construct
  source 1..20: mol_type = other RNA
acttagaatatccctataag

SEQ ID NO: 352 - RNA, 20 nt, synthetic construct
  misc_feature 1..20: note = Synthetic Construct
  source 1..20: mol_type = other RNA
acttagaatatccctgtaag

SEQ ID NO: 353 - RNA, 20 nt, synthetic construct
  misc_feature 1..20: note = Synthetic Construct
  source 1..20: mol_type = other RNA
agaaaccggaatgggatctc

SEQ ID NO: 354 - RNA, 20 nt, synthetic construct
  misc_feature 1..20: note = Synthetic Construct
  source 1..20: mol_type = other RNA
agaaactggaatgggatctc

SEQ ID NO: 355 - RNA, 20 nt, synthetic construct
  misc_feature 1..20: note = Synthetic Construct
  source 1..20: mol_type = other RNA
agaaagccccccgatggttg

SEQ ID NO: 356 - RNA, 20 nt, synthetic construct
  misc_feature 1..20: note = Synthetic Construct
  source 1..20: mol_type = other RNA
agaaggccccccgatggttg

SEQ ID NO: 357 - RNA, 20 nt, synthetic construct
  misc_feature 1..20: note = Synthetic Construct
  source 1..20: mol_type = other RNA
agaatatccctataaggctg

SEQ ID NO: 358 - RNA, 20 nt, synthetic construct
  misc_feature 1..20: note = Synthetic Construct
  source 1..20: mol_type = other RNA
agaatatccctgtaaggctg

SEQ ID NO: 359 - RNA, 20 nt, synthetic construct
  misc_feature 1..20: note = Synthetic Construct
  source 1..20: mol_type = other RNA
agacagagctgggactagtg

SEQ ID NO: 360 - RNA, 20 nt, synthetic construct
  misc_feature 1..20: note = Synthetic Construct
  source 1..20: mol_type = other RNA
agacagcggcagagactatg

SEQ ID NO: 361 - RNA, 20 nt, synthetic construct
  misc_feature 1..20: note = Synthetic Construct
  source 1..20: mol_type = other RNA
agacagctcgcagagactat

SEQ ID NO: 362 - RNA, 20 nt, synthetic construct
  misc_feature 1..20: note = Synthetic Construct
  source 1..20: mol_type = other RNA
agaccaacgtaactgggcac

SEQ ID NO: 363 - RNA, 20 nt, synthetic construct
  misc_feature 1..20: note = Synthetic Construct
  source 1..20: mol_type = other RNA
agacctcccccagcaggtta

SEQ ID NO: 364 - RNA, 20 nt, synthetic construct
  misc_feature 1..20: note = Synthetic Construct
  source 1..20: mol_type = other RNA
agactgcaaaggctgcaggc

SEQ ID NO: 365 - RNA, 20 nt, synthetic construct
  misc_feature 1..20: note = Synthetic Construct
  source 1..20: mol_type = other RNA
agagctgggactagtgccca

SEQ ID NO: 366 - RNA, 20 nt, synthetic construct
  misc_feature 1..20: note = Synthetic Construct
  source 1..20: mol_type = other RNA
agagctgggactggtgccca

SEQ ID NO: 367 - RNA, 20 nt, synthetic construct
  misc_feature 1..20: note = Synthetic Construct
  source 1..20: mol_type = other RNA
agaggcccggcctggggcaa

SEQ ID NO: 368 - RNA, 20 nt, synthetic construct
  misc_feature 1..20: note = Synthetic Construct
  source 1..20: mol_type = other RNA
agatcagactcatgtgactg

SEQ ID NO: 369 - RNA, 20 nt, synthetic construct
  misc_feature 1..20: note = Synthetic Construct
  source 1..20: mol_type = other RNA
agatcccattccagtttctc

SEQ ID NO: 370 - RNA, 20 nt, synthetic construct
  misc_feature 1..20: note = Synthetic Construct
  source 1..20: mol_type = other RNA
agatcccattccggtttctc

SEQ ID NO: 371 - RNA, 20 nt, synthetic construct
  misc_feature 1..20: note = Synthetic Construct
  source 1..20: mol_type = other RNA
agatggtctggatggagaaa

SEQ ID NO: 372 - RNA, 20 nt, synthetic construct
  misc_feature 1..20: note = Synthetic Construct
  source 1..20: mol_type = other RNA
agcaacagggccagggctgg

SEQ ID NO: 373 - RNA, 20 nt, synthetic construct
  misc_feature 1..20: note = Synthetic Construct
  source 1..20: mol_type = other RNA
agcagtcacatgagtctgat

SEQ ID NO: 374 - RNA, 20 nt, synthetic construct
  misc_feature 1..20: note = Synthetic Construct
  source 1..20: mol_type = other RNA
agcagtcacgagtctgatct

SEQ ID NO: 375 - RNA, 20 nt, synthetic construct
  misc_feature 1..20: note = Synthetic Construct
  source 1..20: mol_type = other RNA
agccaaccatcggggggcct

SEQ ID NO: 376 - RNA, 20 nt, synthetic construct
  misc_feature 1..20: note = Synthetic Construct
  source 1..20: mol_type = other RNA
agccaaccatcggggggctt

SEQ ID NO: 377 - RNA, 20 nt, synthetic construct
  misc_feature 1..20: note = Synthetic Construct
  source 1..20: mol_type = other RNA
agcccagccccggccctgtt

SEQ ID NO: 378 - RNA, 20 nt, synthetic construct
  misc_feature 1..20: note = Synthetic Construct
  source 1..20: mol_type = other RNA
agcccagccctggccctgtt

SEQ ID NO: 379 - RNA, 20 nt, synthetic construct
  misc_feature 1..20: note = Synthetic Construct
  source 1..20: mol_type = other RNA
agccccccgatggttggctc

SEQ ID NO: 380 - RNA, 20 nt, synthetic construct
  misc_feature 1..20: note = Synthetic Construct
  source 1..20: mol_type = other RNA
agccccggccctgttgctgc

SEQ ID NO: 381 - RNA, 20 nt, synthetic construct
  misc_feature 1..20: note = Synthetic Construct
  source 1..20: mol_type = other RNA
agccctggccctgttgctgc

SEQ ID NO: 382 - RNA, 20 nt, synthetic construct
  misc_feature 1..20: note = Synthetic Construct
  source 1..20: mol_type = other RNA
agcctggggttgagggcagg

SEQ ID NO: 383 - RNA, 20 nt, synthetic construct
  misc_feature 1..20: note = Synthetic Construct
  source 1..20: mol_type = other RNA
agccttacagggatattcta

SEQ ID NO: 384 - RNA, 20 nt, synthetic construct
  misc_feature 1..20: note = Synthetic Construct
  source 1..20: mol_type = other RNA
agccttatagggatattcta

SEQ ID NO: 385 - RNA, 20 nt, synthetic construct
  misc_feature 1..20: note = Synthetic Construct
  source 1..20: mol_type = other RNA
agcggcagagactatgtgtc

SEQ ID NO: 386 - RNA, 20 nt, synthetic construct
  misc_feature 1..20: note = Synthetic Construct
  source 1..20: mol_type = other RNA
agctcgcagagactatgtgt

SEQ ID NO: 387 - RNA, 20 nt, synthetic construct
  misc_feature 1..20: note = Synthetic Construct
  source 1..20: mol_type = other RNA
agctctgtccagaaagacct

SEQ ID NO: 388 - RNA, 20 nt, synthetic construct
  misc_feature 1..20: note = Synthetic Construct
  source 1..20: mol_type = other RNA
agctctgtccagaaagactt

SEQ ID NO: 389 - RNA, 20 nt, synthetic construct
  misc_feature 1..20: note = Synthetic Construct
  source 1..20: mol_type = other RNA
agctgcaggctccgcaaggg

SEQ ID NO: 390 - RNA, 20 nt, synthetic construct
  misc_feature 1..20: note = Synthetic Construct
  source 1..20: mol_type = other RNA
agctgggactagtgcccagt

SEQ ID NO: 391 - RNA, 20 nt, synthetic construct
  misc_feature 1..20: note = Synthetic Construct
  source 1..20: mol_type = other RNA
agctgggactggtgcccagt

SEQ ID NO: 392 - RNA, 20 nt, synthetic construct
  misc_feature 1..20: note = Synthetic Construct
  source 1..20: mol_type = other RNA
aggaaggaagtctttctgga

SEQ ID NO: 393 - RNA, 20 nt, synthetic construct
  misc_feature 1..20: note = Synthetic Construct
  source 1..20: mol_type = other RNA
aggaaggaggtctttctgga

SEQ ID NO: 394 - RNA, 20 nt, synthetic construct
  misc_feature 1..20: note = Synthetic Construct
  source 1..20: mol_type = other RNA
aggaagtctttctggacaga

SEQ ID NO: 395 - RNA, 20 nt, synthetic construct
  misc_feature 1..20: note = Synthetic Construct
  source 1..20: mol_type = other RNA
aggagacagagctgggactg

SEQ ID NO: 396 - RNA, 20 nt, synthetic construct
  misc_feature 1..20: note = Synthetic Construct
  source 1..20: mol_type = other RNA
aggaggtctttctggacaga

SEQ ID NO: 397 - RNA, 20 nt, synthetic construct
  misc_feature 1..20: note = Synthetic Construct
  source 1..20: mol_type = other RNA
aggatggcagtggagatcag

SEQ ID NO: 398 - RNA, 20 nt, synthetic construct
  misc_feature 1..20: note = Synthetic Construct
  source 1..20: mol_type = other RNA
aggcccagcctggggcaagg

SEQ ID NO: 399 - RNA, 20 nt, synthetic construct
  misc_feature 1..20: note = Synthetic Construct
  source 1..20: mol_type = other RNA
aggccccccgatggttggct

SEQ ID NO: 400 - RNA, 20 nt, synthetic construct
  misc_feature 1..20: note = Synthetic Construct
  source 1..20: mol_type = other RNA
aggcccggcctggggcaagg

SEQ ID NO: 401 - RNA, 20 nt, synthetic construct
  misc_feature 1..20: note = Synthetic Construct
  source 1..20: mol_type = other RNA
aggcctctgccccctacccc

SEQ ID NO: 402 - RNA, 20 nt, synthetic construct
  misc_feature 1..20: note = Synthetic Construct
  source 1..20: mol_type = other RNA
aggcctctgccccctgcccc

SEQ ID NO: 403 - RNA, 20 nt, synthetic construct
  misc_feature 1..20: note = Synthetic Construct
  source 1..20: mol_type = other RNA
aggccttgccccaggccggg

SEQ ID NO: 404 - RNA, 20 nt, synthetic construct
  misc_feature 1..20: note = Synthetic Construct
  source 1..20: mol_type = other RNA
aggccttgccccaggctggg

SEQ ID NO: 405 - RNA, 20 nt, synthetic construct
  misc_feature 1..20: note = Synthetic Construct
  source 1..20: mol_type = other RNA
aggctgataagcccagccct

SEQ ID NO: 406 - RNA, 20 nt, synthetic construct
  misc_feature 1..20: note = Synthetic Construct
  source 1..20: mol_type = other RNA
aggctgcaggctccgcaagg

SEQ ID NO: 407 - RNA, 20 nt, synthetic construct
  misc_feature 1..20: note = Synthetic Construct
  source 1..20: mol_type = other RNA
agggactgcgaggcatcagg

SEQ ID NO: 408 - RNA, 20 nt, synthetic construct
  misc_feature 1..20: note = Synthetic Construct
  source 1..20: mol_type = other RNA
agggagaaagccccccgatg

SEQ ID NO: 409 - RNA, 20 nt, synthetic construct
  misc_feature 1..20: note = Synthetic Construct
  source 1..20: mol_type = other RNA
agggagccaaccatcggggg

SEQ ID NO: 410 - RNA, 20 nt, synthetic construct
  misc_feature 1..20: note = Synthetic Construct
  source 1..20: mol_type = other RNA
agggccagggctgggcttat

SEQ ID NO: 411 - RNA, 20 nt, synthetic construct
  misc_feature 1..20: note = Synthetic Construct
  source 1..20: mol_type = other RNA
agggccggggctgggcttat

SEQ ID NO: 412 - RNA, 20 nt, synthetic construct
  misc_feature 1..20: note = Synthetic Construct
  source 1..20: mol_type = other RNA
aggggtgagccctgggtgac

SEQ ID NO: 413 - RNA, 20 nt, synthetic construct
  misc_feature 1..20: note = Synthetic Construct
  source 1..20: mol_type = other RNA
aggtctttctggacagagct

SEQ ID NO: 414 - RNA, 20 nt, synthetic construct
  misc_feature 1..20: note = Synthetic Construct
  source 1..20: mol_type = other RNA
aggtgtcacccagggctcac

SEQ ID NO: 415 - RNA, 20 nt, synthetic construct
  misc_feature 1..20: note = Synthetic Construct
  source 1..20: mol_type = other RNA
aggttcaggccttgccccag

SEQ ID NO: 416 - RNA, 20 nt, synthetic construct
  misc_feature 1..20: note = Synthetic Construct
  source 1..20: mol_type = other RNA
agtcacatgagtctgatctc

SEQ ID NO: 417 - RNA, 20 nt, synthetic construct
  misc_feature 1..20: note = Synthetic Construct
  source 1..20: mol_type = other RNA
agtcacgagtctgatctcca

SEQ ID NO: 418 - RNA, 20 nt, synthetic construct
  misc_feature 1..20: note = Synthetic Construct
  source 1..20: mol_type = other RNA
agtccccgagccaggaaact

SEQ ID NO: 419 - RNA, 20 nt, synthetic construct
  misc_feature 1..20: note = Synthetic Construct
  source 1..20: mol_type = other RNA
agtccctgagccaggaaact

SEQ ID NO: 420 - RNA, 20 nt, synthetic construct
  misc_feature 1..20: note = Synthetic Construct
  source 1..20: mol_type = other RNA
agtctctgccgctgtctttg

SEQ ID NO: 421 - RNA, 20 nt, synthetic construct
  misc_feature 1..20: note = Synthetic Construct
  source 1..20: mol_type = other RNA
agtctctgcgagctgtcttt

SEQ ID NO: 422 - RNA, 20 nt, synthetic construct
  misc_feature 1..20: note = Synthetic Construct
  source 1..20: mol_type = other RNA
agtctttctggacagagctg

SEQ ID NO: 423 - RNA, 20 nt, synthetic construct
  misc_feature 1..20: note = Synthetic Construct
  source 1..20: mol_type = other RNA
agtgcccagttacgttggtc

SEQ ID NO: 424 - RNA, 20 nt, synthetic construct
  misc_feature 1..20: note = Synthetic Construct
  source 1..20: mol_type = other RNA
agtggagatcagactcatgt

SEQ ID NO: 425 - RNA, 20 nt, synthetic construct
  misc_feature 1..20: note = Synthetic Construct
  source 1..20: mol_type = other RNA
agtggagatcagactcgtga

SEQ ID NO: 426 - RNA, 20 nt, synthetic construct
  misc_feature 1..20: note = Synthetic Construct
  source 1..20: mol_type = other RNA
agtttcctggctcagggact

SEQ ID NO: 427 - RNA, 20 nt, synthetic construct
  misc_feature 1..20: note = Synthetic Construct
  source 1..20: mol_type = other RNA
agtttcctggctcggggact

SEQ ID NO: 428 - RNA, 20 nt, synthetic construct
  misc_feature 1..20: note = Synthetic Construct
  source 1..20: mol_type = other RNA
agtttctccatccagaccat

SEQ ID NO: 429 - RNA, 20 nt, synthetic construct
  misc_feature 1..20: note = Synthetic Construct
  source 1..20: mol_type = other RNA
ataagcccagccccggccct

SEQ ID NO: 430 - RNA, 20 nt, synthetic construct
  misc_feature 1..20: note = Synthetic Construct
  source 1..20: mol_type = other RNA
ataagcccagccctggccct

SEQ ID NO: 431 - RNA, 20 nt, synthetic construct
  misc_feature 1..20: note = Synthetic Construct
  source 1..20: mol_type = other RNA
atagggatattctaagttgt

SEQ ID NO: 432 - RNA, 20 nt, synthetic construct
  misc_feature 1..20: note = Synthetic Construct
  source 1..20: mol_type = other RNA
atagtctctgcgagctgtct

SEQ ID NO: 433 - RNA, 20 nt, synthetic construct
  misc_feature 1..20: note = Synthetic Construct
  source 1..20: mol_type = other RNA
atcagactcgtgactgctgg

SEQ ID NO: 434 - RNA, 20 nt, synthetic construct
  misc_feature 1..20: note = Synthetic Construct
  source 1..20: mol_type = other RNA
atcagactgcaaaggctgca

SEQ ID NO: 435 - RNA, 20 nt, synthetic construct
  misc_feature 1..20: note = Synthetic Construct
  source 1..20: mol_type = other RNA
atcagctctgtccagaaaga

SEQ ID NO: 436 - RNA, 20 nt, synthetic construct
  misc_feature 1..20: note = Synthetic Construct
  source 1..20: mol_type = other RNA
atcaggggtgaaccctgggt

SEQ ID NO: 437 - RNA, 20 nt, synthetic construct
  misc_feature 1..20: note = Synthetic Construct
  source 1..20: mol_type = other RNA
atcagtaacctgctgaggga

SEQ ID NO: 438 - RNA, 20 nt, synthetic construct
  misc_feature 1..20: note = Synthetic Construct
  source 1..20: mol_type = other RNA
atcagtaacctgctggggga

SEQ ID NO: 439 - RNA, 20 nt, synthetic construct
  misc_feature 1..20: note = Synthetic Construct
  source 1..20: mol_type = other RNA
atcccacaggcctctgcccc

SEQ ID NO: 440 - RNA, 20 nt, synthetic construct
  misc_feature 1..20: note = Synthetic Construct
  source 1..20: mol_type = other RNA
atcccattccagtttctcca

SEQ ID NO: 441 - RNA, 20 nt, synthetic construct
  misc_feature 1..20: note = Synthetic Construct
  source 1..20: mol_type = other RNA
atcccattccggtttctcca

SEQ ID NO: 442 - RNA, 20 nt, synthetic construct
  misc_feature 1..20: note = Synthetic Construct
  source 1..20: mol_type = other RNA
atccctataaggctggaggc

SEQ ID NO: 443 - RNA, 20 nt, synthetic construct
  misc_feature 1..20: note = Synthetic Construct
  source 1..20: mol_type = other RNA
atcggggggccttctcccta

SEQ ID NO: 444 - RNA, 20 nt, synthetic construct
  misc_feature 1..20: note = Synthetic Construct
  source 1..20: mol_type = other RNA
atcggggggctttctcccta

SEQ ID NO: 445 - RNA, 20 nt, synthetic construct
  misc_feature 1..20: note = Synthetic Construct
  source 1..20: mol_type = other RNA
atgagtctgatctccactgc

SEQ ID NO: 446 - RNA, 20 nt, synthetic construct
  misc_feature 1..20: note = Synthetic Construct
  source 1..20: mol_type = other RNA
atgcctcgcagtccccgagc

SEQ ID NO: 447 - RNA, 20 nt, synthetic construct
  misc_feature 1..20: note = Synthetic Construct
  source 1..20: mol_type = other RNA
atgcctcgcagtccctgagc

SEQ ID NO: 448 - RNA, 20 nt, synthetic construct
  misc_feature 1..20: note = Synthetic Construct
  source 1..20: mol_type = other RNA
atggagaaaccggaatggga

SEQ ID NO: 449 - RNA, 20 nt, synthetic construct
  misc_feature 1..20: note = Synthetic Construct
  source 1..20: mol_type = other RNA
atggagaaactggaatggga

SEQ ID NO: 450 - RNA, 20 nt, synthetic construct
  misc_feature 1..20: note = Synthetic Construct
  source 1..20: mol_type = other RNA
atgtgctcaaagacagctcg

SEQ ID NO: 451 - RNA, 20 nt, synthetic construct
  misc_feature 1..20: note = Synthetic Construct
  source 1..20: mol_type = other RNA
attccagtttctccatccag

SEQ ID NO: 452 - RNA, 20 nt, synthetic construct
  misc_feature 1..20: note = Synthetic Construct
  source 1..20: mol_type = other RNA
attccggtttctccatccag

SEQ ID NO: 453 - RNA, 20 nt, synthetic construct
  misc_feature 1..20: note = Synthetic Construct
  source 1..20: mol_type = other RNA
atttagggagaaagcccccc

SEQ ID NO: 454 - RNA, 20 nt, synthetic construct
  misc_feature 1..20: note = Synthetic Construct
  source 1..20: mol_type = other RNA
atttagggagaaggcccccc

SEQ ID NO: 455 - RNA, 20 nt, synthetic construct
  misc_feature 1..20: note = Synthetic Construct
  source 1..20: mol_type = other RNA
caaagacagcggcagagact

SEQ ID NO: 456 - RNA, 20 nt, synthetic construct
  misc_feature 1..20: note = Synthetic Construct
  source 1..20: mol_type = other RNA
caaagacagctcgcagagac

SEQ ID NO: 457 - RNA, 20 nt, synthetic construct
  misc_feature 1..20: note = Synthetic Construct
  source 1..20: mol_type = other RNA
caacagggccagggctgggc

SEQ ID NO: 458 - RNA, 20 nt, synthetic construct
  misc_feature 1..20: note = Synthetic Construct
  source 1..20: mol_type = other RNA
caacagggccggggctgggc

SEQ ID NO: 459 - RNA, 20 nt, synthetic construct
  misc_feature 1..20: note = Synthetic Construct
  source 1..20: mol_type = other RNA
caaccatcggggggctttct

SEQ ID NO: 460 - RNA, 20 nt, synthetic construct
  misc_feature 1..20: note = Synthetic Construct
  source 1..20: mol_type = other RNA
caacgtaactgggcaccagt

SEQ ID NO: 461 - RNA, 20 nt, synthetic construct
  misc_feature 1..20: note = Synthetic Construct
  source 1..20: mol_type = other RNA
caacgtaactgggcactagt

SEQ ID NO: 462 - RNA, 20 nt, synthetic construct
  misc_feature 1..20: note = Synthetic Construct
  source 1..20: mol_type = other RNA
cacaacttagaatatcccta

SEQ ID NO: 463 - RNA, 20 nt, synthetic construct
  misc_feature 1..20: note = Synthetic Construct
  source 1..20: mol_type = other RNA
cacaacttagaatatccctg

SEQ ID NO: 464 - RNA, 20 nt, synthetic construct
  misc_feature 1..20: note = Synthetic Construct
  source 1..20: mol_type = other RNA
cacaggcctctgccccctac

SEQ ID NO: 465 - RNA, 20 nt, synthetic construct
  misc_feature 1..20: note = Synthetic Construct
  source 1..20: mol_type = other RNA
cacaggcctctgccccctgc

SEQ ID NO: 466 - RNA, 20 nt, synthetic construct
  misc_feature 1..20: note = Synthetic Construct
  source 1..20: mol_type = other RNA
cacatagtctctgccgctgt

SEQ ID NO: 467 - RNA, 20 nt, synthetic construct
  misc_feature 1..20: note = Synthetic Construct
  source 1..20: mol_type = other RNA
cacatagtctctgcgagctg

SEQ ID NO: 468 - RNA, 20 nt, synthetic construct
  misc_feature 1..20: note = Synthetic Construct
  source 1..20: mol_type = other RNA
caccagtcccagctctgtct

SEQ ID NO: 469 - RNA, 20 nt, synthetic construct
  misc_feature 1..20: note = Synthetic Construct
  source 1..20: mol_type = other RNA
cacccagggctcacccctga

SEQ ID NO: 470 - RNA, 20 nt, synthetic construct
  misc_feature 1..20: note = Synthetic Construct
  source 1..20: mol_type = other RNA
cacccagggttcacccctga

SEQ ID NO: 471 - RNA, 20 nt, synthetic construct
  misc_feature 1..20: note = Synthetic Construct
  source 1..20: mol_type = other RNA
cacccttgcggagcctgcag

SEQ ID NO: 472 - RNA, 20 nt, synthetic construct
  misc_feature 1..20: note = Synthetic Construct
  source 1..20: mol_type = other RNA
cacggggatttagggagaag

SEQ ID NO: 473 - RNA, 20 nt, synthetic construct
  misc_feature 1..20: note = Synthetic Construct
  source 1..20: mol_type = other RNA
cactagtcccagctctgtct

SEQ ID NO: 474 - RNA, 20 nt, synthetic construct
  misc_feature 1..20: note = Synthetic Construct
  source 1..20: mol_type = other RNA
cagaaagacctccttccttt

SEQ ID NO: 475 - RNA, 20 nt, synthetic construct
  misc_feature 1..20: note = Synthetic Construct
  source 1..20: mol_type = other RNA
cagaaagacttccttccttt

SEQ ID NO: 476 - RNA, 20 nt, synthetic construct
  misc_feature 1..20: note = Synthetic Construct
  source 1..20: mol_type = other RNA
cagactcatgtgactgctgg

SEQ ID NO: 477 - RNA, 20 nt, synthetic construct
  misc_feature 1..20: note = Synthetic Construct
  source 1..20: mol_type = other RNA
cagagctgggactagtgccc

SEQ ID NO: 478 - RNA, 20 nt, synthetic construct
  misc_feature 1..20: note = Synthetic Construct
  source 1..20: mol_type = other RNA
cagagctgggactggtgccc

SEQ ID NO: 479 - RNA, 20 nt, synthetic construct
  misc_feature 1..20: note = Synthetic Construct
  source 1..20: mol_type = other RNA
cagatggtctggatggagaa

SEQ ID NO: 480 - RNA, 20 nt, synthetic construct
  misc_feature 1..20: note = Synthetic Construct
  source 1..20: mol_type = other RNA
cagcaacagggccagggctg

SEQ ID NO: 481 - RNA, 20 nt, synthetic construct
  misc_feature 1..20: note = Synthetic Construct
  source 1..20: mol_type = other RNA
cagcaacagggccggggctg

SEQ ID NO: 482 - RNA, 20 nt, synthetic construct
  misc_feature 1..20: note = Synthetic Construct
  source 1..20: mol_type = other RNA
cagcagtcacatgagtctga

SEQ ID NO: 483 - RNA, 20 nt, synthetic construct
  misc_feature 1..20: note = Synthetic Construct
  source 1..20: mol_type = other RNA
cagcagtcacgagtctgatc

SEQ ID NO: 484 - RNA, 20 nt, synthetic construct
  misc_feature 1..20: note = Synthetic Construct
  source 1..20: mol_type = other RNA
cagccccggccctgttgctg

SEQ ID NO: 485 - RNA, 20 nt, synthetic construct
  misc_feature 1..20: note = Synthetic Construct
  source 1..20: mol_type = other RNA
cagccctggccctgttgctg

SEQ ID NO: 486 - RNA, 20 nt, synthetic construct
  misc_feature 1..20: note = Synthetic Construct
  source 1..20: mol_type = other RNA
cagcctatcaggggtgaacc

SEQ ID NO: 487 - RNA, 20 nt, synthetic construct
  misc_feature 1..20: note = Synthetic Construct
  source 1..20: mol_type = other RNA
cagcctatcaggggtgagcc

SEQ ID NO: 488 - RNA, 20 nt, synthetic construct
  misc_feature 1..20: note = Synthetic Construct
  source 1..20: mol_type = other RNA
cagcctggggcaaggcctga

SEQ ID NO: 489 - RNA, 20 nt, synthetic construct
  misc_feature 1..20: note = Synthetic Construct
  source 1..20: mol_type = other RNA
cagcctggggttgagggcag

SEQ ID NO: 490 - RNA, 20 nt, synthetic construct
  misc_feature 1..20: note = Synthetic Construct
  source 1..20: mol_type = other RNA
cagccttacagggatattct

SEQ ID NO: 491 - RNA, 20 nt, synthetic construct
  misc_feature 1..20: note = Synthetic Construct
  source 1..20: mol_type = other RNA
cagccttatagggatattct

SEQ ID NO: 492 - RNA, 20 nt, synthetic construct
  misc_feature 1..20: note = Synthetic Construct
  source 1..20: mol_type = other RNA
cagcggcagagactatgtgt

SEQ ID NO: 493 - RNA, 20 nt, synthetic construct
  misc_feature 1..20: note = Synthetic Construct
  source 1..20: mol_type = other RNA
cagctcgcagagactatgtg

SEQ ID NO: 494 - RNA, 20 nt, synthetic construct
  misc_feature 1..20: note = Synthetic Construct
  source 1..20: mol_type = other RNA
cagctctgtccagaaagacc

SEQ ID NO: 495 - RNA, 20 nt, synthetic construct
  misc_feature 1..20: note = Synthetic Construct
  source 1..20: mol_type = other RNA
cagctctgtccagaaagact

SEQ ID NO: 496 - RNA, 20 nt, synthetic construct
  misc_feature 1..20: note = Synthetic Construct
  source 1..20: mol_type = other RNA
caggccgggcctctgggtac

SEQ ID NO: 497 - RNA, 20 nt, synthetic construct
  misc_feature 1..20: note = Synthetic Construct
  source 1..20: mol_type = other RNA
caggcctctgccccctaccc

SEQ ID NO: 498 - RNA, 20 nt, synthetic construct
  misc_feature 1..20: note = Synthetic Construct
  source 1..20: mol_type = other RNA
caggccttgccccaggccgg

SEQ ID NO: 499 - RNA, 20 nt, synthetic construct
  misc_feature 1..20: note = Synthetic Construct
  source 1..20: mol_type = other RNA
caggccttgccccaggctgg

SEQ ID NO: 500 - RNA, 20 nt, synthetic construct
  misc_feature 1..20: note = Synthetic Construct
  source 1..20: mol_type = other RNA
caggctgggcctctgggtac

SEQ ID NO: 501 - RNA, 20 nt, synthetic construct
  misc_feature 1..20: note = Synthetic Construct
  source 1..20: mol_type = other RNA
cagggactgcgaggcatcag

SEQ ID NO: 502 - RNA, 20 nt, synthetic construct
  misc_feature 1..20: note = Synthetic Construct
  source 1..20: mol_type = other RNA
cagggatattctaagttgtg

SEQ ID NO: 503 - RNA, 20 nt, synthetic construct
  misc_feature 1..20: note = Synthetic Construct
  source 1..20: mol_type = other RNA
cagggccggggctgggctta

SEQ ID NO: 504 - RNA, 20 nt, synthetic construct
  misc_feature 1..20: note = Synthetic Construct
  source 1..20: mol_type = other RNA
cagggctcacccctgatagg

SEQ ID NO: 505 - RNA, 20 nt, synthetic construct
  misc_feature 1..20: note = Synthetic Construct
  source 1..20: mol_type = other RNA
cagggctgggcttatcagcc

SEQ ID NO: 506 - RNA, 20 nt, synthetic construct
  misc_feature 1..20: note = Synthetic Construct
  source 1..20: mol_type = other RNA
caggggcagggggcagaggc

SEQ ID NO: 507 - RNA, 20 nt, synthetic construct
  misc_feature 1..20: note = Synthetic Construct
  source 1..20: mol_type = other RNA
cagggggcagaggcctgtgg

SEQ ID NO: 508 - RNA, 20 nt, synthetic construct
  misc_feature 1..20: note = Synthetic Construct
  source 1..20: mol_type = other RNA
caggggtagggggcagaggc

SEQ ID NO: 509 - RNA, 20 nt, synthetic construct
  misc_feature 1..20: note = Synthetic Construct
  source 1..20: mol_type = other RNA
caggggtgaaccctgggtga

SEQ ID NO: 510 - RNA, 20 nt, synthetic construct
  misc_feature 1..20: note = Synthetic Construct
  source 1..20: mol_type = other RNA
caggggtgagccctgggtga

SEQ ID NO: 511 - RNA, 20 nt, synthetic construct
  misc_feature 1..20: note = Synthetic Construct
  source 1..20: mol_type = other RNA
cagggttcacccctgatagg

SEQ ID NO: 512 - RNA, 20 nt, synthetic construct
  misc_feature 1..20: note = Synthetic Construct
  source 1..20: mol_type = other RNA
caggtacccagaggcccagc

SEQ ID NO: 513 - RNA, 20 nt, synthetic construct
  misc_feature 1..20: note = Synthetic Construct
  source 1..20: mol_type = other RNA
caggtacccagaggcccggc

SEQ ID NO: 514 - RNA, 20 nt, synthetic construct
  misc_feature 1..20: note = Synthetic Construct
  source 1..20: mol_type = other RNA
caggtcctatcagtaacctg

SEQ ID NO: 515 - RNA, 20 nt, synthetic construct
  misc_feature 1..20: note = Synthetic Construct
  source 1..20: mol_type = other RNA
caggtgtcacccagggctca

SEQ ID NO: 516 - RNA, 20 nt, synthetic construct
  misc_feature 1..20: note = Synthetic Construct
  source 1..20: mol_type = other RNA
caggtgtcacccagggttca

SEQ ID NO: 517 - RNA, 20 nt, synthetic construct
  misc_feature 1..20: note = Synthetic Construct
  source 1..20: mol_type = other RNA
cagtaacctgctgagggagg

SEQ ID NO: 518 - RNA, 20 nt, synthetic construct
  misc_feature 1..20: note = Synthetic Construct
  source 1..20: mol_type = other RNA
cagtaacctgctgggggagg

SEQ ID NO: 519 - RNA, 20 nt, synthetic construct
  misc_feature 1..20: note = Synthetic Construct
  source 1..20: mol_type = other RNA
cagtcacatgagtctgatct

SEQ ID NO: 520 - RNA, 20 nt, synthetic construct
  misc_feature 1..20: note = Synthetic Construct
  source 1..20: mol_type = other RNA
cagtcacgagtctgatctcc

SEQ ID NO: 521 - RNA, 20 nt, synthetic construct
  misc_feature 1..20: note = Synthetic Construct
  source 1..20: mol_type = other RNA
cagtcccagctctgtctcct

SEQ ID NO: 522 - RNA, 20 nt, synthetic construct
  misc_feature 1..20: note = Synthetic Construct
  source 1..20: mol_type = other RNA
cagtggagatcagactcgtg

SEQ ID NO: 523 - RNA, 20 nt, synthetic construct
  misc_feature 1..20: note = Synthetic Construct
  source 1..20: mol_type = other RNA
cagtttctccatccagacca

SEQ ID NO: 524 - RNA, 20 nt, synthetic construct
  misc_feature 1..20: note = Synthetic Construct
  source 1..20: mol_type = other RNA
catagtctctgcgagctgtc

SEQ ID NO: 525 - RNA, 20 nt, synthetic construct
  misc_feature 1..20: note = Synthetic Construct
  source 1..20: mol_type = other RNA
catcagctctgtccagaaag

SEQ ID NO: 526 - RNA, 20 nt, synthetic construct
  misc_feature 1..20: note = Synthetic Construct
  source 1..20: mol_type = other RNA
catcccacaggcctctgccc

SEQ ID NO: 527 - RNA, 20 nt, synthetic construct
  misc_feature 1..20: note = Synthetic Construct
  source 1..20: mol_type = other RNA
catcggggggccttctccct

SEQ ID NO: 528 - RNA, 20 nt, synthetic construct
  misc_feature 1..20: note = Synthetic Construct
  source 1..20: mol_type = other RNA
catcggggggctttctccct

SEQ ID NO: 529 - RNA, 20 nt, synthetic construct
  misc_feature 1..20: note = Synthetic Construct
  source 1..20: mol_type = other RNA
catgagtctgatctccactg

SEQ ID NO: 530 - RNA, 20 nt, synthetic construct
  misc_feature 1..20: note = Synthetic Construct
  source 1..20: mol_type = other RNA
catggcccccttccagcagt

SEQ ID NO: 531 - RNA, 20 nt, synthetic construct
  misc_feature 1..20: note = Synthetic Construct
  source 1..20: mol_type = other RNA
catgtgactgctggaagggg

SEQ ID NO: 532 - RNA, 20 nt, synthetic construct
  misc_feature 1..20: note = Synthetic Construct
  source 1..20: mol_type = other RNA
ccaaccatcggggggccttc

SEQ ID NO: 533 - RNA, 20 nt, synthetic construct
  misc_feature 1..20: note = Synthetic Construct
  source 1..20: mol_type = other RNA
ccaaccatcggggggctttc

SEQ ID NO: 534 - RNA, 20 nt, synthetic construct
  misc_feature 1..20: note = Synthetic Construct
  source 1..20: mol_type = other RNA
ccaacgtaactgggcaccag

SEQ ID NO: 535 - RNA, 20 nt, synthetic construct
  misc_feature 1..20: note = Synthetic Construct
  source 1..20: mol_type = other RNA
ccaacgtaactgggcactag

SEQ ID NO: 536 - RNA, 20 nt, synthetic construct
  misc_feature 1..20: note = Synthetic Construct
  source 1..20: mol_type = other RNA
ccacaggcctctgccccctg

SEQ ID NO: 537 - RNA, 20 nt, synthetic construct
  misc_feature 1..20: note = Synthetic Construct
  source 1..20: mol_type = other RNA
ccacggggatttagggagaa

SEQ ID NO: 538 - RNA, 20 nt, synthetic construct
  misc_feature 1..20: note = Synthetic Construct
  source 1..20: mol_type = other RNA
ccagaaagacctccttcctt

SEQ ID NO: 539 - RNA, 20 nt, synthetic construct
  misc_feature 1..20: note = Synthetic Construct
  source 1..20: mol_type = other RNA
ccagaaagacttccttcctt

SEQ ID NO: 540 - RNA, 20 nt, synthetic construct
  misc_feature 1..20: note = Synthetic Construct
  source 1..20: mol_type = other RNA
ccagacctcccccagcaggt

SEQ ID NO: 541 - RNA, 20 nt, synthetic construct
  misc_feature 1..20: note = Synthetic Construct
  source 1..20: mol_type = other RNA
ccagacctccctcagcaggt

SEQ ID NO: 542 - RNA, 20 nt, synthetic construct
  misc_feature 1..20: note = Synthetic Construct
  source 1..20: mol_type = other RNA
ccagaggcccagcctggggc

SEQ ID NO: 543 - RNA, 20 nt, synthetic construct
  misc_feature 1..20: note = Synthetic Construct
  source 1..20: mol_type = other RNA
ccagaggcccggcctggggc

SEQ ID NO: 544 - RNA, 20 nt, synthetic construct
  misc_feature 1..20: note = Synthetic Construct
  source 1..20: mol_type = other RNA
ccagcaggttactgatagga

SEQ ID NO: 545 - RNA, 20 nt, synthetic construct
  misc_feature 1..20: note = Synthetic Construct
  source 1..20: mol_type = other RNA
ccagcagtcacatgagtctg

SEQ ID NO: 546 - RNA, 20 nt, synthetic construct
  misc_feature 1..20: note = Synthetic Construct
  source 1..20: mol_type = other RNA
ccagccccggccctgttgct

SEQ ID NO: 547 - RNA, 20 nt, synthetic construct
  misc_feature 1..20: note = Synthetic Construct
  source 1..20: mol_type = other RNA
ccagccctggccctgttgct

SEQ ID NO: 548 - RNA, 20 nt, synthetic construct
  misc_feature 1..20: note = Synthetic Construct
  source 1..20: mol_type = other RNA
ccagcctatcaggggtgaac

SEQ ID NO: 549 - RNA, 20 nt, synthetic construct
  misc_feature 1..20: note = Synthetic Construct
  source 1..20: mol_type = other RNA
ccagcctatcaggggtgagc

SEQ ID NO: 550 - RNA, 20 nt, synthetic construct
  misc_feature 1..20: note = Synthetic Construct
  source 1..20: mol_type = other RNA
ccagcctggggcaaggcctg

SEQ ID NO: 551 - RNA, 20 nt, synthetic construct
  misc_feature 1..20: note = Synthetic Construct
  source 1..20: mol_type = other RNA
ccagccttatagggatattc

SEQ ID NO: 552 - RNA, 20 nt, synthetic construct
  misc_feature 1..20: note = Synthetic Construct
  source 1..20: mol_type = other RNA
ccaggccgggcctctgggta

SEQ ID NO: 553 - RNA, 20 nt, synthetic construct
  misc_feature 1..20: note = Synthetic Construct
  source 1..20: mol_type = other RNA
ccaggctgggcctctgggta

SEQ ID NO: 554 - RNA, 20 nt, synthetic construct
  misc_feature 1..20: note = Synthetic Construct
  source 1..20: mol_type = other RNA
ccagggctcacccctgatag

SEQ ID NO: 555 - RNA, 20 nt, synthetic construct
  misc_feature 1..20: note = Synthetic Construct
  source 1..20: mol_type = other RNA
ccagggctgggcttatcagc

SEQ ID NO: 556 - RNA, 20 nt, synthetic construct
  misc_feature 1..20: note = Synthetic Construct
  source 1..20: mol_type = other RNA
ccagggttcacccctgatag

SEQ ID NO: 557 - RNA, 20 nt, synthetic construct
  misc_feature 1..20: note = Synthetic Construct
  source 1..20: mol_type = other RNA
ccagtcccagctctgtctcc

SEQ ID NO: 558 - RNA, 20 nt, synthetic construct
  misc_feature 1..20: note = Synthetic Construct
  source 1..20: mol_type = other RNA
ccagtttctccatccagacc

SEQ ID NO: 559 - RNA, 20 nt, synthetic construct
  misc_feature 1..20: note = Synthetic Construct
  source 1..20: mol_type = other RNA
ccatcagctctgtccagaaa

SEQ ID NO: 560 - RNA, 20 nt, synthetic construct
  misc_feature 1..20: note = Synthetic Construct
  source 1..20: mol_type = other RNA
ccatcggggggccttctccc

SEQ ID NO: 561 - RNA, 20 nt, synthetic construct
  misc_feature 1..20: note = Synthetic Construct
  source 1..20: mol_type = other RNA
cccacaggcctctgccccct

SEQ ID NO: 562 - RNA, 20 nt, synthetic construct
  misc_feature 1..20: note = Synthetic Construct
  source 1..20: mol_type = other RNA
cccagacctccctcagcagg

SEQ ID NO: 563 - RNA, 20 nt, synthetic construct
  misc_feature 1..20: note = Synthetic Construct
  source 1..20: mol_type = other RNA
cccagcaggttactgatagg

SEQ ID NO: 564 - RNA, 20 nt, synthetic construct
  misc_feature 1..20: note = Synthetic Construct
  source 1..20: mol_type = other RNA
cccagccccggccctgttgc

SEQ ID NO: 565 - RNA, 20 nt, synthetic construct
  misc_feature 1..20: note = Synthetic Construct
  source 1..20: mol_type = other RNA
cccagcctatcaggggtgaa

SEQ ID NO: 566 - RNA, 20 nt, synthetic construct
  misc_feature 1..20: note = Synthetic Construct
  source 1..20: mol_type = other RNA
cccagcctggggcaaggcct

SEQ ID NO: 567 - RNA, 20 nt, synthetic construct
  misc_feature 1..20: note = Synthetic Construct
  source 1..20: mol_type = other RNA
cccaggccgggcctctgggt

SEQ ID NO: 568 - RNA, 20 nt, synthetic construct
  misc_feature 1..20: note = Synthetic Construct
  source 1..20: mol_type = other RNA
cccagggctcacccctgata

SEQ ID NO: 569 - RNA, 20 nt, synthetic construct
  misc_feature 1..20: note = Synthetic Construct
  source 1..20: mol_type = other RNA
cccattccagtttctccatc

SEQ ID NO: 570 - RNA, 20 nt, synthetic construct
  misc_feature 1..20: note = Synthetic Construct
  source 1..20: mol_type = other RNA
ccccagcaggttactgatag

SEQ ID NO: 571 - RNA, 20 nt, synthetic construct
  misc_feature 1..20: note = Synthetic Construct
  source 1..20: mol_type = other RNA
ccccagcctatcaggggtga

SEQ ID NO: 572 - RNA, 20 nt, synthetic construct
  misc_feature 1..20: note = Synthetic Construct
  source 1..20: mol_type = other RNA
ccccaggccgggcctctggg

SEQ ID NO: 573 - RNA, 20 nt, synthetic construct
  misc_feature 1..20: note = Synthetic Construct
  source 1..20: mol_type = other RNA
ccccaggctgggcctctggg

SEQ ID NO: 574 - RNA, 20 nt, synthetic construct
  misc_feature 1..20: note = Synthetic Construct
  source 1..20: mol_type = other RNA
ccccctacccctgccctcaa

SEQ ID NO: 575 - RNA, 20 nt, synthetic construct
  misc_feature 1..20: note = Synthetic Construct
  source 1..20: mol_type = other RNA
ccccctgcccctgccctcaa

SEQ ID NO: 576 - RNA, 20 nt, synthetic construct
  misc_feature 1..20: note = Synthetic Construct
  source 1..20: mol_type = other RNA
cccccttccagcagtcacat

SEQ ID NO: 577 - RNA, 20 nt, synthetic construct
  misc_feature 1..20: note = Synthetic Construct
  source 1..20: mol_type = other RNA
cccccttccagcagtcacga

SEQ ID NO: 578 - RNA, 20 nt, synthetic construct
  misc_feature 1..20: note = Synthetic Construct
  source 1..20: mol_type = other RNA
ccccgagccaggaaactgag

SEQ ID NO: 579 - RNA, 20 nt, synthetic construct
  misc_feature 1..20: note = Synthetic Construct
  source 1..20: mol_type = other RNA
cccctacccctgccctcaac

SEQ ID NO: 580 - RNA, 20 nt, synthetic construct
  misc_feature 1..20: note = Synthetic Construct
  source 1..20: mol_type = other RNA
cccctgcccctgccctcaac

SEQ ID NO: 581 - RNA, 20 nt, synthetic construct
  misc_feature 1..20: note = Synthetic Construct
  source 1..20: mol_type = other RNA
ccccttccagcagtcacatg

SEQ ID NO: 582 - RNA, 20 nt, synthetic construct
  misc_feature 1..20: note = Synthetic Construct
  source 1..20: mol_type = other RNA
cccgagccaggaaactgaga

SEQ ID NO: 583 - RNA, 20 nt, synthetic construct
  misc_feature 1..20: note = Synthetic Construct
  source 1..20: mol_type = other RNA
cccggccctgttgctgctca

SEQ ID NO: 584 - RNA, 20 nt, synthetic construct
  misc_feature 1..20: note = Synthetic Construct
  source 1..20: mol_type = other RNA
ccctacccctgccctcaacc

SEQ ID NO: 585 - RNA, 20 nt, synthetic construct
  misc_feature 1..20: note = Synthetic Construct
  source 1..20: mol_type = other RNA
ccctataaggctggaggcca

SEQ ID NO: 586 - RNA, 20 nt, synthetic construct
  misc_feature 1..20: note = Synthetic Construct
  source 1..20: mol_type = other RNA
ccctgagccaggaaactgag

SEQ ID NO: 587 - RNA, 20 nt, synthetic construct
  misc_feature 1..20: note = Synthetic Construct
  source 1..20: mol_type = other RNA
ccctgatcagactgcaaagg

SEQ ID NO: 588 - RNA, 20 nt, synthetic construct
  misc_feature 1..20: note = Synthetic Construct
  source 1..20: mol_type = other RNA
ccctgcccctgccctcaacc

SEQ ID NO: 589 - RNA, 20 nt, synthetic construct
  misc_feature 1..20: note = Synthetic Construct
  source 1..20: mol_type = other RNA
ccctgcctggagatcccatt

SEQ ID NO: 590 - RNA, 20 nt, synthetic construct
  misc_feature 1..20: note = Synthetic Construct
  source 1..20: mol_type = other RNA
ccctgtaaggctggaggcca

SEQ ID NO: 591 - RNA, 20 nt, synthetic construct
  misc_feature 1..20: note = Synthetic Construct
  source 1..20: mol_type = other RNA
cccttccagcagtcacatga

SEQ ID NO: 592 - RNA, 20 nt, synthetic construct
  misc_feature 1..20: note = Synthetic Construct
  source 1..20: mol_type = other RNA
cccttccagcagtcacgagt

SEQ ID NO: 593 - RNA, 20 nt, synthetic construct
  misc_feature 1..20: note = Synthetic Construct
  source 1..20: mol_type = other RNA
cccttgcggagcctgcagcc

SEQ ID NO: 594 - RNA, 20 nt, synthetic construct
  misc_feature 1..20: note = Synthetic Construct
  source 1..20: mol_type = other RNA
ccgagccaggaaactgagaa

SEQ ID NO: 595 - RNA, 20 nt, synthetic construct
  misc_feature 1..20: note = Synthetic Construct
  source 1..20: mol_type = other RNA
ccgcggacaggtgtcaccca

SEQ ID NO: 596 - RNA, 20 nt, synthetic construct
  misc_feature 1..20: note = Synthetic Construct
  source 1..20: mol_type = other RNA
ccgctgtctttgagcacatc

SEQ ID NO: 597 - RNA, 20 nt, synthetic construct
  misc_feature 1..20: note = Synthetic Construct
  source 1..20: mol_type = other RNA
ccggcctggggcaaggcctg

SEQ ID NO: 598 - RNA, 20 nt, synthetic construct
  misc_feature 1..20: note = Synthetic Construct
  source 1..20: mol_type = other RNA
ccgggcctctgggtacctga

SEQ ID NO: 599 - RNA, 20 nt, synthetic construct
  misc_feature 1..20: note = Synthetic Construct
  source 1..20: mol_type = other RNA
ccggggctgggcttatcagc

SEQ ID NO: 600 - RNA, 20 nt, synthetic construct
  misc_feature 1..20: note = Synthetic Construct
  source 1..20: mol_type = other RNA
ccggtttctccatccagacc

SEQ ID NO: 601 - RNA, 20 nt, synthetic construct
  misc_feature 1..20: note = Synthetic Construct
  source 1..20: mol_type = other RNA
cctataaggctggaggccag

SEQ ID NO: 602 - RNA, 20 nt, synthetic construct
  misc_feature 1..20: note = Synthetic Construct
  source 1..20: mol_type = other RNA
cctcagcaggttactgatag

SEQ ID NO: 603 - RNA, 20 nt, synthetic construct
  misc_feature 1..20: note = Synthetic Construct
  source 1..20: mol_type = other RNA
cctcaggtacccagaggccc

SEQ ID NO: 604 - RNA, 20 nt, synthetic construct
  misc_feature 1..20: note = Synthetic Construct
  source 1..20: mol_type = other RNA
cctccagccttacagggata

SEQ ID NO: 605 - RNA, 20 nt, synthetic construct
  misc_feature 1..20: note = Synthetic Construct
  source 1..20: mol_type = other RNA
cctccagccttatagggata

SEQ ID NO: 606 - RNA, 20 nt, synthetic construct
  misc_feature 1..20: note = Synthetic Construct
  source 1..20: mol_type = other RNA
cctcccccagcaggttactg

SEQ ID NO: 607 - RNA, 20 nt, synthetic construct
  misc_feature 1..20: note = Synthetic Construct
  source 1..20: mol_type = other RNA
cctccctcagcaggttactg

SEQ ID NO: 608 - RNA, 20 nt, synthetic construct
  misc_feature 1..20: note = Synthetic Construct
  source 1..20: mol_type = other RNA
cctcgcagtccctgagccag

SEQ ID NO: 609 - RNA, 20 nt, synthetic construct
  misc_feature 1..20: note = Synthetic Construct
  source 1..20: mol_type = other RNA
cctctgccccctacccctgc

SEQ ID NO: 610 - RNA, 20 nt, synthetic construct
  misc_feature 1..20: note = Synthetic Construct
  source 1..20: mol_type = other RNA
cctctgccccctgcccctgc

SEQ ID NO: 611 - RNA, 20 nt, synthetic construct
  misc_feature 1..20: note = Synthetic Construct
  source 1..20: mol_type = other RNA
cctgagccaggaaactgaga

SEQ ID NO: 612 - RNA, 20 nt, synthetic construct
  misc_feature 1..20: note = Synthetic Construct
  source 1..20: mol_type = other RNA
cctgatcagactgcaaaggc

SEQ ID NO: 613 - RNA, 20 nt, synthetic construct
  misc_feature 1..20: note = Synthetic Construct
  source 1..20: mol_type = other RNA
cctgatgcctcgcagtcccc

SEQ ID NO: 614 - RNA, 20 nt, synthetic construct
  misc_feature 1..20: note = Synthetic Construct
  source 1..20: mol_type = other RNA
cctgatgcctcgcagtccct

SEQ ID NO: 615 - RNA, 20 nt, synthetic construct
  misc_feature 1..20: note = Synthetic Construct
  source 1..20: mol_type = other RNA
cctgcagcctttgcagtctg

SEQ ID NO: 616 - RNA, 20 nt, synthetic construct
  misc_feature 1..20: note = Synthetic Construct
  source 1..20: mol_type = other RNA
cctgcccctgccctcaaccc

SEQ ID NO: 617 - RNA, 20 nt, synthetic construct
  misc_feature 1..20: note = Synthetic Construct
  source 1..20: mol_type = other RNA
cctgctgagggaggtctggg

SEQ ID NO: 618 - RNA, 20 nt, synthetic construct
  misc_feature 1..20: note = Synthetic Construct
  source 1..20: mol_type = other RNA
cctgctgggggaggtctggg

SEQ ID NO: 619 - RNA, 20 nt, synthetic construct
  misc_feature 1..20: note = Synthetic Construct
  source 1..20: mol_type = other RNA
cctggagatcccattccagt

SEQ ID NO: 620 - RNA, 20 nt, synthetic construct
  misc_feature 1..20: note = Synthetic Construct
  source 1..20: mol_type = other RNA
cctggagatcccattccggt

SEQ ID NO: 621 - RNA, 20 nt, synthetic construct
  misc_feature 1..20: note = Synthetic Construct
  source 1..20: mol_type = other RNA
cctggccctgttgctgctca

SEQ ID NO: 622 - RNA, 20 nt, synthetic construct
  misc_feature 1..20: note = Synthetic Construct
  source 1..20: mol_type = other RNA
cctggctcagggactgcgag

SEQ ID NO: 623 - RNA, 20 nt, synthetic construct
  misc_feature 1..20: note = Synthetic Construct
  source 1..20: mol_type = other RNA
cctggctcggggactgcgag

SEQ ID NO: 624 - RNA, 20 nt, synthetic construct
  misc_feature 1..20: note = Synthetic Construct
  source 1..20: mol_type = other RNA
cctggggttgagggcagggg

SEQ ID NO: 625 - RNA, 20 nt, synthetic construct
  misc_feature 1..20: note = Synthetic Construct
  source 1..20: mol_type = other RNA
cctgtaaggctggaggccag

SEQ ID NO: 626 - RNA, 20 nt, synthetic construct
  misc_feature 1..20: note = Synthetic Construct
  source 1..20: mol_type = other RNA
ccttacagggatattctaag

SEQ ID NO: 627 - RNA, 20 nt, synthetic construct
  misc_feature 1..20: note = Synthetic Construct
  source 1..20: mol_type = other RNA
ccttatagggatattctaag

SEQ ID NO: 628 - RNA, 20 nt, synthetic construct
  misc_feature 1..20: note = Synthetic Construct
  source 1..20: mol_type = other RNA
ccttctccctaaatccccgt

SEQ ID NO: 629 - RNA, 20 nt, synthetic construct
  misc_feature 1..20: note = Synthetic Construct
  source 1..20: mol_type = other RNA
ccttgcggagcctgcagcct

SEQ ID NO: 630 - RNA, 20 nt, synthetic construct
  misc_feature 1..20: note = Synthetic Construct
  source 1..20: mol_type = other RNA
cctttgcagtctgatcaggg

SEQ ID NO: 631 - RNA, 20 nt, synthetic construct
  misc_feature 1..20: note = Synthetic Construct
  source 1..20: mol_type = other RNA
cgagccaggaaactgagaag

SEQ ID NO: 632 - RNA, 20 nt, synthetic construct
  misc_feature 1..20: note = Synthetic Construct
  source 1..20: mol_type = other RNA
cgagctgtctttgagcacat

SEQ ID NO: 633 - RNA, 20 nt, synthetic construct
  misc_feature 1..20: note = Synthetic Construct
  source 1..20: mol_type = other RNA
cgagtctgatctccactgcc

SEQ ID NO: 634 - RNA, 20 nt, synthetic construct
  misc_feature 1..20: note = Synthetic Construct
  source 1..20: mol_type = other RNA
cgcagagactatgtgtccca

SEQ ID NO: 635 - RNA, 20 nt, synthetic construct
  misc_feature 1..20: note = Synthetic Construct
  source 1..20: mol_type = other RNA
cgcagtccccgagccaggaa

SEQ ID NO: 636 - RNA, 20 nt, synthetic construct
  misc_feature 1..20: note = Synthetic Construct
  source 1..20: mol_type = other RNA
cgcagtccctgagccaggaa

SEQ ID NO: 637 - RNA, 20 nt, synthetic construct
  misc_feature 1..20: note = Synthetic Construct
  source 1..20: mol_type = other RNA
cgccccagcctatcaggggt

SEQ ID NO: 638 - RNA, 20 nt, synthetic construct
  misc_feature 1..20: note = Synthetic Construct
  source 1..20: mol_type = other RNA
cgcggacaggtgtcacccag

SEQ ID NO: 639 - RNA, 20 nt, synthetic construct
  misc_feature 1..20: note = Synthetic Construct
  source 1..20: mol_type = other RNA
cgctgtctttgagcacatcc

SEQ ID NO: 640 - RNA, 20 nt, synthetic construct
  misc_feature 1..20: note = Synthetic Construct
  source 1..20: mol_type = other RNA
cggacaggtgtcacccaggg

SEQ ID NO: 641 - RNA, 20 nt, synthetic construct
  misc_feature 1..20: note = Synthetic Construct
  source 1..20: mol_type = other RNA
cggagcctgcagcctttgca

SEQ ID NO: 642 - RNA, 20 nt, synthetic construct
  misc_feature 1..20: note = Synthetic Construct
  source 1..20: mol_type = other RNA
cggcagagactatgtgtccc

SEQ ID NO: 643 - RNA, 20 nt, synthetic construct
  misc_feature 1..20: note = Synthetic Construct
  source 1..20: mol_type = other RNA
cggccctgttgctgctcact

SEQ ID NO: 644 - RNA, 20 nt, synthetic construct
  misc_feature 1..20: note = Synthetic Construct
  source 1..20: mol_type = other RNA
cggcctggggcaaggcctga

SEQ ID NO: 645 - RNA, 20 nt, synthetic construct
  misc_feature 1..20: note = Synthetic Construct
  source 1..20: mol_type = other RNA
cgggcctctgggtacctgag

SEQ ID NO: 646 - RNA, 20 nt, synthetic construct
  misc_feature 1..20: note = Synthetic Construct
  source 1..20: mol_type = other RNA
cggggactgcgaggcatcag

SEQ ID NO: 647 - RNA, 20 nt, synthetic construct
  misc_feature 1..20: note = Synthetic Construct
  source 1..20: mol_type = other RNA
cggggatttagggagaaagc

SEQ ID NO: 648 - RNA, 20 nt, synthetic construct
  misc_feature 1..20: note = Synthetic Construct
  source 1..20: mol_type = other RNA
cggggatttagggagaaggc

SEQ ID NO: 649 - RNA, 20 nt, synthetic construct
  misc_feature 1..20: note = Synthetic Construct
  source 1..20: mol_type = other RNA
cggggctgggcttatcagcc

SEQ ID NO: 650 - RNA, 20 nt, synthetic construct
  misc_feature 1..20: note = Synthetic Construct
  source 1..20: mol_type = other RNA
cggggggccttctccctaaa

SEQ ID NO: 651 - RNA, 20 nt, synthetic construct
  misc_feature 1..20: note = Synthetic Construct
  source 1..20: mol_type = other RNA
cggggggctttctccctaaa

SEQ ID NO: 652 - RNA, 20 nt, synthetic construct
  misc_feature 1..20: note = Synthetic Construct
  source 1..20: mol_type = other RNA
cggtttctccatccagacca

SEQ ID NO: 653 - RNA, 20 nt, synthetic construct
  misc_feature 1..20: note = Synthetic Construct
  source 1..20: mol_type = other RNA
cgtaactgggcaccagtccc

SEQ ID NO: 654 - RNA, 20 nt, synthetic construct
  misc_feature 1..20: note = Synthetic Construct
  source 1..20: mol_type = other RNA
cgtaactgggcactagtccc

SEQ ID NO: 655 - RNA, 20 nt, synthetic construct
  misc_feature 1..20: note = Synthetic Construct
  source 1..20: mol_type = other RNA
cgtgactgctggaagggggc

SEQ ID NO: 656 - RNA, 20 nt, synthetic construct
  misc_feature 1..20: note = Synthetic Construct
  source 1..20: mol_type = other RNA
cgtggatgtgctcaaagaca

SEQ ID NO: 657 - RNA, 20 nt, synthetic construct
  misc_feature 1..20: note = Synthetic Construct
  source 1..20: mol_type = other RNA
ctagtcccagctctgtctcc

SEQ ID NO: 658 - RNA, 20 nt, synthetic construct
  misc_feature 1..20: note = Synthetic Construct
  source 1..20: mol_type = other RNA
ctagtgcccagttacgttgg

SEQ ID NO: 659 - RNA, 20 nt, synthetic construct
  misc_feature 1..20: note = Synthetic Construct
  source 1..20: mol_type = other RNA
ctatcaggggtgaaccctgg

SEQ ID NO: 660 - RNA, 20 nt, synthetic construct
  misc_feature 1..20: note = Synthetic Construct
  source 1..20: mol_type = other RNA
ctatcaggggtgagccctgg

SEQ ID NO: 661 - RNA, 20 nt, synthetic construct
  misc_feature 1..20: note = Synthetic Construct
  source 1..20: mol_type = other RNA
ctatcagtaacctgctgagg

SEQ ID NO: 662 - RNA, 20 nt, synthetic construct
  misc_feature 1..20: note = Synthetic Construct
  source 1..20: mol_type = other RNA
ctatcagtaacctgctgggg

SEQ ID NO: 663 - RNA, 20 nt, synthetic construct
  misc_feature 1..20: note = Synthetic Construct
  source 1..20: mol_type = other RNA
ctatcctgatgcctcgcagt

SEQ ID NO: 664 - RNA, 20 nt, synthetic construct
  misc_feature 1..20: note = Synthetic Construct
  source 1..20: mol_type = other RNA
ctcaaagacagcggcagaga

SEQ ID NO: 665 - RNA, 20 nt, synthetic construct
  misc_feature 1..20: note = Synthetic Construct
  source 1..20: mol_type = other RNA
ctcaaagacagctcgcagag

SEQ ID NO: 666 - RNA, 20 nt, synthetic construct
  misc_feature 1..20: note = Synthetic Construct
  source 1..20: mol_type = other RNA
ctcacccctgataggctggg

SEQ ID NO: 667 - RNA, 20 nt, synthetic construct
  misc_feature 1..20: note = Synthetic Construct
  source 1..20: mol_type = other RNA
ctcaggtacccagaggccca

SEQ ID NO: 668 - RNA, 20 nt, synthetic construct
  misc_feature 1..20: note = Synthetic Construct
  source 1..20: mol_type = other RNA
ctcagtttcctggctcaggg

SEQ ID NO: 669 - RNA, 20 nt, synthetic construct
  misc_feature 1..20: note = Synthetic Construct
  source 1..20: mol_type = other RNA
ctcagtttcctggctcgggg

SEQ ID NO: 670 - RNA, 20 nt, synthetic construct
  misc_feature 1..20: note = Synthetic Construct
  source 1..20: mol_type = other RNA
ctccagccttacagggatat

SEQ ID NO: 671 - RNA, 20 nt, synthetic construct
  misc_feature 1..20: note = Synthetic Construct
  source 1..20: mol_type = other RNA
ctccagccttatagggatat

SEQ ID NO: 672 - RNA, 20 nt, synthetic construct
  misc_feature 1..20: note = Synthetic Construct
  source 1..20: mol_type = other RNA
ctcctggaaggcccagacct

SEQ ID NO: 673 - RNA, 20 nt, synthetic construct
  misc_feature 1..20: note = Synthetic Construct
  source 1..20: mol_type = other RNA
ctcgcagagactatgtgtcc

SEQ ID NO: 674 - RNA, 20 nt, synthetic construct
  misc_feature 1..20: note = Synthetic Construct
  source 1..20: mol_type = other RNA
ctcgcagtccccgagccagg

SEQ ID NO: 675 - RNA, 20 nt, synthetic construct
  misc_feature 1..20: note = Synthetic Construct
  source 1..20: mol_type = other RNA
ctcgcagtccctgagccagg

SEQ ID NO: 676 - RNA, 20 nt, synthetic construct
  misc_feature 1..20: note = Synthetic Construct
  source 1..20: mol_type = other RNA
ctcgtgactgctggaagggg

SEQ ID NO: 677 - RNA, 20 nt, synthetic construct
  misc_feature 1..20: note = Synthetic Construct
  source 1..20: mol_type = other RNA
ctctgccgctgtctttgagc

SEQ ID NO: 678 - RNA, 20 nt, synthetic construct
  misc_feature 1..20: note = Synthetic Construct
  source 1..20: mol_type = other RNA
ctctgcgagctgtctttgag

SEQ ID NO: 679 - RNA, 20 nt, synthetic construct
  misc_feature 1..20: note = Synthetic Construct
  source 1..20: mol_type = other RNA
ctctgtccagaaagacctcc

SEQ ID NO: 680 - RNA, 20 nt, synthetic construct
  misc_feature 1..20: note = Synthetic Construct
  source 1..20: mol_type = other RNA
ctctgtccagaaagacttcc

SEQ ID NO: 681 - RNA, 20 nt, synthetic construct
  misc_feature 1..20: note = Synthetic Construct
  source 1..20: mol_type = other RNA
ctgagccaggaaactgagaa

SEQ ID NO: 682 - RNA, 20 nt, synthetic construct
  misc_feature 1..20: note = Synthetic Construct
  source 1..20: mol_type = other RNA
ctgagggaggtctgggcctt

SEQ ID NO: 683 - RNA, 20 nt, synthetic construct
  misc_feature 1..20: note = Synthetic Construct
  source 1..20: mol_type = other RNA
ctgataagcccagccccggc

SEQ ID NO: 684 - RNA, 20 nt, synthetic construct
  misc_feature 1..20: note = Synthetic Construct
  source 1..20: mol_type = other RNA
ctgatcagactgcaaaggct

SEQ ID NO: 685 - RNA, 20 nt, synthetic construct
  misc_feature 1..20: note = Synthetic Construct
  source 1..20: mol_type = other RNA
ctgatgcctcgcagtccccg

SEQ ID NO: 686 - RNA, 20 nt, synthetic construct
  misc_feature 1..20: note = Synthetic Construct
  source 1..20: mol_type = other RNA
ctgatgcctcgcagtccctg

SEQ ID NO: 687 - RNA, 20 nt, synthetic construct
  misc_feature 1..20: note = Synthetic Construct
  source 1..20: mol_type = other RNA
ctgcaaaggctgcaggctcc

SEQ ID NO: 688 - RNA, 20 nt, synthetic construct
  misc_feature 1..20: note = Synthetic Construct
  source 1..20: mol_type = other RNA
ctgcagcctttgcagtctga

SEQ ID NO: 689 - RNA, 20 nt, synthetic construct
  misc_feature 1..20: note = Synthetic Construct
  source 1..20: mol_type = other RNA
ctgccccctacccctgccct

SEQ ID NO: 690 - RNA, 20 nt, synthetic construct
  misc_feature 1..20: note = Synthetic Construct
  source 1..20: mol_type = other RNA
ctgccccctgcccctgccct

SEQ ID NO: 691 - RNA, 20 nt, synthetic construct
  misc_feature 1..20: note = Synthetic Construct
  source 1..20: mol_type = other RNA
ctgccgctgtctttgagcac

SEQ ID NO: 692 - RNA, 20 nt, synthetic construct
  misc_feature 1..20: note = Synthetic Construct
  source 1..20: mol_type = other RNA
ctgcctggagatcccattcc

SEQ ID NO: 693 - RNA, 20 nt, synthetic construct
  misc_feature 1..20: note = Synthetic Construct
  source 1..20: mol_type = other RNA
ctgcgagctgtctttgagca

SEQ ID NO: 694 - RNA, 20 nt, synthetic construct
  misc_feature 1..20: note = Synthetic Construct
  source 1..20: mol_type = other RNA
ctgctgggggaggtctgggc

SEQ ID NO: 695 - RNA, 20 nt, synthetic construct
  misc_feature 1..20: note = Synthetic Construct
  source 1..20: mol_type = other RNA
ctggaaggcccagacctccc

SEQ ID NO: 696 - RNA, 20 nt, synthetic construct
  misc_feature 1..20: note = Synthetic Construct
  source 1..20: mol_type = other RNA
ctggagatcccattccagtt

SEQ ID NO: 697 - RNA, 20 nt, synthetic construct
  misc_feature 1..20: note = Synthetic Construct
  source 1..20: mol_type = other RNA
ctggcctccagccttacagg

SEQ ID NO: 698 - RNA, 20 nt, synthetic construct
  misc_feature 1..20: note = Synthetic Construct
  source 1..20: mol_type = other RNA
ctggcctccagccttatagg

SEQ ID NO: 699 - RNA, 20 nt, synthetic construct
  misc_feature 1..20: note = Synthetic Construct
  source 1..20: mol_type = other RNA
ctggctcagggactgcgagg

SEQ ID NO: 700 - RNA, 20 nt, synthetic construct
  misc_feature 1..20: note = Synthetic Construct
  source 1..20: mol_type = other RNA
ctggctcggggactgcgagg

SEQ ID NO: 701 - RNA, 20 nt, synthetic construct
  misc_feature 1..20: note = Synthetic Construct
  source 1..20: mol_type = other RNA
ctgggacacatagtctctgc

SEQ ID NO: 702 - RNA, 20 nt, synthetic construct
  misc_feature 1..20: note = Synthetic Construct
  source 1..20: mol_type = other RNA
ctgggactagtgcccagtta

SEQ ID NO: 703 - RNA, 20 nt, synthetic construct
  misc_feature 1..20: note = Synthetic Construct
  source 1..20: mol_type = other RNA
ctgggcaccagtcccagctc

SEQ ID NO: 704 - RNA, 20 nt, synthetic construct
  misc_feature 1..20: note = Synthetic Construct
  source 1..20: mol_type = other RNA
ctgggcactagtcccagctc

SEQ ID NO: 705 - RNA, 20 nt, synthetic construct
  misc_feature 1..20: note = Synthetic Construct
  source 1..20: mol_type = other RNA
ctgggcctctgggtacctga

SEQ ID NO: 706 - RNA, 20 nt, synthetic construct
  misc_feature 1..20: note = Synthetic Construct
  source 1..20: mol_type = other RNA
ctggtgcccagttacgttgg

SEQ ID NO: 707 - RNA, 20 nt, synthetic construct
  misc_feature 1..20: note = Synthetic Construct
  source 1..20: mol_type = other RNA
ctgtccagaaagacctcctt

SEQ ID NO: 708 - RNA, 20 nt, synthetic construct
  misc_feature 1..20: note = Synthetic Construct
  source 1..20: mol_type = other RNA
ctgtccagaaagacttcctt

SEQ ID NO: 709 - RNA, 20 nt, synthetic construct
  misc_feature 1..20: note = Synthetic Construct
  source 1..20: mol_type = other RNA
cttagaatatccctataagg

SEQ ID NO: 710 - RNA, 20 nt, synthetic construct
  misc_feature 1..20: note = Synthetic Construct
  source 1..20: mol_type = other RNA
cttccagcagtcacatgagt

SEQ ID NO: 711 - RNA, 20 nt, synthetic construct
  misc_feature 1..20: note = Synthetic Construct
  source 1..20: mol_type = other RNA
cttccagcagtcacgagtct

SEQ ID NO: 712 - RNA, 20 nt, synthetic construct
  misc_feature 1..20: note = Synthetic Construct
  source 1..20: mol_type = other RNA
ctttcacaacttagaatatc

SEQ ID NO: 713 - RNA, 20 nt, synthetic construct
  misc_feature 1..20: note = Synthetic Construct
  source 1..20: mol_type = other RNA
ctttctccctaaatccccgt

SEQ ID NO: 714 - RNA, 20 nt, synthetic construct
  misc_feature 1..20: note = Synthetic Construct
  source 1..20: mol_type = other RNA
ctttgcagtctgatcaggga

SEQ ID NO: 715 - RNA, 20 nt, synthetic construct
  misc_feature 1..20: note = Synthetic Construct
  source 1..20: mol_type = other RNA
gaaccctgggtgacacctgt

SEQ ID NO: 716 - RNA, 20 nt, synthetic construct
  misc_feature 1..20: note = Synthetic Construct
  source 1..20: mol_type = other RNA
gaaggaagtctttctggaca

SEQ ID NO: 717 - RNA, 20 nt, synthetic construct
  misc_feature 1..20: note = Synthetic Construct
  source 1..20: mol_type = other RNA
gaaggaggtctttctggaca

SEQ ID NO: 718 - RNA, 20 nt, synthetic construct
  misc_feature 1..20: note = Synthetic Construct
  source 1..20: mol_type = other RNA
gaaggcccagacctccccca

SEQ ID NO: 719 - RNA, 20 nt, synthetic construct
  misc_feature 1..20: note = Synthetic Construct
  source 1..20: mol_type = other RNA
gaaggccccccgatggttgg

SEQ ID NO: 720 - RNA, 20 nt, synthetic construct
  misc_feature 1..20: note = Synthetic Construct
  source 1..20: mol_type = other RNA
gaagtccctgatcagactgc

SEQ ID NO: 721 - RNA, 20 nt, synthetic construct
  misc_feature 1..20: note = Synthetic Construct
  source 1..20: mol_type = other RNA
gacacatagtctctgccgct

SEQ ID NO: 722 - RNA, 20 nt, synthetic construct
  misc_feature 1..20: note = Synthetic Construct
  source 1..20: mol_type = other RNA
gacacatagtctctgcgagc

SEQ ID NO: 723 - RNA, 20 nt, synthetic construct
  misc_feature 1..20: note = Synthetic Construct
  source 1..20: mol_type = other RNA
gacagagctgggactagtgc

SEQ ID NO: 724 - RNA, 20 nt, synthetic construct
  misc_feature 1..20: note = Synthetic Construct
  source 1..20: mol_type = other RNA
gacagagctgggactggtgc

SEQ ID NO: 725 - RNA, 20 nt, synthetic construct
  misc_feature 1..20: note = Synthetic Construct
  source 1..20: mol_type = other RNA
gacagcggcagagactatgt

SEQ ID NO: 726 - RNA, 20 nt, synthetic construct
  misc_feature 1..20: note = Synthetic Construct
  source 1..20: mol_type = other RNA
gacagctcgcagagactatg

SEQ ID NO: 727 - RNA, 20 nt, synthetic construct
  misc_feature 1..20: note = Synthetic Construct
  source 1..20: mol_type = other RNA
gacaggtgtcacccagggct

SEQ ID NO: 728 - RNA, 20 nt, synthetic construct
  misc_feature 1..20: note = Synthetic Construct
  source 1..20: mol_type = other RNA
gacaggtgtcacccagggtt

SEQ ID NO: 729 - RNA, 20 nt, synthetic construct
  misc_feature 1..20: note = Synthetic Construct
  source 1..20: mol_type = other RNA
gaccaacgtaactgggcacc

SEQ ID NO: 730 - RNA, 20 nt, synthetic construct
  misc_feature 1..20: note = Synthetic Construct
  source 1..20: mol_type = other RNA
gaccaacgtaactgggcact

SEQ ID NO: 731 - RNA, 20 nt, synthetic construct
  misc_feature 1..20: note = Synthetic Construct
  source 1..20: mol_type = other RNA
gaccagtgagcagcaacagg

SEQ ID NO: 732 - RNA, 20 nt, synthetic construct
  misc_feature 1..20: note = Synthetic Construct
  source 1..20: mol_type = other RNA
gacctcaggtacccagaggc

SEQ ID NO: 733 - RNA, 20 nt, synthetic construct
  misc_feature 1..20: note = Synthetic Construct
  source 1..20: mol_type = other RNA
gactagtgcccagttacgtt

SEQ ID NO: 734 - RNA, 20 nt, synthetic construct
  misc_feature 1..20: note = Synthetic Construct
  source 1..20: mol_type = other RNA
gactgcaaaggctgcaggct

SEQ ID NO: 735 - RNA, 20 nt, synthetic construct
  misc_feature 1..20: note = Synthetic Construct
  source 1..20: mol_type = other RNA
gactggtgcccagttacgtt

SEQ ID NO: 736 - RNA, 20 nt, synthetic construct
  misc_feature 1..20: note = Synthetic Construct
  source 1..20: mol_type = other RNA
gagaaagccccccgatggtt

SEQ ID NO: 737 - RNA, 20 nt, synthetic construct
  misc_feature 1..20: note = Synthetic Construct
  source 1..20: mol_type = other RNA
gagaaggccccccgatggtt

SEQ ID NO: 738 - RNA, 20 nt, synthetic construct
  misc_feature 1..20: note = Synthetic Construct
  source 1..20: mol_type = other RNA
gagacagagctgggactagt

SEQ ID NO: 739 - RNA, 20 nt, synthetic construct
  misc_feature 1..20: note = Synthetic Construct
  source 1..20: mol_type = other RNA
gagacagagctgggactggt

SEQ ID NO: 740 - RNA, 20 nt, synthetic construct
  misc_feature 1..20: note = Synthetic Construct
  source 1..20: mol_type = other RNA
gagaccaacgtaactgggca

SEQ ID NO: 741 - RNA, 20 nt, synthetic construct
  misc_feature 1..20: note = Synthetic Construct
  source 1..20: mol_type = other RNA
gagatcagactcatgtgact

SEQ ID NO: 742 - RNA, 20 nt, synthetic construct
  misc_feature 1..20: note = Synthetic Construct
  source 1..20: mol_type = other RNA
gagatcccattccagtttct

SEQ ID NO: 743 - RNA, 20 nt, synthetic construct
  misc_feature 1..20: note = Synthetic Construct
  source 1..20: mol_type = other RNA
gagatcccattccggtttct

SEQ ID NO: 744 - RNA, 20 nt, synthetic construct
  misc_feature 1..20: note = Synthetic Construct
  source 1..20: mol_type = other RNA
gagcagcaacagggccaggg

SEQ ID NO: 745 - RNA, 20 nt, synthetic construct
  misc_feature 1..20: note = Synthetic Construct
  source 1..20: mol_type = other RNA
gagcagcaacagggccgggg

SEQ ID NO: 746 - RNA, 20 nt, synthetic construct
  misc_feature 1..20: note = Synthetic Construct
  source 1..20: mol_type = other RNA
gagccaaccatcggggggcc

SEQ ID NO: 747 - RNA, 20 nt, synthetic construct
  misc_feature 1..20: note = Synthetic Construct
  source 1..20: mol_type = other RNA
gagccaaccatcggggggct

SEQ ID NO: 748 - RNA, 20 nt, synthetic construct
  misc_feature 1..20: note = Synthetic Construct
  source 1..20: mol_type = other RNA
gagccctgggtgacacctgt

SEQ ID NO: 749 - RNA, 20 nt, synthetic construct
  misc_feature 1..20: note = Synthetic Construct
  source 1..20: mol_type = other RNA
gagcctgcagcctttgcagt

SEQ ID NO: 750 - RNA, 20 nt, synthetic construct
  misc_feature 1..20: note = Synthetic Construct
  source 1..20: mol_type = other RNA
gagctgggactggtgcccag

SEQ ID NO: 751 - RNA, 20 nt, synthetic construct
  misc_feature 1..20: note = Synthetic Construct
  source 1..20: mol_type = other RNA
gagctgtctttgagcacatc

SEQ ID NO: 752 - RNA, 20 nt, synthetic construct
  misc_feature 1..20: note = Synthetic Construct
  source 1..20: mol_type = other RNA
gaggcccagcctggggcaag

SEQ ID NO: 753 - RNA, 20 nt, synthetic construct
  misc_feature 1..20: note = Synthetic Construct
  source 1..20: mol_type = other RNA
gaggcccggcctggggcaag

SEQ ID NO: 754 - RNA, 20 nt, synthetic construct
  misc_feature 1..20: note = Synthetic Construct
  source 1..20: mol_type = other RNA
gagggcaggggcagggggca

SEQ ID NO: 755 - RNA, 20 nt, synthetic construct
  misc_feature 1..20: note = Synthetic Construct
  source 1..20: mol_type = other RNA
gagggcaggggtagggggca

SEQ ID NO: 756 - RNA, 20 nt, synthetic construct
  misc_feature 1..20: note = Synthetic Construct
  source 1..20: mol_type = other RNA
gagtctgatctccactgcca

SEQ ID NO: 757 - RNA, 20 nt, synthetic construct
  misc_feature 1..20: note = Synthetic Construct
  source 1..20: mol_type = other RNA
gataagcccagccccggccc

SEQ ID NO: 758 - RNA, 20 nt, synthetic construct
  misc_feature 1..20: note = Synthetic Construct
  source 1..20: mol_type = other RNA
gataagcccagccctggccc

SEQ ID NO: 759 - RNA, 20 nt, synthetic construct
  misc_feature 1..20: note = Synthetic Construct
  source 1..20: mol_type = other RNA
gatcagactcgtgactgctg

SEQ ID NO: 760 - RNA, 20 nt, synthetic construct
  misc_feature 1..20: note = Synthetic Construct
  source 1..20: mol_type = other RNA
gatcccattccggtttctcc

SEQ ID NO: 761 - RNA, 20 nt, synthetic construct
  misc_feature 1..20: note = Synthetic Construct
  source 1..20: mol_type = other RNA
gatgcctcgcagtccccgag

SEQ ID NO: 762 - RNA, 20 nt, synthetic construct
  misc_feature 1..20: note = Synthetic Construct
  source 1..20: mol_type = other RNA
gatgcctcgcagtccctgag

SEQ ID NO: 763 - RNA, 20 nt, synthetic construct
  misc_feature 1..20: note = Synthetic Construct
  source 1..20: mol_type = other RNA
gatggagaaaccggaatggg

SEQ ID NO: 764 - RNA, 20 nt, synthetic construct
  misc_feature 1..20: note = Synthetic Construct
  source 1..20: mol_type = other RNA
gatggagaaactggaatggg

SEQ ID NO: 765 - RNA, 20 nt, synthetic construct
  misc_feature 1..20: note = Synthetic Construct
  source 1..20: mol_type = other RNA
gatggcagtggagatcagac

SEQ ID NO: 766 - RNA, 20 nt, synthetic construct
  misc_feature 1..20: note = Synthetic Construct
  source 1..20: mol_type = other RNA
gatgtgctcaaagacagcgg

SEQ ID NO: 767 - RNA, 20 nt, synthetic construct
  misc_feature 1..20: note = Synthetic Construct
  source 1..20: mol_type = other RNA
gatgtgctcaaagacagctc

SEQ ID NO: 768 - RNA, 20 nt, synthetic construct
  misc_feature 1..20: note = Synthetic Construct
  source 1..20: mol_type = other RNA
gcaaaggctgcaggctccgc

SEQ ID NO: 769 - RNA, 20 nt, synthetic construct
  misc_feature 1..20: note = Synthetic Construct
  source 1..20: mol_type = other RNA
gcaacagggccagggctggg

SEQ ID NO: 770 - RNA, 20 nt, synthetic construct
  misc_feature 1..20: note = Synthetic Construct
  source 1..20: mol_type = other RNA
gcaacagggccggggctggg

SEQ ID NO: 771 - RNA, 20 nt, synthetic construct
  misc_feature 1..20: note = Synthetic Construct
  source 1..20: mol_type = other RNA
gcaccagtcccagctctgtc

SEQ ID NO: 772 - RNA, 20 nt, synthetic construct
  misc_feature 1..20: note = Synthetic Construct
  source 1..20: mol_type = other RNA
gcacccttgcggagcctgca

SEQ ID NO: 773 - RNA, 20 nt, synthetic construct
  misc_feature 1..20: note = Synthetic Construct
  source 1..20: mol_type = other RNA
gcaggggtagggggcagagg

SEQ ID NO: 774 - RNA, 20 nt, synthetic construct
  misc_feature 1..20: note = Synthetic Construct
  source 1..20: mol_type = other RNA
gcagtcacatgagtctgatc

SEQ ID NO: 775 - RNA, 20 nt, synthetic construct
  misc_feature 1..20: note = Synthetic Construct
  source 1..20: mol_type = other RNA
gcagtcacgagtctgatctc

SEQ ID NO: 776 - RNA, 20 nt, synthetic construct
  misc_feature 1..20: note = Synthetic Construct
  source 1..20: mol_type = other RNA
gcagtccccgagccaggaaa

SEQ ID NO: 777 - RNA, 20 nt, synthetic construct
  misc_feature 1..20: note = Synthetic Construct
  source 1..20: mol_type = other RNA
gcagtccctgagccaggaaa

SEQ ID NO: 778 - RNA, 20 nt, synthetic construct
  misc_feature 1..20: note = Synthetic Construct
  source 1..20: mol_type = other RNA
gcagtggagatcagactcgt

SEQ ID NO: 779 - RNA, 20 nt, synthetic construct
  misc_feature 1..20: note = Synthetic Construct
  source 1..20: mol_type = other RNA
gccaaccatcggggggcctt

SEQ ID NO: 780 - RNA, 20 nt, synthetic construct
  misc_feature 1..20: note = Synthetic Construct
  source 1..20: mol_type = other RNA
gccaaccatcggggggcttt

SEQ ID NO: 781 - RNA, 20 nt, synthetic construct
  misc_feature 1..20: note = Synthetic Construct
  source 1..20: mol_type = other RNA
gccagggctgggcttatcag

SEQ ID NO: 782 - RNA, 20 nt, synthetic construct
  misc_feature 1..20: note = Synthetic Construct
  source 1..20: mol_type = other RNA
gcccagacctcccccagcag

SEQ ID NO: 783 - RNA, 20 nt, synthetic construct
  misc_feature 1..20: note = Synthetic Construct
  source 1..20: mol_type = other RNA
gcccagacctccctcagcag

SEQ ID NO: 784 - RNA, 20 nt, synthetic construct
  misc_feature 1..20: note = Synthetic Construct
  source 1..20: mol_type = other RNA
gcccagccccggccctgttg

SEQ ID NO: 785 - RNA, 20 nt, synthetic construct
  misc_feature 1..20: note = Synthetic Construct
  source 1..20: mol_type = other RNA
gcccagccctggccctgttg

SEQ ID NO: 786 - RNA, 20 nt, synthetic construct
  misc_feature 1..20: note = Synthetic Construct
  source 1..20: mol_type = other RNA
gccccagcctatcaggggtg

SEQ ID NO: 787 - RNA, 20 nt, synthetic construct
  misc_feature 1..20: note = Synthetic Construct
  source 1..20: mol_type = other RNA
gccccaggccgggcctctgg

SEQ ID NO: 788 - RNA, 20 nt, synthetic construct
  misc_feature 1..20: note = Synthetic Construct
  source 1..20: mol_type = other RNA
gccccaggctgggcctctgg

SEQ ID NO: 789 - RNA, 20 nt, synthetic construct
  misc_feature 1..20: note = Synthetic Construct
  source 1..20: mol_type = other RNA
gccccctacccctgccctca

SEQ ID NO: 790 - RNA, 20 nt, synthetic construct
  misc_feature 1..20: note = Synthetic Construct
  source 1..20: mol_type = other RNA
gccccctgcccctgccctca

SEQ ID NO: 791 - RNA, 20 nt, synthetic construct
  misc_feature 1..20: note = Synthetic Construct
  source 1..20: mol_type = other RNA
gccccggccctgttgctgct

SEQ ID NO: 792 - RNA, 20 nt, synthetic construct
  misc_feature 1..20: note = Synthetic Construct
  source 1..20: mol_type = other RNA
gccctggccctgttgctgct

SEQ ID NO: 793 - RNA, 20 nt, synthetic construct
  misc_feature 1..20: note = Synthetic Construct
  source 1..20: mol_type = other RNA
gccctgggtgacacctgtcc

SEQ ID NO: 794 - RNA, 20 nt, synthetic construct
  misc_feature 1..20: note = Synthetic Construct
  source 1..20: mol_type = other RNA
gccggggctgggcttatcag

SEQ ID NO: 795 - RNA, 20 nt, synthetic construct
  misc_feature 1..20: note = Synthetic Construct
  source 1..20: mol_type = other RNA
gcctccagccttacagggat

SEQ ID NO: 796 - RNA, 20 nt, synthetic construct
  misc_feature 1..20: note = Synthetic Construct
  source 1..20: mol_type = other RNA
gcctccagccttatagggat

SEQ ID NO: 797 - RNA, 20 nt, synthetic construct
  misc_feature 1..20: note = Synthetic Construct
  source 1..20: mol_type = other RNA
gcctctgccccctacccctg

SEQ ID NO: 798 - RNA, 20 nt, synthetic construct
  misc_feature 1..20: note = Synthetic Construct
  source 1..20: mol_type = other RNA
gcctctgccccctgcccctg

SEQ ID NO: 799 - RNA, 20 nt, synthetic construct
  misc_feature 1..20: note = Synthetic Construct
  source 1..20: mol_type = other RNA
gcctgcagcctttgcagtct

SEQ ID NO: 800 - RNA, 20 nt, synthetic construct
  misc_feature 1..20: note = Synthetic Construct
  source 1..20: mol_type = other RNA
gcctggagatcccattccag

SEQ ID NO: 801 - RNA, 20 nt, synthetic construct
  misc_feature 1..20: note = Synthetic Construct
  source 1..20: mol_type = other RNA
gcctggagatcccattccgg

SEQ ID NO: 802 - RNA, 20 nt, synthetic construct
  misc_feature 1..20: note = Synthetic Construct
  source 1..20: mol_type = other RNA
gcctggggttgagggcaggg

SEQ ID NO: 803 - RNA, 20 nt, synthetic construct
  misc_feature 1..20: note = Synthetic Construct
  source 1..20: mol_type = other RNA
gccttacagggatattctaa

SEQ ID NO: 804 - RNA, 20 nt, synthetic construct
  misc_feature 1..20: note = Synthetic Construct
  source 1..20: mol_type = other RNA
gccttatagggatattctaa

SEQ ID NO: 805 - RNA, 20 nt, synthetic construct
  misc_feature 1..20: note = Synthetic Construct
  source 1..20: mol_type = other RNA
gccttgccccaggccgggcc

SEQ ID NO: 806 - RNA, 20 nt, synthetic construct
  misc_feature 1..20: note = Synthetic Construct
  source 1..20: mol_type = other RNA
gccttgccccaggctgggcc

SEQ ID NO: 807 - RNA, 20 nt, synthetic construct
  misc_feature 1..20: note = Synthetic Construct
  source 1..20: mol_type = other RNA
gcctttgcagtctgatcagg

SEQ ID NO: 808 - RNA, 20 nt, synthetic construct
  misc_feature 1..20: note = Synthetic Construct
  source 1..20: mol_type = other RNA
gcgagctgtctttgagcaca

SEQ ID NO: 809 - RNA, 20 nt, synthetic construct
  misc_feature 1..20: note = Synthetic Construct
  source 1..20: mol_type = other RNA
gcggagcctgcagcctttgc

SEQ ID NO: 810 - RNA, 20 nt, synthetic construct
  misc_feature 1..20: note = Synthetic Construct
  source 1..20: mol_type = other RNA
gcggcagagactatgtgtcc

SEQ ID NO: 811 - RNA, 20 nt, synthetic construct
  misc_feature 1..20: note = Synthetic Construct
  source 1..20: mol_type = other RNA
gctcaaagacagcggcagag

SEQ ID NO: 812 - RNA, 20 nt, synthetic construct
  misc_feature 1..20: note = Synthetic Construct
  source 1..20: mol_type = other RNA
gctcaaagacagctcgcaga

SEQ ID NO: 813 - RNA, 20 nt, synthetic construct
  misc_feature 1..20: note = Synthetic Construct
  source 1..20: mol_type = other RNA
gctcgcagagactatgtgtc

SEQ ID NO: 814 - RNA, 20 nt, synthetic construct
  misc_feature 1..20: note = Synthetic Construct
  source 1..20: mol_type = other RNA
gctctgtccagaaagacctc

SEQ ID NO: 815 - RNA, 20 nt, synthetic construct
  misc_feature 1..20: note = Synthetic Construct
  source 1..20: mol_type = other RNA
gctctgtccagaaagacttc

SEQ ID NO: 816 - RNA, 20 nt, synthetic construct
  misc_feature 1..20: note = Synthetic Construct
  source 1..20: mol_type = other RNA
gctgagggaggtctgggcct

SEQ ID NO: 817 - RNA, 20 nt, synthetic construct
  misc_feature 1..20: note = Synthetic Construct
  source 1..20: mol_type = other RNA
gctgataagcccagccccgg

SEQ ID NO: 818 - RNA, 20 nt, synthetic construct
  misc_feature 1..20: note = Synthetic Construct
  source 1..20: mol_type = other RNA
gctgataagcccagccctgg

SEQ ID NO: 819 - RNA, 20 nt, synthetic construct
  misc_feature 1..20: note = Synthetic Construct
  source 1..20: mol_type = other RNA
gctgggactagtgcccagtt

SEQ ID NO: 820 - RNA, 20 nt, synthetic construct
  misc_feature 1..20: note = Synthetic Construct
  source 1..20: mol_type = other RNA
gctgggactggtgcccagtt

SEQ ID NO: 821 - RNA, 20 nt, synthetic construct
  misc_feature 1..20: note = Synthetic Construct
  source 1..20: mol_type = other RNA
gctgggggaggtctgggcct

SEQ ID NO: 822 - RNA, 20 nt, synthetic construct
  misc_feature 1..20: note = Synthetic Construct
  source 1..20: mol_type = other RNA
ggaaggcccagacctccccc

SEQ ID NO: 823 - RNA, 20 nt, synthetic construct
  misc_feature 1..20: note = Synthetic Construct
  source 1..20: mol_type = other RNA
ggaaggcccagacctccctc

SEQ ID NO: 824 - RNA, 20 nt, synthetic construct
  misc_feature 1..20: note = Synthetic Construct
  source 1..20: mol_type = other RNA
ggacacatagtctctgccgc

SEQ ID NO: 825 - RNA, 20 nt, synthetic construct
  misc_feature 1..20: note = Synthetic Construct
  source 1..20: mol_type = other RNA
ggacacatagtctctgcgag

SEQ ID NO: 826 - RNA, 20 nt, synthetic construct
  misc_feature 1..20: note = Synthetic Construct
  source 1..20: mol_type = other RNA
ggacaggtgtcacccagggc

SEQ ID NO: 827 - RNA, 20 nt, synthetic construct
  misc_feature 1..20: note = Synthetic Construct
  source 1..20: mol_type = other RNA
ggacaggtgtcacccagggt

SEQ ID NO: 828 - RNA, 20 nt, synthetic construct
  misc_feature 1..20: note = Synthetic Construct
  source 1..20: mol_type = other RNA
ggacttctcagtttcctggc

SEQ ID NO: 829 - RNA, 20 nt, synthetic construct
  misc_feature 1..20: note = Synthetic Construct
  source 1..20: mol_type = other RNA
ggagaaaccggaatgggatc

SEQ ID NO: 830 - RNA, 20 nt, synthetic construct
  misc_feature 1..20: note = Synthetic Construct
  source 1..20: mol_type = other RNA
ggagaaactggaatgggatc

SEQ ID NO: 831 - RNA, 20 nt, synthetic construct
  misc_feature 1..20: note = Synthetic Construct
  source 1..20: mol_type = other RNA
ggagacagagctgggactag

SEQ ID NO: 832 - RNA, 20 nt, synthetic construct
  misc_feature 1..20: note = Synthetic Construct
  source 1..20: mol_type = other RNA
ggagacagagctgggactgg

SEQ ID NO: 833 - RNA, 20 nt, synthetic construct
  misc_feature 1..20: note = Synthetic Construct
  source 1..20: mol_type = other RNA
ggagatcagactcatgtgac

SEQ ID NO: 834 - RNA, 20 nt, synthetic construct
  misc_feature 1..20: note = Synthetic Construct
  source 1..20: mol_type = other RNA
ggagatcccattccagtttc

SEQ ID NO: 835 - RNA, 20 nt, synthetic construct
  misc_feature 1..20: note = Synthetic Construct
  source 1..20: mol_type = other RNA
ggagatcccattccggtttc

SEQ ID NO: 836 - RNA, 20 nt, synthetic construct
  misc_feature 1..20: note = Synthetic Construct
  source 1..20: mol_type = other RNA
ggagccaaccatcggggggc

SEQ ID NO: 837 - RNA, 20 nt, synthetic construct
  misc_feature 1..20: note = Synthetic Construct
  source 1..20: mol_type = other RNA
ggaggctgataagcccagcc

SEQ ID NO: 838 - RNA, 20 nt, synthetic construct
  misc_feature 1..20: note = Synthetic Construct
  source 1..20: mol_type = other RNA
ggaggtctttctggacagag

SEQ ID NO: 839 - RNA, 20 nt, synthetic construct
  misc_feature 1..20: note = Synthetic Construct
  source 1..20: mol_type = other RNA
ggatggagaaaccggaatgg

SEQ ID NO: 840 - RNA, 20 nt, synthetic construct
  misc_feature 1..20: note = Synthetic Construct
  source 1..20: mol_type = other RNA
ggatggcagtggagatcaga

SEQ ID NO: 841 - RNA, 20 nt, synthetic construct
  misc_feature 1..20: note = Synthetic Construct
  source 1..20: mol_type = other RNA
ggatgtgctcaaagacagcg

SEQ ID NO: 842 - RNA, 20 nt, synthetic construct
  misc_feature 1..20: note = Synthetic Construct
  source 1..20: mol_type = other RNA
ggatgtgctcaaagacagct

SEQ ID NO: 843 - RNA, 20 nt, synthetic construct
  misc_feature 1..20: note = Synthetic Construct
  source 1..20: mol_type = other RNA
ggatttagggagaaagcccc

SEQ ID NO: 844 - RNA, 20 nt, synthetic construct
  misc_feature 1..20: note = Synthetic Construct
  source 1..20: mol_type = other RNA
ggatttagggagaaggcccc

SEQ ID NO: 845 - RNA, 20 nt, synthetic construct
  misc_feature 1..20: note = Synthetic Construct
  source 1..20: mol_type = other RNA
ggcaccagtcccagctctgt

SEQ ID NO: 846 - RNA, 20 nt, synthetic construct
  misc_feature 1..20: note = Synthetic Construct
  source 1..20: mol_type = other RNA
ggcactagtcccagctctgt

SEQ ID NO: 847 - RNA, 20 nt, synthetic construct
  misc_feature 1..20: note = Synthetic Construct
  source 1..20: mol_type = other RNA
ggcagagactatgtgtccca

SEQ ID NO: 848 - RNA, 20 nt, synthetic construct
  misc_feature 1..20: note = Synthetic Construct
  source 1..20: mol_type = other RNA
ggcaggggcagggggcagag

SEQ ID NO: 849 - RNA, 20 nt, synthetic construct
  misc_feature 1..20: note = Synthetic Construct
  source 1..20: mol_type = other RNA
ggcaggggtagggggcagag

SEQ ID NO: 850 - RNA, 20 nt, synthetic construct
  misc_feature 1..20: note = Synthetic Construct
  source 1..20: mol_type = other RNA
ggccacggggatttagggag

SEQ ID NO: 851 - RNA, 20 nt, synthetic construct
  misc_feature 1..20: note = Synthetic Construct
  source 1..20: mol_type = other RNA
ggccagggctgggcttatca

SEQ ID NO: 852 - RNA, 20 nt, synthetic construct
  misc_feature 1..20: note = Synthetic Construct
  source 1..20: mol_type = other RNA
ggcccagacctcccccagca

SEQ ID NO: 853 - RNA, 20 nt, synthetic construct
  misc_feature 1..20: note = Synthetic Construct
  source 1..20: mol_type = other RNA
ggcccagacctccctcagca

SEQ ID NO: 854 - RNA, 20 nt, synthetic construct
  misc_feature 1..20: note = Synthetic Construct
  source 1..20: mol_type = other RNA
ggccgggcctctgggtacct

SEQ ID NO: 855 - RNA, 20 nt, synthetic construct
  misc_feature 1..20: note = Synthetic Construct
  source 1..20: mol_type = other RNA
ggcctccagccttacaggga

SEQ ID NO: 856 - RNA, 20 nt, synthetic construct
  misc_feature 1..20: note = Synthetic Construct
  source 1..20: mol_type = other RNA
ggcctccagccttataggga

SEQ ID NO: 857 - RNA, 20 nt, synthetic construct
  misc_feature 1..20: note = Synthetic Construct
  source 1..20: mol_type = other RNA
ggcctctgccccctacccct

SEQ ID NO: 858 - RNA, 20 nt, synthetic construct
  misc_feature 1..20: note = Synthetic Construct
  source 1..20: mol_type = other RNA
ggcctctgccccctgcccct

SEQ ID NO: 859 - RNA, 20 nt, synthetic construct
  misc_feature 1..20: note = Synthetic Construct
  source 1..20: mol_type = other RNA
ggcctggggcaaggcctgaa

SEQ ID NO: 860 - RNA, 20 nt, synthetic construct
  misc_feature 1..20: note = Synthetic Construct
  source 1..20: mol_type = other RNA
ggccttctccctaaatcccc

SEQ ID NO: 861 - RNA, 20 nt, synthetic construct
  misc_feature 1..20: note = Synthetic Construct
  source 1..20: mol_type = other RNA
ggccttgccccaggccgggc

SEQ ID NO: 862 - RNA, 20 nt, synthetic construct
  misc_feature 1..20: note = Synthetic Construct
  source 1..20: mol_type = other RNA
ggctcagggactgcgaggca

SEQ ID NO: 863 - RNA, 20 nt, synthetic construct
  misc_feature 1..20: note = Synthetic Construct
  source 1..20: mol_type = other RNA
ggctcggggactgcgaggca

SEQ ID NO: 864 - RNA, 20 nt, synthetic construct
  misc_feature 1..20: note = Synthetic Construct
  source 1..20: mol_type = other RNA
ggctgataagcccagccccg

SEQ ID NO: 865 - RNA, 20 nt, synthetic construct
  misc_feature 1..20: note = Synthetic Construct
  source 1..20: mol_type = other RNA
ggctgataagcccagccctg

SEQ ID NO: 866 - RNA, 20 nt, synthetic construct
  misc_feature 1..20: note = Synthetic Construct
  source 1..20: mol_type = other RNA
ggctgcaggctccgcaaggg

SEQ ID NO: 867 - RNA, 20 nt, synthetic construct
  misc_feature 1..20: note = Synthetic Construct
  source 1..20: mol_type = other RNA
ggctgggcctctgggtacct

SEQ ID NO: 868 - RNA, 20 nt, synthetic construct
  misc_feature 1..20: note = Synthetic Construct
  source 1..20: mol_type = other RNA
ggctttctccctaaatcccc

SEQ ID NO: 869 - RNA, 20 nt, synthetic construct
  misc_feature 1..20: note = Synthetic Construct
  source 1..20: mol_type = other RNA
gggacacatagtctctgccg

SEQ ID NO: 870 - RNA, 20 nt, synthetic construct
  misc_feature 1..20: note = Synthetic Construct
  source 1..20: mol_type = other RNA
gggacacatagtctctgcga

SEQ ID NO: 871 - RNA, 20 nt, synthetic construct
  misc_feature 1..20: note = Synthetic Construct
  source 1..20: mol_type = other RNA
gggactagtgcccagttacg

SEQ ID NO: 872 - RNA, 20 nt, synthetic construct
  misc_feature 1..20: note = Synthetic Construct
  source 1..20: mol_type = other RNA
gggactggtgcccagttacg

SEQ ID NO: 873 - RNA, 20 nt, synthetic construct
  misc_feature 1..20: note = Synthetic Construct
  source 1..20: mol_type = other RNA
gggacttctcagtttcctgg

SEQ ID NO: 874 - RNA, 20 nt, synthetic construct
  misc_feature 1..20: note = Synthetic Construct
  source 1..20: mol_type = other RNA
gggagaaagccccccgatgg

SEQ ID NO: 875 - RNA, 20 nt, synthetic construct
  misc_feature 1..20: note = Synthetic Construct
  source 1..20: mol_type = other RNA
gggagaaggccccccgatgg

SEQ ID NO: 876 - RNA, 20 nt, synthetic construct
  misc_feature 1..20: note = Synthetic Construct
  source 1..20: mol_type = other RNA
gggagccaaccatcgggggg

SEQ ID NO: 877 - RNA, 20 nt, synthetic construct
  misc_feature 1..20: note = Synthetic Construct
  source 1..20: mol_type = other RNA
gggatttagggagaaagccc

SEQ ID NO: 878 - RNA, 20 nt, synthetic construct
  misc_feature 1..20: note = Synthetic Construct
  source 1..20: mol_type = other RNA
gggatttagggagaaggccc

SEQ ID NO: 879 - RNA, 20 nt, synthetic construct
  misc_feature 1..20: note = Synthetic Construct
  source 1..20: mol_type = other RNA
gggcaccagtcccagctctg

SEQ ID NO: 880 - RNA, 20 nt, synthetic construct
  misc_feature 1..20: note = Synthetic Construct
  source 1..20: mol_type = other RNA
gggcactagtcccagctctg

SEQ ID NO: 881 - RNA, 20 nt, synthetic construct
  misc_feature 1..20: note = Synthetic Construct
  source 1..20: mol_type = other RNA
gggcaggggcagggggcaga

SEQ ID NO: 882 - RNA, 20 nt, synthetic construct
  misc_feature 1..20: note = Synthetic Construct
  source 1..20: mol_type = other RNA
gggcaggggtagggggcaga

SEQ ID NO: 883 - RNA, 20 nt, synthetic construct
  misc_feature 1..20: note = Synthetic Construct
  source 1..20: mol_type = other RNA
gggccagggctgggcttatc

SEQ ID NO: 884 - RNA, 20 nt, synthetic construct
  misc_feature 1..20: note = Synthetic Construct
  source 1..20: mol_type = other RNA
gggccggggctgggcttatc

SEQ ID NO: 885 - RNA, 20 nt, synthetic construct
  misc_feature 1..20: note = Synthetic Construct
  source 1..20: mol_type = other RNA
ggggactgcgaggcatcagg

SEQ ID NO: 886 - RNA, 20 nt, synthetic construct
  misc_feature 1..20: note = Synthetic Construct
  source 1..20: mol_type = other RNA
ggggatttagggagaaagcc

SEQ ID NO: 887 - RNA, 20 nt, synthetic construct
  misc_feature 1..20: note = Synthetic Construct
  source 1..20: mol_type = other RNA
ggggcagggggcagaggcct

SEQ ID NO: 888 - RNA, 20 nt, synthetic construct
  misc_feature 1..20: note = Synthetic Construct
  source 1..20: mol_type = other RNA
ggggccttctccctaaatcc

SEQ ID NO: 889 - RNA, 20 nt, synthetic construct
  misc_feature 1..20: note = Synthetic Construct
  source 1..20: mol_type = other RNA
ggggctttctccctaaatcc

SEQ ID NO: 890 - RNA, 20 nt, synthetic construct
  misc_feature 1..20: note = Synthetic Construct
  source 1..20: mol_type = other RNA
gggggccttctccctaaatc

SEQ ID NO: 891 - RNA, 20 nt, synthetic construct
  misc_feature 1..20: note = Synthetic Construct
  source 1..20: mol_type = other RNA
gggggctttctccctaaatc

SEQ ID NO: 892 - RNA, 20 nt, synthetic construct
  misc_feature 1..20: note = Synthetic Construct
  source 1..20: mol_type = other RNA
ggggggctttctccctaaat

SEQ ID NO: 893 - RNA, 20 nt, synthetic construct
  misc_feature 1..20: note = Synthetic Construct
  source 1..20: mol_type = other RNA
ggggtgaaccctgggtgaca

SEQ ID NO: 894 - RNA, 20 nt, synthetic construct
  misc_feature 1..20: note = Synthetic Construct
  source 1..20: mol_type = other RNA
ggggtgagccctgggtgaca

SEQ ID NO: 895 - RNA, 20 nt, synthetic construct
  misc_feature 1..20: note = Synthetic Construct
  source 1..20: mol_type = other RNA
gggtgaaccctgggtgacac

SEQ ID NO: 896 - RNA, 20 nt, synthetic construct
  misc_feature 1..20: note = Synthetic Construct
  source 1..20: mol_type = other RNA
gggtgagccctgggtgacac

SEQ ID NO: 897 - RNA, 20 nt, synthetic construct
  misc_feature 1..20: note = Synthetic Construct
  source 1..20: mol_type = other RNA
ggtctggatggagaaaccgg

SEQ ID NO: 898 - RNA, 20 nt, synthetic construct
  misc_feature 1..20: note = Synthetic Construct
  source 1..20: mol_type = other RNA
ggtctggatggagaaactgg

SEQ ID NO: 899 - RNA, 20 nt, synthetic construct
  misc_feature 1..20: note = Synthetic Construct
  source 1..20: mol_type = other RNA
ggtctttctggacagagctg

SEQ ID NO: 900 - RNA, 20 nt, synthetic construct
  misc_feature 1..20: note = Synthetic Construct
  source 1..20: mol_type = other RNA
ggtgaaccctgggtgacacc

SEQ ID NO: 901 - RNA, 20 nt, synthetic construct
  misc_feature 1..20: note = Synthetic Construct
  source 1..20: mol_type = other RNA
ggtgagccctgggtgacacc

SEQ ID NO: 902 - RNA, 20 nt, synthetic construct
  misc_feature 1..20: note = Synthetic Construct
  source 1..20: mol_type = other RNA
ggttgagggcaggggtaggg

SEQ ID NO: 903 - RNA, 20 nt, synthetic construct
  misc_feature 1..20: note = Synthetic Construct
  source 1..20: mol_type = other RNA
gtaactgggcaccagtccca

SEQ ID NO: 904 - RNA, 20 nt, synthetic construct
  misc_feature 1..20: note = Synthetic Construct
  source 1..20: mol_type = other RNA
gtcacatgagtctgatctcc

SEQ ID NO: 905 - RNA, 20 nt, synthetic construct
  misc_feature 1..20: note = Synthetic Construct
  source 1..20: mol_type = other RNA
gtcacccagggctcacccct

SEQ ID NO: 906 - RNA, 20 nt, synthetic construct
  misc_feature 1..20: note = Synthetic Construct
  source 1..20: mol_type = other RNA
gtcacccagggttcacccct

SEQ ID NO: 907 - RNA, 20 nt, synthetic construct
  misc_feature 1..20: note = Synthetic Construct
  source 1..20: mol_type = other RNA
gtccagaaagacctccttcc

SEQ ID NO: 908 - RNA, 20 nt, synthetic construct
  misc_feature 1..20: note = Synthetic Construct
  source 1..20: mol_type = other RNA
gtccctgatcagactgcaaa

SEQ ID NO: 909 - RNA, 20 nt, synthetic construct
  misc_feature 1..20: note = Synthetic Construct
  source 1..20: mol_type = other RNA
gtctctgcgagctgtctttg

SEQ ID NO: 910 - RNA, 20 nt, synthetic construct
  misc_feature 1..20: note = Synthetic Construct
  source 1..20: mol_type = other RNA
gtctggatggagaaaccgga

SEQ ID NO: 911 - RNA, 20 nt, synthetic construct
  misc_feature 1..20: note = Synthetic Construct
  source 1..20: mol_type = other RNA
gtctggatggagaaactgga

SEQ ID NO: 912 - RNA, 20 nt, synthetic construct
  misc_feature 1..20: note = Synthetic Construct
  source 1..20: mol_type = other RNA
gtgaaccctgggtgacacct

SEQ ID NO: 913 - RNA, 20 nt, synthetic construct
  misc_feature 1..20: note = Synthetic Construct
  source 1..20: mol_type = other RNA
gtgagcagcaacagggccag

SEQ ID NO: 914 - RNA, 20 nt, synthetic construct
  misc_feature 1..20: note = Synthetic Construct
  source 1..20: mol_type = other RNA
gtgagcagcaacagggccgg

SEQ ID NO: 915 - RNA, 20 nt, synthetic construct
  misc_feature 1..20: note = Synthetic Construct
  source 1..20: mol_type = other RNA
gtgagccctgggtgacacct

SEQ ID NO: 916 - RNA, 20 nt, synthetic construct
  misc_feature 1..20: note = Synthetic Construct
  source 1..20: mol_type = other RNA
gtgctcaaagacagctcgca

SEQ ID NO: 917 - RNA, 20 nt, synthetic construct
  misc_feature 1..20: note = Synthetic Construct
  source 1..20: mol_type = other RNA
gtggagatcagactcatgtg

SEQ ID NO: 918 - RNA, 20 nt, synthetic construct
  misc_feature 1..20: note = Synthetic Construct
  source 1..20: mol_type = other RNA
gtggagatcagactcgtgac

SEQ ID NO: 919 - RNA, 20 nt, synthetic construct
  misc_feature 1..20: note = Synthetic Construct
  source 1..20: mol_type = other RNA
gtgtcacccagggctcaccc

SEQ ID NO: 920 - RNA, 20 nt, synthetic construct
  misc_feature 1..20: note = Synthetic Construct
  source 1..20: mol_type = other RNA
gtgtcacccagggttcaccc

SEQ ID NO: 921 - RNA, 20 nt, synthetic construct
  misc_feature 1..20: note = Synthetic Construct
  source 1..20: mol_type = other RNA
gttgagggcaggggcagggg

SEQ ID NO: 922 - RNA, 20 nt, synthetic construct
  misc_feature 1..20: note = Synthetic Construct
  source 1..20: mol_type = other RNA
gttgagggcaggggtagggg

SEQ ID NO: 923 - RNA, 20 nt, synthetic construct
  misc_feature 1..20: note = Synthetic Construct
  source 1..20: mol_type = other RNA
taactgggcaccagtcccag

SEQ ID NO: 924 - RNA, 20 nt, synthetic construct
  misc_feature 1..20: note = Synthetic Construct
  source 1..20: mol_type = other RNA
taactgggcactagtcccag

SEQ ID NO: 925 - RNA, 20 nt, synthetic construct
  misc_feature 1..20: note = Synthetic Construct
  source 1..20: mol_type = other RNA
taagcccagccccggccctg

SEQ ID NO: 926 - RNA, 20 nt, synthetic construct
  misc_feature 1..20: note = Synthetic Construct
  source 1..20: mol_type = other RNA
taagcccagccctggccctg

SEQ ID NO: 927 - RNA, 20 nt, synthetic construct
  misc_feature 1..20: note = Synthetic Construct
  source 1..20: mol_type = other RNA
tacccagaggcccagcctgg

SEQ ID NO: 928 - RNA, 20 nt, synthetic construct
  misc_feature 1..20: note = Synthetic Construct
  source 1..20: mol_type = other RNA
tacccagaggcccggcctgg

SEQ ID NO: 929 - RNA, 20 nt, synthetic construct
  misc_feature 1..20: note = Synthetic Construct
  source 1..20: mol_type = other RNA
tacccctgccctcaacccca

SEQ ID NO: 930 - RNA, 20 nt, synthetic construct
  misc_feature 1..20: note = Synthetic Construct
  source 1..20: mol_type = other RNA
tagggagaaagccccccgat

SEQ ID NO: 931 - RNA, 20 nt, synthetic construct
  misc_feature 1..20: note = Synthetic Construct
  source 1..20: mol_type = other RNA
tagggagaaggccccccgat

SEQ ID NO: 932 - RNA, 20 nt, synthetic construct
  misc_feature 1..20: note = Synthetic Construct
  source 1..20: mol_type = other RNA
tagggagccaaccatcgggg

SEQ ID NO: 933 - RNA, 20 nt, synthetic construct
  misc_feature 1..20: note = Synthetic Construct
  source 1..20: mol_type = other RNA
tagggatattctaagttgtg

SEQ ID NO: 934 - RNA, 20 nt, synthetic construct
  misc_feature 1..20: note = Synthetic Construct
  source 1..20: mol_type = other RNA
tagggggcagaggcctgtgg

SEQ ID NO: 935 - RNA, 20 nt, synthetic construct
  misc_feature 1..20: note = Synthetic Construct
  source 1..20: mol_type = other RNA
tagtcccagctctgtctcct

SEQ ID NO: 936 - RNA, 20 nt, synthetic construct
  misc_feature 1..20: note = Synthetic Construct
  source 1..20: mol_type = other RNA
tagtctctgcgagctgtctt

SEQ ID NO: 937 - RNA, 20 nt, synthetic construct
  misc_feature 1..20: note = Synthetic Construct
  source 1..20: mol_type = other RNA
tagtgcccagttacgttggt

SEQ ID NO: 938 - RNA, 20 nt, synthetic construct
  misc_feature 1..20: note = Synthetic Construct
  source 1..20: mol_type = other RNA
tatccctataaggctggagg

SEQ ID NO: 939 - RNA, 20 nt, synthetic construct
  misc_feature 1..20: note = Synthetic Construct
  source 1..20: mol_type = other RNA
tatccctgtaaggctggagg

SEQ ID NO: 940 - RNA, 20 nt, synthetic construct
  misc_feature 1..20: note = Synthetic Construct
  source 1..20: mol_type = other RNA
tatcctgatgcctcgcagtc

SEQ ID NO: 941 - RNA, 20 nt, synthetic construct
  misc_feature 1..20: note = Synthetic Construct
  source 1..20: mol_type = other RNA
tcaaagacagcggcagagac

SEQ ID NO: 942 - RNA, 20 nt, synthetic construct
  misc_feature 1..20: note = Synthetic Construct
  source 1..20: mol_type = other RNA
tcaaagacagctcgcagaga

SEQ ID NO: 943 - RNA, 20 nt, synthetic construct
  misc_feature 1..20: note = Synthetic Construct
  source 1..20: mol_type = other RNA
tcacaacttagaatatccct

SEQ ID NO: 944 - RNA, 20 nt, synthetic construct
  misc_feature 1..20: note = Synthetic Construct
  source 1..20: mol_type = other RNA
tcacccagggctcacccctg

SEQ ID NO: 945 - RNA, 20 nt, synthetic construct
  misc_feature 1..20: note = Synthetic Construct
  source 1..20: mol_type = other RNA
tcacccagggttcacccctg

SEQ ID NO: 946 - RNA, 20 nt, synthetic construct
  misc_feature 1..20: note = Synthetic Construct
  source 1..20: mol_type = other RNA
tcacgagtctgatctccact

SEQ ID NO: 947 - RNA, 20 nt, synthetic construct
  misc_feature 1..20: note = Synthetic Construct
  source 1..20: mol_type = other RNA
tcagactcatgtgactgctg

SEQ ID NO: 948 - RNA, 20 nt, synthetic construct
  misc_feature 1..20: note = Synthetic Construct
  source 1..20: mol_type = other RNA
tcagactgcaaaggctgcag

SEQ ID NO: 949 - RNA, 20 nt, synthetic construct
  misc_feature 1..20: note = Synthetic Construct
  source 1..20: mol_type = other RNA
tcagcaggttactgatagga

SEQ ID NO: 950 - RNA, 20 nt, synthetic construct
  misc_feature 1..20: note = Synthetic Construct
  source 1..20: mol_type = other RNA
tcagctctgtccagaaagac

SEQ ID NO: 951 - RNA, 20 nt, synthetic construct
  misc_feature 1..20: note = Synthetic Construct
  source 1..20: mol_type = other RNA
tcaggccttgccccaggccg

SEQ ID NO: 952 - RNA, 20 nt, synthetic construct
  misc_feature 1..20: note = Synthetic Construct
  source 1..20: mol_type = other RNA
tcaggccttgccccaggctg

SEQ ID NO: 953 - RNA, 20 nt, synthetic construct
  misc_feature 1..20: note = Synthetic Construct
  source 1..20: mol_type = other RNA
tcaggggtgaaccctgggtg

SEQ ID NO: 954 - RNA, 20 nt, synthetic construct
  misc_feature 1..20: note = Synthetic Construct
  source 1..20: mol_type = other RNA
tcaggggtgagccctgggtg

SEQ ID NO: 955 - RNA, 20 nt, synthetic construct
  misc_feature 1..20: note = Synthetic Construct
  source 1..20: mol_type = other RNA
tcaggtacccagaggcccag

SEQ ID NO: 956 - RNA, 20 nt, synthetic construct
  misc_feature 1..20: note = Synthetic Construct
  source 1..20: mol_type = other RNA
tcaggtacccagaggcccgg

SEQ ID NO: 957 - RNA, 20 nt, synthetic construct
  misc_feature 1..20: note = Synthetic Construct
  source 1..20: mol_type = other RNA
tcagtaacctgctgggggag

SEQ ID NO: 958 - RNA, 20 nt, synthetic construct
  misc_feature 1..20: note = Synthetic Construct
  source 1..20: mol_type = other RNA
tcagtttcctggctcaggga

SEQ ID NO: 959 - RNA, 20 nt, synthetic construct
  misc_feature 1..20: note = Synthetic Construct
  source 1..20: mol_type = other RNA
tcagtttcctggctcgggga

SEQ ID NO: 960 - RNA, 20 nt, synthetic construct
  misc_feature 1..20: note = Synthetic Construct
  source 1..20: mol_type = other RNA
tccagaaagacctccttcct

SEQ ID NO: 961 - RNA, 20 nt, synthetic construct
  misc_feature 1..20: note = Synthetic Construct
  source 1..20: mol_type = other RNA
tccagaaagacttccttcct

SEQ ID NO: 962 - RNA, 20 nt, synthetic construct
  misc_feature 1..20: note = Synthetic Construct
  source 1..20: mol_type = other RNA
tccagcagtcacgagtctga

SEQ ID NO: 963 - RNA, 20 nt, synthetic construct
  misc_feature 1..20: note = Synthetic Construct
  source 1..20: mol_type = other RNA
tccagccttacagggatatt

SEQ ID NO: 964 - RNA, 20 nt, synthetic construct
  misc_feature 1..20: note = Synthetic Construct
  source 1..20: mol_type = other RNA
tccagccttatagggatatt

SEQ ID NO: 965 - RNA, 20 nt, synthetic construct
  misc_feature 1..20: note = Synthetic Construct
  source 1..20: mol_type = other RNA
tcccacaggcctctgccccc

SEQ ID NO: 966 - RNA, 20 nt, synthetic construct
  misc_feature 1..20: note = Synthetic Construct
  source 1..20: mol_type = other RNA
tccctgagccaggaaactga

SEQ ID NO: 967 - RNA, 20 nt, synthetic construct
  misc_feature 1..20: note = Synthetic Construct
  source 1..20: mol_type = other RNA
tccctgatcagactgcaaag

SEQ ID NO: 968 - RNA, 20 nt, synthetic construct
  misc_feature 1..20: note = Synthetic Construct
  source 1..20: mol_type = other RNA
tccggtttctccatccagac

SEQ ID NO: 969 - RNA, 20 nt, synthetic construct
  misc_feature 1..20: note = Synthetic Construct
  source 1..20: mol_type = other RNA
tcctggaaggcccagacctc

SEQ ID NO: 970 - RNA, 20 nt, synthetic construct
  misc_feature 1..20: note = Synthetic Construct
  source 1..20: mol_type = other RNA
tcctggctcagggactgcga

SEQ ID NO: 971 - RNA, 20 nt, synthetic construct
  misc_feature 1..20: note = Synthetic Construct
  source 1..20: mol_type = other RNA
tcctggctcggggactgcga

SEQ ID NO: 972 - RNA, 20 nt, synthetic construct
  misc_feature 1..20: note = Synthetic Construct
  source 1..20: mol_type = other RNA
tcgcagagactatgtgtccc

SEQ ID NO: 973 - RNA, 20 nt, synthetic construct
  misc_feature 1..20: note = Synthetic Construct
  source 1..20: mol_type = other RNA
tcgcagtccccgagccagga

SEQ ID NO: 974 - RNA, 20 nt, synthetic construct
  misc_feature 1..20: note = Synthetic Construct
  source 1..20: mol_type = other RNA
tcgcagtccctgagccagga

SEQ ID NO: 975 - RNA, 20 nt, synthetic construct
  misc_feature 1..20: note = Synthetic Construct
  source 1..20: mol_type = other RNA
tcggggggccttctccctaa

SEQ ID NO: 976 - RNA, 20 nt, synthetic construct
  misc_feature 1..20: note = Synthetic Construct
  source 1..20: mol_type = other RNA
tcggggggctttctccctaa

SEQ ID NO: 977 - RNA, 20 nt, synthetic construct
  misc_feature 1..20: note = Synthetic Construct
  source 1..20: mol_type = other RNA
tcgtgactgctggaaggggg

SEQ ID NO: 978 - RNA, 20 nt, synthetic construct
  misc_feature 1..20: note = Synthetic Construct
  source 1..20: mol_type = other RNA
tctcagtttcctggctcagg

SEQ ID NO: 979 - RNA, 20 nt, synthetic construct
  misc_feature 1..20: note = Synthetic Construct
  source 1..20: mol_type = other RNA
tctcagtttcctggctcggg

SEQ ID NO: 980 - RNA, 20 nt, synthetic construct
  misc_feature 1..20: note = Synthetic Construct
  source 1..20: mol_type = other RNA
tctctgccgctgtctttgag

SEQ ID NO: 981 - RNA, 20 nt, synthetic construct
  misc_feature 1..20: note = Synthetic Construct
  source 1..20: mol_type = other RNA
tctctgcgagctgtctttga

SEQ ID NO: 982 - RNA, 20 nt, synthetic construct
  misc_feature 1..20: note = Synthetic Construct
  source 1..20: mol_type = other RNA
tctgccccctacccctgccc

SEQ ID NO: 983 - RNA, 20 nt, synthetic construct
  misc_feature 1..20: note = Synthetic Construct
  source 1..20: mol_type = other RNA
tctgccccctgcccctgccc

SEQ ID NO: 984 - RNA, 20 nt, synthetic construct
  misc_feature 1..20: note = Synthetic Construct
  source 1..20: mol_type = other RNA
tctgccgctgtctttgagca

SEQ ID NO: 985 - RNA, 20 nt, synthetic construct
  misc_feature 1..20: note = Synthetic Construct
  source 1..20: mol_type = other RNA
tctgcgagctgtctttgagc

SEQ ID NO: 986 - RNA, 20 nt, synthetic construct
  misc_feature 1..20: note = Synthetic Construct
  source 1..20: mol_type = other RNA
tctgtccagaaagacctcct

SEQ ID NO: 987 - RNA, 20 nt, synthetic construct
  misc_feature 1..20: note = Synthetic Construct
  source 1..20: mol_type = other RNA
tctgtccagaaagacttcct

SEQ ID NO: 988 - RNA, 20 nt, synthetic construct
  misc_feature 1..20: note = Synthetic Construct
  source 1..20: mol_type = other RNA
tgaaccctgggtgacacctg

SEQ ID NO: 989 - RNA, 20 nt, synthetic construct
  misc_feature 1..20: note = Synthetic Construct
  source 1..20: mol_type = other RNA
tgagcagcaacagggccagg

SEQ ID NO: 990 - RNA, 20 nt, synthetic construct
  misc_feature 1..20: note = Synthetic Construct
  source 1..20: mol_type = other RNA
tgagcagcaacagggccggg

SEQ ID NO: 991 - RNA, 20 nt, synthetic construct
  misc_feature 1..20: note = Synthetic Construct
  source 1..20: mol_type = other RNA
tgagccaggaaactgagaag

SEQ ID NO: 992 - RNA, 20 nt, synthetic construct
  misc_feature 1..20: note = Synthetic Construct
  source 1..20: mol_type = other RNA
tgagggaggtctgggccttc

SEQ ID NO: 993 - RNA, 20 nt, synthetic construct
  misc_feature 1..20: note = Synthetic Construct
  source 1..20: mol_type = other RNA
tgagtctgatctccactgcc

SEQ ID NO: 994 - RNA, 20 nt, synthetic construct
  misc_feature 1..20: note = Synthetic Construct
  source 1..20: mol_type = other RNA
tgataagcccagccccggcc

SEQ ID NO: 995 - RNA, 20 nt, synthetic construct
  misc_feature 1..20: note = Synthetic Construct
  source 1..20: mol_type = other RNA
tgataagcccagccctggcc

SEQ ID NO: 996 - RNA, 20 nt, synthetic construct
  misc_feature 1..20: note = Synthetic Construct
  source 1..20: mol_type = other RNA
tgatcagactgcaaaggctg

SEQ ID NO: 997 - RNA, 20 nt, synthetic construct
  misc_feature 1..20: note = Synthetic Construct
  source 1..20: mol_type = other RNA
tgatgcctcgcagtccccga

SEQ ID NO: 998 - RNA, 20 nt, synthetic construct
  misc_feature 1..20: note = Synthetic Construct
  source 1..20: mol_type = other RNA
tgatgcctcgcagtccctga

SEQ ID NO: 999 - RNA, 20 nt, synthetic construct
  misc_feature 1..20: note = Synthetic Construct
  source 1..20: mol_type = other RNA
tgcaaaggctgcaggctccg

SEQ ID NO: 1000 - RNA, 20 nt, synthetic construct
  misc_feature 1..20: note = Synthetic Construct
  source 1..20: mol_type = other RNA
tgcacccttgcggagcctgc

SEQ ID NO: 1001 - RNA, 20 nt, synthetic construct
  misc_feature 1..20: note = Synthetic Construct
  source 1..20: mol_type = other RNA
tgcagcctttgcagtctgat

SEQ ID NO: 1002 - RNA, 20 nt, synthetic construct
  misc_feature 1..20: note = Synthetic Construct
  source 1..20: mol_type = other RNA
tgccccaggccgggcctctg

SEQ ID NO: 1003 - RNA, 20 nt, synthetic construct
  misc_feature 1..20: note = Synthetic Construct
  source 1..20: mol_type = other RNA
tgccccaggctgggcctctg

SEQ ID NO: 1004 - RNA, 20 nt, synthetic construct
  misc_feature 1..20: note = Synthetic Construct
  source 1..20: mol_type = other RNA
tgccccctacccctgccctc

SEQ ID NO: 1005 - RNA, 20 nt, synthetic construct
  misc_feature 1..20: note = Synthetic Construct
  source 1..20: mol_type = other RNA
tgcccctgccctcaacccca

SEQ ID NO: 1006 - RNA, 20 nt, synthetic construct
  misc_feature 1..20: note = Synthetic Construct
  source 1..20: mol_type = other RNA
tgccgctgtctttgagcaca

SEQ ID NO: 1007 - RNA, 20 nt, synthetic construct
  misc_feature 1..20: note = Synthetic Construct
  source 1..20: mol_type = other RNA
tgcctggagatcccattccg

SEQ ID NO: 1008 - RNA, 20 nt, synthetic construct
  misc_feature 1..20: note = Synthetic Construct
  source 1..20: mol_type = other RNA
tgcgagctgtctttgagcac

SEQ ID NO: 1009 - RNA, 20 nt, synthetic construct
  misc_feature 1..20: note = Synthetic Construct
  source 1..20: mol_type = other RNA
tgcggagcctgcagcctttg

SEQ ID NO: 1010 - RNA, 20 nt, synthetic construct
  misc_feature 1..20: note = Synthetic Construct
  source 1..20: mol_type = other RNA
tgctcaaagacagcggcaga

SEQ ID NO: 1011 - RNA, 20 nt, synthetic construct
  misc_feature 1..20: note = Synthetic Construct
  source 1..20: mol_type = other RNA
tgctcaaagacagctcgcag

SEQ ID NO: 1012 - RNA, 20 nt, synthetic construct
  misc_feature 1..20: note = Synthetic Construct
  source 1..20: mol_type = other RNA
tgctgagggaggtctgggcc

SEQ ID NO: 1013 - RNA, 20 nt, synthetic construct
  misc_feature 1..20: note = Synthetic Construct
  source 1..20: mol_type = other RNA
tgctgggggaggtctgggcc

SEQ ID NO: 1014 - RNA, 20 nt, synthetic construct
  misc_feature 1..20: note = Synthetic Construct
  source 1..20: mol_type = other RNA
tggaaggcccagacctcccc

SEQ ID NO: 1015 - RNA, 20 nt, synthetic construct
  misc_feature 1..20: note = Synthetic Construct
  source 1..20: mol_type = other RNA
tggaaggcccagacctccct

SEQ ID NO: 1016 - RNA, 20 nt, synthetic construct
  misc_feature 1..20: note = Synthetic Construct
  source 1..20: mol_type = other RNA
tggagaaaccggaatgggat

SEQ ID NO: 1017 - RNA, 20 nt, synthetic construct
  misc_feature 1..20: note = Synthetic Construct
  source 1..20: mol_type = other RNA
tggagaaactggaatgggat

SEQ ID NO: 1018 - RNA, 20 nt, synthetic construct
  misc_feature 1..20: note = Synthetic Construct
  source 1..20: mol_type = other RNA
tggagaccaacgtaactggg

SEQ ID NO: 1019 - RNA, 20 nt, synthetic construct
  misc_feature 1..20: note = Synthetic Construct
  source 1..20: mol_type = other RNA
tggagatcagactcatgtga

SEQ ID NO: 1020 - RNA, 20 nt, synthetic construct
  misc_feature 1..20: note = Synthetic Construct
  source 1..20: mol_type = other RNA
tggagatcagactcgtgact

SEQ ID NO: 1021 - RNA, 20 nt, synthetic construct
  misc_feature 1..20: note = Synthetic Construct
  source 1..20: mol_type = other RNA
tggatgtgctcaaagacagc

SEQ ID NO: 1022 - RNA, 20 nt, synthetic construct
  misc_feature 1..20: note = Synthetic Construct
  source 1..20: mol_type = other RNA
tggcagtggagatcagactc

SEQ ID NO: 1023 - RNA, 20 nt, synthetic construct
  misc_feature 1..20: note = Synthetic Construct
  source 1..20: mol_type = other RNA
tggcctccagccttacaggg

SEQ ID NO: 1024 - RNA, 20 nt, synthetic construct
  misc_feature 1..20: note = Synthetic Construct
  source 1..20: mol_type = other RNA
tggcctccagccttataggg

SEQ ID NO: 1025 - RNA, 20 nt, synthetic construct
  misc_feature 1..20: note = Synthetic Construct
  source 1..20: mol_type = other RNA
tggctcagggactgcgaggc

SEQ ID NO: 1026 - RNA, 20 nt, synthetic construct
  misc_feature 1..20: note = Synthetic Construct
  source 1..20: mol_type = other RNA
tggctcggggactgcgaggc

SEQ ID NO: 1027 - RNA, 20 nt, synthetic construct
  misc_feature 1..20: note = Synthetic Construct
  source 1..20: mol_type = other RNA
tgggacacatagtctctgcc

SEQ ID NO: 1028 - RNA, 20 nt, synthetic construct
  misc_feature 1..20: note = Synthetic Construct
  source 1..20: mol_type = other RNA
tgggacacatagtctctgcg

SEQ ID NO: 1029 - RNA, 20 nt, synthetic construct
  misc_feature 1..20: note = Synthetic Construct
  source 1..20: mol_type = other RNA
tgggaggctgataagcccag

SEQ ID NO: 1030 - RNA, 20 nt, synthetic construct
  misc_feature 1..20: note = Synthetic Construct
  source 1..20: mol_type = other RNA
tgggcactagtcccagctct

SEQ ID NO: 1031 - RNA, 20 nt, synthetic construct
  misc_feature 1..20: note = Synthetic Construct
  source 1..20: mol_type = other RNA
tgggcctctgggtacctgag

SEQ ID NO: 1032 - RNA, 20 nt, synthetic construct
  misc_feature 1..20: note = Synthetic Construct
  source 1..20: mol_type = other RNA
tgggggaggtctgggccttc

SEQ ID NO: 1033 - RNA, 20 nt, synthetic construct
  misc_feature 1..20: note = Synthetic Construct
  source 1..20: mol_type = other RNA
tggtctggatggagaaaccg

SEQ ID NO: 1034 - RNA, 20 nt, synthetic construct
  misc_feature 1..20: note = Synthetic Construct
  source 1..20: mol_type = other RNA
tggtctggatggagaaactg

SEQ ID NO: 1035 - RNA, 20 nt, synthetic construct
  misc_feature 1..20: note = Synthetic Construct
  source 1..20: mol_type = other RNA
tggtgcccagttacgttggt

SEQ ID NO: 1036 - RNA, 20 nt, synthetic construct
  misc_feature 1..20: note = Synthetic Construct
  source 1..20: mol_type = other RNA
tgtccagaaagacctccttc

SEQ ID NO: 1037 - RNA, 20 nt, synthetic construct
  misc_feature 1..20: note = Synthetic Construct
  source 1..20: mol_type = other RNA
tgtccagaaagacttccttc

SEQ ID NO: 1038 - RNA, 20 nt, synthetic construct
  misc_feature 1..20: note = Synthetic Construct
  source 1..20: mol_type = other RNA
tgtgctcaaagacagcggca

SEQ ID NO: 1039 - RNA, 20 nt, synthetic construct
  misc_feature 1..20: note = Synthetic Construct
  source 1..20: mol_type = other RNA
tgtgctcaaagacagctcgc

SEQ ID NO: 1040 - RNA, 20 nt, synthetic construct
  misc_feature 1..20: note = Synthetic Construct
  source 1..20: mol_type = other RNA
ttacagggatattctaagtt

SEQ ID NO: 1041 - RNA, 20 nt, synthetic construct
  misc_feature 1..20: note = Synthetic Construct
  source 1..20: mol_type = other RNA
ttatagggatattctaagtt

SEQ ID NO: 1042 - RNA, 20 nt, synthetic construct
  misc_feature 1..20: note = Synthetic Construct
  source 1..20: mol_type = other RNA
ttcacaacttagaatatccc

SEQ ID NO: 1043 - RNA, 20 nt, synthetic construct
  misc_feature 1..20: note = Synthetic Construct
  source 1..20: mol_type = other RNA
ttcacccctgataggctggg

SEQ ID NO: 1044 - RNA, 20 nt, synthetic construct
  misc_feature 1..20: note = Synthetic Construct
  source 1..20: mol_type = other RNA
ttccagcagtcacgagtctg

SEQ ID NO: 1045 - RNA, 20 nt, synthetic construct
  misc_feature 1..20: note = Synthetic Construct
  source 1..20: mol_type = other RNA
ttccagtttctccatccaga

SEQ ID NO: 1046 - RNA, 20 nt, synthetic construct
  misc_feature 1..20: note = Synthetic Construct
  source 1..20: mol_type = other RNA
ttccggtttctccatccaga

SEQ ID NO: 1047 - RNA, 20 nt, synthetic construct
  misc_feature 1..20: note = Synthetic Construct
  source 1..20: mol_type = other RNA
ttgagggcaggggcaggggg

SEQ ID NO: 1048 - RNA, 20 nt, synthetic construct
  misc_feature 1..20: note = Synthetic Construct
  source 1..20: mol_type = other RNA
ttgagggcaggggtaggggg

SEQ ID NO: 1049 - RNA, 20 nt, synthetic construct
  misc_feature 1..20: note = Synthetic Construct
  source 1..20: mol_type = other RNA
ttgcggagcctgcagccttt

SEQ ID NO: 1050 - RNA, 20 nt, synthetic construct
  misc_feature 1..20: note = Synthetic Construct
  source 1..20: mol_type = other RNA
ttggaaaggaaggaagtctt

SEQ ID NO: 1051 - RNA, 20 nt, synthetic construct
  misc_feature 1..20: note = Synthetic Construct
  source 1..20: mol_type = other RNA
ttggaaaggaaggaggtctt

SEQ ID NO: 1052 - RNA, 20 nt, synthetic construct
  misc_feature 1..20: note = Synthetic Construct
  source 1..20: mol_type = other RNA
tttagggagaaggccccccg

SEQ ID NO: 1053 - RNA, 20 nt, synthetic construct
  misc_feature 1..20: note = Synthetic Construct
  source 1..20: mol_type = other RNA
tttcacaacttagaatatcc

SEQ ID NO: 1054 - RNA, 20 nt, synthetic construct
  misc_feature 1..20: note = Synthetic Construct
  source 1..20: mol_type = other RNA
tttcctggctcagggactgc

SEQ ID NO: 1055 - RNA, 20 nt, synthetic construct
  misc_feature 1..20: note = Synthetic Construct
  source 1..20: mol_type = other RNA
tttggaaaggaaggaagtct

SEQ ID NO: 1056 - RNA, 20 nt, synthetic construct
  misc_feature 1..20: note = Synthetic Construct
  source 1..20: mol_type = other RNA
tttggaaaggaaggaggtct

SEQ ID NO: 1057 - RNA, 20 nt, synthetic construct
  misc_feature 1..20: note = Synthetic Construct
  source 1..20: mol_type = other RNA
cttgccccaggccgggcctc

SEQ ID NO: 1058 - RNA, 20 nt, synthetic construct
  misc_feature 1..20: note = Synthetic Construct
  source 1..20: mol_type = other RNA
tctggatggagaaaccggaa

SEQ ID NO: 1059 - RNA, 20 nt, synthetic construct
  misc_feature 1..20: note = Synthetic Construct
  source 1..20: mol_type = other RNA
aaaagctgcaggctccgcaa

SEQ ID NO: 1060 - RNA, 20 nt, synthetic construct
  misc_feature 1..20: note = Synthetic Construct
  source 1..20: mol_type = other RNA
aaagacagcagcagagacta

SEQ ID NO: 1061 - RNA, 20 nt, synthetic construct
  misc_feature 1..20: note = Synthetic Construct
  source 1..20: mol_type = other RNA
acctccttcctttccaaaat

SEQ ID NO: 1062 - RNA, 20 nt, synthetic construct
  misc_feature 1..20: note = Synthetic Construct
  source 1..20: mol_type = other RNA
actaatattttggaaaggaa

SEQ ID NO: 1063 - RNA, 20 nt, synthetic construct
  misc_feature 1..20: note = Synthetic Construct
  source 1..20: mol_type = other RNA
acttccttcctttccaaaat

SEQ ID NO: 1064 - RNA, 20 nt, synthetic construct
  misc_feature 1..20: note = Synthetic Construct
  source 1..20: mol_type = other RNA
agcagcaacagggccagggc

SEQ ID NO: 1065 - RNA, 20 nt, synthetic construct
  misc_feature 1..20: note = Synthetic Construct
  source 1..20: mol_type = other RNA
agcctatcaggggtgaaccc

SEQ ID NO: 1066 - RNA, 20 nt, synthetic construct
  misc_feature 1..20: note = Synthetic Construct
  source 1..20: mol_type = other RNA
agcctgcagcttttgcagtc

SEQ ID NO: 1067 - RNA, 20 nt, synthetic construct
  misc_feature 1..20: note = Synthetic Construct
  source 1..20: mol_type = other RNA
agcttttgcagtctgatcag

SEQ ID NO: 1068 - RNA, 20 nt, synthetic construct
  misc_feature 1..20: note = Synthetic Construct
  source 1..20: mol_type = other RNA
aggccgggcctctgggtacc

SEQ ID NO: 1069 - RNA, 20 nt, synthetic construct
  misc_feature 1..20: note = Synthetic Construct
  source 1..20: mol_type = other RNA
agggctcacccctgataggc

SEQ ID NO: 1070 - RNA, 20 nt, synthetic construct
  misc_feature 1..20: note = Synthetic Construct
  source 1..20: mol_type = other RNA
aggtacccagaggcccagcc

SEQ ID NO: 1071 - RNA, 20 nt, synthetic construct
  misc_feature 1..20: note = Synthetic Construct
  source 1..20: mol_type = other RNA
atagtctctgctgctgtctt

SEQ ID NO: 1072 - RNA, 20 nt, synthetic construct
  misc_feature 1..20: note = Synthetic Construct
  source 1..20: mol_type = other RNA
atgtgctcaaagacagcagc

SEQ ID NO: 1073 - RNA, 20 nt, synthetic construct
  misc_feature 1..20: note = Synthetic Construct
  source 1..20: mol_type = other RNA
caaaagctgcaggctccgca

SEQ ID NO: 1074 - RNA, 20 nt, synthetic construct
  misc_feature 1..20: note = Synthetic Construct
  source 1..20: mol_type = other RNA
cagactgcaaaagctgcagg

SEQ ID NO: 1075 - RNA, 20 nt, synthetic construct
  misc_feature 1..20: note = Synthetic Construct
  source 1..20: mol_type = other RNA
cagcttttgcagtctgatca

SEQ ID NO: 1076 - RNA, 20 nt, synthetic construct
  misc_feature 1..20: note = Synthetic Construct
  source 1..20: mol_type = other RNA
catagtctctgctgctgtct

SEQ ID NO: 1077 - RNA, 20 nt, synthetic construct
  misc_feature 1..20: note = Synthetic Construct
  source 1..20: mol_type = other RNA
cccagaggcccggcctgggg

SEQ ID NO: 1078 - RNA, 20 nt, synthetic construct
  misc_feature 1..20: note = Synthetic Construct
  source 1..20: mol_type = other RNA
ccccggccctgttgctgctc

SEQ ID NO: 1079 - RNA, 20 nt, synthetic construct
  misc_feature 1..20: note = Synthetic Construct
  source 1..20: mol_type = other RNA
ctcggggactgcgaggcatc

SEQ ID NO: 1080 - RNA, 20 nt, synthetic construct
  misc_feature 1..20: note = Synthetic Construct
  source 1..20: mol_type = other RNA
ctctgccccctacccctgcc

SEQ ID NO: 1081 - RNA, 20 nt, synthetic construct
  misc_feature 1..20: note = Synthetic Construct
  source 1..20: mol_type = other RNA
cttctcagtttcctggctcg

SEQ ID NO: 1082 - RNA, 20 nt, synthetic construct
  misc_feature 1..20: note = Synthetic Construct
  source 1..20: mol_type = other RNA
cttgcggagcctgcagcttt

SEQ ID NO: 1083 - RNA, 20 nt, synthetic construct
  misc_feature 1..20: note = Synthetic Construct
  source 1..20: mol_type = other RNA
gaaaggaaggaagtctttct

SEQ ID NO: 1084 - RNA, 20 nt, synthetic construct
  misc_feature 1..20: note = Synthetic Construct
  source 1..20: mol_type = other RNA
gactcgtgactgctggaagg

SEQ ID NO: 1085 - RNA, 20 nt, synthetic construct
  misc_feature 1..20: note = Synthetic Construct
  source 1..20: mol_type = other RNA
gagaaaccggaatgggatct

SEQ ID NO: 1086 - RNA, 20 nt, synthetic construct
  misc_feature 1..20: note = Synthetic Construct
  source 1..20: mol_type = other RNA
gatcagactcatgtgactgc

SEQ ID NO: 1087 - RNA, 20 nt, synthetic construct
  misc_feature 1..20: note = Synthetic Construct
  source 1..20: mol_type = other RNA
gatcagactgcaaaagctgc

SEQ ID NO: 1088 - RNA, 20 nt, synthetic construct
  misc_feature 1..20: note = Synthetic Construct
  source 1..20: mol_type = other RNA
gcagcttttgcagtctgatc

SEQ ID NO: 1089 - RNA, 20 nt, synthetic construct
  misc_feature 1..20: note = Synthetic Construct
  source 1..20: mol_type = other RNA
gctgctgtctttgagcacat

SEQ ID NO: 1090 - RNA, 20 nt, synthetic construct
  misc_feature 1..20: note = Synthetic Construct
  source 1..20: mol_type = other RNA
ggagcctgcagcttttgcag

SEQ ID NO: 1091 - RNA, 20 nt, synthetic construct
  misc_feature 1..20: note = Synthetic Construct
  source 1..20: mol_type = other RNA
gggctttctccctaaatccc

SEQ ID NO: 1092 - RNA, 20 nt, synthetic construct
  misc_feature 1..20: note = Synthetic Construct
  source 1..20: mol_type = other RNA
gtaacctgctgggggaggtc

SEQ ID NO: 1093 - RNA, 20 nt, synthetic construct
  misc_feature 1..20: note = Synthetic Construct
  source 1..20: mol_type = other RNA
gtctctgctgctgtctttga

SEQ ID NO: 1094 - RNA, 20 nt, synthetic construct
  misc_feature 1..20: note = Synthetic Construct
  source 1..20: mol_type = other RNA
gtgctcaaagacagcagcag

SEQ ID NO: 1095 - RNA, 20 nt, synthetic construct
  misc_feature 1..20: note = Synthetic Construct
  source 1..20: mol_type = other RNA
taatattttggaaaggaagg

SEQ ID NO: 1096 - RNA, 20 nt, synthetic construct
  misc_feature 1..20: note = Synthetic Construct
  source 1..20: mol_type = other RNA
tagtctctgctgctgtcttt

SEQ ID NO: 1097 - RNA, 20 nt, synthetic construct
  misc_feature 1..20: note = Synthetic Construct
  source 1..20: mol_type = other RNA
tcccccagcaggttactgat

SEQ ID NO: 1098 - RNA, 20 nt, synthetic construct
  misc_feature 1..20: note = Synthetic Construct
  source 1..20: mol_type = other RNA
tcctatcagtaacctgctgg

SEQ ID NO: 1099 - RNA, 20 nt, synthetic construct
  misc_feature 1..20: note = Synthetic Construct
  source 1..20: mol_type = other RNA
tctggcctccagccttacag

SEQ ID NO: 1100 - RNA, 20 nt, synthetic construct
  misc_feature 1..20: note = Synthetic Construct
  source 1..20: mol_type = other RNA
tgagggcaggggtagggggc

SEQ ID NO: 1101 - RNA, 20 nt, synthetic construct
  misc_feature 1..20: note = Synthetic Construct
  source 1..20: mol_type = other RNA
tgtcacccagggctcacccc

SEQ ID NO: 1102 - RNA, 20 nt, synthetic construct
  misc_feature 1..20: note = Synthetic Construct
  source 1..20: mol_type = other RNA
aaaatctggcctccagcctt

SEQ ID NO: 1103 - RNA, 20 nt, synthetic construct
  misc_feature 1..20: note = Synthetic Construct
  source 1..20: mol_type = other RNA
aaactggaatgggatctcca

SEQ ID NO: 1104 - RNA, 20 nt, synthetic construct
  misc_feature 1..20: note = Synthetic Construct
  source 1..20: mol_type = other RNA
aaagacctccttcctttcca

SEQ ID NO: 1105 - RNA, 20 nt, synthetic construct
  misc_feature 1..20: note = Synthetic Construct
  source 1..20: mol_type = other RNA
aaagacttccttcctttcca

SEQ ID NO: 1106 - RNA, 20 nt, synthetic construct
  misc_feature 1..20: note = Synthetic Construct
  source 1..20: mol_type = other RNA
aacgtaactgggcaccagtc

SEQ ID NO: 1107 - RNA, 20 nt, synthetic construct
  misc_feature 1..20: note = Synthetic Construct
  source 1..20: mol_type = other RNA
aagacagcagcagagactat

SEQ ID NO: 1108 - RNA, 20 nt, synthetic construct
  misc_feature 1..20: note = Synthetic Construct
  source 1..20: mol_type = other RNA
aagacctccttcctttccaa

SEQ ID NO: 1109 - RNA, 20 nt, synthetic construct
  misc_feature 1..20: note = Synthetic Construct
  source 1..20: mol_type = other RNA
aagacttccttcctttccaa

SEQ ID NO: 1110 - RNA, 20 nt, synthetic construct
  misc_feature 1..20: note = Synthetic Construct
  source 1..20: mol_type = other RNA
aagcccagccccggccctgt

SEQ ID NO: 1111 - RNA, 20 nt, synthetic construct
  misc_feature 1..20: note = Synthetic Construct
  source 1..20: mol_type = other RNA
aatattttggaaaggaagga

SEQ ID NO: 1112 - RNA, 20 nt, synthetic construct
  misc_feature 1..20: note = Synthetic Construct
  source 1..20: mol_type = other RNA
acacatagtctctgctgctg

SEQ ID NO: 1113 - RNA, 20 nt, synthetic construct
  misc_feature 1..20: note = Synthetic Construct
  source 1..20: mol_type = other RNA
acagcagcagagactatgtg

SEQ ID NO: 1114 - RNA, 20 nt, synthetic construct
  misc_feature 1..20: note = Synthetic Construct
  source 1..20: mol_type = other RNA
acagggatattctaagttgt

SEQ ID NO: 1115 - RNA, 20 nt, synthetic construct
  misc_feature 1..20: note = Synthetic Construct
  source 1..20: mol_type = other RNA
acatagtctctgctgctgtc

SEQ ID NO: 1116 - RNA, 20 nt, synthetic construct
  misc_feature 1..20: note = Synthetic Construct
  source 1..20: mol_type = other RNA
accagtctgatctccactgc

SEQ ID NO: 1117 - RNA, 20 nt, synthetic construct
  misc_feature 1..20: note = Synthetic Construct
  source 1..20: mol_type = other RNA
accctgggtgacacctgtcc

SEQ ID NO: 1118 - RNA, 20 nt, synthetic construct
  misc_feature 1..20: note = Synthetic Construct
  source 1..20: mol_type = other RNA
actagtgcccagttacgttg

SEQ ID NO: 1119 - RNA, 20 nt, synthetic construct
  misc_feature 1..20: note = Synthetic Construct
  source 1..20: mol_type = other RNA
actgcaaaagctgcaggctc

SEQ ID NO: 1120 - RNA, 20 nt, synthetic construct
  misc_feature 1..20: note = Synthetic Construct
  source 1..20: mol_type = other RNA
actggtgactgctggaaggg

SEQ ID NO: 1121 - RNA, 20 nt, synthetic construct
  misc_feature 1..20: note = Synthetic Construct
  source 1..20: mol_type = other RNA
agacagagctgggactggtg

SEQ ID NO: 1122 - RNA, 20 nt, synthetic construct
  misc_feature 1..20: note = Synthetic Construct
  source 1..20: mol_type = other RNA
agacagcagcagagactatg

SEQ ID NO: 1123 - RNA, 20 nt, synthetic construct
  misc_feature 1..20: note = Synthetic Construct
  source 1..20: mol_type = other RNA
agacctccctcagcaggtta

SEQ ID NO: 1124 - RNA, 20 nt, synthetic construct
  misc_feature 1..20: note = Synthetic Construct
  source 1..20: mol_type = other RNA
agacctccttcctttccaaa

SEQ ID NO: 1125 - RNA, 20 nt, synthetic construct
  misc_feature 1..20: note = Synthetic Construct
  source 1..20: mol_type = other RNA
agactgcaaaagctgcaggc

SEQ ID NO: 1126 - RNA, 20 nt, synthetic construct
  misc_feature 1..20: note = Synthetic Construct
  source 1..20: mol_type = other RNA
agactggtgactgctggaag

SEQ ID NO: 1127 - RNA, 20 nt, synthetic construct
  misc_feature 1..20: note = Synthetic Construct
  source 1..20: mol_type = other RNA
agacttccttcctttccaaa

SEQ ID NO: 1128 - RNA, 20 nt, synthetic construct
  misc_feature 1..20: note = Synthetic Construct
  source 1..20: mol_type = other RNA
agaggcccagcctggggcaa

SEQ ID NO: 1129 - RNA, 20 nt, synthetic construct
  misc_feature 1..20: note = Synthetic Construct
  source 1..20: mol_type = other RNA
agatcagactggtgactgct

SEQ ID NO: 1130 - RNA, 20 nt, synthetic construct
  misc_feature 1..20: note = Synthetic Construct
  source 1..20: mol_type = other RNA
agcaacagggccggggctgg

SEQ ID NO: 1131 - RNA, 20 nt, synthetic construct
  misc_feature 1..20: note = Synthetic Construct
  source 1..20: mol_type = other RNA
agcagagactatgtgtccca

SEQ ID NO: 1132 - RNA, 20 nt, synthetic construct
  misc_feature 1..20: note = Synthetic Construct
  source 1..20: mol_type = other RNA
agcagcagagactatgtgtc

SEQ ID NO: 1133 - RNA, 20 nt, synthetic construct
  misc_feature 1..20: note = Synthetic Construct
  source 1..20: mol_type = other RNA
agcagtcaccagtctgatct

SEQ ID NO: 1134 - RNA, 20 nt, synthetic construct
  misc_feature 1..20: note = Synthetic Construct
  source 1..20: mol_type = other RNA
agcctggggcaaggcctgaa

SEQ ID NO: 1135 - RNA, 20 nt, synthetic construct
  misc_feature 1..20: note = Synthetic Construct
  source 1..20: mol_type = other RNA
aggagacagagctgggacta

SEQ ID NO: 1136 - RNA, 20 nt, synthetic construct
  misc_feature 1..20: note = Synthetic Construct
  source 1..20: mol_type = other RNA
aggctgataagcccagcccc

SEQ ID NO: 1137 - RNA, 20 nt, synthetic construct
  misc_feature 1..20: note = Synthetic Construct
  source 1..20: mol_type = other RNA
agggagaaggccccccgatg

SEQ ID NO: 1138 - RNA, 20 nt, synthetic construct
  misc_feature 1..20: note = Synthetic Construct
  source 1..20: mol_type = other RNA
aggggtgaaccctgggtgac

SEQ ID NO: 1139 - RNA, 20 nt, synthetic construct
  misc_feature 1..20: note = Synthetic Construct
  source 1..20: mol_type = other RNA
aggtgtcacccagggttcac

SEQ ID NO: 1140 - RNA, 20 nt, synthetic construct
  misc_feature 1..20: note = Synthetic Construct
  source 1..20: mol_type = other RNA
agtcaccagtctgatctcca

SEQ ID NO: 1141 - RNA, 20 nt, synthetic construct
  misc_feature 1..20: note = Synthetic Construct
  source 1..20: mol_type = other RNA
agtctctgctgctgtctttg

SEQ ID NO: 1142 - RNA, 20 nt, synthetic construct
  misc_feature 1..20: note = Synthetic Construct
  source 1..20: mol_type = other RNA
agtggagatcagactggtga

SEQ ID NO: 1143 - RNA, 20 nt, synthetic construct
  misc_feature 1..20: note = Synthetic Construct
  source 1..20: mol_type = other RNA
ataaggctggaggccagatt

SEQ ID NO: 1144 - RNA, 20 nt, synthetic construct
  misc_feature 1..20: note = Synthetic Construct
  source 1..20: mol_type = other RNA
atattttggaaaggaaggaa

SEQ ID NO: 1145 - RNA, 20 nt, synthetic construct
  misc_feature 1..20: note = Synthetic Construct
  source 1..20: mol_type = other RNA
atattttggaaaggaaggag

SEQ ID NO: 1146 - RNA, 20 nt, synthetic construct
  misc_feature 1..20: note = Synthetic Construct
  source 1..20: mol_type = other RNA
atcagactgcaaaagctgca

SEQ ID NO: 1147 - RNA, 20 nt, synthetic construct
  misc_feature 1..20: note = Synthetic Construct
  source 1..20: mol_type = other RNA
atcagactggtgactgctgg

SEQ ID NO: 1148 - RNA, 20 nt, synthetic construct
  misc_feature 1..20: note = Synthetic Construct
  source 1..20: mol_type = other RNA
atcaggggtgagccctgggt

SEQ ID NO: 1149 - RNA, 20 nt, synthetic construct
  misc_feature 1..20: note = Synthetic Construct
  source 1..20: mol_type = other RNA
atccctgtaaggctggaggc

SEQ ID NO: 1150 - RNA, 20 nt, synthetic construct
  misc_feature 1..20: note = Synthetic Construct
  source 1..20: mol_type = other RNA
attttggaaaggaaggaagt

SEQ ID NO: 1151 - RNA, 20 nt, synthetic construct
  misc_feature 1..20: note = Synthetic Construct
  source 1..20: mol_type = other RNA
attttggaaaggaaggaggt

SEQ ID NO: 1152 - RNA, 20 nt, synthetic construct
  misc_feature 1..20: note = Synthetic Construct
  source 1..20: mol_type = other RNA
caaagacagcagcagagact

SEQ ID NO: 1153 - RNA, 20 nt, synthetic construct
  misc_feature 1..20: note = Synthetic Construct
  source 1..20: mol_type = other RNA
caaccatcggggggccttct

SEQ ID NO: 1154 - RNA, 20 nt, synthetic construct
  misc_feature 1..20: note = Synthetic Construct
  source 1..20: mol_type = other RNA
cacatagtctctgctgctgt

SEQ ID NO: 1155 - RNA, 20 nt, synthetic construct
  misc_feature 1..20: note = Synthetic Construct
  source 1..20: mol_type = other RNA
caccagtctgatctccactg

SEQ ID NO: 1156 - RNA, 20 nt, synthetic construct
  misc_feature 1..20: note = Synthetic Construct
  source 1..20: mol_type = other RNA
cacgagtctgatctccactg

SEQ ID NO: 1157 - RNA, 20 nt, synthetic construct
  misc_feature 1..20: note = Synthetic Construct
  source 1..20: mol_type = other RNA
cacggggatttagggagaaa

SEQ ID NO: 1158 - RNA, 20 nt, synthetic construct
  misc_feature 1..20: note = Synthetic Construct
  source 1..20: mol_type = other RNA
cagactggtgactgctggaa

SEQ ID NO: 1159 - RNA, 20 nt, synthetic construct
  misc_feature 1..20: note = Synthetic Construct
  source 1..20: mol_type = other RNA
cagcagagactatgtgtccc

SEQ ID NO: 1160 - RNA, 20 nt, synthetic construct
  misc_feature 1..20: note = Synthetic Construct
  source 1..20: mol_type = other RNA
cagcagcagagactatgtgt

SEQ ID NO: 1161 - RNA, 20 nt, synthetic construct
  misc_feature 1..20: note = Synthetic Construct
  source 1..20: mol_type = other RNA
cagcagtcaccagtctgatc

SEQ ID NO: 1162 - RNA, 20 nt, synthetic construct
  misc_feature 1..20: note = Synthetic Construct
  source 1..20: mol_type = other RNA
caggcctctgccccctgccc

SEQ ID NO: 1163 - RNA, 20 nt, synthetic construct
  misc_feature 1..20: note = Synthetic Construct
  source 1..20: mol_type = other RNA
cagggccagggctgggctta

SEQ ID NO: 1164 - RNA, 20 nt, synthetic construct
  misc_feature 1..20: note = Synthetic Construct
  source 1..20: mol_type = other RNA
cagtcaccagtctgatctcc

SEQ ID NO: 1165 - RNA, 20 nt, synthetic construct
  misc_feature 1..20: note = Synthetic Construct
  source 1..20: mol_type = other RNA
cagtctgatctccactgcca

SEQ ID NO: 1166 - RNA, 20 nt, synthetic construct
  misc_feature 1..20: note = Synthetic Construct
  source 1..20: mol_type = other RNA
cagtggagatcagactggtg

SEQ ID NO: 1167 - RNA, 20 nt, synthetic construct
  misc_feature 1..20: note = Synthetic Construct
  source 1..20: mol_type = other RNA
ccacaggcctctgcccccta

SEQ ID NO: 1168 - RNA, 20 nt, synthetic construct
  misc_feature 1..20: note = Synthetic Construct
  source 1..20: mol_type = other RNA
ccagcagtcaccagtctgat

SEQ ID NO: 1169 - RNA, 20 nt, synthetic construct
  misc_feature 1..20: note = Synthetic Construct
  source 1..20: mol_type = other RNA
ccagcagtcacgagtctgat

SEQ ID NO: 1170 - RNA, 20 nt, synthetic construct
  misc_feature 1..20: note = Synthetic Construct
  source 1..20: mol_type = other RNA
ccagccttacagggatattc

SEQ ID NO: 1171 - RNA, 20 nt, synthetic construct
  misc_feature 1..20: note = Synthetic Construct
  source 1..20: mol_type = other RNA
ccagtctgatctccactgcc

SEQ ID NO: 1172 - RNA, 20 nt, synthetic construct
  misc_feature 1..20: note = Synthetic Construct
  source 1..20: mol_type = other RNA
ccatcggggggctttctccc

SEQ ID NO: 1173 - RNA, 20 nt, synthetic construct
  misc_feature 1..20: note = Synthetic Construct
  source 1..20: mol_type = other RNA
cccagacctcccccagcagg

SEQ ID NO: 1174 - RNA, 20 nt, synthetic construct
  misc_feature 1..20: note = Synthetic Construct
  source 1..20: mol_type = other RNA
cccagccctggccctgttgc

SEQ ID NO: 1175 - RNA, 20 nt, synthetic construct
  misc_feature 1..20: note = Synthetic Construct
  source 1..20: mol_type = other RNA
cccagcctatcaggggtgag

SEQ ID NO: 1176 - RNA, 20 nt, synthetic construct
  misc_feature 1..20: note = Synthetic Construct
  source 1..20: mol_type = other RNA
cccaggctgggcctctgggt

SEQ ID NO: 1177 - RNA, 20 nt, synthetic construct
  misc_feature 1..20: note = Synthetic Construct
  source 1..20: mol_type = other RNA
cccagggttcacccctgata

SEQ ID NO: 1178 - RNA, 20 nt, synthetic construct
  misc_feature 1..20: note = Synthetic Construct
  source 1..20: mol_type = other RNA
cccattccggtttctccatc

SEQ ID NO: 1179 - RNA, 20 nt, synthetic construct
  misc_feature 1..20: note = Synthetic Construct
  source 1..20: mol_type = other RNA
cccccttccagcagtcacca

SEQ ID NO: 1180 - RNA, 20 nt, synthetic construct
  misc_feature 1..20: note = Synthetic Construct
  source 1..20: mol_type = other RNA
ccccttccagcagtcaccag

SEQ ID NO: 1181 - RNA, 20 nt, synthetic construct
  misc_feature 1..20: note = Synthetic Construct
  source 1..20: mol_type = other RNA
cccggcctggggcaaggcct

SEQ ID NO: 1182 - RNA, 20 nt, synthetic construct
  misc_feature 1..20: note = Synthetic Construct
  source 1..20: mol_type = other RNA
ccctgatcagactgcaaaag

SEQ ID NO: 1183 - RNA, 20 nt, synthetic construct
  misc_feature 1..20: note = Synthetic Construct
  source 1..20: mol_type = other RNA
cccttccagcagtcaccagt

SEQ ID NO: 1184 - RNA, 20 nt, synthetic construct
  misc_feature 1..20: note = Synthetic Construct
  source 1..20: mol_type = other RNA
cccttgcggagcctgcagct

SEQ ID NO: 1185 - RNA, 20 nt, synthetic construct
  misc_feature 1..20: note = Synthetic Construct
  source 1..20: mol_type = other RNA
cctacccctgccctcaaccc

SEQ ID NO: 1186 - RNA, 20 nt, synthetic construct
  misc_feature 1..20: note = Synthetic Construct
  source 1..20: mol_type = other RNA
cctccttcctttccaaaata

SEQ ID NO: 1187 - RNA, 20 nt, synthetic construct
  misc_feature 1..20: note = Synthetic Construct
  source 1..20: mol_type = other RNA
cctcgcagtccccgagccag

SEQ ID NO: 1188 - RNA, 20 nt, synthetic construct
  misc_feature 1..20: note = Synthetic Construct
  source 1..20: mol_type = other RNA
cctgatcagactgcaaaagc

SEQ ID NO: 1189 - RNA, 20 nt, synthetic construct
  misc_feature 1..20: note = Synthetic Construct
  source 1..20: mol_type = other RNA
cctgcagcttttgcagtctg

SEQ ID NO: 1190 - RNA, 20 nt, synthetic construct
  misc_feature 1..20: note = Synthetic Construct
  source 1..20: mol_type = other RNA
ccttccagcagtcaccagtc

SEQ ID NO: 1191 - RNA, 20 nt, synthetic construct
  misc_feature 1..20: note = Synthetic Construct
  source 1..20: mol_type = other RNA
ccttgcggagcctgcagctt

SEQ ID NO: 1192 - RNA, 20 nt, synthetic construct
  misc_feature 1..20: note = Synthetic Construct
  source 1..20: mol_type = other RNA
cggagcctgcagcttttgca

SEQ ID NO: 1193 - RNA, 20 nt, synthetic construct
  misc_feature 1..20: note = Synthetic Construct
  source 1..20: mol_type = other RNA
ctaatattttggaaaggaag

SEQ ID NO: 1194 - RNA, 20 nt, synthetic construct
  misc_feature 1..20: note = Synthetic Construct
  source 1..20: mol_type = other RNA
ctataaggctggaggccaga

SEQ ID NO: 1195 - RNA, 20 nt, synthetic construct
  misc_feature 1..20: note = Synthetic Construct
  source 1..20: mol_type = other RNA
ctcaaagacagcagcagaga

SEQ ID NO: 1196 - RNA, 20 nt, synthetic construct
  misc_feature 1..20: note = Synthetic Construct
  source 1..20: mol_type = other RNA
ctcagcaggttactgatagg

SEQ ID NO: 1197 - RNA, 20 nt, synthetic construct
  misc_feature 1..20: note = Synthetic Construct
  source 1..20: mol_type = other RNA
ctcaggtacccagaggcccg

SEQ ID NO: 1198 - RNA, 20 nt, synthetic construct
  misc_feature 1..20: note = Synthetic Construct
  source 1..20: mol_type = other RNA
ctccttcctttccaaaatat

SEQ ID NO: 1199 - RNA, 20 nt, synthetic construct
  misc_feature 1..20: note = Synthetic Construct
  source 1..20: mol_type = other RNA
ctctgctgctgtctttgagc

SEQ ID NO: 1200 - RNA, 20 nt, synthetic construct
  misc_feature 1..20: note = Synthetic Construct
  source 1..20: mol_type = other RNA
ctgataagcccagccctggc

SEQ ID NO: 1201 - RNA, 20 nt, synthetic construct
  misc_feature 1..20: note = Synthetic Construct
  source 1..20: mol_type = other RNA
ctgatcagactgcaaaagct

SEQ ID NO: 1202 - RNA, 20 nt, synthetic construct
  misc_feature 1..20: note = Synthetic Construct
  source 1..20: mol_type = other RNA
ctgcaaaagctgcaggctcc

SEQ ID NO: 1203 - RNA, 20 nt, synthetic construct
  misc_feature 1..20: note = Synthetic Construct
  source 1..20: mol_type = other RNA
ctgcagcttttgcagtctga

SEQ ID NO: 1204 - RNA, 20 nt, synthetic construct
  misc_feature 1..20: note = Synthetic Construct
  source 1..20: mol_type = other RNA
ctgctgagggaggtctgggc

SEQ ID NO: 1205 - RNA, 20 nt, synthetic construct
  misc_feature 1..20: note = Synthetic Construct
  source 1..20: mol_type = other RNA
ctgctgctgtctttgagcac

SEQ ID NO: 1206 - RNA, 20 nt, synthetic construct
  misc_feature 1..20: note = Synthetic Construct
  source 1..20: mol_type = other RNA
ctgctgtctttgagcacatc

SEQ ID NO: 1207 - RNA, 20 nt, synthetic construct
  misc_feature 1..20: note = Synthetic Construct
  source 1..20: mol_type = other RNA
ctggagatcccattccggtt

SEQ ID NO: 1208 - RNA, 20 nt, synthetic construct
  misc_feature 1..20: note = Synthetic Construct
  source 1..20: mol_type = other RNA
ctgggactggtgcccagtta

SEQ ID NO: 1209 - RNA, 20 nt, synthetic construct
  misc_feature 1..20: note = Synthetic Construct
  source 1..20: mol_type = other RNA
ctgggggaggtctgggcctt

SEQ ID NO: 1210 - RNA, 20 nt, synthetic construct
  misc_feature 1..20: note = Synthetic Construct
  source 1..20: mol_type = other RNA
ctggtgactgctggaagggg

SEQ ID NO: 1211 - RNA, 20 nt, synthetic construct
  misc_feature 1..20: note = Synthetic Construct
  source 1..20: mol_type = other RNA
ctgtaaggctggaggccaga

SEQ ID NO: 1212 - RNA, 20 nt, synthetic construct
  misc_feature 1..20: note = Synthetic Construct
  source 1..20: mol_type = other RNA
cttagaatatccctgtaagg

SEQ ID NO: 1213 - RNA, 20 nt, synthetic construct
  misc_feature 1..20: note = Synthetic Construct
  source 1..20: mol_type = other RNA
cttccagcagtcaccagtct

SEQ ID NO: 1214 - RNA, 20 nt, synthetic construct
  misc_feature 1..20: note = Synthetic Construct
  source 1..20: mol_type = other RNA
cttccttcctttccaaaata

SEQ ID NO: 1215 - RNA, 20 nt, synthetic construct
  misc_feature 1..20: note = Synthetic Construct
  source 1..20: mol_type = other RNA
cttttgcagtctgatcaggg

SEQ ID NO: 1216 - RNA, 20 nt, synthetic construct
  misc_feature 1..20: note = Synthetic Construct
  source 1..20: mol_type = other RNA
gaaagacctccttcctttcc

SEQ ID NO: 1217 - RNA, 20 nt, synthetic construct
  misc_feature 1..20: note = Synthetic Construct
  source 1..20: mol_type = other RNA
gaaagacttccttcctttcc

SEQ ID NO: 1218 - RNA, 20 nt, synthetic construct
  misc_feature 1..20: note = Synthetic Construct
  source 1..20: mol_type = other RNA
gaaagccccccgatggttgg

SEQ ID NO: 1219 - RNA, 20 nt, synthetic construct
  misc_feature 1..20: note = Synthetic Construct
  source 1..20: mol_type = other RNA
gaaggcccagacctccctca

SEQ ID NO: 1220 - RNA, 20 nt, synthetic construct
  misc_feature 1..20: note = Synthetic Construct
  source 1..20: mol_type = other RNA
gacacatagtctctgctgct

SEQ ID NO: 1221 - RNA, 20 nt, synthetic construct
  misc_feature 1..20: note = Synthetic Construct
  source 1..20: mol_type = other RNA
gacagcagcagagactatgt

SEQ ID NO: 1222 - RNA, 20 nt, synthetic construct
  misc_feature 1..20: note = Synthetic Construct
  source 1..20: mol_type = other RNA
gacctccttcctttccaaaa

SEQ ID NO: 1223 - RNA, 20 nt, synthetic construct
  misc_feature 1..20: note = Synthetic Construct
  source 1..20: mol_type = other RNA
gactgcaaaagctgcaggct

SEQ ID NO: 1224 - RNA, 20 nt, synthetic construct
  misc_feature 1..20: note = Synthetic Construct
  source 1..20: mol_type = other RNA
gactggtgactgctggaagg

SEQ ID NO: 1225 - RNA, 20 nt, synthetic construct
  misc_feature 1..20: note = Synthetic Construct
  source 1..20: mol_type = other RNA
gacttccttcctttccaaaa

SEQ ID NO: 1226 - RNA, 20 nt, synthetic construct
  misc_feature 1..20: note = Synthetic Construct
  source 1..20: mol_type = other RNA
gagatcagactggtgactgc

SEQ ID NO: 1227 - RNA, 20 nt, synthetic construct
  misc_feature 1..20: note = Synthetic Construct
  source 1..20: mol_type = other RNA
gagcctgcagcttttgcagt

SEQ ID NO: 1228 - RNA, 20 nt, synthetic construct
  misc_feature 1..20: note = Synthetic Construct
  source 1..20: mol_type = other RNA
gagctgggactagtgcccag

SEQ ID NO: 1229 - RNA, 20 nt, synthetic construct
  misc_feature 1..20: note = Synthetic Construct
  source 1..20: mol_type = other RNA
gatcagactggtgactgctg

SEQ ID NO: 1230 - RNA, 20 nt, synthetic construct
  misc_feature 1..20: note = Synthetic Construct
  source 1..20: mol_type = other RNA
gatcccattccagtttctcc

SEQ ID NO: 1231 - RNA, 20 nt, synthetic construct
  misc_feature 1..20: note = Synthetic Construct
  source 1..20: mol_type = other RNA
gatgtgctcaaagacagcag

SEQ ID NO: 1232 - RNA, 20 nt, synthetic construct
  misc_feature 1..20: note = Synthetic Construct
  source 1..20: mol_type = other RNA
gcaaaagctgcaggctccgc

SEQ ID NO: 1233 - RNA, 20 nt, synthetic construct
  misc_feature 1..20: note = Synthetic Construct
  source 1..20: mol_type = other RNA
gcactagtcccagctctgtc

SEQ ID NO: 1234 - RNA, 20 nt, synthetic construct
  misc_feature 1..20: note = Synthetic Construct
  source 1..20: mol_type = other RNA
gcagcagagactatgtgtcc

SEQ ID NO: 1235 - RNA, 20 nt, synthetic construct
  misc_feature 1..20: note = Synthetic Construct
  source 1..20: mol_type = other RNA
gcaggggcagggggcagagg

SEQ ID NO: 1236 - RNA, 20 nt, synthetic construct
  misc_feature 1..20: note = Synthetic Construct
  source 1..20: mol_type = other RNA
gcagtcaccagtctgatctc

SEQ ID NO: 1237 - RNA, 20 nt, synthetic construct
  misc_feature 1..20: note = Synthetic Construct
  source 1..20: mol_type = other RNA
gcagtggagatcagactcat

SEQ ID NO: 1238 - RNA, 20 nt, synthetic construct
  misc_feature 1..20: note = Synthetic Construct
  source 1..20: mol_type = other RNA
gcagtggagatcagactggt

SEQ ID NO: 1239 - RNA, 20 nt, synthetic construct
  misc_feature 1..20: note = Synthetic Construct
  source 1..20: mol_type = other RNA
gcccccttccagcagtcacc

SEQ ID NO: 1240 - RNA, 20 nt, synthetic construct
  misc_feature 1..20: note = Synthetic Construct
  source 1..20: mol_type = other RNA
gcccccttccagcagtcacg

SEQ ID NO: 1241 - RNA, 20 nt, synthetic construct
  misc_feature 1..20: note = Synthetic Construct
  source 1..20: mol_type = other RNA
gcccctgccctcaaccccag

SEQ ID NO: 1242 - RNA, 20 nt, synthetic construct
  misc_feature 1..20: note = Synthetic Construct
  source 1..20: mol_type = other RNA
gcctgcagcttttgcagtct

SEQ ID NO: 1243 - RNA, 20 nt, synthetic construct
  misc_feature 1..20: note = Synthetic Construct
  source 1..20: mol_type = other RNA
gcggagcctgcagcttttgc

SEQ ID NO: 1244 - RNA, 20 nt, synthetic construct
  misc_feature 1..20: note = Synthetic Construct
  source 1..20: mol_type = other RNA
gctcaaagacagcagcagag

SEQ ID NO: 1245 - RNA, 20 nt, synthetic construct
  misc_feature 1..20: note = Synthetic Construct
  source 1..20: mol_type = other RNA
gcttttgcagtctgatcagg

SEQ ID NO: 1246 - RNA, 20 nt, synthetic construct
  misc_feature 1..20: note = Synthetic Construct
  source 1..20: mol_type = other RNA
ggaagtctttctggacagag

SEQ ID NO: 1247 - RNA, 20 nt, synthetic construct
  misc_feature 1..20: note = Synthetic Construct
  source 1..20: mol_type = other RNA
ggacacatagtctctgctgc

SEQ ID NO: 1248 - RNA, 20 nt, synthetic construct
  misc_feature 1..20: note = Synthetic Construct
  source 1..20: mol_type = other RNA
ggagatcagactcgtgactg

SEQ ID NO: 1249 - RNA, 20 nt, synthetic construct
  misc_feature 1..20: note = Synthetic Construct
  source 1..20: mol_type = other RNA
ggagatcagactggtgactg

SEQ ID NO: 1250 - RNA, 20 nt, synthetic construct
  misc_feature 1..20: note = Synthetic Construct
  source 1..20: mol_type = other RNA
ggatggagaaactggaatgg

SEQ ID NO: 1251 - RNA, 20 nt, synthetic construct
  misc_feature 1..20: note = Synthetic Construct
  source 1..20: mol_type = other RNA
ggatgtgctcaaagacagca

SEQ ID NO: 1252 - RNA, 20 nt, synthetic construct
  misc_feature 1..20: note = Synthetic Construct
  source 1..20: mol_type = other RNA
ggcagtggagatcagactgg

SEQ ID NO: 1253 - RNA, 20 nt, synthetic construct
  misc_feature 1..20: note = Synthetic Construct
  source 1..20: mol_type = other RNA
ggccccccgatggttggctc

SEQ ID NO: 1254 - RNA, 20 nt, synthetic construct
  misc_feature 1..20: note = Synthetic Construct
  source 1..20: mol_type = other RNA
ggccggggctgggcttatca

SEQ ID NO: 1255 - RNA, 20 nt, synthetic construct
  misc_feature 1..20: note = Synthetic Construct
  source 1..20: mol_type = other RNA
ggccttgccccaggctgggc

SEQ ID NO: 1256 - RNA, 20 nt, synthetic construct
  misc_feature 1..20: note = Synthetic Construct
  source 1..20: mol_type = other RNA
gggacacatagtctctgctg

SEQ ID NO: 1257 - RNA, 20 nt, synthetic construct
  misc_feature 1..20: note = Synthetic Construct
  source 1..20: mol_type = other RNA
ggggatttagggagaaggcc

SEQ ID NO: 1258 - RNA, 20 nt, synthetic construct
  misc_feature 1..20: note = Synthetic Construct
  source 1..20: mol_type = other RNA
ggggggccttctccctaaat

SEQ ID NO: 1259 - RNA, 20 nt, synthetic construct
  misc_feature 1..20: note = Synthetic Construct
  source 1..20: mol_type = other RNA
ggggtagggggcagaggcct

SEQ ID NO: 1260 - RNA, 20 nt, synthetic construct
  misc_feature 1..20: note = Synthetic Construct
  source 1..20: mol_type = other RNA
ggtgactgctggaagggggc

SEQ ID NO: 1261 - RNA, 20 nt, synthetic construct
  misc_feature 1..20: note = Synthetic Construct
  source 1..20: mol_type = other RNA
ggtgcccagttacgttggtc

SEQ ID NO: 1262 - RNA, 20 nt, synthetic construct
  misc_feature 1..20: note = Synthetic Construct
  source 1..20: mol_type = other RNA
ggttgagggcaggggcaggg

SEQ ID NO: 1263 - RNA, 20 nt, synthetic construct
  misc_feature 1..20: note = Synthetic Construct
  source 1..20: mol_type = other RNA
ggtttctccatccagaccat

SEQ ID NO: 1264 - RNA, 20 nt, synthetic construct
  misc_feature 1..20: note = Synthetic Construct
  source 1..20: mol_type = other RNA
gtaaaatctggcctccagcc

SEQ ID NO: 1265 - RNA, 20 nt, synthetic construct
  misc_feature 1..20: note = Synthetic Construct
  source 1..20: mol_type = other RNA
gtaactgggcactagtccca

SEQ ID NO: 1266 - RNA, 20 nt, synthetic construct
  misc_feature 1..20: note = Synthetic Construct
  source 1..20: mol_type = other RNA
gtaaggctggaggccagatt

SEQ ID NO: 1267 - RNA, 20 nt, synthetic construct
  misc_feature 1..20: note = Synthetic Construct
  source 1..20: mol_type = other RNA
gtcaccagtctgatctccac

SEQ ID NO: 1268 - RNA, 20 nt, synthetic construct
  misc_feature 1..20: note = Synthetic Construct
  source 1..20: mol_type = other RNA
gtccagaaagacttccttcc

SEQ ID NO: 1269 - RNA, 20 nt, synthetic construct
  misc_feature 1..20: note = Synthetic Construct
  source 1..20: mol_type = other RNA
gtggagatcagactggtgac

SEQ ID NO: 1270 - RNA, 20 nt, synthetic construct
  misc_feature 1..20: note = Synthetic Construct
  source 1..20: mol_type = other RNA
taaaatctggcctccagcct

SEQ ID NO: 1271 - RNA, 20 nt, synthetic construct
  misc_feature 1..20: note = Synthetic Construct
  source 1..20: mol_type = other RNA
tataaggctggaggccagat

SEQ ID NO: 1272 - RNA, 20 nt, synthetic construct
  misc_feature 1..20: note = Synthetic Construct
  source 1..20: mol_type = other RNA
tattttggaaaggaaggaag

SEQ ID NO: 1273 - RNA, 20 nt, synthetic construct
  misc_feature 1..20: note = Synthetic Construct
  source 1..20: mol_type = other RNA
tattttggaaaggaaggagg

SEQ ID NO: 1274 - RNA, 20 nt, synthetic construct
  misc_feature 1..20: note = Synthetic Construct
  source 1..20: mol_type = other RNA
tcaaagacagcagcagagac

SEQ ID NO: 1275 - RNA, 20 nt, synthetic construct
  misc_feature 1..20: note = Synthetic Construct
  source 1..20: mol_type = other RNA
tcacatgagtctgatctcca

SEQ ID NO: 1276 - RNA, 20 nt, synthetic construct
  misc_feature 1..20: note = Synthetic Construct
  source 1..20: mol_type = other RNA
tcaccagtctgatctccact

SEQ ID NO: 1277 - RNA, 20 nt, synthetic construct
  misc_feature 1..20: note = Synthetic Construct
  source 1..20: mol_type = other RNA
tcagactgcaaaagctgcag

SEQ ID NO: 1278 - RNA, 20 nt, synthetic construct
  misc_feature 1..20: note = Synthetic Construct
  source 1..20: mol_type = other RNA
tcagactggtgactgctgga

SEQ ID NO: 1279 - RNA, 20 nt, synthetic construct
  misc_feature 1..20: note = Synthetic Construct
  source 1..20: mol_type = other RNA
tcagtaacctgctgagggag

SEQ ID NO: 1280 - RNA, 20 nt, synthetic construct
  misc_feature 1..20: note = Synthetic Construct
  source 1..20: mol_type = other RNA
tcatgtgactgctggaaggg

SEQ ID NO: 1281 - RNA, 20 nt, synthetic construct
  misc_feature 1..20: note = Synthetic Construct
  source 1..20: mol_type = other RNA
tccagcagtcacatgagtct

SEQ ID NO: 1282 - RNA, 20 nt, synthetic construct
  misc_feature 1..20: note = Synthetic Construct
  source 1..20: mol_type = other RNA
tccagcagtcaccagtctga

SEQ ID NO: 1283 - RNA, 20 nt, synthetic construct
  misc_feature 1..20: note = Synthetic Construct
  source 1..20: mol_type = other RNA
tccagtttctccatccagac

SEQ ID NO: 1284 - RNA, 20 nt, synthetic construct
  misc_feature 1..20: note = Synthetic Construct
  source 1..20: mol_type = other RNA
tccccgagccaggaaactga

SEQ ID NO: 1285 - RNA, 20 nt, synthetic construct
  misc_feature 1..20: note = Synthetic Construct
  source 1..20: mol_type = other RNA
tccctgatcagactgcaaaa

SEQ ID NO: 1286 - RNA, 20 nt, synthetic construct
  misc_feature 1..20: note = Synthetic Construct
  source 1..20: mol_type = other RNA
tctctgctgctgtctttgag

SEQ ID NO: 1287 - RNA, 20 nt, synthetic construct
  misc_feature 1..20: note = Synthetic Construct
  source 1..20: mol_type = other RNA
tctgctgctgtctttgagca

SEQ ID NO: 1288 - RNA, 20 nt, synthetic construct
  misc_feature 1..20: note = Synthetic Construct
  source 1..20: mol_type = other RNA
tgagccctgggtgacacctg

SEQ ID NO: 1289 - RNA, 20 nt, synthetic construct
  misc_feature 1..20: note = Synthetic Construct
  source 1..20: mol_type = other RNA
tgatcagactgcaaaagctg

SEQ ID NO: 1290 - RNA, 20 nt, synthetic construct
  misc_feature 1..20: note = Synthetic Construct
  source 1..20: mol_type = other RNA
tgcaaaagctgcaggctccg

SEQ ID NO: 1291 - RNA, 20 nt, synthetic construct
  misc_feature 1..20: note = Synthetic Construct
  source 1..20: mol_type = other RNA
tgcagcttttgcagtctgat

SEQ ID NO: 1292 - RNA, 20 nt, synthetic construct
  misc_feature 1..20: note = Synthetic Construct
  source 1..20: mol_type = other RNA
tgccccctgcccctgccctc

SEQ ID NO: 1293 - RNA, 20 nt, synthetic construct
  misc_feature 1..20: note = Synthetic Construct
  source 1..20: mol_type = other RNA
tgcctggagatcccattcca

SEQ ID NO: 1294 - RNA, 20 nt, synthetic construct
  misc_feature 1..20: note = Synthetic Construct
  source 1..20: mol_type = other RNA
tgcggagcctgcagcttttg

SEQ ID NO: 1295 - RNA, 20 nt, synthetic construct
  misc_feature 1..20: note = Synthetic Construct
  source 1..20: mol_type = other RNA
tgctcaaagacagcagcaga

SEQ ID NO: 1296 - RNA, 20 nt, synthetic construct
  misc_feature 1..20: note = Synthetic Construct
  source 1..20: mol_type = other RNA
tgctgctgtctttgagcaca

SEQ ID NO: 1297 - RNA, 20 nt, synthetic construct
  misc_feature 1..20: note = Synthetic Construct
  source 1..20: mol_type = other RNA
tgctgtctttgagcacatcc

SEQ ID NO: 1298 - RNA, 20 nt, synthetic construct
  misc_feature 1..20: note = Synthetic Construct
  source 1..20: mol_type = other RNA
tggagatcagactggtgact

SEQ ID NO: 1299 - RNA, 20 nt, synthetic construct
  misc_feature 1..20: note = Synthetic Construct
  source 1..20: mol_type = other RNA
tggcagtggagatcagactg

SEQ ID NO: 1300 - RNA, 20 nt, synthetic construct
  misc_feature 1..20: note = Synthetic Construct
  source 1..20: mol_type = other RNA
tggccctgttgctgctcact

SEQ ID NO: 1301 - RNA, 20 nt, synthetic construct
  misc_feature 1..20: note = Synthetic Construct
  source 1..20: mol_type = other RNA
tgggacacatagtctctgct

SEQ ID NO: 1302 - RNA, 20 nt, synthetic construct
  misc_feature 1..20: note = Synthetic Construct
  source 1..20: mol_type = other RNA
tgggcaccagtcccagctct

SEQ ID NO: 1303 - RNA, 20 nt, synthetic construct
  misc_feature 1..20: note = Synthetic Construct
  source 1..20: mol_type = other RNA
tggtgactgctggaaggggg

SEQ ID NO: 1304 - RNA, 20 nt, synthetic construct
  misc_feature 1..20: note = Synthetic Construct
  source 1..20: mol_type = other RNA
tgtaaggctggaggccagat

SEQ ID NO: 1305 - RNA, 20 nt, synthetic construct
  misc_feature 1..20: note = Synthetic Construct
  source 1..20: mol_type = other RNA
tgtgctcaaagacagcagca

SEQ ID NO: 1306 - RNA, 20 nt, synthetic construct
  misc_feature 1..20: note = Synthetic Construct
  source 1..20: mol_type = other RNA
ttccagcagtcaccagtctg

SEQ ID NO: 1307 - RNA, 20 nt, synthetic construct
  misc_feature 1..20: note = Synthetic Construct
  source 1..20: mol_type = other RNA
ttccttcctttccaaaatat

SEQ ID NO: 1308 - RNA, 20 nt, synthetic construct
  misc_feature 1..20: note = Synthetic Construct
  source 1..20: mol_type = other RNA
ttgcggagcctgcagctttt

SEQ ID NO: 1309 - RNA, 20 nt, synthetic construct
  misc_feature 1..20: note = Synthetic Construct
  source 1..20: mol_type = other RNA
tttagggagaaagccccccg

SEQ ID NO: 1310 - RNA, 20 nt, synthetic construct
  misc_feature 1..20: note = Synthetic Construct
  source 1..20: mol_type = other RNA
tttcctggctcggggactgc

SEQ ID NO: 1311 - RNA, 20 nt, synthetic construct
  misc_feature 1..20: note = Synthetic Construct
  source 1..20: mol_type = other RNA
ttttgcagtctgatcaggga

SEQ ID NO: 1312 - RNA, 20 nt, synthetic construct
  misc_feature 1..20: note = Synthetic Construct
  source 1..20: mol_type = other RNA
ttttggaaaggaaggaagtc

SEQ ID NO: 1313 - RNA, 20 nt, synthetic construct
  misc_feature 1..20: note = Synthetic Construct
  source 1..20: mol_type = other RNA
ttttggaaaggaaggaggtc